(19) United States
(12) Patent Application Publication
(10) Pub. No.: US 2020/0040084 A1
(43) Pub. Date: Feb. 6, 2020

(54) COMPOSITIONS AND METHODS RELATED TO ENGINEERED FC-ANTIGEN BINDING DOMAIN CONSTRUCTS

(71) Applicant: Momenta Pharmaceuticals, Inc., Cambridge, MA (US)

(72) Inventors: Jonathan C. Lansing, Cambridge, MA (US); Daniel Ortiz, Cambridge, MA (US); Carlos J. Bosques, Cambridge, MA (US)

(21) Appl. No.: 16/474,572

(22) PCT Filed: Jan. 5, 2018
(86) PCT No.: PCT/US18/12689

§ 371 Date: Jun. 28, 2019

Related U.S. Application Data
(60) Provisional application No. 62/443,523, filed on Jan. 6, 2017.

(51) Int. Cl. C07K 16/28

(52) U.S. Cl. CPC C07K 16/2827; A61K 45/06

(57) ABSTRACT

The present disclosure relates to compositions and methods of engineered Fc-antigen binding domain constructs, where the Fc-antigen binding domain constructs include at least two Fc domains and at least one antigen binding domain.

### CROSS-REFERENCE TO RELATED APPLICATION

This application claims the benefit of the filing date of U.S. Provisional Application No. 62/443,523, filed on Jan. 6, 2017, the contents of which is incorporated herein by reference in its entirety.

### BACKGROUND OF THE DISCLOSURE

Many therapeutic antibodies function by recruiting elements of the innate immune system through the effector function of the Fc domains, such as antibody-dependent cytotoxicity (ADCC), antibody-dependent cellular phagocytosis (ADCP), and complement-dependent cytotoxicity (CDC). There continues to be a need for improved therapeutic proteins.

### SUMMARY OF THE DISCLOSURE

The present disclosure features compositions and methods for combining the target-specificity of an antigen binding domain with at least two Fc domains to generate new therapeutics with unique biological activity.

In some instances, the present disclosure contemplates combining an antigen binding domain of a known single Fc-domain containing therapeutic, e.g., a known therapeutic antibody, with at least two Fc domains to generate a novel therapeutic with a biological activity greater than that of the known Fc-domain containing therapeutic. To generate such constructs, the disclosure provides various methods for the assembly of constructs having at least two, e.g., multiple, Fc domains, and to control homodimerization and heterodimerization of such, to assemble molecules of discrete size from a limited number of polypeptide chains, which polypeptides are also a subject of the present disclosure. The properties of these constructs allow for the efficient generation of substantially homogenous pharmaceutical compositions. Such homogeneity in a pharmaceutical composition is desirable in order to ensure the safety, efficacy, uniformity, and reliability of the pharmaceutical composition.

In a first aspect, the disclosure features an Fc-antigen binding domain construct including enhanced effector function, where the Fc-antigen binding domain construct includes an antigen binding domain and a first Fc domain joined to a second Fc domain by a linker, where the Fc-antigen binding domain construct has enhanced effector function in an antibody-dependent cytotoxicity (ADCC) assay, an antibody-dependent cellular phagocytosis (ADCP), and/or complement-dependent cytotoxicity (CDC) assay relative to a construct having a single Fc domain and the antigen binding domain.

In a second aspect, the disclosure features a composition including a substantially homogenous population of an Fc-antigen binding domain construct including an antigen binding domain and a first Fc domain joined to a second Fc domain by a linker.

In a third aspect, the disclosure features an Fc-antigen binding domain construct including an antigen binding domain and a first Fc domain joined to a second Fc domain by a linker, where the Fc-antigen binding domain construct includes a biological activity that is not exhibited by a construct having a single Fc domain and the antigen binding domain.

In a fourth aspect, the disclosure features a composition including a substantially homogenous population of an Fc-antigen binding domain construct including a) a first polypeptide including i) a first Fc domain monomer, ii) a second Fc domain monomer, and iii) a linker joining the first Fc domain monomer and the second Fc domain monomer b) a second polypeptide including a third Fc domain monomer c) a third polypeptide including a fourth Fc domain monomer; and d) an antigen binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain.

In some embodiments of the fourth aspect, the antigen binding domain is joined to the first polypeptide and the second polypeptide or the third polypeptide, or to the second polypeptide and the third polypeptide, or the antigen binding domain is joined to the first polypeptide, the second polypeptide, and the third polypeptide.

In a fifth aspect, the disclosure features an Fc-antigen binding domain construct including enhanced effector function, where the Fc-antigen binding domain construct includes: a) a first polypeptide including i) a first Fc domain monomer, ii) a second Fc domain monomer, and iii) a linker joining the first Fc domain monomer and the second Fc domain monomer, b) a second polypeptide including a third Fc domain monomer c) a third polypeptide including a fourth Fc domain monomer, and d) an antigen binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain, and where the Fc-antigen binding domain construct has enhanced effector function in an antibody-dependent cytotoxicity (ADCC) assay, an antibody-dependent cellular phagocytosis (ADCP), and/or complement-dependent cytotoxicity (CDC) assay relative to a construct having a single Fc domain and the antigen binding domain.

In some embodiments of the fifth aspect, the single Fc domain construct is an antibody.

In a sixth aspect, the disclosure features an Fc-antigen binding domain construct including: a) a first polypeptide including i) a first Fc domain monomer, ii) a second Fc domain monomer, and iii) a linker joining the first Fc domain monomer and the second Fc domain monomer, b) a second polypeptide including a third Fc domain monomer c) a third polypeptide including a fourth Fc domain monomer and d) an antigen binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain, and where the Fc-antigen binding domain construct includes a biological activity that is not exhibited by a construct having a single Fc domain and the antigen binding domain.

In some embodiments of the sixth aspect, the biological activity is an Fc receptor mediated effector function, such as ADCC, ADCP and/or CDC activity (e.g., ADCC and ADCP activity, ADCC and CDC activity, ADCP and CDC activity, or ADCC, ADCP, and CDC activity).

In a seventh aspect, the disclosure features an Fc-antigen binding domain construct including: a) a first polypeptide including: i) a first Fc domain monomer, ii) a second Fc domain monomer, and iii) a spacer joining the first Fc domain monomer and the second Fc domain monomer; b) a second polypeptide including a third Fc domain monomer; c) a third polypeptide including a fourth Fc domain monomer; and d) an antigen binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain.

In some embodiments of the fifth, sixth, and seventh aspects of the disclosure, the antigen binding domain is joined to the first polypeptide and the second polypeptide or the third polypeptide, or to the second polypeptide and the third polypeptide, or the antigen binding domain is joined to the first polypeptide, the second polypeptide, and the third polypeptide.

In some embodiments of the first, second, third and fourth aspects of the disclosure, the antigen binding domain is a Fab.

In some embodiments of the fourth, fifth, sixth, and seventh aspects of the disclosure, the antigen binding domain is part of the amino acid sequence of the first, second, or third polypeptide, and, in some embodiments, the antigen binding domain is a scFv.

In some embodiments of the fourth, fifth, sixth, and seventh aspects of the disclosure, the antigen binding domain includes a V_{H} domain and a C_{H}1 domain, and where the V_{H} and C_{H}1 domains are part of the amino acid sequence of the first, second, or third polypeptide. In some embodiments, the antigen binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a V_{H} domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the fourth, fifth, sixth, and seventh aspects of the disclosure, the antigen binding domain includes a set of CDR-H1, CDR-H2, CDR-H3, CDR-L1, CDR-L2, and CDR-L3 sequences set forth in Table 1, the antigen binding domain includes CDR-H1, CDR-H2. CDR-H3, CDR-L1, CDR-L2, and CDR-L3 sequences from a set of a V_{H} and a V_{L} sequence of an antibody set forth in Table 2, the antigen binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the antigen binding domain includes a set of a V_{H} and a V_{L} sequences of an antibody set forth in Table 2.

In some embodiments of the fourth, fifth, sixth, and seventh aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the fourth, fifth, sixth, and seventh aspects of the disclosure, the first Fc domain monomer and the third Fc domain monomer include complementary dimerization selectivity modules that promote dimerization between the first Fc domain monomer and the third Fc domain monomer.

In some embodiments of the fourth, fifth, sixth, and seventh aspects of the disclosure, the second Fc domain monomer and the fourth Fc domain monomer include complementary dimerization selectivity modules that promote dimerization between the second Fc domain monomer and the fourth Fc domain monomer.

In some embodiments of the fourth, fifth, sixth, and seventh aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the fourth, fifth, sixth, and seventh aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the fourth, fifth, sixth, and seventh aspects of the disclosure, the second polypeptide and the third polypeptide have the same amino acid sequence.

In some embodiments of the fourth, fifth, sixth, and seventh aspects of the disclosure, one or more linker in the Fc-antigen binding domain construct is a bond.

In some embodiments of the fourth, fifth, sixth, and seventh aspects of the disclosure, one or more linker in the Fc-antigen binding domain construct is a spacer. In some embodiments, the spacer includes a polypeptide having the sequence GGGGGGGGGGGGGGGGGGGG, GGGGS, GGSG, SGGG, GSGS, GSGSGS, GSGSGSGS, GSGSGSGSGS, GSGSGSGSGSGS, GGSGGS, GGSGGSGGS, GGSGGSGGSGGS, GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG, or GGGGGGGGGGGGGGGG. In some embodiments, the spacer is a glycine spacer, for example, one consisting of 4 to 30, 8 to 30, or 12 to 30 glycine residues, such as a spacer consisting of 20 glycine residues.

In some embodiments of the fourth, fifth, sixth, and seventh aspects of the disclosure, the antigen binding domain is joined to the Fc domain monomer by a linker. In some embodiments, the linker is a spacer.

In some embodiments of the fourth, fifth, sixth, and seventh aspects of the disclosure, at least one of the Fc domains includes at least one amino acid modification at position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R. I253S, I253T, I253V, I253W, and I253Y. In some embodiments, each amino acid modification at position I253 is I253A.

In some embodiments of the fourth, fifth, sixth, and seventh aspects of the disclosure, at least one of the Fc domains includes at least one amino acid modification at position R292. In some embodiments, each amino acid modification at position R292 is independently selected from R292D, R292E, R292L, R292P, R292Q, R292R, R292T, and R292Y. In some embodiments, each amino acid modification at position R292 is R292P.

In some embodiments of the fourth, fifth, sixth, and seventh aspects of the disclosure, one or more of the Fc domain monomers includes an IgG hinge domain, an IgG C_{H2} antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the fourth, fifth, sixth, and seventh aspects of the disclosure, the N-terminal Asp in each of the fourth, fifth, sixth, and seventh polypeptides is mutated to Gln.

In some embodiments of the fourth, fifth, sixth, and seventh aspects of the disclosure, one or more of the fourth, fifth, sixth, and seventh polypeptides lack a C-terminal lysine. In some embodiments, each of the fourth, fifth, sixth, and seventh polypeptides lacks a C-terminal lysine.

In some embodiments of the fourth, fifth, sixth, and seventh aspects of the disclosure, the Fc-antigen binding domain construct further includes an albumin-binding peptide joined to the N-terminus or C-terminus of one or more of the polypeptides by a linker.

In an eighth aspect, the disclosure features a cell culture medium including a population of Fc-antigen binding domain constructs, where at least 50% of the Fc-antigen binding domain constructs, on a molar basis, are structurally identical, and where the Fc-antigen binding domain constructs are present in the culture medium at a concentration of at least 0.1 mg/L, 10 mg/L, 25 mg/L, 50 mg/L, 75 mg/L, or 100 mg/L.

In some embodiments of the eighth aspect of the disclosure, at least 75%%, at least 85%, or at least 95% of the Fc-antigen binding domain constructs, on a molar basis, are structurally identical.

In a ninth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer, and d) an antigen binding domain joined to the first polypeptide, second polypeptide, or third polypeptide; where the first Fc domain monomer and the third Fc domain monomer combine to form a first Fc domain and the second Fc domain monomer and the fourth Fc domain monomer combine to form a second Fc domain.

In some embodiments of the ninth aspect of the disclosure at least 75%, at least 85%, or at least 95% of the Fc-antigen binding domain constructs, on a molar basis, include the first Fc domain, the second Fc domain, and the antigen binding domain.

In a tenth aspect, the disclosure features a method of manufacturing an Fc-antigen binding domain construct, the method including: a) culturing a host cell expressing: (1) a first polypeptide including i) a first Fc domain monomer, ii) a second Fc domain monomer, and iii) a linker joining the first Fc domain monomer and the second Fc domain monomer (2) a second polypeptide including a third Fc domain monomer (3) a third polypeptide including a fourth Fc domain monomer; and (4) an antigen binding domain; where the first Fc domain monomer and the third Fc domain monomer combine to form a first Fc domain and the second Fc domain monomer and the fourth Fc domain monomer combine to form a second Fc domain; where the antigen binding domain is joined to the first polypeptide, second polypeptide, or third polypeptide, thereby forming an Fc-antigen binding domain construct; and where at least 50% of the Fc-antigen binding domain constructs in a cell culture supernatant, on a molar basis, are structurally identical, and b) purifying the Fc-antigen binding domain construct from the cell culture supernatant.

In some embodiments of the ninth and tenth aspects of the disclosure, the antigen binding domain is joined to the first polypeptide and the second polypeptide or the third polypeptide, or to the second polypeptide and the third polypeptide, or the antigen binding domain is joined to the first polypeptide, the second polypeptide, and the third polypeptide.

In some embodiments of the ninth and tenth aspects of the disclosure, the antigen binding domain is a Fab.

In some embodiments of the ninth and tenth aspects of the disclosure, the antigen binding domain is part of the amino acid sequence of the first, second, or third polypeptide, and, in some embodiments, the antigen binding domain is a scFv.

In some embodiments of the ninth and tenth aspects of the disclosure, the antigen binding domain includes a V_{H} domain and a C_{H}1 domain, and where the V_{H} and C_{H}1 domains are part of the amino acid sequence of the first, second, or third polypeptide. In some embodiments, the antigen binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a V_{H} domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the ninth and tenth aspects of the disclosure, the antigen binding domain includes a set of CDR-H1, CDR-H2, CDR-H3, CDR-L1, CDR-L2, and CDR-L3 sequences set forth in Table 1, the antigen binding domain includes CDR-H1, CDR-H2, CDR-H3, CDR-L1, CDR-L2, and CDR-L3 sequences from a set of a V_{H} and a V_{L} sequences of an antibody set forth in Table 2, the antigen binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the antigen binding domain includes a set of a V_{H} and a V_{L} sequence of an antibody set forth in Table 2.

In some embodiments of the ninth and tenth aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the ninth and tenth aspects of the disclosure, the first Fc domain monomer and the third Fc domain monomer include complementary dimerization selectivity modules that promote dimerization between the first Fc domain monomer and the third Fc domain monomer.

In some embodiments of the ninth and tenth aspects of the disclosure, the second Fc domain monomer and the fourth Fc domain monomer include complementary dimerization selectivity modules that promote dimerization between the second Fc domain monomer and the fourth Fc domain monomer.

In some embodiments of the ninth and tenth aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the ninth and tenth aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the ninth and tenth aspects of the disclosure, the second polypeptide and the third polypeptide have the same amino acid sequence.

In some embodiments of the ninth and tenth aspects of the disclosure, one or more linker in the Fc-antigen binding domain construct is a bond.

In some embodiments of the ninth and tenth aspects of the disclosure, one or more linker in the Fc-antigen binding domain construct is a spacer. In some embodiments, the spacer includes a polypeptide having the sequence GGGGGGGGGGGGGGGGGGGG, GGGGS, GGSG, SGGG, GSGS, GSGSGS, GSGSGSGS, GSGSGSGSGS, GSGSGSGSGSGS, GGSGGS, GGSGGSGGS, GGSGGSGGSGGS, GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG, or GGGGGGGGGGGGGGGG. In some embodiments, the spacer is a glycine spacer, for example, one consisting of 4 to 30, 8 to 30, or 12 to 30 glycine residues, such as a spacer consisting of 20 glycine residues.

In some embodiments of the ninth and tenth aspects of the disclosure, the antigen binding domain is joined to the Fc domain monomer by a linker. In some embodiments, the linker is a spacer.

In some embodiments of the ninth and tenth aspects of the disclosure, at least one of the Fc domains includes at least one amino acid modification at position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification at position I253 is I253A.

In some embodiments of the ninth and tenth aspects of the disclosure, at least one of the Fc domains includes at least one amino acid modification at position R292. In some embodiments, each amino acid modification at position R292 is independently selected from R292D, R292E, R292L, R292P, R292Q, R292R, R292T, and R292Y. In some embodiments, each amino acid modification at position R292 is R292P.

In some embodiments of the ninth and tenth aspects of the disclosure, one or more of the Fc domain monomers includes an IgG hinge domain, an IgG C_{H}2 antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the ninth and tenth aspects of the disclosure, the N-terminal Asp in each of the first, second, third, and fourth polypeptides is mutated to Gln.

In some embodiments of the ninth and tenth aspects of the disclosure, one or more of the first, second, third, and fourth polypeptides lack a C-terminal lysine. In some embodiments, each of the first, second, third, and fourth polypeptides lacks a C-terminal lysine.

In some embodiments of the ninth and tenth aspects of the disclosure, the Fc-antigen binding domain construct further includes an albumin-binding peptide joined to the N-terminus or C-terminus of one or more of the polypeptides by a linker.

In an eleventh aspect, the disclosure features a composition including a substantially homogenous population of an Fc-antigen binding domain construct including: a) a first polypeptide including i) a first Fc domain monomer, ii) a second Fc domain monomer, and iii) a linker joining the first Fc domain monomer and the second Fc domain monomer, b) a second polypeptide including a third Fc domain monomer; c) a third polypeptide including a fourth Fc domain monomer, d) a first antigen binding domain joined to the first polypeptide; and e) a second antigen binding domain joined to the second polypeptide and/or third polypeptide; where the first Fc domain monomer and the third Fc domain monomer combine to form a first Fc domain and the second Fc domain monomer and the fourth Fc domain monomer combine to form a second Fc domain, and where the first and the second antigen binding domains bind different antigens.

In some embodiments of the eleventh aspect of the disclosure, the first Fc domain monomer and the third Fc domain monomer include complementary dimerization selectivity modules that promote dimerization between the first Fc domain monomer and the third Fc domain monomer, where the second Fc domain monomer and the fourth Fc domain monomer include complementary dimerization selectivity modules that promote dimerization between the second Fc domain monomer and the fourth Fc domain monomer, and where the second polypeptide and the third polypeptide have different amino acid sequences.

In some embodiments of the eleventh aspect of the disclosure, the first antigen binding domain is joined to the first polypeptide and the second antigen binding domain is joined to the second polypeptide and the third polypeptide.

In some embodiments of the eleventh aspect of the disclosure each of the second Fc domain monomer and the fourth Fc domain monomer includes E357K and K370D, and each of the first Fc domain monomer and the third Fc domain monomer includes K370D and E357K

In a twelfth aspect, the disclosure features a composition including a substantially homogenous population of an Fc-antigen binding domain construct including: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; d) a first antigen binding domain joined to the first polypeptide; e) a second antigen binding domain joined to the second polypeptide; and f) a third antigen binding domain joined to the third polypeptide; where the first Fc domain monomer and the third Fc domain monomer combine to form a first Fc domain and the second Fc domain monomer and the fourth Fc domain monomer combine to form a second Fc domain, and where the first, the second, and the third antigen binding domains bind different antigens.

In some embodiments of the twelfth aspect of the disclosure, the first Fc domain monomer and the third Fc domain monomer include complementary dimerization selectivity modules that promote dimerization between the first Fc domain monomer and the third Fc domain monomer, where the second Fc domain monomer and the fourth Fc domain monomer include complementary dimerization selectivity modules that promote dimerization between the second Fc domain monomer and the fourth Fc domain monomer, and where the second polypeptide and the third polypeptide have different amino acid sequences.

In some embodiments of the twelfth aspect of the disclosure, each of the second Fc domain monomer and the fourth Fc domain monomer includes D399K and K409D, and each of the first Fc domain monomer and the third Fc domain monomer includes E357K and K370D.

In some embodiments of the eleventh and twelfth aspects of the disclosure, the first or second antigen binding is a Fab. In some embodiments of the eleventh and twelfth aspects of the disclosure, the first and second antigen binding domain is a Fab. In some embodiments of the ninth aspect of the disclosure, the first, second, and third antigen binding domain is a Fab.

In some embodiments of the eleventh and twelfth aspects of the disclosure, the first or second antigen binding is a scFv. In some embodiments of the eleventh and twelfth aspects of the disclosure, the first and second antigen binding domain is a scFv. In some embodiments of the ninth aspect of the disclosure, the first, second, and third antigen binding domain is a scFv.

In some embodiments of the eleventh aspect of the disclosure, the first or second antigen binding domain includes a V_{H} domain and a C_{H}1 domain, and where the V_{H} and C_{H}1 domains are part of the amino acid sequence of the first, second, or third polypeptide. In some embodiments, the antigen binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a V_{H} domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the twelfth aspect of the disclosure, the first, second, or third antigen binding domain includes a V_{H} domain and a C_{H}1 domain, and where the V_{H} and C_{H}1 domains are part of the amino acid sequence of the first, second, or third polypeptide. In some embodiments, the antigen binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a V_{H} domain including a sequence of an antibody set forth in Table 2, the V_{H} domain includes CDR-H1, CDR-H2, and CDR-H3 of a V_{H} sequence of an antibody set forth in Table 2, and the V sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the eleventh aspect of the disclosure, the first or second antigen binding domain includes a set of CDR-H1, CDR-H2, CDR-H3, CDR-L1, CDR-L2, and CDR-L3 sequences set forth in Table 1, the antigen binding domain includes CDR-H1, CDR-H2. CDR-H3, CDR-L1, CDR-L2, and CDR-L3 sequences from a set of a V_{H} and a V_{L} sequence of an antibody set forth in Table 2, the antigen binding domain includes a V_{H} domain including CDR-H1. CDR-H2, and CDR-H3 of a V_{H} sequence of an antibody set forth in Table 2, and a V_{L} domain including CDR-L1, CDR-L2, and CDR-L3 of a V_{L} sequences of an antibody set forth in Table 2, where the V_{H} and the V_{L} domain sequences, excluding the CDR-H1, CDR-H2. CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the antigen binding domain includes a set of a V_{H} and a V_{L} sequence of an antibody set forth in Table 2.

In some embodiments of the twelfth aspect of the disclosure, the first, second, or third antigen binding domain includes a set of CDR-H1, CDR-H2, CDR-H3, CDR-L1, CDR-L2, and CDR-L3 sequences set forth in Table 1, the antigen binding domain includes CDR-H1, CDR-H2. CDR-H3, CDR-L1, CDR-L2, and CDR-L3 sequences from a set of a V_{H} and a V_{L} sequence of an antibody set forth in Table 2, the antigen binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the antigen binding domain includes a set of a V_{H} and a V_{L} sequences of an antibody set forth in Table 2.

In some embodiments of the eleventh and twelfth aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the eleventh and twelfth aspects of the disclosure, the first Fc domain monomer and the third Fc domain monomer include complementary dimerization selectivity modules that promote dimerization between the first Fc domain monomer and the third Fc domain monomer.

In some embodiments of the eleventh and twelfth aspects of the disclosure, the second Fc domain monomer and the fourth Fc domain monomer include complementary dimerization selectivity modules that promote dimerization between the second Fc domain monomer and the fourth Fc domain monomer.

In some embodiments of the eleventh and twelfth aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the eleventh and twelfth aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the eleventh and twelfth aspects of the disclosure, one or more linker in the Fc-antigen binding domain construct is a bond.

In some embodiments of the eleventh and twelfth aspects of the disclosure, one or more linker in the Fc-antigen binding domain construct is a spacer. In some embodiments, the spacer includes a polypeptide having the sequence GGGGGGGGGGGGGGGGGGGG, GGGGS, GGSG, SGGG, GSGS, GSGSGS, GSGSGSGS, GSGSGSGSGS, GSGSGSGSGSGS, GGSGGS, GGSGGSGGS, GGSGGSGGSGGS, GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG, or GGGGGGGGGGGGGGGG. In some embodiments, the spacer is a glycine spacer, for example, one consisting of 4 to 30, 8 to 30, or 12 to 30 glycine residues, such as a spacer consisting of 20 glycine residues.

In some embodiments of the eleventh and twelfth aspects of the disclosure, one or more of the antigen binding domains is joined to the Fc domain monomer by a linker. In some embodiments, the linker is a spacer.

In some embodiments of the eleventh and twelfth aspects of the disclosure, at least one of the Fc domains includes at least one amino acid modification at position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification at position I253 is I253A.

In some embodiments of the eleventh and twelfth aspects of the disclosure, at least one of the Fc domains includes at least one amino acid modification at position R292. In some embodiments, each amino acid modification at position R292 is independently selected from R292D, R292E, R292L, R292P, R292Q, R292R, R292T, and R292Y. In some embodiments, each amino acid modification at position R292 is R292P.

In some embodiments of the eleventh and twelfth aspects of the disclosure, one or more of the Fc domain monomers includes an IgG hinge domain, an IgG C_{H}2 antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the eleventh and twelfth aspects of the disclosure, the N-terminal Asp in each of the first, second, third, and fourth polypeptides is mutated to Gln.

In some embodiments of the eleventh and twelfth aspects of the disclosure, one or more of the first, second, third, and fourth polypeptides lack a C-terminal lysine. In some embodiments, each of the first, second, third, and fourth polypeptides lacks a C-terminal lysine.

In some embodiments of the eleventh and twelfth aspects of the disclosure, the Fc-antigen binding domain construct further includes an albumin-binding peptide joined to the N-terminus or C-terminus of one or more of the polypeptides by a linker.

In a thirteenth aspect, the disclosure features a composition including a substantially homogenous population of an Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including i) a third Fc domain monomer, ii) a fourth Fc domain monomer, and iv) a second linker joining the third Fc domain monomer and the fourth Fc domain monomer; and c) a third polypeptide including a fifth Fc domain monomer; d) a fourth polypeptide including an sixth Fc domain monomer; and d) an antigen binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain.

In some embodiments of the thirteenth aspect of the disclosure, each of the first and third Fc domain monomers includes a complementary dimerization selectivity module that promote dimerization between the first Fc domain monomer and the third Fc domain monomer, each of the second and fifth Fc domain monomers includes a complementary dimerization selectivity module that promote dimerization between the second Fc domain monomer and the fifth Fc domain monomer, and each of the fourth and sixth Fc domain monomers includes a complementary dimerization selectivity module that promote dimerization between the fourth Fc domain monomer and the sixth Fc domain monomer.

In an fourteenth aspect, the disclosure features a composition including a substantially homogenous population of an Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including i) a third Fc domain monomer, ii) a fourth Fc domain monomer, and iv) a second linker joining the third Fc domain monomer and the fourth Fc domain monomer; and c) a third polypeptide including a fifth Fc domain monomer, d) a fourth polypeptide including an sixth Fc domain monomer, and e) an antigen binding domain joined to the first polypeptide, second polypeptide, third polypeptide, or fourth polypeptide; where the second Fc domain monomer and the fourth Fc domain monomer combine to form a first Fc domain and the first Fc domain monomer and the fifth Fc domain monomer combine to form a second Fc domain, the third Fc domain monomer and the sixth Fc domain monomer combine to form a third Fc domain.

In some embodiments of the fourteenth aspect of the disclosure, each of the second and fourth Fc domain monomers includes a complementary dimerization selectivity module that promote dimerization between the second Fc domain monomer and the fourth Fc domain monomer, each of the first and fifth Fc domain monomers includes a complementary dimerization selectivity module that promote dimerization between the first Fc domain monomer and the fifth Fc domain monomer, and each of the third and sixth Fc domain monomers includes a complementary dimerization selectivity module that promote dimerization between the third Fc domain monomer and the sixth Fc domain monomer.

In a fifteenth aspect, the disclosure features a composition including a substantially homogenous population of an Fc-antigen binding domain construct including: a) a first polypeptide including i) a first Fc domain monomer, ii) a second Fc domain monomer, iii) a third Fc domain monomer, iv) a first linker joining the first Fc domain monomer and the second Fc domain monomer, and v) a second linker joining the second Fc domain monomer and the third Fc domain monomer; b) a second polypeptide including i) a fourth Fc domain monomer, ii) a fifth Fc domain monomer, iii) a sixth Fc domain monomer, iv) a third linker joining the fourth Fc domain monomer and the fifth Fc domain monomer and v) a fourth linker joining the fifth Fc domain monomer and the sixth Fc domain monomer, c) a third polypeptide including a seventh Fc domain monomer d) a fourth polypeptide including an eighth Fc domain monomer, e) a fifth polypeptide including a ninth Fc domain monomer, f) a sixth polypeptide including a tenth Fc domain monomer; and g) an antigen binding domain joined to the first polypeptide, second polypeptide, third polypeptide, fourth polypeptide, fifth polypeptide, or sixth polypeptide; where the second Fc domain monomer and the fifth Fc domain monomer combine to form a first Fc domain and the first Fc domain monomer and the seventh Fc domain monomer combine to form a second Fc domain, the fourth Fc domain monomer and the eighth Fc domain monomer combine to form a third Fc domain, the third Fc domain monomer and the ninth Fc domain monomer combine to form a fourth Fc domain, and the sixth Fc domain monomer and the tenth Fc domain monomer combine to form a fifth Fc domain.

In some embodiments of the fifteenth aspect of the disclosure, each of the second and fifth Fc domain monomers includes a complementary dimerization selectivity module that promote dimerization between the second Fc domain monomer and the fifth Fc domain monomer, each of the first and seventh Fc domain monomers includes a complementary dimerization selectivity module that promote dimerization between the first Fc domain monomer and the seventh Fc domain monomer, each of the fourth and eighth Fc domain monomers includes a complementary dimerization selectivity module that promote dimerization between the fourth Fc domain monomer and the eighth Fc domain monomer, each of the third and ninth Fc domain monomers includes a complementary dimerization selectivity module that promote dimerization between the third Fc domain monomer and the ninth Fc domain monomer, and each of the sixth and tenth Fc domain monomers includes a complementary dimerization selectivity module that promote dimerization between the sixth Fc domain monomer and the tenth Fc domain monomer.

In some embodiments of the thirteenth, fourteenth, and fifteenth aspects of the disclosure, the antigen binding domain is a Fab.

In some embodiments of the thirteenth, fourteenth, and fifteenth aspects of the disclosure, the antigen binding domain is part of the amino acid sequence of one or more of the polypeptides, and, in some embodiments, the antigen binding domain is a scFv.

In some embodiments of the thirteenth, fourteenth, and fifteenth aspects of the disclosure, the antigen binding domain includes a V_{H} domain and a C_{H}1 domain, and where the V_{H} and C_{H}1 domains are part of the amino acid sequence of the first, second, or third polypeptide. In some embodiments, the antigen binding domain further includes a V_{L} domain, where, in some embodiments the Fc-antigen binding domain construct includes a fourth polypeptide including the V_{L} domain. In some embodiments, the V_{H} domain includes a set of CDR-H1, CDR-H2 and CDR-H3 sequences set forth in Table 1, the V_{H} domain includes CDR-H1, CDR-H2, and CDR-H3 of a V_{H} domain including a sequence of an antibody set forth in Table 2, the V_{H} domain includes CDR-H1, CDR-H2, and CDR-H3 of a V_{H} sequence of an antibody set forth in Table 2, and the V_{H} sequence, excluding the CDR-H1, CDR-H2, and CDR-H3 sequence, is at least 95% identical, at least 97% identical, at least 99% identical, or at least 99.5% identical to the V_{H} sequence of an antibody set forth in Table 2, or the V_{H} domain includes a V_{H} sequence of an antibody set forth in Table 2.

In some embodiments of the thirteenth, fourteenth, and fifteenth aspects of the disclosure, the antigen binding domain includes a set of CDR-H1, CDR-H2, CDR-H3. CDR-L1, CDR-L2, and CDR-L3 sequences set forth in Table 1, the antigen binding domain includes CDR-H1, CDR-H2. CDR-H3, CDR-L1, CDR-L2, and CDR-L3 sequences from a set of a V_{H} and a V_{L} sequences of an antibody set forth in Table 2, the antigen binding domain includes a V_{H} domain including CDR-H1, CDR-H2, and CDR-H3 of a V_{H} sequence of an antibody set forth in Table 2, and a V_{L} domain including CDR-L1, CDR-L2, and CDR-L3 of a V_{L} sequence of an antibody set forth in Table 2, where the V_{H} and the V_{L} domain sequences, excluding the CDR-H1, CDR-H2, CDR-H3, CDR-L1, CDR-L2, and CDR-L3 sequences, are at least 95% identical, at least 97% identical, at least 99% identical, or at least 99.5% identical to the V_{H} and V_{L} sequences of an antibody set forth in Table 2, or the antigen binding domain includes a set of a V_{H} and a V_{L} sequences of an antibody set forth in Table 2.

In some embodiments of the thirteenth, fourteenth, and fifteenth aspects of the disclosure, the Fc-antigen binding domain construct, further includes an IgG C_{L} antibody constant domain and an IgG C_{H}1 antibody constant domain, where the IgG C_{H}1 antibody constant domain is attached to the N-terminus of the first polypeptide or the second polypeptide by way of a linker.

In some embodiments of the thirteenth, fourteenth, and fifteenth aspects of the disclosure, the dimerization selectivity modules include an engineered cavity into the C_{H}3 domain of one of the Fc domain monomers and an engineered protuberance into the C_{H}3 domain of the other of the Fc domain monomers, where the engineered cavity and the engineered protuberance are positioned to form a protuberance-into-cavity pair of Fc domain monomers. In some embodiments, the engineered protuberance includes at least one modification selected from S354C, T366W, T366Y, T394W, T394F, and F405W, and the engineered cavity includes at least one modification selected from Y349C, T366S, L368A, Y407V, Y407T, Y407A, F405A, and T394S. In some embodiments, one of the Fc domain monomers includes Y407V and Y349C and the other of the Fc domain monomers includes T366W and S354C.

In some embodiments of the thirteenth, fourteenth, and fifteenth aspects of the disclosure, the dimerization selectivity modules include a negatively-charged amino acid into the C_{H}3 domain of one of the domain monomers and a positively-charged amino acid into the C_{H}3 domain of the other of the Fc domain monomers, where the negatively-charged amino acid and the positively-charged amino acid are positioned to promote formation of an Fc domain. In some embodiments, each of the first Fc domain monomer and third Fc domain monomer includes D399K and either K409D or K409E, each of the first Fc domain monomer and third Fc domain monomer includes K392D and D399K, each of the first Fc domain monomer and third Fc domain monomer includes E357K and K370E, each of the first Fc domain monomer and third Fc domain monomer includes D356K and K439D, each of the first Fc domain monomer and third Fc domain monomer includes K392E and D399K, each of the first Fc domain monomer and third Fc domain monomer includes E357K and K370D, each of the first Fc domain monomer and third Fc domain monomer includes D356K and K439E, each of the second Fc domain monomer and fourth Fc domain monomer includes S354C and T366W and the third and fourth polypeptides each include Y349C, T366S, L368A, and Y407V, each of the third and fourth polypeptides includes S354C and T366W and the second Fc domain monomer and fourth Fc domain monomer each include Y349C, T366S, L368A, and Y407V, each of the second Fc domain monomer and fourth Fc domain monomer includes E357K or E357R and the third and fourth polypeptides each include K370D or K370E, each of the second Fc domain monomer and fourth Fc domain monomer include K370D or K370E and the third and fourth polypeptides each include E357K or 357R, each of the second Fc domain monomer and fourth Fc domain monomer include K409D or K409E and the third and fourth polypeptides each include D399K or D399R, or each of the second Fc domain monomer and fourth Fc domain monomer include D399K or D399R and the third and fourth polypeptides each include K409D or K409E.

In some embodiments of the thirteenth, fourteenth, and fifteenth aspects of the disclosure, one or more linker in the Fc-antigen binding domain construct is a bond.

In some embodiments of the thirteenth, fourteenth, and fifteenth aspects of the disclosure, one or more linker in the Fc-antigen binding domain construct is a spacer. In some embodiments, the spacer includes a polypeptide having the sequence GGGGGGGGGGGGGGGGGGGG, GGGGS, GGSG, SGGG, GSGS, GSGSGS, GSGSGSGS, GSGSGSGSGS, GSGSGSGSGSGS, GGSGGS, GGSGGSGGS, GGSGGSGGSGGS, GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG, or GGGGGGGGGGGGGGGG. In some embodiments, the spacer is a glycine spacer, for example, one consisting of 4 to 30, 8 to 30, or 12 to 30 glycine residues, such as a spacer consisting of 20 glycine residues.

In some embodiments of the thirteenth, fourteenth, and fifteenth aspects of the disclosure, the antigen binding domain is joined to the Fc domain monomer by a linker. In some embodiments, the linker is a spacer.

In some embodiments of the thirteenth, fourteenth, and fifteenth aspects of the disclosure, at least one of the Fc domains includes at least one amino acid modification at position I253. In some embodiments, the each amino acid modification at position I253 is independently selected from I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification at position I253 is I253A.

In some embodiments of the thirteenth, fourteenth, and fifteenth aspects of the disclosure, at least one of the Fc domains includes at least one amino acid modification at position R292. In some embodiments, each amino acid modification at position R292 is independently selected from R292D, R292E, R292L. R292P, R292Q, R292R, R292T. and R292Y. In some embodiments, each amino acid modification at position R292 is R292P.

In some embodiments of the thirteenth, fourteenth, and fifteenth aspects of the disclosure, one or more of the Fc domain monomers includes an IgG hinge domain, an IgG C_{H}2 antibody constant domain, and an IgG C_{H}3 antibody constant domain. In some embodiments, each of the Fc domain monomers includes an IgG hinge domain, an IgG C_{H}2 antibody constant domain, and an IgG C_{H}3 antibody constant domain. In some embodiments, the IgG is of a subtype selected from the group consisting of IgG1, IgG2a, IgG2b, IgG3, and IgG4.

In some embodiments of the thirteenth, fourteenth, and fifteenth aspects of the disclosure, the N-terminal Asp in each of the polypeptides is mutated to Gln.

In some embodiments of the thirteenth, fourteenth, and fifteenth aspects of the disclosure, one or more of the polypeptides lack a C-terminal lysine. In some embodiments, each of the polypeptides lacks a C-terminal lysine.

In some embodiments of the thirteenth, fourteenth, and fifteenth aspects of the disclosure, the Fc-antigen binding domain construct further includes an albumin-binding peptide joined to the N-terminus or C-terminus of one or more of the polypeptides by a linker.

In a sixteenth aspect, the disclosure features an Fc-antigen binding domain construct including: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; and d) a first antigen binding domain joined to the first polypeptide; and e) a second antigen binding domain joined to the second polypeptide and/or third polypeptide; where the first Fc domain monomer and the third Fc domain monomer combine to form a first Fc domain and the second Fc domain monomer and the fourth Fc domain monomer combine to form a second Fc domain, where the first and the second antigen binding domains bind different antigens, and where the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain.

In a seventeenth aspect, the disclosure features an Fc-antigen binding domain construct including:

a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; d) a first antigen binding domain joined to the first polypeptide; and e) a second antigen binding domain joined to the second polypeptide and/or third polypeptide; where the first Fc domain monomer and the third Fc domain monomer combine to form a first Fc domain and the second Fc domain monomer and the fourth Fc domain monomer combine to form a second Fc domain, and where the first and the second antigen binding domains bind different antigens, and where the Fc-antigen binding domain construct includes a biological activity that is not exhibited by a construct having a single Fc domain and the antigen binding domain.

In an eighteenth aspect, the disclosure features an Fc-antigen binding domain construct including:

a) a first polypeptide including i) a first Fc domain monomer, ii) a second Fc domain monomer, and iii) a spacer joining the first Fc domain monomer and the second Fc domain monomer, b) a second polypeptide including a third Fc domain monomer; c) a third polypeptide including a fourth Fc domain monomer; and d) a first antigen binding domain joined to the first polypeptide; and e) a second antigen binding domain joined to the second polypeptide and/or third polypeptide, where the first Fc domain monomer and the third Fc domain monomer combine to form a first Fc domain and the second Fc domain monomer and the fourth Fc domain monomer combine to form a second Fc domain, and where the first and the second antigen binding domains bind different antigens.

In a nineteenth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; and d) a first antigen binding domain joined to the first polypeptide; and e) a second antigen binding domain joined to the second polypeptide and/or third polypeptide; where the first Fc domain monomer and the third Fc domain monomer combine to form a first Fc domain and the second Fc domain monomer and the fourth Fc domain monomer combine to form a second Fc domain, and where the first and the second antigen binding domains bind different antigens.

In a twentieth aspect, the disclosure features a method of manufacturing an Fc-antigen binding domain construct, the method including: a) culturing a host cell expressing: (1) a first polypeptide including i) a first Fc domain monomer, ii) a second Fc domain monomer, and iii) a linker joining the first Fc domain monomer and the second Fc domain monomer, (2) a second polypeptide including a third Fc domain monomer (3) a third polypeptide including a fourth Fc domain monomer, (4) a first antigen binding domain joined to the first polypeptide; and (5) a second antigen binding domain joined to the second polypeptide and/or third polypeptide; where the first Fc domain monomer and the third Fc domain monomer combine to form a first Fc domain and the second Fc domain monomer and the fourth Fc domain monomer combine to form a second Fc domain; where the antigen binding domain is joined to the first polypeptide, second polypeptide, or third polypeptide, thereby forming an Fc-antigen binding domain construct, where the first and the second antigen binding domains bind different antigens, and where at least 50% of the Fc-antigen binding domain constructs in a cell culture supernatant, on a molar basis, are structurally identical, and b) purifying the Fc-antigen binding domain construct from the cell culture supernatant.

In some embodiments of the sixteenth, seventeenth, eighteenth, nineteenth, and twentieth aspects of the disclosure, the first Fc domain monomer and the third Fc domain monomer include complementary dimerization selectivity modules that promote dimerization between the first Fc domain monomer and the third Fc domain monomer, where the second Fc domain monomer and the fourth Fc domain monomer include complementary dimerization selectivity modules that promote dimerization between the second Fc domain monomer and the fourth Fc domain monomer, and where the second polypeptide and the third polypeptide have different amino acid sequences.

In a twenty first aspect, the disclosure features an Fc-antigen binding domain construct including:

a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; d) a first antigen binding domain joined to the first polypeptide; e) a second antigen binding domain joined to the second polypeptide; and f) a third antigen binding domain joined to the third polypeptide; where the first Fc domain monomer and the third Fc domain monomer combine to form a first Fc domain and the second Fc domain monomer and the fourth Fc domain monomer combine to form a second Fc domain, and where the first, the second, and the third antigen binding domains bind different antigens, and where the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain.

In a twenty second aspect, the disclosure features an Fc-antigen binding domain construct including: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; d) a first antigen binding domain joined to the first polypeptide; e) a second antigen binding domain joined to the second polypeptide; and f) a third antigen binding domain joined to the third polypeptide; where the first Fc domain monomer and the third Fc domain monomer combine to form a first Fc domain and the second Fc domain monomer and the fourth Fc domain monomer combine to form a second Fc domain, and where the first, the second, and the third antigen binding domains bind different antigens, and where the Fc-antigen binding domain construct includes a biological activity that is not exhibited by a construct having a single Fc domain and the antigen binding domain.

In a twenty third aspect, the disclosure features an Fc-antigen binding domain construct including: a) a first polypeptide including i) a first Fc domain monomer, ii) a second Fc domain monomer, and iii) a spacer joining the first Fc domain monomer and the second Fc domain monomer; b) a second polypeptide including a third Fc domain monomer; c) a third polypeptide including a fourth Fc domain monomer; d) a first antigen binding domain joined to the first polypeptide; e) a second antigen binding domain joined to the second polypeptide; and f) a third antigen binding domain joined to the third polypeptide; where the first Fc domain monomer and the third Fc domain monomer combine to form a first Fc domain and the second Fc domain monomer and the fourth Fc domain monomer combine to form a second Fc domain, and where the first, the second, and the third antigen binding domains bind different antigens.

In a twenty fourth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, and iii) a linker joining the first Fc domain monomer and the second Fc domain monomer; b) a second polypeptide including a third Fc domain monomer; c) a third polypeptide including a fourth Fc domain monomer; d) a first antigen binding domain joined to the first polypeptide; e) a second antigen binding domain joined to the second polypeptide; and f) a third antigen binding domain joined to the third polypeptide; where the first Fc domain monomer and the third Fc domain monomer combine to form a first Fc domain and the second Fc domain monomer and the fourth Fc domain monomer combine to form a second Fc domain, and where the first, the second, and the third antigen binding domains bind different antigens.

In an twenty fifth aspect, the disclosure features a method of manufacturing an Fc-antigen binding domain construct, the method including: a) culturing a host cell expressing: (1) a first polypeptide including i) a first Fc domain monomer, ii) a second Fc domain monomer, and iii) a linker joining the first Fc domain monomer and the second Fc domain monomer; (2) a second polypeptide including a third Fc domain monomer; (3) a third polypeptide including a fourth Fc domain monomer; (4) a first antigen binding domain joined to the first polypeptide; (5) a second antigen binding domain joined to the second polypeptide; and (6) a third antigen binding domain joined to the third polypeptide; where the first Fc domain monomer and the third Fc domain monomer combine to form a first Fc domain and the second Fc domain monomer and the fourth Fc domain monomer combine to form a second Fc domain; where the antigen binding domain is joined to the first polypeptide, second polypeptide, or third polypeptide, thereby forming an Fc-antigen binding domain construct, where the first and the second antigen binding domains bind different antigens, and where at least 50% of the Fc-antigen binding domain constructs in a cell culture supernatant, on a molar basis, are structurally identical, and b) purifying the Fc-antigen binding domain construct from the cell culture supernatant.

In some embodiments of the twenty first, twenty second, twenty third, twenty fourth, and twenty fifth aspect of the disclosure, the first Fc domain monomer and the third Fc domain monomer include complementary dimerization selectivity modules that promote dimerization between the first Fc domain monomer and the third Fc domain monomer, where the second Fc domain monomer and the fourth Fc domain monomer include complementary dimerization selectivity modules that promote dimerization between the second Fc domain monomer and the fourth Fc domain monomer, and where the second polypeptide and the third polypeptide have different amino acid sequences.

In a twenty sixth aspect, the disclosure features an Fc-antigen binding domain construct including: a) a first polypeptide including i) a first Fc domain monomer, ii) a second Fc domain monomer, and iii) a first linker joining the first Fc domain monomer and the second Fc domain monomer, and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer; d) a fourth polypeptide including an sixth Fc domain monomer; and d) an antigen binding domain joined to the first polypeptide, second polypeptide, third polypeptide, or fourth polypeptide, where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain, and where the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain.

In a twenty seventh aspect, the disclosure features a Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer; d) a fourth polypeptide including an sixth Fc domain monomer; and e) an antigen binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain, and where the Fc-antigen binding domain construct includes a biological activity that is not exhibited by a construct having a single Fc domain and the antigen binding domain.

In a twenty eighth aspect, the disclosure features an Fc-antigen binding domain construct including: a) a first polypeptide including i) a first Fc domain monomer, ii) a second Fc domain monomer, and iii) a first spacer joining the first Fc domain monomer and the second Fc domain monomer, and b) a second polypeptide including iv) a third Fc domain monomer, v) a fourth Fc domain monomer, and vi) a second spacer joining the third Fc domain monomer and the fourth Fc domain monomer and c) a third polypeptide including a fifth Fc domain monomer, d) a fourth polypeptide including an sixth Fc domain monomer; and e) an antigen binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain.

In a twenty ninth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, and iii) a first linker joining the first Fc domain monomer and the second Fc domain monomer; and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer d) a fourth polypeptide including an sixth Fc domain monomer; and e) an antigen binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain.

In a thirtieth aspect, the disclosure features a method of manufacturing an Fc-antigen binding domain construct, the method including: a) culturing a host cell expressing: (1) a first polypeptide including i) a first Fc domain monomer, ii) a second Fc domain monomer, and iii) a first linker joining the first Fc domain monomer and the second Fc domain monomer; and (2) a second polypeptide including iv) a third Fc domain monomer, v) a fourth Fc domain monomer, and vi) a second linker joining the third Fc domain monomer and the fourth Fc domain monomer; and (3) a third polypeptide including a fifth Fc domain monomer; (4) a fourth polypeptide including an sixth Fc domain monomer; and (5) an antigen binding domain joined to the first polypeptide, second polypeptide, third polypeptide, or fourth polypeptide; where the first Fc domain monomer and the third Fc domain monomer combine to form a first Fc domain and the second Fc domain monomer and the fifth Fc domain monomer combine to form a second Fc domain, the fourth Fc domain monomer and the sixth Fc domain monomer combine to form a third Fc domain, and where at least 50% of the Fc-antigen binding domain constructs in a cell culture supernatant, on a molar basis, are structurally identical, and b) purifying the Fc-antigen binding domain construct from the cell culture supernatant.

In some embodiments of the twenty sixth, twenty seventh, twenty eighth, twenty ninth, and thirtieth aspect of the disclosure, each of the first and third Fc domain monomers includes a complementary dimerization selectivity module that promote dimerization between the first Fc domain monomer and the third Fc domain monomer, each of the second and fifth Fc domain monomers includes a complementary dimerization selectivity module that promote dimerization between the second Fc domain monomer and the fifth Fc domain monomer, and each of the fourth and sixth Fc domain monomers includes a complementary dimerization selectivity module that promote dimerization between the fourth Fc domain monomer and the sixth Fc domain monomer.

In a thirty first aspect, the disclosure features an Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including iv) a third Fc domain monomer, v) a fourth Fc domain monomer, and vi) a second linker joining the third Fc domain monomer and the fourth Fc domain monomer: and c) a third polypeptide including a fifth Fc domain monomer; d) a fourth polypeptide including an sixth Fc domain monomer and e) an antigen binding domain joined to the first polypeptide, second polypeptide, third polypeptide, or fourth polypeptide; where the second Fc domain monomer and the fourth Fc domain monomer combine to form a first Fc domain and the first Fc domain monomer and the fifth Fc domain monomer combine to form a second Fc domain, the third Fc domain monomer and the sixth Fc domain monomer combine to form a third Fc domain, and where the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain.

In a thirty second aspect, the disclosure features an Fc-antigen binding domain construct including: a) a first polypeptide including i) a first Fc domain monomer, ii) a second Fc domain monomer, and iii) a first linker joining the first Fc domain monomer and the second Fc domain monomer; and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer; d) a fourth polypeptide including an sixth Fc domain monomer; and e) an antigen binding domain joined to the first polypeptide, second polypeptide, third polypeptide, or fourth polypeptide; where the second Fc domain monomer and the fourth Fc domain monomer combine to form a first Fc domain and the first Fc domain monomer and the fifth Fc domain monomer combine to form a second Fc domain, the third Fc domain monomer and the sixth Fc domain monomer combine to form a third Fc domain, and where the Fc-antigen binding domain construct includes a biological activity that is not exhibited by a construct having a single Fc domain and the antigen binding domain.

In a thirty third aspect, the disclosure features an Fc-antigen binding domain construct including: a) a first polypeptide including i) a first Fc domain monomer, ii) a second Fc domain monomer, and iii) a first spacer joining the first Fc domain monomer and the second Fc domain monomer, and b) a second polypeptide including iv) a third Fc domain monomer, v) a fourth Fc domain monomer, and vi) a second spacer joining the third Fc domain monomer and the fourth Fc domain monomer; and c) a third polypeptide including a fifth Fc domain monomer, d) a fourth polypeptide including an sixth Fc domain monomer; and e) an antigen binding domain joined to the first polypeptide, second polypeptide, third polypeptide, or fourth polypeptide, where the second Fc domain monomer and the fourth Fc domain monomer combine to form a first Fc domain and the first Fc domain monomer and the fifth Fc domain monomer combine to form a second Fc domain, the third Fc domain monomer and the sixth Fc domain monomer combine to form a third Fc domain.

In a thirty fourth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, and iii) a first linker joining the first Fc domain monomer and the second Fc domain monomer and b) a second polypeptide including iv) a third Fc domain monomer, v) a fourth Fc domain monomer, and vi) a second linker joining the third Fc domain monomer and the fourth Fc domain monomer; and c) a third polypeptide including a fifth Fc domain monomer d) a fourth polypeptide including an sixth Fc domain monomer; and e) an antigen binding domain joined to the first polypeptide, second polypeptide, third polypeptide, or fourth polypeptide; where the second Fc domain monomer and the fourth Fc domain monomer combine to form a first Fc domain and the first Fc domain monomer and the fifth Fc domain monomer combine to form a second Fc domain, the third Fc domain monomer and the sixth Fc domain monomer combine to form a third Fc domain.

In a thirty fifth aspect, the disclosure features a method of manufacturing an Fc-antigen binding domain construct, the method including: a) culturing a host cell expressing: (1) a first polypeptide including i) a first Fc domain monomer, ii) a second Fc domain monomer, and iii) a first linker joining the first Fc domain monomer and the second Fc domain monomer; and (2) a second polypeptide including iv) a third Fc domain monomer, v) a fourth Fc domain monomer, and vi) a second linker joining the third Fc domain monomer and the fourth Fc domain monomer; and (3) a third polypeptide including a fifth Fc domain monomer; (4) a fourth polypeptide including an sixth Fc domain monomer; and (5) an antigen binding domain joined to the first polypeptide, second polypeptide, third polypeptide, or fourth polypeptide; where the second Fc domain monomer and the fourth Fc domain monomer combine to form a first Fc domain and the first Fc domain monomer and the fifth Fc domain monomer combine to form a second Fc domain, the third Fc domain monomer and the sixth Fc domain monomer combine to form a third Fc domain, and where at least 50% of the Fc-antigen binding domain constructs in a cell culture supernatant, on a molar basis, are structurally identical, and b) purifying the Fc-antigen binding domain construct from the cell culture supernatant.

In some embodiments of the thirty first, thirty second, thirty third, thirty fourth, or thirty fifth aspect of the disclosure, each of the second and fourth Fc domain monomers includes a complementary dimerization selectivity module that promote dimerization between the second Fc domain monomer and the fourth Fc domain monomer, each of the first and fifth Fc domain monomers includes a complementary dimerization selectivity module that promote dimerization between the first Fc domain monomer and the fifth Fc domain monomer, and each of the third and sixth Fc domain monomers includes a complementary dimerization selectivity module that promote dimerization between the third Fc domain monomer and the sixth Fc domain monomer.

In a thirty sixth aspect, the disclosure features an Fc-antigen binding domain construct including:

a) a first polypeptide including i) a first Fc domain monomer, ii) a second Fc domain monomer, iii) a third Fc domain monomer, iv) a first linker joining the first Fc domain monomer and the second Fc domain monomer; and v) a second linker joining the second Fc domain monomer and the third Fc domain monomer; b) a second polypeptide including vi) a fourth Fc domain monomer, vii) a fifth Fc domain monomer, viii) a sixth Fc domain monomer, ix) a third linker joining the fourth Fc domain monomer and the fifth Fc domain monomer; and x) a fourth linker joining the fifth Fc domain monomer and the sixth Fc domain monomer; c) a third polypeptide including a seventh Fc domain monomer; d) a fourth polypeptide including an eighth Fc domain monomer; e) a fifth polypeptide including a ninth Fc domain monomer; f) a sixth polypeptide including a tenth Fc domain monomer; and g) an antigen binding domain joined to the first polypeptide, second polypeptide, third polypeptide, fourth polypeptide, fifth polypeptide, or sixth polypeptide, where the second Fc domain monomer and the fifth Fc domain monomer combine to form a first Fc domain and the first Fc domain monomer and the seventh Fc domain monomer combine to form a second Fc domain, the fourth Fc domain monomer and the eighth Fc domain monomer combine to form a third Fc domain, the third Fc domain monomer and the ninth Fc domain monomer combine to form a fourth Fc domain, and the sixth Fc domain monomer and the tenth Fc domain monomer combine to form a fifth Fc domain, and where the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain.

In a thirty seventh aspect, the disclosure features an Fc-antigen binding domain construct including: a) a first polypeptide including i) a first Fc domain monomer, ii) a second Fc domain monomer, iii) a third Fc domain monomer, iv) a first linker joining the first Fc domain monomer and the second Fc domain monomer; and v) a second linker joining the second Fc domain monomer and the third Fc domain monomer; b) a second polypeptide including vi) a fourth Fc domain monomer, vii) a fifth Fc domain monomer, viii) a sixth Fc domain monomer, ix) a third linker joining the fourth Fc domain monomer and the fifth Fc domain monomer; and x) a fourth linker joining the fifth Fc domain monomer and the sixth Fc domain monomer; c) a third polypeptide including a seventh Fc domain monomer; d) a fourth polypeptide including an eighth Fc domain monomer; e) a fifth polypeptide including a ninth Fc domain monomer; f) a sixth polypeptide including a tenth Fc domain monomer; and g) an antigen binding domain joined to the first polypeptide, second polypeptide, third polypeptide, fourth polypeptide, fifth polypeptide, or sixth polypeptide; where the second Fc domain monomer and the fifth Fc domain monomer combine to form a first Fc domain and the first Fc domain monomer and the seventh Fc domain monomer combine to form a second Fc domain, the fourth Fc domain monomer and the eighth Fc domain monomer combine to form a third Fc domain, the third Fc domain monomer and the ninth Fc domain monomer combine to form a fourth Fc domain, and the sixth Fc domain monomer and the tenth Fc domain monomer combine to form a fifth Fc domain, and where the Fc-antigen binding domain construct includes a biological activity that is not exhibited by a construct having a single Fc domain and the antigen binding domain.

In a thirty eighth aspect, the disclosure features an Fc-antigen binding domain construct including: a) a first polypeptide including i) a first Fc domain monomer, ii) a second Fc domain monomer, iii) a third Fc domain monomer, iv) a first spacer joining the first Fc domain monomer and the second Fc domain monomer; and v) a second spacer joining the second Fc domain monomer and the third Fc domain monomer; b) a second polypeptide including vi) a fourth Fc domain monomer, vii) a fifth Fc domain monomer, viii) a sixth Fc domain monomer, ix) a third spacer joining the fourth Fc domain monomer and the fifth Fc domain monomer; and x) a fourth spacer joining the fifth Fc domain monomer and the sixth Fc domain monomer; c) a third polypeptide including a seventh Fc domain monomer, d) a fourth polypeptide including an eighth Fc domain monomer, e) a fifth polypeptide including a ninth Fc domain monomer, f) a sixth polypeptide including a tenth Fc domain monomer and g) an antigen binding domain joined to the first polypeptide, second polypeptide, third polypeptide, fourth polypeptide, fifth polypeptide, or sixth polypeptide; where the second Fc domain monomer and the fifth Fc domain monomer combine to form a first Fc domain and the first Fc domain monomer and the seventh Fc domain monomer combine to form a second Fc domain, the fourth Fc domain monomer and the eighth Fc domain monomer combine to form a third Fc domain, the third Fc domain monomer and the ninth Fc domain monomer combine to form a fourth Fc domain, and the sixth Fc domain monomer and the tenth Fc domain monomer combine to form a fifth Fc domain.

In a thirty ninth aspect, the disclosure features a cell culture medium including a population of Fc-antigen binding domain constructs, where at least 50% of the Fc-antigen binding domain constructs, on a molar basis, include: a) a first polypeptide including i) a first Fc domain monomer, ii) a second Fc domain monomer, iii) a third Fc domain monomer, iv) a first spacer joining the first Fc domain monomer and the second Fc domain monomer, and v) a second spacer joining the second Fc domain monomer and the third Fc domain monomer; b) a second polypeptide including vi) a fourth Fc domain monomer, vii) a fifth Fc domain monomer, viii) a sixth Fc domain monomer, ix) a third spacer joining the fourth Fc domain monomer and the fifth Fc domain monomer, and x) a fourth spacer joining the fifth Fc domain monomer and the sixth Fc domain monomer, c) a third polypeptide including a seventh Fc domain monomer, d) a fourth polypeptide including an eighth Fc domain monomer; e) a fifth polypeptide including a ninth Fc domain monomer; f) a sixth polypeptide including a tenth Fc domain monomer; and g) an antigen binding domain joined to the first polypeptide, second polypeptide, third polypeptide, fourth polypeptide, fifth polypeptide, or sixth polypeptide; where the second Fc domain monomer and the fifth Fc domain monomer combine to form a first Fc domain and the first Fc domain monomer and the seventh Fc domain monomer combine to form a second Fc domain, the fourth Fc domain monomer and the eighth Fc domain monomer combine to form a third Fc domain, the third Fc domain monomer and the ninth Fc domain monomer combine to form a fourth Fc domain, and the sixth Fc domain monomer and the tenth Fc domain monomer combine to form a fifth Fc domain.

In a fortieth aspect, the disclosure features a method of manufacturing an Fc-antigen binding domain construct, the method including: a) culturing a host cell expressing: (1) a first polypeptide including i) a first Fc domain monomer, ii) a second Fc domain monomer, iii) a third Fc domain monomer, iv) a first spacer joining the first Fc domain monomer and the second Fc domain monomer; and v) a second spacer joining the second Fc domain monomer and the third Fc domain monomer (2) a second polypeptide including vi) a fourth Fc domain monomer, vii) a fifth Fc domain monomer, viii) a sixth Fc domain monomer, ix) a third spacer joining the fourth Fc domain monomer and the fifth Fc domain monomer; and x) a fourth spacer joining the fifth Fc domain monomer and the sixth Fc domain monomer (3) a third polypeptide including a seventh Fc domain monomer (4) a fourth polypeptide including an eighth Fc domain monomer, (5) a fifth polypeptide including a ninth Fc domain monomer (6) a sixth polypeptide including a tenth Fc domain monomer; and (7) an antigen binding domain joined to the first polypeptide, second polypeptide, third polypeptide, fourth polypeptide, fifth polypeptide, or sixth polypeptide; where the second Fc domain monomer and the fifth Fc domain monomer combine to form a first Fc domain and the first Fc domain monomer and the seventh Fc domain monomer combine to form a second Fc domain, the fourth Fc domain monomer and the eighth Fc domain monomer combine to form a third Fc domain, the third Fc domain monomer and the ninth Fc domain monomer combine to form a fourth Fc domain, and the sixth Fc domain monomer and the tenth Fc domain monomer combine to form a fifth Fc domain, and where at least 50% of the Fc-antigen binding domain constructs in a cell culture supernatant, on a molar basis, are structurally identical, and b) purifying the Fc-antigen binding domain construct from the cell culture supernatant.

In some embodiments of the thirty sixth, thirty seventh, thirty eighth, thirty ninth, and fortieth aspects of the disclosure, each of the second and fifth Fc domain monomers includes a complementary dimerization selectivity module that promote dimerization between the second Fc domain monomer and the fifth Fc domain monomer, each of the first and seventh Fc domain monomers includes a complementary dimerization selectivity module that promote dimerization between the first Fc domain monomer and the seventh Fc domain monomer, each of the fourth and eighth Fc domain monomers includes a complementary dimerization selectivity module that promote dimerization between the fourth Fc domain monomer and the eighth Fc domain monomer, each of the third and ninth Fc domain monomers includes a complementary dimerization selectivity module that promote dimerization between the third Fc domain monomer and the ninth Fc domain monomer, and each of the sixth and tenth Fc domain monomers includes a complementary dimerization selectivity module that promote dimerization between the sixth Fc domain monomer and the tenth Fc domain monomer.

In some embodiments of all aspects of the disclosure, the Fc-antigen binding domain construct has reduced fucosylation. Thus, in some embodiments, less than 40%, 30%, 20%, 15%, 10% or 5% of the Fc domain monomers in a composition comprising an Fc-antigen binding domain construct are fucosylated.

In some embodiments of all aspects of the disclosure, the Fc domain monomer comprises the amino acid sequence of FIG. 53A (SEQ ID NO: 43) with up to 10 (9, 8, 7, 6, 5, 4, 3, 2 or 1) single amino acid changes in the C_{H}3 domain.

In some embodiments of all aspects of the disclosure, the Fc domain monomer comprises the amino acid sequence of FIG. 53B (SEQ ID NO: 45) with up to 10 (9, 8, 7, 6, 5, 4, 3, 2 or 1) single amino acid changes in the C_{H}3 domain.

In some embodiments of all aspects of the disclosure, the Fc domain monomer comprises the amino acid sequence of FIG. 53C (SEQ ID NO: 47) with up to 10 (9, 8, 7, 6, 5, 4, 3, 2 or 1) single amino acid changes in the C_{H}3 domain.

In some embodiments of all aspects of the disclosure, the Fc domain monomer comprises the amino acid sequence of FIG. 53D (SEQ ID NO: 42) with up to 10 (9, 8, 7, 6, 5, 4, 3, 2 or 1) single amino acid changes in the C_{H}3 domain.

In some embodiments of all aspects of the disclosure, for example, when the Fc domain monomer is at the carboxy-terminal end of a polypeptide, the Fc domain monomer does not include K447. In other embodiments, for example, when the Fc domain monomer is not at the carboxy-terminal end of a polypeptide, the Fc domain monomer includes K447.

In some embodiments of all aspects of the disclosure, for example, when the Fc domain monomer is amino terminal to a linker, the Fc domain monomer does not include the portion of the hinge from E216 to C220, inclusive, but does include the portion of the hinge from D221 to L235, inclusive. In other embodiments, for example, when the Fc domain monomer is carboxy-terminal to a CH1 domain, the Fc domain monomer includes the portion of the hinge from E216 to L235, inclusive. In some embodiments of all aspects of the disclosure, a hinge domain, for example a hinge domain at the amino terminus of a polypeptide, has an Asp to Gln mutation at Kabat position 221.

As noted above, the Fc-antigen binding domain constructs of the disclosure are assembled from polypeptides, including polypeptides comprising two or more IgG1 Fc domain monomers, and such polypeptides are an aspect of the present disclosure.

In a forty first aspect, the disclosure features a polypeptide comprising an antigen binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises mutations forming an engineered protuberance.

In various embodiments of the forty first aspect: the antigen binding domain comprises an antibody heavy chain variable domain; the antigen binding domain comprises an antibody light chain variable domain; the first IgG1 Fc domain monomer comprises two or four reverse charge mutations and the second IgG1 Fc domain monomer comprises mutations forming an engineered protuberance; the first IgG1 Fc domain monomer comprises mutations forming an engineered protuberance and the second IgG1 Fc domain monomer comprises two or four reverse charge mutations; both the first IgG1 Fc domain monomer and the second IgG constant domain monomer comprise mutations forming an engineered protuberance; the polypeptide comprises a third linker and a third IgG1 Fc domain monomer wherein the first IgG1 Fc domain monomer, the second IgG1 Fc domain monomer and the third IgG1 Fc domain monomer each comprise mutations forming an engineered protuberance; the polypeptide comprises a third linker and a third IgG1 Fc domain monomer wherein both the first IgG1 Fc domain monomer and the second IgG1 Fc domain monomer each comprise mutations forming an engineered protuberance and the third IgG1 Fc domain monomer comprises two or four reverse charge mutations; the polypeptide comprises a third linker and third IgG1 Fc domain monomer wherein both the first IgG1 Fc domain monomer and the third IgG1 Fc domain monomer each comprise mutations forming an engineered protuberance and the second IgG1 domain monomer comprises two or four reverse charge mutations; the polypeptide comprises a third linker and a third IgG1 Fc domain monomer wherein both the second IgG1 Fc domain monomer and the third IgG1 Fc domain monomer each comprise mutations forming an engineered protuberance and the first IgG1 domain monomer comprises two or four reverse charge mutations.

In various embodiments of the forty first aspect: the IgG1 Fc domain monomers comprising mutations forming an engineered protuberance further comprise one, two or three reverse charge mutations; the mutations forming an engineered protuberance and the reverse charge mutations are in the CH3 domain; the mutations are within the sequence from Kabat position G341 to Kabat position K447, inclusive; the mutations are single amino acid changes; the second linker and the optional third linker comprise or consist of an amino acid sequence selected from the group consisting of: GGGGGGGGGGGGGGGGGGGG, GGGGS, GGSG, SGGG, GSGS, GSGSGS, GSGSGSGS, GSGSGSGSGS, GSGSGSGSGSGS, GGSGGS, GGSGGSGGS, GGSGGSGGSGGS, GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG and GGGGGGGGGGGGGGGG; the second linker and the optional third linker is a glycine spacer; the second linker and the optional third linker independently consist of 4 to 30, 4 to 20, 8 to 30, 8 to 20, 12 to 20 or 12 to 30 glycine residues; the second linker and the optional third linker consist of 20 glycine residues; at least one of the Fc domain monomers comprises a single amino acid mutation at Kabat position I253 each amino acid mutation at Kabat position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y; each amino acid mutation at position I253 is I253A; at least one of the Fc domain monomers comprises a single amino acid mutation at Kabat position R292; each amino acid mutation at Kabat position R292 is independently selected from the group consisting of R292D, R292E, R292L, R292P, R292Q, R292R. R292T, and R292Y; each amino acid mutation at position R292 is R292P; each Fc domain monomer independently comprises or consists of an amino acid sequence selected from the group consisting of EPKSCDKTHTCPPCPAPELL and DKTHTCPPCPAPELL; the hinge portion of the second Fc domain monomer and the third Fc domain monomer have the amino acid sequence DKTHTCPPCPAPELL; the hinge portion of the first Fc domain monomer has the amino acid sequence EPKSCDKTHTCPPCPAPEL; the hinge portion of the first Fc domain monomer has the amino acid sequence EPKSCDKTHTCPPCPAPEL and the hinge portion of the second Fc domain monomer and the third Fc domain monomer have the amino acid sequence DKTHTCPPCPAPELL; the CH2 domains of each Fc domain monomer independently comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVDVSHEDPEVKFNWYVDGVEVHNAKTKPREEQYNSTYRWS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVVDVSHEDPEVKFNWYVDGVEVH NAKTKPREEQYNSTYRWS VLTVLHQDWLNGKEYKCKVSNKALPAPIEKTISKAK with no more than two single amino acid substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 10 single amino acid substitutions; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 8 single amino acid substitutions; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 6 single amino acid substitutions; wherein the CH3 domains of each Fc domain monomer independently comprise the amino acid sequence: GQPREPQVYTLPPSRDELTKNQVSLTCLVKGFYPSDIAVEWESNGQPEN NYKTTPPVLDSDGSFFLYSK LTVDKSRWQQGNVFSCSVMHEALHNHYTQKSLSLSPG with no more than 5 single amino acid substitutions; the single amino acid substitutions are selected from the group consisting of: T366Y, T366W, T394W, T394Y, F405W, F405A, Y407A, S354C, Y349T, T394F, K409D, K409E, K392D, K392E, K370D, K370E, D399K, D399R, E357K, E357R, D356K, and D356K; each of the Fc domain monomers independently comprises the amino acid sequence of any of SEQ ID NOs:42, 43, 45, and 45 having up to 10 single amino acid substitutions; up to 6 of the single amino acid substitutions are reverse charge mutations in the CH3 domain or are mutations forming an engineered protuberance; the single amino acid substitutions are within the sequence from Kabat position G341 to Kabat position K447, inclusive; at least one of the mutations forming an engineered protuberance is selected from the group consisting of T366Y, T366W, T394W, T394Y, F405W, F405A, Y407A, S354C, Y349T, and T394F; the two or four reverse charge mutations are selected from: K409D, K409E, K392D, K392E, K370D, K370E, D399K, D399R, E357K, E357R, D356K, and D356K; the antigen binding domain is a scFv; the antigen binding domain comprises a VH domain and a CH1 domain; the antigen binding domain further comprises a VL domain; the VH domain comprises a set of CDR-H1, CDR-H2 and CDR-H3 sequences set forth in Table 1; the VH domain comprises CDR-H1, CDR-H2, and CDR-H3 of a VH domain comprising a sequence of an antibody set forth in Table 2; the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2; the VH domain comprises a V_{H} sequence of an antibody set forth in Table 2; the antigen binding domain comprises a set of CDR-H1, CDR-H2, CDR-H3, CDR-L1, CDR-L2, and CDR-L3 sequences set forth in Table 1; the antigen binding domain comprises CDR-H1, CDR-H2, CDR-H3, CDR-L1, CDR-L2, and CDR-L3 sequences from a set of a VH and a VL sequence of an antibody set forth in Table 2; the antigen binding domain comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2; the antigen binding domain comprises a set of a VH and a VL sequence of an antibody set forth in Table 2; the antigen binding domain comprises an IgG CL antibody constant domain and an IgG CH1 antibody constant domain; the antigen binding domain comprises a VH domain and CH1 domain and can bind to a polypeptide comprising a VL domain and a CL domain to form a Fab.

Also described is a polypeptide complex comprising two copies of the polypeptide of described above joined by disulfide bonds between cysteine residues within the hinge of first or second IgG1 Fc domain monomers.

Also described is a polypeptide complex comprising a polypeptide described above joined to a second polypeptide comprising and IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide.

In various embodiments of the complexes: the second polypeptide monomer comprises mutations forming an engineered cavity; the mutations forming the engineered cavity are selected from the group consisting of: Y407T, Y407A, F405A, T394S, T394W/Y407A, T366W/T394S, T366S/L368A/Y407V/Y349C, S364H/F405A; the second polypeptide comprises the amino acid sequence of any of SEQ ID NOs: 42, 43, 45, and 45 having up to 10 single amino acid substitutions.

In a forty second aspect, the disclosure features: a polypeptide comprising: an antigen binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises one, two or three reverse charge amino acid mutations.

In various embodiments of the forty second aspect: the antigen binding domain comprises an antibody heavy chain variable domain; the antigen binding domain comprises an antibody light chain variable domain; the first IgG1 Fc domain monomer comprises a set of two reverse charge mutations selected from those in Table 5 or a set of four reverse charge mutation selected from those in Table 6 and the second IgG1 Fc domain monomer comprises one, two or three reverse charge amino acid mutations selected from Table 4; the first IgG1 Fc domain monomer comprises one, two or three reverse charge amino acid mutations selected from Table 4 and the second IgG1 Fc domain monomer comprises a set of two reverse charge mutations selected from those in Table 5 or a set of four reverse charge mutation selected from those in Table 6; both the first IgG1 Fc domain monomer and the second IgG constant domain monomer comprise one, two or three reverse charge amino acid mutations selected from Table 4; the polypeptide further comprises a third linker and a third IgG1 Fc domain monomer wherein the first IgG1 Fc domain monomer, the second IgG1 Fc domain monomer and the third IgG1 Fc domain monomer each comprise one, two or three reverse charge amino acid mutations selected from Table 4; the polypeptide further comprises a third linker and a third IgG1 Fc domain monomer wherein both the first IgG1 Fc domain monomer and the second IgG1 Fc domain monomer each comprise one, two or three reverse charge amino acid mutations selected from Table 4 and the third IgG1 Fc domain monomer comprises a set of two reverse charge mutations selected from those in Table 5 or a set of four reverse charge mutation selected from those in Table 6; the polypeptide further comprises a third linker and third IgG1 Fc domain monomer wherein both the first IgG1 Fc domain monomer and the third IgG1 Fc domain monomer each comprise one, two or three reverse charge amino acid mutations selected from Table 4 and the second IgG1 domain monomer comprises a set of two reverse charge mutations selected from those in Table 5 or a set of four reverse charge mutation selected from those in Table 6; the polypeptide further comprises a third linker and a third IgG1 Fc domain monomer wherein both the second IgG1 Fc domain monomer and the third IgG1 Fc domain monomer each comprise one, two or three reverse charge amino acid mutations selected from Table 4 and the first IgG1 domain monomer comprises a set of two reverse charge mutations selected from those in Table 5 or a set of four reverse charge mutation selected from those in Table 6; the IgG1 Fc domain monomers comprising one, two or three reverse charge amino acid mutations selected from Table 4 have identical CH3 domains; one, two or three reverse charge amino acid mutations selected from Table 4 are in the CH3 domain; the mutations are within the sequence from Kabat position G341 to Kabat position K447, inclusive; the mutations are each single amino acid changes; the mutations are within the sequence from Kabat position G341 to Kabat position K446, inclusive; the mutations are single amino acid changes; the second linker and the optional third linker comprise or consist of an amino acid sequence selected from the group consisting of: GGGGGGGGGGGGGGGGGGGG, GGGGS, GGSG, SGGG, GSGS, GSGSGS, GSGSGSGS, GSGSGSGSGSGSGS, GGSGS, GGSGGSGS, GGSGGSGGSGGS GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG and GGGGGGGGGGGGGGGG; the second linker and the optional third linker is a glycine spacer; the second linker and the optional third linker independently consist of 4 to 30, 4 to 20, 8 to 30, 8 to 20, 12 to 20 or 12 to 30 glycine residues; the second linker and the optional third linker consist of 20 glycine residues; at least one of the Fc domain monomers comprises a single amino acid mutation at Kabat position I253 each amino acid mutation at Kabat position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y; each amino acid mutation at position I253 is I253A; at least one of the Fc domain monomers comprises a single amino acid mutation at Kabat position R292; each amino acid mutation at Kabat position R292 is independently selected from the group consisting of R292D, R292E, R292L, R292P, R292Q, R292R, R292T, and R292Y; each amino acid mutation at position R292 is R292P; each Fc domain monomer independently comprises or consists of an amino acid sequence selected from the group consisting of EPKSCDKTHTCPPCPAPELL and DKTHTCPPCPAPELL; the hinge portion of the second Fc domain monomer and the third Fc domain monomer have the amino acid sequence DKTHTCPPCPAPELL; the hinge portion of the first Fc domain monomer has the amino acid sequence EPKSCDKTHTCPPCPAPEL; the hinge portion of the first Fc domain monomer has the amino acid sequence EPKSCDKTHTCPPCPAPEL and the hinge portion of the second Fc domain monomer and the third Fc domain monomer have the amino acid sequence DKTHTCPPCPAPELL; the CH2 domains of each Fc domain monomer independently comprise the amino acid sequence: GGPSVFLFPPKPKDTLMISRTPEVCVVVDVSHEDPEVKFNWYVDGVEVHNAKTKPREEQYNSTYRVVS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK with no more than two single amino acid substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 10 single amino acid substitutions; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 8 single amino acid substitutions; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 6 single amino acid substitutions; wherein the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 5 single amino acid substitutions; the single amino acid substitutions are selected from the group consisting of: T366Y, T366W, T394W, T394Y, F405W, F405A, Y407A, S354C, Y349T, T394F, K409D, K409E, K392D, K392E, K370D, K370E, D399K, D399R, E357K, E357R, D356K, and D356K; each of the Fc domain monomers independently comprises the amino acid sequence of any of SEQ ID NOs:42, 43, 45, and 45 having up to 10 single amino acid substitutions; up to 6 of the single amino acid substitutions are reverse charge mutations in the CH3 domain or are mutations forming an engineered protuberance; the single amino acid substitutions are within the sequence from Kabat position G341 to Kabat position K447, inclusive; at least one of the mutations forming an engineered protuberance is selected from the group consisting of T366Y, T366W, T394W, T394Y, F405W, F405A, Y407A, S354C, Y349T, and T394F; the two or four reverse charge mutations are selected from: K409D, K409E, K392D, K392E, K370D, K370E, D399K, D399R, E357K, E357R, D356K, and D356K; the antigen binding domain is a scFv; the antigen binding domain comprises a VH domain and a CH1 domain; the antigen binding domain further comprises a VL domain; the VH domain comprises a set of CDR-H1, CDR-H2 and CDR-H3 sequences set forth in Table 1; the VH domain comprises CDR-H1, CDR-H2, and CDR-H3 of a VH domain comprising a sequence of an antibody set forth in Table 2; the VH domain comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2; the VH domain comprises a VH sequence of an antibody set forth in Table 2; the antigen binding domain comprises a set of CDR-H1, CDR-H2, CDR-H3, CDR-L1, CDR-L2, and CDR-L3 sequences set forth in Table 1; the antigen binding domain comprises CDR-H1, CDR-H2, CDR-H3, CDR-L1, CDR-L2, and CDR-L3 sequences from a set of a VH and a VL sequence of an antibody set forth in Table 2; the antigen binding domain comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2; the antigen binding domain comprises a set of a VH and a VL sequence of an antibody set forth in Table 2; the antigen binding domain comprises an IgG CL antibody constant domain and an IgG CH1 antibody constant domain; the antigen binding domain comprises a VH domain and CH1 domain and can bind to a polypeptide comprising a VL domain and a CL domain to form a Fab.

Also described is a polypeptide complex comprising two copies of any of the polypeptides described above joined by disulfide bonds between cysteine residues within the hinge of first or second IgG1 Fc domain monomers.

Also described is a polypeptide complex comprising a polypeptide described above joined to a second polypeptide comprising and IgG1 Fc domain monomer comprising a hinge domain, a CH2 domain and a CH3 domain, wherein the polypeptide and the second polypeptide are joined by disulfide bonds between cysteine residues within the hinge domain of the first, second or third IgG1 Fc domain monomer of the polypeptide and the hinge domain of the second polypeptide. In various embodiments: the second polypeptide monomer comprises one, two or three reverse charge mutations; the second polypeptide monomer comprises one, two or three reverse charge mutations selected from Table 4 and are complementary to the one, two or three reverse charge mutations selected Table 4 in the polypeptide; the second polypeptide comprises the amino acid sequence of any of SEQ ID NOs: 42, 43, 45, and 45 having up to 10 single amino acid substitutions.

In a forty third aspect, the disclosure features a polypeptide comprising: a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises mutations forming an engineered protuberance.

In various embodiments of the forty third aspect: the polypeptide further comprises: an antibody heavy chain variable domain and CH1 domain amino terminal to the first IgG1 monomer or an scFv amino terminal to the first IgG1 monomer; the first IgG1 Fc domain monomer comprises two or four reverse charge mutations and the second IgG1 Fc domain monomer comprises mutations forming an engineered protuberance; the first IgG1 Fc domain monomer comprises mutations forming an engineered protuberance and the second IgG1 Fc domain monomer comprises two or four reverse charge mutations; both the first IgG1 Fc domain monomer and the second IgG constant domain monomer comprise mutations forming an engineered protuberance; the polypeptide comprises a third linker and a third IgG1 Fc domain monomer wherein the first IgG1 Fc domain monomer, the second IgG1 Fc domain monomer and the third IgG1 Fc domain monomer each comprise mutations forming an engineered protuberance; the polypeptide comprises a third linker and a third IgG1 Fc domain monomer wherein both the first IgG1 Fc domain monomer and the second IgG1 Fc domain monomer each comprise mutations forming an engineered protuberance and the third IgG1 Fc domain monomer comprises two or four reverse charge mutations; the polypeptide comprises a third linker and third IgG1 Fc domain monomer wherein both the first IgG1 Fc domain monomer and the third IgG1 Fc domain monomer each comprise mutations forming an engineered protuberance and the second IgG1 domain monomer comprises two or four reverse charge mutations; the polypeptide comprises a third linker and a third IgG1 Fc domain monomer wherein both the second IgG1 Fc domain monomer and the third IgG1 Fc domain monomer each comprise mutations forming an engineered protuberance and the first IgG1 domain monomer comprises two or four reverse charge mutations.

In various embodiments of the forty third aspect: the IgG1 Fc domain monomers comprising mutations forming an engineered protuberance further comprise one, two or three reverse charge mutations;

the mutations forming an engineered protuberance and the reverse charge mutations are in the CH3 domain; the mutations are within the sequence from Kabat position G341 to Kabat position K447, inclusive; the mutations are single amino acid changes; the second linker and the optional third linker comprise or consist of an amino acid sequence selected from the group consisting of: GGGGGGGGGGGGGGGGGGGG, GGGGS, GGSG, SGGG, GSGS, GSGSGS, GSGSGSGS, GSGSGSGSGS, GSGSGSGSGSGS, GGSGGS, GGSGGSGGS, GGSGGSGGSGGS, GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG and GGGGGGGGGGGGGGGG; the second linker and the optional third linker is a glycine spacer; the second linker and the optional third linker independently consist of 4 to 30, 4 to 20, 8 to 30, 8 to 20, 12 to 20 or 12 to 30 glycine residues; the second linker and the optional third linker consist of 20 glycine residues; at least one of the Fc domain monomers comprises a single amino acid mutation at Kabat position I253 each amino acid mutation at Kabat position I253 is independently selected from the group consisting of I253A. I253C, I253D, I253E, I253F, I253G, I253H, I253I, I253K, I253L, I253M, I253N, I253P, I253Q, I253R, I253S, I253T, I253V, I253W, and I253Y; each amino acid mutation at position I253 is I253A; at least one of the Fc domain monomers comprises a single amino acid mutation at Kabat position R292; each amino acid mutation at Kabat position R292 is independently selected from the group consisting of R292D, R292E, R292L, R292P, R292Q. R292R, R292T, and R292Y; each amino acid mutation at position R292 is R292P; each Fc domain monomer independently comprises or consists of an amino acid sequence selected from the group consisting of EPKSCDKTHTCPPCPAPELL and DKTHTCPPCPAPELL; the hinge portion of the second Fc domain monomer and the third Fc domain monomer have the amino acid sequence DKTHTCPPCPAPELL; the hinge portion of the first Fc domain monomer has the amino acid sequence EPKSCDKTHTCPPCPAPEL; the hinge portion of the first Fc domain monomer has the amino acid sequence EPKSCDKTHTCPPCPAPEL and the hinge portion of the second Fc domain monomer and the third Fc domain monomer have the amino acid sequence DKTHTCPPCPAPELL; the CH2 domains of each Fc domain monomer independently comprise the amino acid sequence: GGPSVFLFPPKPKDTLMISRTPEVTCVVVDVSHEDPEVKFNWYVDGVEVH NAKTKPREEQYNSTYRWS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK with no more than two single amino acid substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAK; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 10 single amino acid substitutions; the CH3 domains of each Fc domain monomer independently comprise the amino acid sequence: GQPREPQVYTLPPSRDELTKNQVSLTCLVKGFYPSDIAVEWESNGQPEN NYKTTPPVLDSDGSFFLYSK LTVDKSRWQQGNVFSCSVMHEALHNHYTQKSLSLSPG with no more than 8 single amino acid substitutions; the CH3 domains of each Fc domain monomer independently comprise the amino acid sequence: GQPREPQVYTLPPSRDELTKNQVSLTCLVKGFYPSDIAVEWESNGQPEN NYKTTPPVLDSDGSFFLYSK LTVDKSRWQQGNVFSCSVMHEALHNHYTQKSLSLSPG with no more than 6 single amino acid substitutions; wherein the CH3 domains of each Fc domain monomer independently comprise the amino acid sequence: GQPREPQVYTLPPSRDELTKNQVSLTCLVKGFYPSDIAVEWESNGQPEN NYKTTPPVLDSDGSFFLYSK LTVDKSRWQQGNVFSCSVMHEALHNHYTQKSLSLSPG with no more than 5 single amino acid substitutions; the single amino acid substitutions are selected from the group consisting of: T366Y, T366W, T394W, T394Y, F405W, F405A. Y407A, S354C, Y349T, T394F, K409D, K409E, K392D, K392E, K370D, K370E, D399K, D399R, E357K, E357R, D356K, and D356K; each of the Fc domain monomers independently comprises the amino acid sequence of any of SEQ ID NOs:42, 43, 45, and 45 having up to single amino acid substitutions; up to 6 of the single amino acid substitutions are reverse charge mutations in the CH3 domain or are mutations forming an engineered protuberance; the single amino acid substitutions are within the sequence from Kabat position G341 to Kabat position K447, inclusive; at least one of the mutations forming an engineered protuberance is selected from the group consisting of T366Y, T366W, T394W, T394Y, F405W, F405A, Y407A, S354C, Y349T, and T394F; the two or four reverse charge mutations are selected from: K409D, K409E, K392D, K392E, K370D, K370E, D399K, D399R, E357K, E357R, D356K, and D356K.

In a forty fourth aspect the disclosure features a polypeptide comprising: a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain, wherein at least one Fc domain monomer comprises one, two or three reverse charge amino acid mutations.

In various embodiments of the forty fourth aspect: the polypeptide further comprises an antibody heavy chain variable domain and CH1 domain amino terminal to the first IgG1 Fc domain monomer or scFv amino terminal to the first IgG1 Fc domain monomer; the first IgG1 Fc domain monomer comprises a set of two reverse charge mutations selected from those in Table 5 or a set of four reverse charge mutation selected from those in Table 6 and the second IgG1 Fc domain monomer comprises one, two or three reverse charge amino acid mutations selected from Table 4; the first IgG1 Fc domain monomer comprises one, two or three reverse charge amino acid mutations selected from Table 4 and the second IgG1 Fc domain monomer comprises a set of two reverse charge mutations selected from those in Table 5 or a set of four reverse charge mutation selected from those in Table 6; both the first IgG1 Fc domain monomer and the second IgG constant domain monomer comprise one, two or three reverse charge amino acid mutations selected from Table 4; the polypeptide further comprises a third linker and a third IgG1 Fc domain monomer wherein the first IgG1 Fc domain monomer, the second IgG1 Fc domain monomer and the third IgG1 Fc domain monomer each comprise one, two or three reverse charge amino acid mutations selected from Table 4; the polypeptide further comprises a third linker and a third IgG1 Fc domain monomer wherein both the first IgG1 Fc domain monomer and the second IgG1 Fc domain monomer each comprise one, two or three reverse charge amino acid mutations selected from Table 4 and the third IgG1 Fc domain monomer comprises a set of two reverse charge mutations selected from those in Table 5 or a set of four reverse charge mutation selected from those in Table 6; the polypeptide further comprises a third linker and third IgG1 Fc domain monomer wherein both the first IgG1 Fc domain monomer and the third IgG1 Fc domain monomer each comprise one, two or three reverse charge amino acid mutations selected from Table 4 and the second IgG1 domain monomer comprises a set of two reverse charge mutations selected from those in Table 5 or a set of four reverse charge mutation selected from those in Table 6; the polypeptide further comprises a third linker and a third IgG1 Fc domain monomer wherein both the second IgG1 Fc domain monomer and the third IgG1 Fc domain monomer each comprise one, two or three reverse charge amino acid mutations selected from Table 4 and the first IgG1 domain monomer comprises a set of two reverse charge mutations selected from those in Table 5 or a set of four reverse charge mutation selected from those in Table 6; the IgG1 Fc domain monomers comprising one, two or three reverse charge amino acid mutations selected from Table 4 have identical CH3 domains; one, two or three reverse charge amino acid mutations selected from Table 4 are in the CH3 domain; the mutations are within the sequence from Kabat position G341 to Kabat position K447, inclusive; the mutations are each single amino acid changes; the mutations are within the sequence from Kabat position G341 to Kabat position K446, inclusive; the mutations are single amino acid changes; the second linker and the optional third linker comprise or consist of an amino acid sequence selected from the group consisting of: GGGGGGGGGGGGGGGGGGGG, GGGGS, GGSG, SGGG, GSGS, GSGSGS, GSGSGSGS, GSGSGSGSGS, GSGSGSGSGSGS, GGSGGS, GGSGGSGGS, GGSGGSGGSGGS, GGSG, GGSG, GGSGGGSG, GGSGGGSGGGSGGGGGSGGGGSGGGGSGGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGSGGGSGGGS, SGGGSGGGSGGGSGGGSGGG, GGSGGGSGGGSGGGSGGS, GGGG, GGGGGGGG, GGGGGGGGGGGG and GGGGGGGGGGGGGGGG; the second linker and the optional third linker is a glycine spacer; the second linker and the optional third linker independently consist of 4 to 30, 4 to 20, 8 to 30, 8 to 20, 12 to 20 or 12 to 30 glycine residues; the second linker and the optional third linker consist of 20 glycine residues; at least one of the Fc domain monomers comprises a single amino acid mutation at Kabat position I253 each amino acid mutation at Kabat position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y: each amino acid mutation at position I253 is I253A; at least one of the Fc domain monomers comprises a single amino acid mutation at Kabat position R292; each amino acid mutation at Kabat position R292 is independently selected from the group consisting of R292D, R292E, R292L, R292P, R292Q, R292R, R292T, and R292Y; each amino acid mutation at position R292 is R292P; each Fc domain monomer independently comprises or consists of an amino acid sequence selected from the group consisting of EPKSCDKTHTCPPCPAPELL and DKTHTCPPCPAPELL; the hinge portion of the second Fc domain monomer and the third Fc domain monomer have the amino acid sequence DKTHTCPPCPAPELL; the hinge portion of the first Fc domain monomer has the amino acid sequence EPKSCDKTHTCPPCPAPEL; the hinge portion of the first Fc domain monomer has the amino acid sequence EPKSCDKTHTCPPCPAPEL and the hinge portion of the second Fc domain monomer and the third Fc domain monomer have the amino acid sequence DKTHTCPPCPAPELL: the CH2 domains of each Fc domain monomer independently comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVVDVSHEDPEVKFNWYVDGVEVH NAKTKPREEQYNSTYRWS VLTVLHQDWLNGKEYKCKVSNKALPAPIEKTISKAK with no more than two single amino acid deletions or substitutions; the CH2 domains of each Fc domain monomer are identical and comprise the amino acid sequence: GGPSVFLFPPKPKDTLMISRTPEVTCVVVDVSHEDPEVKFNWYVDGVEVH NAKTKPREEQYNSTYRWS VLTVLHQDWLNGKEYKCKVSNKALPAPIEKTISKAK with no more than two single amino acid substitutions; the CH2 domains of each Fc domain monomer are identical and comprise the amino acid sequence: GGPSVFLFPPKPKDTLMISRTPEVTCVVVDVSHEDPEVKFNWYVDGVEVHNAKTKPREEQYNSTYRWS VLTVLHQDWLNGKEYKCKVSNKALPAPIEKTISKAK; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 10 single amino acid substitutions; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 8 single amino acid substitutions; the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 6 single amino acid substitutions; wherein the CH3 domains of each Fc domain monomer independently comprise the amino acid sequence: GQPREPQVYTLPPSRDELTKNQVSLTCLVKGFYPSDIAVEWESNGQPENNYKTTPPVLDSDGSFFLYSK LTVDKSRWQQGNVFSCSVMHEALHNHYTQKSLSLSPG with no more than 5 single amino acid substitutions; the single amino acid substitutions are selected from the group consisting of: T366Y, T366W, T394W, T394Y, F405W, F405A, Y407A, S354C, Y349T, T394F. K409D, K409E, K392D, K392E, K370D, K370E, D399K, D399R, E357K, E357R, D356K, and D356K; each of the Fc domain monomers independently comprises the amino acid sequence of any of SEQ ID NOs:42, 43, 45, and 45 having up to 10 single amino acid substitutions; up to 6 of the single amino acid substitutions are reverse charge mutations in the CH3 domain or are mutations forming an engineered protuberance; the single amino acid substitutions are within the sequence from Kabat position G341 to Kabat position K447, inclusive; the VH domain or scFv comprises a set of CDR-H1, CDR-H2 and CDR-H3 sequences set forth in Table 1; the VH domain or scFv comprises CDR-H1, CDR-H2, and CDR-H3 of a VH domain comprising a sequence of an antibody set forth in Table 2; the VH domain or scFv comprises CDR-H1, CDR-H2, and CDR-H3 of a VH sequence of an antibody set forth in Table 2, and the VH sequence, excluding the CDR-H1, CDR-H2, and CDR-H3 sequence, is at least 95% or 98% identical to the VH sequence of an antibody set forth in Table 2; the VH domain or scFv comprises a VH sequence of an antibody set forth in Table 2; the VH domain or scFv comprises a set of CDR-H1, CDR-H2, CDR-H3, CDR-L1, CDR-L2, and CDR-L3 sequences set forth in Table 1; the VH domain or scFv comprises CDR-H1, CDR-H2, CDR-H3, CDR-L1, CDR-L2, and CDR-L3 sequences from a set of a VH and a VL sequence of an antibody set forth in Table 2; the VH domain or scFv main comprises a VH domain comprising CDR-H1, CDR-H2, and CDR-H3 of a VH sequence of an antibody set forth in Table 2, and a VL domain comprising CDR-L1, CDR-L2, and CDR-L3 of a VL sequence of an antibody set forth in Table 2, wherein the VH and the VL domain sequences, excluding the CDR-H1, CDR-H2, CDR-H3, CDR-L1, CDR-L2, and CDR-L3 sequences, are at least 95% or 98% identical to the VH and VL sequences of an antibody set forth in Table 2; the VH domain or scFv comprises a set of a VH and a VL sequence of an antibody set forth in Table 2.

Also describes is a nucleic acid molecule encoding any of the forgoing polypeptides of the forty first, forty second, forty third and forty fourth aspects.

Also described is: an expression vector that includes a nucleic acid encoding any of the forgoing polypeptide; host cells containing the nucleic acids or expression vectors; host cells further containing a nucleic acid molecule encoding a polypeptide comprising an antibody VL domain (e.g., a nucleic acid molecule encoding a polypeptide comprising an antibody VL domain and an antibody CL domain); a host cell further containing a nucleic acid molecule encoding a polypeptide comprising an antibody VL domain and an antibody CL domain; a host cells further containing a nucleic acid molecule encoding a polypeptide comprising an IgG1 Fc domain monomer having no more than 10 single amino acid mutations; a host cell further containing a nucleic acid molecule encoding a polypeptide comprising IgG1 Fc domain monomer having no more than 10 single amino acid mutations. In various embodiments: the IgG1 Fc domain monomer comprises the amino acid sequence of any of SEQ ID Nos; 42, 43, 45 and 47 having no more than 10, 8, 6 or 4 single amino acid mutations in the CH3 domain.

Also described is a pharmaceutical composition comprising any of the polypeptide or polypeptide complexes described herein. In various embodiments less than 40%, 30%, 20%, 10%, 5%, 2% of the polypeptides have at least one fucose.

The polypeptides of the of forty first, forty second, forty third and forty fourth aspects of the disclosure are useful as components of the various Fc-antigen binding domain constructs described herein. Thus, the polypeptides of any of the first through fortieth aspects, e.g., those can comprise an antigen binding domain, can comprise or consist of the polypeptides of any of forty first, forty second, forty third and forty fourth aspects of the disclosure.

Other useful polypeptides for use in all aspects of the disclosure include polypeptides comprising an Fc domain monomer (e.g., comprising or consisting of the amino acid sequence of any of SEQ ID Nos: 42, 43, 45 and 47 with no more than 8, 6, 5, 4, or 3 single amino acid substitutions) having one, two or three mutations forming a cavity (e.g., selected from: Y407T Y407A, F405A, T394S, T394W:Y407T, T394S:Y407A, T366W:T394S, F405T, T366S:L368A:Y407V:Y349C, S364H:F405A). These polypeptides can optionally include one, two or three reverse charge mutations from Table 4 or Table 5.

### Definitions

As used herein, the term “Fc domain monomer” refers to a polypeptide chain that includes at least a hinge domain and second and third antibody constant domains (C_{H}2 and C_{H}3) or functional fragments thereof (e.g., at least a hinge domain or functional fragment thereof, a CH2 domain or functional fragment thereof, and a CH3 domain or functional fragment thereof) (e.g., fragments that that capable of (i) dimerizing with another Fc domain monomer to form an Fc domain, and (ii) binding to an Fc receptor). The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., IgG1). The human IgG1 Fc domain monomer is used in the examples described herein. The hinge domain of human IgG1 extends from Kabat D221 to L235, the CH2 domain extends from G236 to K340 and the CH3 domain extends from G341 to K447. In many examples herein the CH3 domain does not include K347. Thus, a CH3 domain can be from G341 to G446. In many examples herein a hinge domain can include E216 to L235. This is true, for example, when the hinge is amino terminal to a CH1 domain or an antigen binding domain. In some case, for example when the hinge is at the amino terminus of a polypeptide, the Asp at Kabat 221 is mutated to Gln. An Fc domain monomer does not include any portion of an immunoglobulin that is capable of acting as an antigen-recognition region, e.g., a variable domain or a complementarity determining region (CDR). Fc domain monomers can contain as many as ten changes from a wild-type Fc domain monomer sequence (e.g., 1-10, 1-8, 1-86, 1-4 amino acid substitutions, additions, or deletions) that alter the interaction between an Fc domain and an Fc receptor. Fc domain monomers can contain as many as ten changes (e.g., single amino acid changes) from a wild-type Fc domain monomer sequence (e.g., 1-10, 1-8, 1-6, 1-4 amino acid substitutions, additions, or deletions) that alter the interaction between Fc domain monomers. In certain embodiments there are up to 10, 8, 6 or 5 single amino acid substitution on the CH3 domain compared to the human IgG1 CH3 domain sequence: GQPREPQVYTLPPSRDELTKNQVSLTCLVKGFYPSDIAVEWESNGQPENNYKTTPPVLDSDGSFFLYSKLTVDKSRWQQGNVFSCSV MHEALHNHYTQKSLSLSPG Examples of suitable changes are known in the art.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, as well as one or more disulfide bonds that form between the hinge domains of the two dimerizing Fc domain monomers.

In the present disclosure, the term “Fc-antigen binding domain construct” refers to associated polypeptide chains forming at least two Fc domains as described herein and including at least one “antigen binding domain.” Fc-antigen binding domain constructs described herein can include Fc domain monomers that have the same or different sequences. For example, an Fc-antigen binding domain construct can have three Fc domains, two of which includes IgG1 or IgG1-derived Fc domain monomers, and a third which includes IgG2 or IgG2-derived Fc domain monomers. In another example, an Fc-antigen binding domain construct can have three Fc domains, two of which include a “protuberance-into-cavity pair” and a third which does not include a “protuberance-into-cavity pair.” An Fc domain forms the minimum structure that binds to an Fc receptor, e.g., FcγRI, FcγRIIa, FcγRIIb, FcγRIIa, FcγRIIIb, or FcγRIV.

As used herein, the term “antigen binding domain” refers to a peptide, a polypeptide, or a set of associated polypeptides that is capable of specifically binding a target molecule. In some embodiments, the “antigen binding domain” is the minimal sequence of an antibody that binds with specificity to the antigen bound by the antibody. Surface plasmon resonance (SPR) or various immunoassays known in the art, e.g., Western Blots or ELISAs, can be used to assess antibody specificity for an antigen. In some embodiments, the “antigen binding domain” includes a variable domain or a complementarity determining region (CDR) of an antibody, e.g., one or more CDRs of an antibody set forth in Table 1, one or more CDRs of an antibody set forth in Table 2, or the VH and/or VL domains of an antibody set forth in Table 2. In some embodiments, the antigen binding domain can include a VH domain and a CH1 domain, optionally with a VL domain. In other embodiments, the antigen binding domain is a Fab fragment of an antibody or a scFv. An antigen binding domain may also be a synthetically engineered peptide that binds a target specifically such as a fibronectin-based binding protein (e.g., a fibronectin type III domain (FN3) monobody).

As used herein, the term “Complementarity Determining Regions” (CDRs) refers to the amino acid residues of an antibody variable domain the presence of which are necessary for antigen binding. Each variable domain typically has three CDR regions identified as CDR-L1, CDR-L2 and CDR-L3, and CDR-H1, CDR-H2, and CDR-H3). Each complementarity determining region may include amino acid residues from a “complementarity determining region” as defined by Kabat (i.e., about residues 24-34 (CDR-L1), 50-56 (CDR-L2), and 89-97 (CDR-L3) in the light chain variable domain and 31-35 (CDR-H1), 50-65 (CDR-H2), and 95-102 (CDR-H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (CDR-L1), 50-52 (CDR-L2), and 91-96 (CDR-L3) in the light chain variable domain and 26-32 (CDR-H1), 53-55 (CDR-H2), and 96-101 (CDR-H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

“Framework regions” (hereinafter FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs include amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR includes amino acids from both a CDR as defined by Kabat and those of a hypervariable loop, the FR residues will be adjusted accordingly.

An “Fv” fragment is an antibody fragment which contains a complete antigen recognition and binding site. This region consists of a dimer of one heavy and one light chain variable domain in tight association, which can be covalent in nature, for example, in a scFv. It is in this configuration that the three CDRs of each variable domain interact to define an antigen binding site on the surface of the V_{H}-V_{L} dimer.

The “Fab” fragment contains a variable and constant domain of the light chain and a variable domain and the first constant domain (C_{H}1) of the heavy chain. F(ab′)₂ antibody fragments include a pair of Fab fragments which are generally covalently linked near their carboxy termini by hinge cysteines.

“Single-chain Fv” or “scFv” antibody fragments include the V_{H} and V_{L} domains of antibody in a single polypeptide chain. Generally, the scFv polypeptide further includes a polypeptide linker between the V_{H} and V_{L} domains, which enables the scFv to form the desired structure for antigen binding.

As used herein, the term “antibody constant domain” refers to a polypeptide that corresponds to a constant region domain of an antibody (e.g., a C_{L} antibody constant domain, a C_{H}1 antibody constant domain, a C_{H}2 antibody constant domain, or a C_{H}3 antibody constant domain).

As used herein, the term “promote” means to encourage and to favor, e.g., to favor the formation of an Fc domain from two Fc domain monomers which have higher binding affinity for each other than for other, distinct Fc domain monomers. As is described herein, two Fc domain monomers that combine to form an Fc domain can have compatible amino acid modifications (e.g., engineered protuberances and engineered cavities, and/or electrostatic steering mutations) at the interface of their respective C_{H}3 antibody constant domains. The compatible amino acid modifications promote or favor the selective interaction of such Fc domain monomers with each other relative to with other Fc domain monomers which lack such amino acid modifications or with incompatible amino acid modifications. This occurs because, due to the amino acid modifications at the interface of the two interacting C_{H}3 antibody constant domains, the Fc domain monomers to have a higher affinity toward each other than to other Fc domain monomers lacking amino acid modifications.

As used herein, the term “dimerization selectivity module” refers to a sequence of the Fc domain monomer that facilitates the favored pairing between two Fc domain monomers. “Complementary” dimerization selectivity modules are dimerization selectivity modules that promote or favor the selective interaction of two Fc domain monomers with each other. Complementary dimerization selectivity modules can have the same or different sequences. Exemplary complementary dimerization selectivity modules are described herein.

As used herein, the term “engineered cavity” refers to the substitution of at least one of the original amino acid residues in the C_{H}3 antibody constant domain with a different amino acid residue having a smaller side chain volume than the original amino acid residue, thus creating a three dimensional cavity in the CH3 antibody constant domain. The term “original amino acid residue” refers to a naturally occurring amino acid residue encoded by the genetic code of a wild-type C_{H}3 antibody constant domain.

As used herein, the term “engineered protuberance” refers to the substitution of at least one of the original amino acid residues in the C_{H}3 antibody constant domain with a different amino acid residue having a larger side chain volume than the original amino acid residue, thus creating a three dimensional protuberance in the C_{H}3 antibody constant domain. The term “original amino acid residues” refers to naturally occurring amino acid residues encoded by the genetic code of a wild-type C_{H}3 antibody constant domain.

As used herein, the term “protuberance-into-cavity pair” describes an Fc domain including two Fc domain monomers, wherein the first Fc domain monomer includes an engineered cavity in its C_{H}3 antibody constant domain, while the second Fc domain monomer includes an engineered protuberance in its C_{H}3 antibody constant domain. In a protuberance-into-cavity pair, the engineered protuberance in the C_{H}3 antibody constant domain of the first Fc domain monomer is positioned such that it interacts with the engineered cavity of the C_{H}3 antibody constant domain of the second Fc domain monomer without significantly perturbing the normal association of the dimer at the inter-C_{H}3 antibody constant domain interface.

As used herein, the term “heterodimer Fc domain” refers to an Fc domain that is formed by the heterodimerization of two Fc domain monomers, wherein the two Fc domain monomers contain different reverse charge mutations (see, e.g., mutations in Table 4) that promote the favorable formation of these two Fc domain monomers. In an Fc construct having three Fc domains—one carboxyl terminal “stem” Fc domain and two amino terminal “branch” Fc domains—each of the amino terminal “branch” Fc domains may be a heterodimeric Fc domain (also called a “branch heterodimeric Fc domain”).

As used herein, the term “structurally identical,” in reference to a population of Fc-antigen binding domain constructs, refers to constructs that are assemblies of the same polypeptide sequences in the same ratio and configuration and does not refer to any post-translational modification, such as glycosylation.

As used herein, the term “homodimeric Fc domain” refers to an Fc domain that is formed by the homodimerization of two Fc domain monomers, wherein the two Fc domain monomers contain the same reverse charge mutations (see, e.g., mutations in Tables 5 and 6). In an Fc construct having three Fc domains—one carboxyl terminal “stem” Fc domain and two amino terminal “branch” Fc domains—the carboxy terminal “stem” Fc domain may be a homodimeric Fc domain (also called a “stem homodimeric Fc domain”).

As used herein, the term “heterodimerizing selectivity module” refers to engineered protuberances, engineered cavities, and certain reverse charge amino acid substitutions that can be made in the CH3 antibody constant domains of Fc domain monomers in order to promote favorable heterodimerization of two Fc domain monomers that have compatible heterodimerizing selectivity modules. Fc domain monomers containing heterodimerizing selectivity modules may combine to form a heterodimeric Fc domain. Examples of heterodimerizing selectivity modules are shown in Tables 3 and 4.

As used herein, the term “homodimerizing selectivity module” refers to reverse charge mutations in an Fc domain monomer in at least two positions within the ring of charged residues at the interface between CH3 domains that promote homodimerization of the Fc domain monomer to form a homodimeric Fc domain. Examples of homodimerizing selectivity modules are shown in Tables 4 and 5.

As used herein, the term “joined” is used to describe the combination or attachment of two or more elements, components, or protein domains, e.g., polypeptides, by means including chemical conjugation, recombinant means, and chemical bonds, e.g., peptide bonds, disulfide bonds and amide bonds. For example, two single polypeptides can be joined to form one contiguous protein structure through chemical conjugation, a chemical bond, a peptide linker, or any other means of covalent linkage. In some embodiments, an antigen binding domain is joined to a Fc domain monomer by being expressed from a contiguous nucleic acid sequence encoding both the antigen binding domain and the Fc domain monomer. In other embodiments, an antigen binding domain is joined to a Fc domain monomer by way of a peptide linker, wherein the N-terminus of the peptide linker is joined to the C-terminus of the antigen binding domain through a chemical bond, e.g., a peptide bond, and the C-terminus of the peptide linker is joined to the N-terminus of the Fc domain monomer through a chemical bond, e.g., a peptide bond.

As used herein, the term “associated” is used to describe the interaction, e.g., hydrogen bonding, hydrophobic interaction, or ionic interaction, between polypeptides (or sequences within one single polypeptide) such that the polypeptides (or sequences within one single polypeptide) are positioned to form an Fc-antigen binding domain construct described herein (e.g., an Fc-antigen binding domain construct having three Fc domains). For example, in some embodiments, four polypeptides, e.g., two polypeptides each including two Fc domain monomers and two polypeptides each including one Fc domain monomer, associate to form an Fc construct that has three Fc domains (e.g., as depicted in FIGS. 50 and 51). The four polypeptides can associate through their respective Fc domain monomers. The association between polypeptides does not include covalent interactions.

As used herein, the term “linker” refers to a linkage between two elements, e.g., protein domains. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 3-200 amino acid, 3-150 amino acid, or 3-100 amino acid sequence) occurring between two polypeptides or polypeptide domains to provide space and/or flexibility between the two polypeptides or polypeptide domains. An amino acid spacer is part of the primary sequence of a polypeptide (e.g., joined to the spaced polypeptides or polypeptide domains via the polypeptide backbone). The formation of disulfide bonds, e.g., between two hinge regions or two Fc domain monomers that form an Fc domain, is not considered a linker.

As used herein, the term “glycine spacer” refers to a linker containing only glycines that joins two Fc domain monomers in tandem series. A glycine spacer may contain at least 4, 8, or 12 glycines (e.g., 4-30, 8-30, or 12-30 glycines; e.g., 12-30, 4, 5, 6, 7, 8, 9, 10, 11, 12, 13, 14, 15, 16, 17, 18, 19, 20, 21, 22, 23, 24, 25, 26, 27, 28, 29, or 30 glycines). In some embodiments, a glycine spacer has the sequence of GGGGGGGGGGGGGGGGGGGG (SEQ ID NO: 27).

As used herein, the term “albumin-binding peptide” refers to an amino acid sequence of 12 to 16 amino acids that has affinity for and functions to bind serum albumin. An albumin-binding peptide can be of different origins, e.g., human, mouse, or rat. In some embodiments of the present disclosure, an albumin-binding peptide is fused to the C-terminus of an Fc domain monomer to increase the serum half-life of the Fc-antigen binding domain construct. An albumin-binding peptide can be fused, either directly or through a linker, to the N- or C-terminus of an Fc domain monomer.

As used herein, the term “purification peptide” refers to a peptide of any length that can be used for purification, isolation, or identification of a polypeptide. A purification peptide may be joined to a polypeptide to aid in purifying the polypeptide and/or isolating the polypeptide from, e.g., a cell lysate mixture. In some embodiments, the purification peptide binds to another moiety that has a specific affinity for the purification peptide. In some embodiments, such moieties which specifically bind to the purification peptide are attached to a solid support, such as a matrix, a resin, or agarose beads. Examples of purification peptides that may be joined to an Fc-antigen binding domain construct are described in detail further herein.

As used herein, the term “multimer” refers to a molecule including at least two associated Fc constructs or Fc-antigen binding domain constructs described herein.

As used herein, the term “polynucleotide” refers to an oligonucleotide, or nucleotide, and fragments or portions thereof, and to DNA or RNA of genomic or synthetic origin, which may be single- or double-stranded, and represent the sense or anti-sense strand. A single polynucleotide is translated into a single polypeptide.

As used herein, the term “polypeptide” describes a single polymer in which the monomers are amino acid residues which are joined together through amide bonds. A polypeptide is intended to encompass any amino acid sequence, either naturally occurring, recombinant, or synthetically produced.

As used herein, the term “amino acid positions” refers to the position numbers of amino acids in a protein or protein domain. The amino acid positions for antibody or Fc-antigen binding domain constructs are numbered using the Kabat numbering system (Kabat et al., Sequences of Proteins of Immunological Interest, National Institutes of Health, Bethesda, Md., ed 5, 1991).

FIGS. 53A-53D depict human IgG1 Fc domains numbered using the Kabat numbering system.

As used herein, the term “amino acid modification” or refers to an alteration of an Fc domain polypeptide sequence that, compared with a reference sequence (e.g., a wild-type, unmutated, or unmodified Fc sequence) may have an effect on the pharmacokinetics (PK) and/or pharmacodynamics (PD) properties, serum half-life, effector functions (e.g., cell lysis (e.g., antibody-dependent cell-mediated toxicity (ADCC) and/or complement dependent cytotoxicity activity (CDC)), phagocytosis (e.g., antibody dependent cellular phagocytosis (ADCP) and/or complement-dependent cellular cytotoxicity (CDCC)), immune activation, and T-cell activation), affinity for Fc receptors (e.g., Fc-gamma receptors (FcγR) (e.g., FcγRI (CD64), FcγRIIa (CD32), FcγRIIb (CD32), FcγRIIa (CD16a), and/or FcγRIIIb (CD16b)), Fc-alpha receptors (FcαR), Fc-epsilon receptors (FcεR), and/or to the neonatal Fc receptor (FcRn)), affinity for proteins involved in the compliment cascade (e.g., C1q), post-translational modifications (e.g., glycosylation, sialylation), aggregation properties (e.g., the ability to form dimers (e.g., homo- and/or heterodimers) and/or multimers), and the biophysical properties (e.g., alters the interaction between C_{H}1 and C_{L}, alters stability, and/or alters sensitivity to temperature and/or pH) of an Fc construct, and may promote improved efficacy of treatment of immunological and inflammatory diseases. An amino acid modification includes amino acid substitutions, deletions, and/or insertions. In some embodiments, an amino acid modification is the modification of a single amino acid. In other embodiment, the amino acid modification is the modification of multiple (e.g., more than one) amino acids. The amino acid modification may include a combination of amino acid substitutions, deletions, and/or insertions. Included in the description of amino acid modifications, are genetic (i.e., DNA and RNA) alterations such as point mutations (e.g., the exchange of a single nucleotide for another), insertions and deletions (e.g., the addition and/or removal of one or more nucleotides) of the nucleotide sequence that codes for an Fc polypeptide.

In certain embodiments, at least one (e.g., one, two, or three) Fc domain within an Fc construct or Fc-antigen binding domain construct includes an amino acid modification. In some instances, the at least one Fc domain includes one or more (e.g., two, three, four, five, six, seven, eight, nine, ten, or twenty or more) amino acid modifications.

As used herein, the term “percent (%) identity” refers to the percentage of amino acid (or nucleic acid) residues of a candidate sequence, e.g., the sequence of an Fc domain monomer in an Fc-antigen binding domain construct described herein, that are identical to the amino acid (or nucleic acid) residues of a reference sequence, e.g., the sequence of a wild-type Fc domain monomer, after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity (e.g., 50% to 100%, 60% to 100%, 70% to 100%, 80% to 100%, 90% to 100%, 92% to 100%, 95% to 100%, 97% to 100%, 99% to 100%, or 99.5% to 100% identity), across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

In some embodiments, an Fc domain monomer in an Fc construct described herein (e.g., an Fc-antigen binding domain construct having three Fc domains) may have a sequence that is at least 95% identical (at least 97%, 99%, or 99.5% identical) to the sequence of a wild-type Fc domain monomer (e.g., SEQ ID NO: 42). In some embodiments, an Fc domain monomer in an Fc construct described herein (e.g., an Fc-antigen binding domain construct having three Fc domains) may have a sequence that is at least 95% identical (at least 97%, 99%, or 99.5% identical) to the sequence of any one of SEQ ID NOs: 44, 46, 48, and 50-53. In certain embodiments, an Fc domain monomer in the Fc construct may have a sequence that is at least 95% identical (at least 97%, 99%, or 99.5% identical) to the sequence of SEQ ID NO: 48, 52, and 53.

In some embodiments, a spacer between two Fc domain monomers may have a sequence that is at least 75% identical (at least 75%, 77%, 79%, 81%, 83%, 85%, 87%, 89%, 91%, 93%, 95%, 97%, 99%, 99.5%, or 100% identical) to the sequence of any one of SEQ ID NOs: 1-36 (e.g., SEQ ID NOs: 17, 18, 26, and 27) described further herein.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell). As described herein, a host cell is used to express one or more polypeptides encoding desired domains which can then combine to form a desired Fc-antigen binding domain construct.

As used herein, the term “pharmaceutical composition” refers to a medicinal or pharmaceutical formulation that contains an active ingredient as well as one or more excipients and diluents to enable the active ingredient to be suitable for the method of administration. The pharmaceutical composition of the present disclosure includes pharmaceutically acceptable components that are compatible with the Fc-antigen binding domain construct. The pharmaceutical composition is typically in aqueous form for intravenous or subcutaneous administration.

As used herein, a “substantially homogenous population” of polypeptides or of an Fc construct is one in which at least 50% of the polypeptides or Fc constructs in a composition (e.g., a cell culture medium or a pharmaceutical composition) have the same number of Fc domains, as determined by non-reducing SDS gel electrophoresis or size exclusion chromatography. A substantially homogenous population of polypeptides or of an Fc construct may be obtained prior to purification, or after Protein A or Protein G purification, or after any Fab or Fc-specific affinity chromatography only. In various embodiments, at least 55%, 60%, 65%, 70%, 75%, 80%, or 85% of the polypeptides or Fc constructs in the composition have the same number of Fc domains. In other embodiments, up to 85%, 90%, 92%, or 95% of the polypeptides or Fc constructs in the composition have the same number of Fc domains.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient. In the present disclosure, the pharmaceutically acceptable carrier must provide adequate pharmaceutical stability to the Fc-antigen binding domain construct. The nature of the carrier differs with the mode of administration. For example, for oral administration, a solid carrier is preferred; for intravenous administration, an aqueous solution carrier (e.g., WFI, and/or a buffered solution) is generally used.

As used herein, “therapeutically effective amount” refers to an amount, e.g., pharmaceutical dose, effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents.

As used herein, the term fragment and the term portion can be used interchangeably.

### BRIEF DESCRIPTION OF THE DRAWINGS

FIG. 1 is an illustration of an Fc-antigen binding domain construct (construct 1) containing two Fc domains and an antigen binding domain. Each Fc domain is a dimer of two Fc domain monomers. Two of the Fc domain monomers (106 and 108) contain a protuberance in its C_{H}3 antibody constant domain, while the other two Fc domain monomers (112 and 114) contain a cavity in the juxtaposed position in its C_{H}3 antibody constant domain. The construct is formed from three Fc domain monomer containing polypeptides. The first polypeptide (102) contains two protuberance-containing Fc domain monomers (106 and 108) linked by a spacer in a tandem series to an antigen binding domain containing a V_{H} domain (110) on the N-terminus. A VL containing domain (104) is joined to the V_{H} domain. Each of the second and third polypeptides (112 and 114) contains a cavity-containing Fc domain monomer.

FIG. 2 is an illustration of an Fc-antigen binding domain construct (construct 2) containing three Fc domains and an antigen binding domain. The construct is formed from four Fc domain monomer containing polypeptides. The first polypeptide (202) contains three protuberance-containing Fc domains (206, 208, and 210) linked by spacers in a tandem series to an antigen binding domain containing a V_{H} domain (212) on the N-terminus. A V_{L} containing domain (204) is joined to the V_{H} domain. Each of the second, third, and fourth polypeptides (214, 216, and 218) contains a cavity-containing Fc domain monomer.

FIG. 3 is an illustration of an Fc-antigen binding domain construct (construct 3) containing two Fc domains and two antigen binding domains. The construct is formed from three Fc domain monomer containing polypeptides. The first polypeptide (302) contains two protuberance-containing Fc domain monomers (304 and 306) linked by a spacer in a tandem series. Each of the second and third polypeptides (320 and 322) contains a cavity-containing Fc domain monomer (310 and 314) joined in tandem to an antigen binding domain containing a V_{H} domain (316 and 318) on the N-terminus. A V_{L} containing domain (308 and 312) is joined to each V_{H} domain.

FIG. 4 is an illustration of an Fc-antigen binding domain construct (construct 4) containing three Fc domains and three antigen binding domains. The construct is formed from four Fc domain monomer containing polypeptides. The first polypeptide (402) contains three protuberance-containing Fc domain monomers (404, 406, and 408) linked by spacers in a tandem series. Each of the second, third, and fourth polypeptides (428, 430, and 432) contains a cavity-containing Fc domain monomer (426, 420, and 414) joined in tandem to an antigen binding domain containing a V_{H} domain (422, 416, and 410) on the N-terminus. A V_{L} containing domain (424, 418, and 412) is joined to each V_{H} domain.

FIG. 5 is an illustration of an Fc-antigen binding domain construct (construct 5) containing two Fc domains and three antigen binding domains. The construct is formed from three Fc domain monomer containing polypeptides. The first polypeptide (502) contains two protuberance-containing Fc domain monomers (508 and 506) linked by a spacer in a tandem series with an antigen binding domain containing a V_{H} domain (510) at the N-terminus. Each of the second and third polypeptides (524 and 526) contains a cavity-containing Fc domain monomer (516 and 522) joined in tandem to an antigen binding domain containing a V_{H} domain (512 and 518) on the N-terminus. A V_{L} containing domain (504, 514, and 520) is joined to each V_{H} domain.

FIG. 6 is an illustration of an Fc-antigen binding domain construct (construct 6) containing three Fc domains and four antigen binding domains. The construct is formed from four Fc monomer containing polypeptides. The first polypeptide (602) contains three protuberance-containing Fc domain monomers (606, 608, and 610) linked by spacers in a tandem series with an antigen binding domain containing a VH domain (612) at the N-terminus. Each of the second, third, and fourth polypeptides (632, 634, and 636) contains a cavity-containing Fc domain monomer (618, 624, and 630) joined in tandem to an antigen binding domain containing a V_{H} domain (616, 622, and 628) on the N-terminus. A V_{L} containing domain (604, 616, 622, and 628) is joined to each V_{H} domain.

FIG. 7 is an illustration of an Fc-antigen binding domain construct (construct 7) containing three Fc domains and two antigen binding domains. This Fc-antigen binding domain construct contains a dimer of two Fc domain monomers (706 and 718), wherein both Fc domain monomers contain different charged amino acids at their C_{H}3-C_{H}3 interface than the WT sequence to promote favorable electrostatic interactions between the two Fc domain monomers. The construct is formed from four Fc domain monomer containing polypeptides. Two polypeptides (702 and 724) each contain a protuberance-containing Fc domain monomer (710 and 720) linked by a spacer in a tandem series to an Fc domain monomer containing different charged amino acids at the C_{H}3-C_{H}3 interface than the WT sequence (706 and 718) and an antigen binding domain containing a V_{H} domain (712 and 714) on the N-terminus. The third and fourth polypeptides (708 and 722) each contain a cavity-containing Fc domain monomer. A V_{L} containing domain (704 and 716) is joined to each V_{H} domain.

FIG. 8 is an illustration of an Fc-antigen binding domain construct (construct 8) containing three Fc domains and two antigen binding domains. The construct is formed of four Fc domain monomer containing polypeptides. Two polypeptides (802 and 828) each contain a protuberance-containing Fc domain monomer (814 and 820) linked by a spacer in a tandem series to an Fc domain monomer containing different charged amino acids at the C_{H}3-C_{H}3 interface than the WT sequence (810 and 816). The third and fourth polypeptides (804 and 826) each contain a cavity-containing Fc domain monomer (808 and 824) joined in tandem to an antigen binding domain containing a V_{H} domain (812 and 818) at the N-terminus. A V_{L} containing domain (806 and 822) is joined to each V_{H} domain.

FIG. 9 is an illustration of an Fc-antigen binding domain construct (construct 9) containing three Fc domains and four antigen binding domains. The construct is formed of four Fc domain monomer containing polypeptides. Two polypeptides (902 and 936) each contain a protuberance-containing Fc domain monomer (918 and 928) linked by a spacer in a tandem series to an Fc domain monomer containing different charged amino acids at the C_{H}3-C_{H}3 interface than the WT sequence (910 and 924) and an antigen binding domain containing a V_{H} domain (908 and 920) at the N-terminus. The third and fourth polypeptides (904 and 934) contain a cavity-containing Fc domain monomer (916 and 932) joined in a tandem series to an antigen binding domain containing a V_{H} domain (912 and 926) at the N-terminus. A V_{L} containing domain (906, 914, 922, and 930) is joined to each V_{H} domain.

FIG. 10 is an illustration of an Fc-antigen binding domain construct (construct 10) containing five Fc domains and two antigen binding domains. The construct is formed of six Fc domain monomer containing polypeptides. Two polypeptides (1002 and 1032) each contain a protuberance-containing Fc domain monomer (1016 and 1030) linked by spacers in a tandem series to another protuberance-containing Fc domain monomer (1014 and 1028), an Fc domain monomer containing different charged amino acids at the C_{H}3-C_{H}3 interface than the WT sequence (1008 and 1022) and an antigen binding domain containing a V_{H} domain (1006 and 1018) at the N-terminus. The third, fourth, fifth, and sixth polypeptides (1012, 1010, 1026, and 1024) each contain a cavity-containing Fc domain monomer. A V_{L} containing domain (1004 and 1020) is joined to each V_{H} domain.

FIG. 11 is an illustration of an Fc-antigen binding domain construct (construct 11) containing five Fc domains and four antigen binding domains. The construct is formed of six Fc domain monomer containing polypeptides. Two polypeptides (1102 and 1148) contain a protuberance-containing Fc domain monomer (1118 and 1132) linked by spacers in a tandem series to another protuberance-containing Fc domain monomer (1120 and 1130) and an Fc domain monomer containing different charged amino acids at the C_{H}3-C_{H}3 interface than the WT sequence (1124 and 1126). The third, fourth, fifth, and sixth polypeptides (1106, 1104, 1144, and 1146) each contain a cavity-containing Fc domain monomer (1116, 1110, 1134, and 1140) joined in a tandem series to an antigen binding domain containing a V_{H} domain (1112, 1122, 1138, and 1128) at the N-terminus. A V_{L} containing domain (1108, 1114, 1135, and 1142) is joined to each V_{H} domain.

FIG. 12 is an illustration of an Fc-antigen binding domain construct (construct 12) containing five Fc domains and six antigen binding domains. The construct is formed of six Fc domain monomer containing polypeptides. Two polypeptides (1202 and 1256) contain a protuberance-containing Fc domain monomer (1224 and 1230) linked by spacers in a tandem series to another protuberance-containing Fc domain monomer (1226 and 1228), an Fc domain monomer containing different charged amino acids at the C_{H}3-C_{H}3 interface than the WT sequence (1210 and 1244), and an antigen binding domain containing a V_{H} domain (1250 and 1248) at the N-terminus. The third, fourth, fifth, and sixth polypeptides (1206, 1204, 1254, and 1252) each contain a cavity-containing Fc domain monomer (1222, 1216, 1232, and 1238) joined in a tandem series to an antigen binding domain containing a V_{H} domain (1218, 1212, 1236, and 1242) at the N-terminus. A V_{L} containing domain (1208, 1214, 1220, 1234, 1240, and 1246) is joined to each V_{H} domain.

FIG. 13 is an illustration of an Fc-antigen binding domain construct (construct 13) containing three Fc domains and two antigen binding domains. The construct is formed of four Fc domain monomer containing polypeptides. Two polypeptides (1302 and 1324) contain an Fc domain monomer containing different charged amino acids at the C_{H}3-C_{H}3 interface than the WT sequence (1308 and 1318) linked by a spacer in a tandem series to a protuberance-containing Fc domain monomer (1312 and 1316) and an antigen binding domain containing a V_{H} domain (1310 and 1314) at the N-terminus. The third and fourth polypeptides (1306 and 1320) contain a cavity-containing Fc domain monomer. A V_{L} containing domain (1304 and 1322) is joined to each V_{H} domain.

FIG. 14 is an illustration of an Fc-antigen binding domain construct (construct 14) containing three Fc domains and two antigen binding domains. The construct is formed of four Fc domain monomer containing polypeptides. Two polypeptides (1404 and 1426) contain an Fc domain monomer containing different charged amino acids at the C_{H}3-C_{H}3 interface than the WT sequence (1308 and 1318) linked by a spacer in a tandem series to a protuberance-containing Fc domain monomer (1414 and 1418). The third and fourth polypeptides (1402 and 1428) each contain a cavity-containing Fc domain monomer (1410 and 1422) joined in a tandem series to an antigen binding domain containing a V_{H} domain (1408 and 1416) at the N-terminus. A V_{L} containing domain (1406 and 1424) is joined to each V_{H} domain.

FIG. 15 is an illustration of an Fc-antigen binding domain construct (construct 15) containing three Fc domains and four antigen binding domains. The construct is formed of four Fc domain monomer containing polypeptides. Two polypeptides (1502 and 1536) contain an Fc domain monomer containing different charged amino acids at the C_{H}3-C_{H}3 interface than the WT sequence (1512 and 1524) linked by a spacer in a tandem series to a protuberance-containing Fc domain monomer (1518 and 1522) and an antigen binding domain containing a V_{H} domain (1514 and 1532) at the N-terminus. The third and fourth polypeptides (1504 and 1534) contain a cavity-containing Fc domain monomer (1510 and 1526) joined in a tandem series to antigen binding domain containing a V_{H} domain (1508 and 1530) at the N-terminus. A V_{L} containing domain (1506, 1516, 1520, and 1528) is joined to each V_{H} domain.

FIG. 16 is an illustration of an Fc-antigen binding domain construct (construct 16) containing five Fc domains and two antigen binding domains. The construct is formed of six Fc domain monomer containing polypeptides. Two polypeptides (1602 and 1632) contain an Fc domain monomer containing different charged amino acids at the C_{H}3-C_{H}3 interface than the WT sequence (1610 and 1624) linked by spacers in a tandem series to a protuberance-containing Fc domain monomer (1612 and 1622), a second protuberance-containing Fc domain monomer (1614 and 1620) and an antigen binding domain containing a V_{H} domain (1616 and 1618) at the N-terminus. The third, fourth, fifth, and sixth polypeptides (1608, 1606, 1626, and 1628) each contain a cavity-containing Fc domain. A V_{L} containing domain (1604 and 1630) is joined to each V_{H} domain.

FIG. 17 is an illustration of an Fc-antigen binding domain construct (construct 17) containing five Fc domains and four antigen binding domains. The construct is formed of six Fc monomer containing polypeptides. Two polypeptides (1702 and 1748) contain an Fc domain monomer containing different charged amino acids at the C_{H}3-C_{H}3 interface than the WT sequence (1718 and 1732) linked by spacers in a tandem series to a protuberance-containing Fc domain monomer (1720 and 1730) and a second protuberance-containing Fc domain monomer (1722 and 1728) at the N-terminus. The third, fourth, fifth, and sixth polypeptides (1706, 1704, 1746, and 1744) contain a cavity-containing Fc domain monomer (1716, 1710, 1734, and 1740) joined in a tandem series to an antigen binding domain containing a V_{H} domain (1712, 1724, 1738, and 1726) at the N-terminus. A V_{L} containing domain (1708, 1714, 1736, and 1742) is joined to each V_{H} domain.

FIG. 18 is an illustration of an Fc-antigen binding domain construct (construct 18) containing five Fc domains and six antigen binding domains. The construct is formed of six Fc domain monomer containing polypeptides. Two polypeptides (1802 and 1856) contain an Fc domain monomer containing different charged amino acids at the C_{H}3-C_{H}3 interface than the WT sequence (1818 and 1838) linked by spacers in a tandem series to a protuberance-containing Fc domain monomer (1820 and 1836), a second protuberance-containing Fc domain monomer (1822 and 1834) and an antigen binding domain containing a V_{H} domain (1826 and 1830) at the N-terminus. The third, fourth, fifth, and sixth polypeptides (1806, 1804, 1854, and 1852) each contain a cavity-containing Fc domain monomer (1816, 1810, 1840, and 1846) joined in a tandem series to an antigen binding domain containing a V_{H} domain (1812, 1828, 1844, and 1850) at the N-terminus. A V_{L} containing domain (1808, 1814, 1824, 1832, 1842, and 1848) is joined to each V_{H} domain.

FIG. 19 is an illustration of an Fc-antigen binding domain construct (construct 19) containing five Fc domains and two antigen binding domains. The construct is formed of six Fc domain monomer containing polypeptides. Two polypeptides (1902 and 1932) contain a protuberance-containing Fc domain monomer (1912 and 1930) linked by spacers in a tandem series to an Fc domain monomer containing different charged amino acids at the C_{H}3-C_{H}3 interface than the WT sequence (1908 and 1926), a protuberance-containing Fc domain monomer (1916 and 1918), and an antigen binding domain containing a V_{H} domain (1914 and 1920) at the N-terminus. The third and fourth polypeptides (1910 and 1928) contain cavity-containing Fc domain monomers and the fifth and sixth polypeptides (1906 and 1924) contain cavity-containing Fc domain monomers. A V_{L} containing domain (1904 and 1922) is joined to each V_{H} domain.

FIG. 20 is an illustration of an Fc-antigen binding domain construct (construct 20) containing five Fc domains and four antigen binding domains. The construct is formed of six Fc domain monomer containing polypeptides. Two polypeptides (2002 and 2048) contain a protuberance-containing Fc domain monomer (2020 and 2022) linked by spacers in a tandem series to an Fc domain monomer containing different charged amino acids at the C_{H}3-C_{H}3 interface than the WT sequence (2012 and 2030), and a protuberance-containing Fc domain monomer (2040 and 2038) at the N-terminus. The third, fourth, fifth, and sixth polypeptides (2006, 2004, 2046, and 2044) each contain a cavity-containing Fc domain monomer (2018. 2010, 2024, and 2032) joined in a tandem series to an antigen binding domain containing a V_{H} domain (2014, 2042, 2028, and 2036) at the N-terminus. A V_{L} containing domain (2008, 2016, 2026, and 2034) is joined to each V_{H} domain.

FIG. 21 is an illustration of an Fc-antigen binding domain construct (construct 21) containing five Fc domains and six antigen binding domains. The construct is formed of six Fc domain monomer containing polypeptides. Two polypeptides (2102 and 2156) contain a protuberance-containing Fc domain monomer (2120 and 2122) linked by spacers in a tandem series to an Fc domain monomer containing different charged amino acids at the C_{H}3-C_{H}3 interface than the WT sequence (2112 and 2130), another protuberance-containing Fc domain monomer (2144 and 2142), and an antigen binding domain containing a V_{H} domain (2148 and 2138) at the N-terminus. The third, fourth, fifth, and sixth polypeptides (2106, 2104, 2154, and 2152) each contain a cavity-containing Fc domain monomer (2118, 2110, 2124, and 2132) joined in a tandem series to an antigen binding domain containing a V_{H} domain (2114, 2150, 2128, and 2136) at the N-terminus. A V_{L} containing domain (2108, 2116, 2126, 2134, 2140, and 2146) is joined to each V_{H} domain.

FIG. 22 is an illustration of an Fc-antigen binding domain construct (construct 22) containing two Fc domains and three antigen binding domains with two different specificities. The construct is formed of three Fc domain monomer containing polypeptides. The first polypeptide (2202) contains a protuberance-containing Fc domain monomer (2208) linked by a spacer in a tandem series to another protuberance-containing Fc domain monomer (2206) and an antigen binding domain of a first specificity containing a V_{H} domain (2222) at the N-terminus. The second and third polypeptides (2226 and 2224) each contain a cavity-containing Fc domain monomer (2210 and 2216) joined in a tandem series to an antigen binding domain of a second specificity containing a V_{H} domain (2214 and 2220) at the N-terminus. A V_{L} containing domain (2204, 2212, and 2218) is joined to each V_{H} domain.

FIG. 23 is an illustration of an Fc-antigen binding domain construct (construct 23) containing three Fc domains and four antigen binding domains with two different specificities. The construct is formed of four Fc domain monomer containing polypeptides. The first polypeptide (2302) contains three protuberance-containing Fc domain monomers (2310, 2308, and 2306) linked by spacers in a tandem series with an antigen binding domain of a first specificity containing a V_{H} domain (2330) at the N-terminus. The second, third, and fourth polypeptides (2336, 2334, and 2332) contain a cavity-containing Fc domain monomer (2312, 2318, and 2324) joined in a tandem series with an antigen binding domain of a second specificity containing a V_{H} domain (2316, 2322, and 2328) at the N-terminus. A V_{L} containing domain (2304, 2314, 2320, and 2326) is joined to each V_{H} domain.

FIG. 24 is an illustration of an Fc-antigen binding domain construct (construct 24) containing three Fc domains and four antigen binding domains with two different specificities. The construct is formed of four Fc domain monomer containing polypeptides. Two polypeptides (2402 and 2436) contain an Fc domain monomer containing different charged amino acids at the C_{H}3-C_{H}3 interface than the WT sequence (2410 and 2412) linked by a spacer in a tandem series to a protuberance-containing Fc domain monomer (2426 and 2424) and an antigen binding domain of a first specificity containing a V_{H} domain (2430 and 2420) at the N-terminus. The third and fourth polypeptides (2404 and 2434) contain a cavity-containing Fc domain monomer (2408 and 2414) joined in a tandem series to an antigen binding domain of a second specificity containing a V_{H} domain (2432 and 2418). A V_{L} containing domain (2406, 2416, 2422, and 2428) is joined to each V_{H} domain.

FIG. 25 is an illustration of an Fc-antigen binding domain construct (construct 25) containing three Fc domains and four antigen binding domains with two different specificities. The construct is formed of four Fc domain monomer containing polypeptides. Two polypeptides (2502 and 2536) contain a protuberance-containing Fc domain monomer (2516 and 2518) linked by a spacer in a tandem series to an Fc domain monomer containing different charged amino acids at the C_{H}3-C_{H}3 interface than the WT sequence (2508 and 2526) and an antigen binding domain of a first specificity containing a V_{H} domain (2532 and 2530) at the N-terminus. The second and third polypeptides (2504 and 2534) contain a cavity-containing Fc domain monomer (2514 and 2520) joined in a tandem series to an antigen binding domain of a second specificity containing a V_{H} domain (2510 and 2524) at the N-terminus. A V_{L} containing domain (2506, 2512, 2522, and 2528) is joined to each V_{H} domain.

FIG. 26 is an illustration of an Fc-antigen binding domain construct (construct 26) containing five Fc domains and six antigen binding domains with two different specificities. The construct is formed of six Fc domain monomer containing polypeptides. Two polypeptides (2602 and 2656) contain an Fc domain monomer containing different charged amino acids at the C_{H}3-C_{H}3 interface than the WT sequence (2618 and 2620) linked by spacers in a tandem series to a protuberance-containing Fc domain monomer (2642 and 2640), a second protuberance-containing Fc domain monomer (2644 and 2638), and an antigen binding domain of a first specificity containing a V_{H} domain (2648 and 2634) at the N-terminus. The third, fourth, fifth, and sixth polypeptides (2606, 2604, 2654, and 2652) contain a cavity-containing Fc domain monomer (2616, 2610, 2622, and 2628) joined in a tandem series to an antigen binding domain of a second specificity containing a V_{H} domain (2612, 2650, 2626, and 2632) at the N-terminus. A V_{L} containing domain (2608, 2614, 2624, 2630, 2636, and 2646) is joined to each V_{H} domain.

FIG. 27 is an illustration of an Fc-antigen binding domain construct (construct 27) containing five Fc domains and six antigen binding domains with two different specificities. The construct is formed of six Fc domain monomer containing polypeptides. Two polypeptides (2702 and 2756) contain a protuberance-containing Fc domain monomer (2720 and 2722) linked by spacers in a tandem series to an Fc domain monomer containing different charged amino acids at the C_{H}3-C_{H}3 interface than the WT sequence (2712 and 2730), a protuberance-containing Fc domain monomer (2744 and 2742) and an antigen binding domain of a first specificity containing a V_{H} domain (2748 and 2738) at the N-terminus. The third, fourth, fifth, and sixth polypeptides (2706, 2704, 2754, and 2752) contain a cavity-containing Fc domain monomer (2718, 2724, 2710, and 2732) joined in tandem to an antigen binding domain of a second specificity containing a V_{H} domain (2714, 2728, 2750, and 2736) at the N-terminus. A V_{L} containing domain (2708, 2716, 2726, 2743, 2740, and 2746) is joined to each V_{H} domain.

FIG. 28 is an illustration of an Fc-antigen binding domain construct (construct 28) containing five Fc domains and six antigen binding domains with two different specificities. The construct is formed of six Fc domain monomer containing polypeptides. Two polypeptides (2802 and 2856) contain a protuberance-containing Fc domain monomer (2824 and 2830) linked by spacers in a tandem series to a second protuberance-containing Fc domain monomer (2826 and 2828), an Fc domain monomer containing different charged amino acids at the C_{H}3-C_{H}3 interface than the WT sequence (2810 and 2844), and an antigen binding domain of a first specificity containing a V_{H} domain (2850 and 2848) at the N-terminus. The third, fourth, fifth, and sixth polypeptides (2806, 2804, 2854, and 2852) contain a cavity-containing Fc domain monomer (2822, 2816, 2832, and 2838) joined in a tandem series to an antigen binding domain of a second specificity containing a V_{H} domain (2818, 2812, 2836, and 2842) at the N-terminus. A V_{L} containing domain (2808, 2814, 2820, 2834, 2840, and 2846) is joined to each VH domain.

FIG. 29 is an illustration of an Fc-antigen binding domain construct (construct 29) containing two Fc domains and two antigen binding domains with two different specificities. The construct is formed of three Fc domain monomer containing polypeptides. The first polypeptide (2902) contains two protuberance-containing Fc domain monomers (2908 and 2906), each with a different set of heterodimerization mutations, linked by a spacer in a tandem series to an antigen binding domain of a first specificity containing a V_{H} domain (2918). The second polypeptide (2920) contains a cavity-containing Fc domain monomer (2910) with a first set of heterodimerization mutations joined in a tandem series to an antigen binding domain of a second specificity containing a V_{H} domain (2914) at the N-terminus. The third polypeptide (2916) contains a cavity-containing Fc domain monomer with a second set of heterodimerization mutations. A V_{L} containing domain (2904 and 2912) is joined to each V_{H} domain.

FIG. 30 is an illustration of an Fc-antigen binding domain construct (construct 30) containing two Fc domains and three antigen binding domains with two different specificities. The construct is formed of three Fc domain monomer containing polypeptides. The first polypeptide (3002) contains two protuberance-containing Fc domain monomers (3008 and 3006), each with a different set of heterodimerization mutations, linked by a spacer in a tandem series to an antigen binding domain of a first specificity containing a V_{H} domain (3022) at the N-terminus. The second polypeptide (3024) contains a cavity-containing Fc domain monomer (3010) with a first set of heterodimerization mutations joined in a tandem series to an antigen binding domain of a second specificity containing a V_{H} domain (3014) at the N-terminus. The third polypeptide (3026) contains a cavity-containing Fc domain monomer (3016) with a first second of heterodimerization mutations joined in a tandem series to an antigen binding domain of a first specificity containing a V_{H} domain (3020) at the N-terminus. A V_{L} containing domain (3004, 3012, and 3018) is joined to each V_{H} domain.

FIG. 31 is an illustration of an Fc-antigen binding domain construct (construct 31) containing two Fc domains and three antigen binding domains with three different specificities. The construct is formed of three Fc domain monomer containing polypeptides. The first polypeptide (3102) contains two protuberance-containing Fc domain monomers (3108 and 3106), each with a different set of heterodimerization mutations, linked by a spacer in a tandem series to an antigen binding domain of a first specificity containing a V_{H} domain (3122) at the N-terminus. The second polypeptide (3126) contains a cavity-containing Fc domain monomer (3110) with a first set of heterodimerization mutations joined in a tandem series to an antigen binding domain of a second specificity containing a V_{H} domain (3114) at the N-terminus. The third polypeptide (3124) contains a cavity-containing Fc domain monomer (3116) with a second set of heterodimerization mutations joined in a tandem series to an antigen binding domain of a third specificity containing a V_{H} domain (3120) at the N-terminus. A V_{L} containing domain (3104, 3112, and 3118) is joined to each V_{H} domain.

FIG. 32 is an illustration of an Fc-antigen binding domain construct (construct 32) containing three Fc domains and three antigen binding domains with two different specificities. The construct is formed of four Fc domain monomer containing polypeptides. The first polypeptide (3202) contains three protuberance-containing Fc domain monomers (3210, 3208, and 3206), the third with a different set of heterodimerization mutations than the first two, linked by spacers in a tandem series to an antigen binding domain of a first specificity containing a V_{H} domain (3226) at the N-terminus. The second and third polypeptides (3230 and 3228) contain a cavity-containing Fc domain monomer (3212 and 3218) with a first set of heterodimerization mutations joined in a tandem series to an antigen binding domain of a second specificity containing a V_{H} domain (3216 and 3222) at the N-terminus. The fourth polypeptide (3224) contains a cavity-containing Fc domain monomer with a second set of heterodimerization mutations. A V_{L} containing domain (3204, 3214, and 3220) is joined to each V_{H} domain.

FIG. 33 is an illustration of an Fc-antigen binding domain construct (construct 33) containing three Fc domains and four antigen binding domains with two different specificities. The construct is formed of four Fc domain monomer containing polypeptides. The first polypeptide (3302) contains three protuberance-containing Fc domain monomers (3310, 3308, and 3306), the third with a different set of heterodimerization mutations than the first two, linked by spacers in a tandem series to an antigen binding domain of a first specificity containing a V_{H} domain (3330) at the N-terminus. The second and third polypeptides (3336 and 3334) contain a cavity-containing Fc domain monomer (3312 and 3318) with a first set of heterodimerization mutations joined in a tandem series to an antigen binding domain of a second specificity containing a V_{H} domain (3316 and 3322) at the N-terminus. The fourth polypeptide (3322) contains a cavity-containing Fc domain monomer (3324) with a second set of heterodimerization mutations joined in a tandem series to an antigen binding domain of a first specificity containing a V_{H} domain (3328) at the N-terminus. A V_{L} containing domain (3304, 3314, 3320, and 3326) is joined to each V_{H} domain.

FIG. 34 is an illustration of an Fc-antigen binding domain construct (construct 34) containing three Fc domains and four antigen binding domains with three different specificities. The construct is formed of four Fc domain monomer containing polypeptides. The first polypeptide (3402) contains three protuberance-containing Fc domain monomers (3410, 3408, and 3406), the third with a different set of heterodimerization mutations than the first two, linked by spacers in a tandem series to an antigen binding domain of a first specificity containing a V_{H} domain (3430) at the N-terminus. The second and third polypeptides (3436 and 3434) contain a cavity-containing Fc domain monomer (3412 and 3418) with a first set of heterodimerization mutations joined in a tandem series to an antigen binding domain of a second specificity containing a V_{H} domain (3416 and 3422) at the N-terminus. The fourth polypeptide (3432) contains a cavity-containing Fc domain monomer (3424) with a second set of heterodimerization mutations joined in a tandem series to an antigen binding domain of a third specificity containing a V_{H} domain (3428) at the N-terminus. A V_{L} containing domain (3404, 3414, 3420, and 3426) is joined to each V_{H} domain.

FIG. 35 is an illustration of an Fc-antigen binding domain construct (construct 35) containing three Fc domains and four antigen binding domains with three different specificities. The construct is formed of four Fc domain monomer containing polypeptides. The first polypeptide (3502) contains an Fc domain monomer containing different charged amino acids at the C_{H}3-C_{H}3 interface than the WT sequence (3510) linked by a spacer in a tandem series to a protuberance-containing Fc domain monomer (3526) with a first set of heterodimerization mutations and an antigen binding domain of a first specificity containing a V_{H} domain (3530) at the N-terminus. The second polypeptide (3536) contains an Fc domain monomer containing different charged amino acids at the C_{H}3-C_{H}3 interface than the WT sequence (3512) linked by a spacer in a tandem series to a protuberance-containing Fc domain monomer (3524) with a second set of heterodimerization mutations and an antigen binding domain of a first specificity containing a V_{H} domain (3520) at the N-terminus. The third polypeptide (3504) contains a cavity-containing Fc domain monomer (3508) with a first set of heterodimerization mutations joined in a tandem series to an antigen binding domain of a second specificity containing a V_{H} domain (3532) at the N-terminus. The fourth polypeptide (3534) contains a cavity-containing Fc domain monomer (3514) with a second set of heterodimerization mutations joined in a tandem series to an antigen binding domain of a third specificity containing a V_{H} domain (3518) at the N-terminus. A V_{L} containing domain (3506, 3516. 3522, and 3528) is joined to each V domain.

FIG. 36 is an illustration of an Fc-antigen binding domain construct (construct 36) containing five Fc domains and four antigen binding domains with two different specificities. The construct is formed of six Fc domain monomer containing polypeptides. Two polypeptides (3602 and 3644) contain a protuberance-containing Fc domain monomer (3614 and 3616), with a first set of heterodimerization mutations, linked by spacers in a tandem series to an Fc domain monomer containing different charged amino acids at the C_{H}3-C_{H}3 interface than the WT sequence (3610 and 3620), another protuberance-containing Fc domain monomer (3634 and 3632), with a second set of heterodimerization mutations, and an antigen binding domain of a first specificity containing a V_{H} domain (3638 and 3628) at the N-terminus. The third and fourth polypeptides (3612 and 3618) contain a cavity-containing Fc domain monomer with a first set of heterodimerization mutations. The fifth and six polypeptides (3604 and 3642) contain a cavity-containing Fc domain monomer (3608 and 3622) with a second set of heterodimerization mutations joined in a tandem series to an antigen binding domain of a second specificity containing a V_{H} domain (3640 and 3626) at the N-terminus. A V_{L} containing domain (3606, 3624, 3630, and 3636) is joined to each V_{H} domain.

FIG. 37 is an illustration of an Fc-antigen binding domain construct (construct 37) containing five Fc domains and six antigen binding domains with three different specificities. The construct is formed of six Fc domain monomer containing polypeptides. Two polypeptides (3702 and 3756) contain a cavity-containing Fc domain monomer (3720 and 3722), with a first set of heterodimerization mutations, linked by spacers in a tandem series to an Fc domain monomer containing different charged amino acids at the C_{H}3-C_{H}3 interface than the WT sequence (3712 and 3730), another protuberance-containing Fc domain monomer (3744 and 3742), with a second set of heterodimerization mutations, and an antigen binding domain of a first specificity containing a V_{H} domain (3748 and 3738) at the N-terminus. The third and fourth polypeptides (3706 and 3754) contain a cavity-containing Fc domain monomer (3718 and 3724) with a first set of heterodimerization mutations joined in a tandem series to an antigen binding domain of a second specificity containing a V_{H} domain (3714 and 3728) at the N-terminus. The fifth and sixth polypeptides (3704 and 3752) contain a cavity-containing Fc domain monomer (3710 and 3732) with a second set of heterodimerization mutations joined in a tandem series to an antigen binding domain of a third specificity containing a V_{H} domain (3750 and 3736) at the N-terminus. A V_{L} containing domain (3708, 3716, 3726, 3234, 3740, and 3746) is joined to each V_{H} domain.

FIG. 38 is an illustration of an Fc-antigen binding domain construct (construct 38) containing three Fc domains and four antigen binding domains with three different specificities. The construct is formed of four Fc domain monomer containing polypeptides. The first polypeptide (3802) contains a protuberance-containing Fc domain monomer (3816), with a first set of heterodimerization mutations, linked by a spacer in a tandem series to an Fc domain monomer containing different charged amino acids at the C_{H}3-C_{H}3 interface than the WT sequence (3808) and an antigen binding domain of a first specificity containing a V_{H} domain (3832) at the N-terminus. The second polypeptide (3836) contains a protuberance-containing Fc domain monomer (3818), with a second set of heterodimerization mutations, linked by a spacer in a tandem series to an Fc domain monomer containing different charged amino acids at the C_{H}3-C_{H}3 interface than the WT sequence (3826) and an antigen binding domain of a first specificity containing a V_{H} domain (3830) at the N-terminus. The third polypeptide (3804) contains a cavity-containing Fc domain monomer (3814) with a first set of heterodimerization mutations joined in a tandem series to an antigen binding domain of a second specificity containing a V_{H} domain (3810) at the N-terminus. The fourth polypeptide (3834) contains a cavity-containing Fc domain monomer (3820) with a second set of heterodimerization mutations joined in a tandem series to an antigen binding domain of a third specificity containing a V_{H} domain (3824) at the N-terminus. A V_{L} containing domain (3806, 3812, 3822, and 3828) is joined to each V_{H} domain.

FIG. 39 is an illustration of an Fc-antigen binding domain construct (construct 39) containing five Fc domains and four antigen binding domains of two different specificities. The construct is formed of six Fc domain monomer containing polypeptides. Two polypeptides (3902 and 3944) contain an Fc domain monomer containing different charged amino acids at the C_{H}3-C_{H}3 interface than the WT sequence (3912 and 3914) linked by spacers in a tandem series to a protuberance-containing Fc domain monomer (3932 and 3930), with a first set of heterodimerization mutations, a second protuberance-containing Fc domain monomer (3934 and 3928) with a second set of heterodimerization mutations, and an antigen binding domain of a first specificity containing a V_{H} domain (3938 and 3924) at the N-terminus. The third and fourth polypeptides (3910 and 3916) contain a cavity-containing Fc domain monomer with a first set of heterodimerization mutations. The fifth and sixth polypeptides (3904 and 3942) contain a cavity-containing Fc domain monomer (3908 and 3918) with a second set of heterodimerization mutations joined in a tandem series to an antigen binding domain of a second specificity containing a V_{H} domain (3940 and 3922) at the N-terminus. A V_{L} containing domain (3906, 3920, 3926, and 3936) is joined to each V_{H} domain.

FIG. 40 is an illustration of an Fc-antigen binding domain construct (construct 40) containing five Fc domains and six antigen binding domains of three different specificities. The construct is formed of six Fc domain monomer containing polypeptides. Two polypeptides (4002 and 4056) contain an Fc domain monomer containing different charged amino acids at the C_{H}3-C_{H}3 interface than the WT sequence (4018 and 4020) linked by spacers in a tandem series to a protuberance-containing Fc domain monomer (4042 and 4040), with a first set of heterodimerization mutations, a second protuberance-containing Fc domain monomer (4044 and 4038), with a second set of heterodimerization mutations, and an antigen binding domain of a first specificity containing a V_{H} domain (4048 and 4034) at the N-terminus. The third and fourth polypeptides (4006 and 4054) contain a cavity-containing Fc domain monomer (4016 and 4022) with a first set of heterodimerization mutations joined in a tandem series to an antigen binding domain of a second specificity containing a V_{H} domain (4012 and 4026) at the N-terminus. The fifth and sixth polypeptides (4004 and 4052) contain a cavity-containing Fc domain monomer (4010 and 4028) with a second set of heterodimerization mutations joined in a tandem series to an antigen binding domain of a third specificity containing a V_{H} domain (4050 and 4032) at the N-terminus. A V_{L} containing domain (4008, 4014, 4024, 4030, 4036, and 4046) is joined to each V_{H} domain.

FIG. 41 is an illustration of an Fc-antigen binding domain construct (construct 41) containing five Fc domains and four antigen binding domains of two different specificities. The construct is formed of six Fc domain monomer containing polypeptides. Two polypeptides (4102 and 4144) contain a protuberance-containing Fc domain monomer (4118 and 4124), with a first set of heterodimerization mutations, linked by spacers in a tandem series to second protuberance-containing Fc domain monomer (4120 and 4122), with a second set of heterodimerization mutations, an Fc domain monomer containing different charged amino acids at the C_{H}3-C_{H}3 interface than the WT sequence (4108 and 4134), and an antigen binding domain of a first specificity containing a V_{H} domain (4140 and 4138) at the N-terminus. The third and fourth polypeptides (4104 and 4142) contain a cavity-containing Fc domain monomer (4116 and 4126) with a first set of heterodimerization mutations joined in a tandem series to an antigen binding domain of a second specificity containing a V_{H} domain (4112 and 4130) at the N-terminus. The fifth and sixth polypeptides (4110 and 4132) contain a cavity-containing Fc domain monomer with a second set of heterodimerization mutations. A V_{L} containing domain (4106, 4114, 4128, and 4136) is joined to each V_{H} domain.

FIG. 42 is an illustration of an Fc-antigen binding domain construct (construct 42) containing five Fc domains and six antigen binding domains of three different specificities. The construct is formed of six Fc domain monomer containing polypeptides. Two polypeptides (4202 and 4256) contain a protuberance-containing Fc domain monomer (4224 and 4230), with a first set of heterodimerization mutations, linked by spacers in a tandem series to a second protuberance-containing Fc domain monomer (4226 and 4228), with a second set of heterodimerization mutations, an Fc domain monomer containing different charged amino acids at the C_{H}3-C_{H}3 interface than the WT sequence (4210 and 4244), and an antigen binding domain of a first specificity containing a V_{H} domain (4250 and 4248) at the N-terminus. The third and fourth polypeptides (4206 and 4254) contain a cavity-containing Fc domain monomer (4222 and 4232) with a first set of heterodimerization mutations joined in a tandem series to an antigen binding domain of a second specificity containing a V_{H} domain (4218 and 4236) at the N-terminus. The fifth and sixth polypeptides (4204 and 4252) contain a cavity-containing Fc domain monomer (4216 and 4238) with a second set of heterodimerzation mutations joined in a tandem series to an antigen binding domain of a third specificity containing a V_{H} domain (4212 and 4242) at the N-terminus. A V_{L} containing domain (4208, 4214, 4220, 4234, 4240, and 4246) is joined to each V_{H} domain.

FIG. 43 is a picture of a non-reducing SDS-PAGE gel showing the expression and purity of Rituximab (Rtxn), an anti-CD20 IgG1 mAB, and constructs 7, 13, 19, and 1.

FIG. 44 is a gel showing expression of various Fc-antigen binding domain constructs. The first three lanes show expression of an Fc-antigen binding domain construct at various ratios of the three separate polypeptides. The fourth lane shows expression of a SIF3 construct (a construct with three Fc domains which does not contain an antigen binding domain) and the fifth lane shows expression of a construct with five Fc domains which does not contain an antigen binding domain. The sixth lane is a molecular weight marker.

FIG. 45 is two graphs showing the results of surface plasmon resonance (SPR) assays. In the graph on the left, the results show that the Fc3Y-SIFbody (a construct having the structure of Construct 13 (FIG. 13)) binds much stronger to its CTLA-4 target than the corresponding parent monoclonal antibody (mAb) while the Fc3I-SIFbody (a construct having the structure of Construct 7 (FIG. 7)) has similar binding affinity as the parent mAb. In the graph on the right, the results show that afucosylating the antibody increases cell-surface FcγRIIa binding ˜10-fold, while using various Fc-antigen binding domain constructs enhances binding ˜300-800 fold.

FIG. 46 shows two graphs showing the results of two cell-surface FcγR binding binding assays. The graph on the left shows binding affinities by various constructs to FcγRIIa. Afucosylation of a mAb with the same CDRs as Gazyva, a commercially available anti-CD20 antibody enhances binding ˜10-fold, while all Fc-antigen binding domain constructs show enhanced binding >100-fold over the parent mAb. The graph on the right shows binding affinities by various constructs to FcγRIIa. Afucosylation of the mAb had no effect on binding, while all Fc-antigen binding domain constructs show enhanced binding >100-fold over the parent mAb.

FIG. 47 is three graphs showing the results of CDC, ADCP, and ADCC assays with various anti-CD20 constructs targeting B cells. The first graph shows that the S3Y Fc-antigen binding domain construct can mediate CDC. The middle graph shows that both the SAI and S3Y Fc-antigen binding domain constructs exhibit >100-fold enhanced potency in an ADCP FcγRIIa reporter assay. The third graph shows that the SAI and S3Y Fc-antigen binding domain constructs exhibit enhanced ADCC activity relative to the fucosylated mAb and similar activity to the afucosylated mAb.

FIG. 48 is three graphs showing the results of CDC, ADCP, and ADCC assays with various anti-CTLA-4 constructs targeting CTLA-4 tranfected HEK cells. The first graph shows that the SAI (an Fc-antigen binding domain construct having the structure shown in FIG. 7) and S3Y (an Fc-antigen binding domain construct having the structure shown in FIG. 13) constructs mediate enhanced CDC. The second graph shows that the SAI and S3Y constructs mediate enhanced ADCP, and the third graph shows that both the SAI and S3Y Fc-antigen binding domain constructs exhibit enhanced ADCC activity relative to the fucosylated mAb and similar activity to the afucosylated mAb.

FIG. 49 is three graphs showing the results of ADCC, ADCP, and CDC assays with various anti-PD-L1 constructs targeting PD-L1 transfected HEK cells. The first graph shows that both the SAI (a construct having the structure of Fc-antigen binding domain construct 7 (FIG. 7)) and S3Y Fc-antigen binding domain (a construct having the structure of Fc-antigen binding domain construct 13 (FIG. 13)) constructs exhibit similar ADCC activity relative to the fucosylated and afucosylated mAbs. The second graph shows that the SAI and S3Y constructs mediate enhanced ADCP, and the third graph shows that the S3Y construct can mediate CDC.

FIG. 50 shows the size distribution by non-reducing SDS-PAGE of Fc construct A and Fc construct B in unpurified media.

FIG. 51 shows the expression and assembly of Fc construct A and another Fc construct having three Fc domains but without electrostatic steering mutations in the “stem” subunits.

FIG. 52 is a schematic representation of three exemplary ways the antigen binding domain can be joined to the Fc domain of an Fc construct. Panel A shows a heavy chain component of an antigen binding domain can be expressed as a fusion protein of an Fc chain and a light chain component can be expressed as a separate polypeptide. Panel B shows an scFv expressed as a fusion protein of the long Fc chain. Panel C shows heavy chain and light chain components expressed separately and exogenously added and joined to the Fc-antigen binding domain construct with a chemical bond.

FIG. 53A depicts the amino acid sequence of a human IgG1 (SEQ ID NO: 43) with Kabat numbering. The hinge region is indicated by a double underline, the CH2 domain is not underlined and the CH3 region is underlined.

FIG. 53B depicts the amino acid sequence of a human IgG1 (SEQ ID NO: 45) with Kabat numbering. The hinge region, which lacks E216-C220, inclusive, is indicated by a double underline, the CH2 domain is not underlined and the CH3 region is underlined and lacks K447.

FIG. 53C depicts the amino acid sequence of a human IgG1 (SEQ ID NO: 47) with Kabat numbering. The hinge region is indicated by a double underline, the CH2 domain is not underlined and the CH3 region is underlined and lacks 447K.

FIG. 53D depicts the amino acid sequence of a human IgG1 (SEQ ID NO: 42) with Kabat numbering. The hinge region, which lacks E216-C220, inclusive, is indicated by a double underline, the CH2 domain is not underlined and the CH3 region is underlined.

FIG. 54 is a graph showing the induction of complement dependent cytotoxicity (CDC) in Daudi cells by anti-CD20 construct 7, anti-CD20 construct 13, Gazyva, and fucosylated anti-CD20 IgG1 antibody (obinutuzumab).

FIG. 55 is a graph showing the induction of FcγRIIa signaling (fold induction) by anti-CD20 construct 7, anti-CD20 construct 13, and fucosylated and afucosylated anti-CD20 IgG1 antibody (obinutuzumab) in an ADCP reporter assay.

FIG. 56 is a graph showing the induction of FcγRIIa signaling (fold induction) by anti-CD2O construct 7, anti-CD20 construct 13, and fucosylated and afucosylated anti-CD20 IgG1 antibody (obinutuzumab) in an ADCC reporter assay.

FIG. 57 is a graph showing the depletion of CD19+ B cells in human whole blood by fucosylated and afucosylated anti-CD20 construct 13 and fucosylated and afucosylated obinutuzumab monoclonal antibody.

FIG. 58 is a graph showing the reduction in tumor growth in a mouse lymphoma model after injection with a single dose of anti-CD20 construct 13, the fucosylated obinutuzumab monoclonal antibody, or the daratumumab monodonal antibody, or after injection with multiple doses of anti-CD20 construct 13.

### DETAILED DESCRIPTION

Many therapeutic antibodies function by recruiting elements of the innate immune system through the effector function of the Fc domains, such as antibody-dependent cytotoxicity (ADCC), antibody-dependent cellular phagocytosis (ADCP), and complement-dependent cytotoxicity (CDC). In some instances, the present disclosure contemplates combining an antigen binding domain of a known single Fc-domain containing therapeutic, e.g., a known therapeutic antibody, with at least two Fc domains to generate a novel therapeutic with unique biological activity. In some instances, a novel therapeutic disclosed herein has a biological activity greater than that of the known Fc-domain containing therapeutic, e.g., a known therapeutic antibody. The presence of at least two Fc domains can enhance effector functions and to activate multiple effector functions, such as ADCC in combination with ADCP and/or CDC, thereby increasing the efficacy of the therapeutic molecules, disclosure In order to generate a product with consistent biological function, control of the number of Fc domains is critical. The disclosure features a set of Fc engineering tools to control homodimerization and heterodimerization of the peptides encoding the Fc domain, to assemble molecules of discrete size from a limited number of polypeptide chains. International Publication Nos. WO/2015/168643, WO2017/151971, WO 2017/205436, and WO 2017/205434 disclose Fc engineering tools and methods for assembling molecules with two or more Fc domains, and are herein incorporated by reference in their entirety. The engineering tools include structural features (for example, glycine linkers) that significantly improve manufacturing outcome. The properties of these constructs allow for the efficient generation of substantially homogenous pharmaceutical compositions. Such homogeneity in a pharmaceutical composition is desirable in order to ensure the safety, efficacy, uniformity, and reliability of the pharmaceutical composition. Having a high degree of homogeneity in a pharmaceutical composition also minimizes potential aggregation or degradation of the pharmaceutical product caused by unwanted materials (e.g., degradation products, and/or aggregated products or multimers), as well as limiting off-target and adverse side effects caused by the unwanted materials.

As described in detail herein, we improved homogeneity of the composition by engineering the Fc domain components of the Fc-antigen binding domain constructs using approaches including the use of spacers including only glycine residues to join two Fc domain monomers in tandem series, the use of polypeptide sequences having the terminal lysine residue removed, and the use of two sets of heterodimerizing selectivity modules: (i) heterodimerizing selectivity modules having different reverse charge mutations and (ii) heterodimerizing selectivity modules having engineered cavities and protuberances.

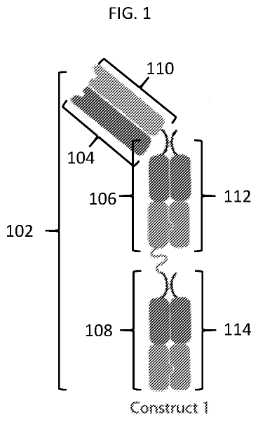

We designed a series of Fc-antigen binding domain constructs in which Fc domains were connected in tandem, using one long peptide chain containing multiple Fc sequences separated by linkers, and multiple copies of a short chain containing a single Fc sequence (Fc-antigen binding domain constructs 1-6; FIG. 1-FIG. 6). Heterodimerizing mutations were introduced into each Fc sequence to ensure assembly into the desired tandem configuration with minimal formation of smaller or larger complexes. Any number of Fc domains can be connected in tandem in this fashion, allowing the creation of constructs with 2, 3, 4, 5, 6, 7, 8, 9, 10, or more Fc domains. For a peptide with N Fc domains, such constructs can be prepared with 1 to N+1 antigen binding domains, depending whether the antigen binding domains are introduced into the long peptide chain, the short peptide chain, or both, respectively.

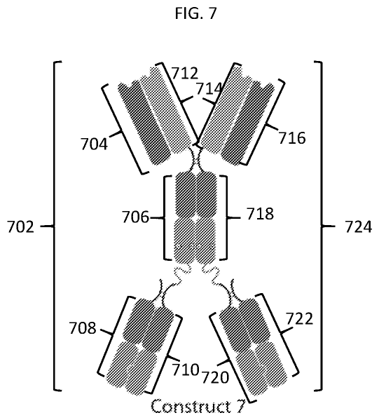

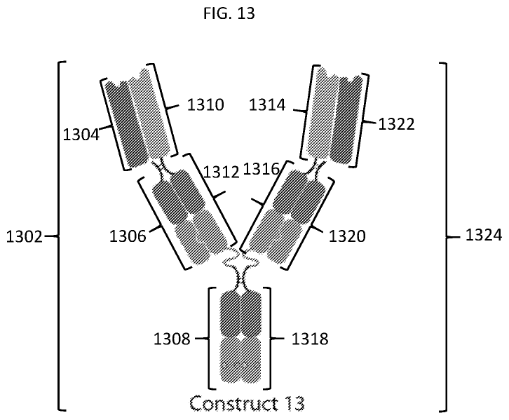

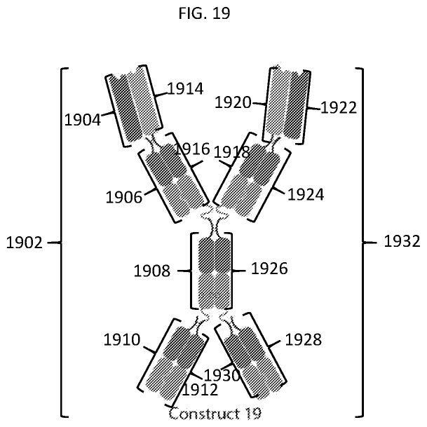

In Fc-antigen binding domain constructs 1-6 (FIG. 1-FIG. 6), Fc domains were connected with a single branch point between the Fc domains. These constructs include two copies of a long peptide chain containing multiple Fc sequences separated by linkers, in which the branching Fc sequence contains homodimerizing mutations and the non-branching Fc domains contain heterodimerizing mutations. Multiple copies of short chains including a single Fc sequence with mutations complementary to the heterodimerizing mutations in the long chains are used to complete the multimeric Fc scaffold. Heterodimerizing Fc domains can be linked to the C-terminal end (e.g., Fc-antigen binding domain constructs 7-12; FIG. 7-FIG. 12), the N-terminal end (e.g., Fc-antigen binding domain constructs 13-18; FIG. 13-FIG. 18), or both ends of the branching Fc domain (e.g., Fc-antigen binding domain constructs 19-21; FIG. 19-FIG. 21). Multiple Fc domains in tandem may be linked to either end of the branching Fc domain. Antigen binding domains may be introduced into the long peptide chains, resulting in two antigen binding domains per assembled protein molecule. Alternatively, antigen binding domains may be introduced into the short peptide chains, resulting in N−1 antigen binding domains per assembled protein molecule, where N is the number of Fc domains in the assembled protein molecule. If antigen binding domains are introduced into both the short and the long peptide chains, the resulting assembled protein molecule contains N+1 antigen binding domains.

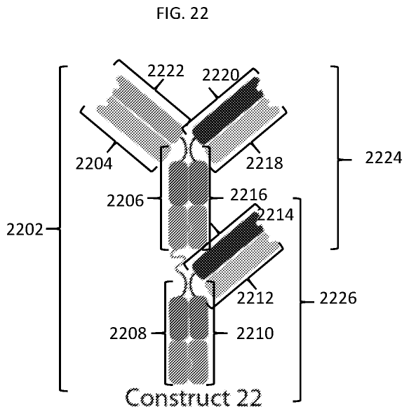

Bispecific constructs may be generated from any of the above designs by using antigen binding domains in which two different antigen binding sequences are linked in tandem. Alternatively, bispecific constructs may be generated from the above Fc scaffolds in which the long chain encodes for one antigen-binding domain, while the short chain encodes for a different antigen-binding domain. The different antigen binding domains may use different light chains, or a common light chain, or may consist of scFv domains. Illustrative examples of this concept are Fc-antigen binding domain constructs 22-28 (FIG. 22-FIG. 28).

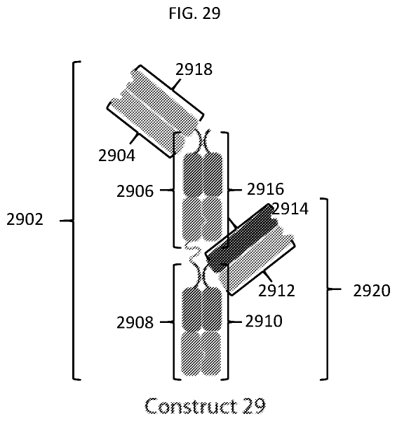

Bi-specific and tri-specific constructs may also be generated by the use of two different sets of heterodimerizing mutations (e.g., Fc-antigen binding domain constructs 29-42; FIG. 29-FIG. 42). Such heterodimerizing sequences need to be designed in such a way that they disfavor association with the other heterodimerizing sequences. Such designs can be accomplished using different electrostatic steering mutations between the two sets of heterodimerizing mutations as described herein. One example of orthogonal electrostatic steering mutations is E357K in the first knob Fc, K370D in first hole Fc, D399K in the second knob Fc, and K409D in the second hole Fc.

Past engineering efforts for monoclonal antibodies (mAbs) and Fc domains included making mutations in the Fc domain to strengthen binding to FcγRIIIa and thus enhancing the antibody-dependent cell-mediated cytotoxicity (ADCC) response, and afucosylation of the Fc domain to strengthen binding to FcγRIIIa and thus enhances the ADCC response.

In comparison to antibodies with mutations in the Fc domain to strengthen binding to FcγRIIIa or afucosylation of the Fc domain, the Fc-antigen binding domain constructs disclosed in this disclosure unexpectedly feature stronger binding to multiple classes of Fcγ receptors and enhanced activity of multiple cytotoxicity pathways. The Fc-antigen binding domain constructs of this disclosure can enhance binding to both FcγRIIa and FcγRIIIa compared to their corresponding fucosylated and afucosylated parent monoclonal antibodies (see, Example 46). Further, the Fc-antigen binding domain constructs of this disclosure unexpectedly feature an ability to mediate the complement-dependent cytotoxicity (CDC) pathway and/or the antibody-dependent cellular phagocytosis (ADCP) pathway in addition to enhancing the ADCC pathway response (see. Example 47).

### I. Fc Domain Monomers

An Fc domain monomer includes a hinge domain, a C_{H}2 antibody constant domain, and a C_{H}3 antibody constant domain. The Fc domain monomer can be of immunoglobulin antibody isotype IgG, IgE, IgM, IgA, or IgD. The Fc domain monomer may also be of any immunoglobulin antibody isotype (e.g., IgG1, IgG2a, IgG2b, IgG3, or IgG4). The Fc domain monomers may also be hybrids, e.g., with the hinge and C_{H}2 from IgG1 and the CH3 from IgA, or with the hinge and C_{H}2 from IgG1 but the CH3 from IgG3. A dimer of Fc domain monomers is an Fc domain (further defined herein) that can bind to an Fc receptor, e.g., FcγRIIa, which is a receptor located on the surface of leukocytes. In the present disclosure, the C_{H}3 antibody constant domain of an Fc domain monomer may contain amino acid substitutions at the interface of the C_{H}3-C_{H}3 antibody constant domains to promote their association with each other. In other embodiments, an Fc domain monomer includes an additional moiety, e.g., an albumin-binding peptide or a purification peptide, attached to the N- or C-terminus. In the present disclosure, an Fc domain monomer does not contain any type of antibody variable region, e.g., V_{H}, V_{L}, a complementarity determining region (CDR), or a hypervariable region (HVR).

In some embodiments, an Fc domain monomer in an Fc-antigen binding domain construct described herein (e.g., an Fc-antigen binding domain construct having three Fc domains) may have a sequence that is at least 95% identical (at least 97%, 99%, or 99.5% identical) to the sequence of SEQ ID NO:42. In some embodiments, an Fc domain monomer in an Fc-antigen binding domain construct described herein (e.g., an Fc-antigen binding domain construct having three Fc domains) may have a sequence that is at least 95% identical (at least 97%, 99%, or 99.5% identical) to the sequence of any one of SEQ ID NOs: 44, 46, 48, and 50-53. In certain embodiments, an Fc domain monomer in the Fc-antigen binding domain construct may have a sequence that is at least 95% identical (at least 97%, 99%, or 99.5% identical) to the sequence of any one of SEQ ID NOs: 48, 52, and 53.

SEQ ID NO: 42
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFS
CSVMHEALHNHYTQKSLSLSPGK
SEQ ID NO: 44
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVCTLPPSRDELTKNQVSLSCAVKGFY
PSDIAVEWESNGQPENNYKTTPPVLDSDGSFFLVSKLTVDKSRWQQGNVFS
CSVMHEALHNHYTQKSLSLSPGK
SEQ ID NO: 46
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVCTLPPSRDELTKNQVSLSCAVKGFY
PSDIAVEWESNGQPENNYKTTPPVLDSDGSFFLVSKLTVDKSRWQQGNVFS
CSVMHEALHNHYTQKSLSLSPG
SEQ ID NO: 48
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVCTLPPSRDELTKNQVSLSCAVDGFY
PSDIAVEWESNGQPENNYKTTPPVLDSDGSFFLVSKLTVDKSRWQQGNVFS
CSVMHEALHNHYTQKSLSLSPG
SEQ ID NO: 50
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPCRDELTKNQVSLWCLVKGFY
PSDIAVEWESNGQPENNYKTTPPVLDSDGSFFLVSKLTVDKSRWQQGNVFS
CSVMHEALHNHYTQKSLSLSPGK
SEQ ID NO: 51
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFY
PSDIAVEWESNGQPENNYKTTPPVLKSDGSFFLYSDLTVDKSRWQQGNVFS
CSVMHEALHNHYTQKSLSLSPGK
SEQ ID NO: 52
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFY
PSDIAVEWESNGQPENNYKTTPPVLKSDGSFFLYSDLTVDKSRWQQGNVFS
CSVMHEALHNHYTQKSLSLSPG
SEQ ID NO: 53
DKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPCRDELTKNQVSLWCLVKGFY
PSDIAVEWESNGQPENNYKTTPPVLDSDGSFFLYSKLTVDKSRWQQGNVFS
CSVMHEALHNHYTQKSLSLSPGK

### II. Fc Domains

As defined herein, an Fc domain includes two Fc domain monomers that are dimerized by the interaction between the C_{H}3 antibody constant domains. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present disclosure binds to an Fcγ receptor (e.g., FcγRI (CD64), FcγRIIa (CD32), FcγRIIb (CD32), FcγRIIa (CD16a), FcγRIIIb (CD16b)), and/or FcγRIV and/or the neonatal Fc receptor (FcRn).

### III. Antigen Binding Domains

Antigen binding domains include one or more peptides or polypeptides that specifically bindi a target molecule. Antigen binding domains may include the antigen binding domain of an antibody. In some embodiments, the antigen binding domain may be a fragment of an antibody or an antibody-construct, e.g., the minimal portion of the antibody that binds to the target antigen. An antigen binding domain may also be a synthetically engineered peptide that binds a target specifically such as a fibronectin-based binding protein (e.g., a FN3 monobody). A fragment antigen-binding (Fab) fragment is a region on an antibody that binds to a target antigen. It is composed of one constant and one variable domain of each of the heavy and the light chain. A Fab fragment includes a V_{H}, V_{L}, CH1 and C_{L} domains. The variable domains V_{H} and V_{L} each contain a set of 3 complementarity-determining regions (CDRs) at the amino terminal end of the monomer. The Fab fragment can be of immunoglobulin antibody isotype IgG, IgE, IgM, IgA, or IgD. The Fab fragment monomer may also be of any immunoglobulin antibody isotype (e.g., IgG1, IgG2a, IgG2b, IgG3, or IgG4). In some embodiments, a Fab fragment may be covalently attached to a second identical Fab fragment following protease treatment (e.g., pepsin) of an immunoglobulin, forming an F(ab′)₂ fragment. In some embodiments, the Fab may be expressed as a single polypeptide, which includes both the variable and constant domains fused, e.g. with a linker between the domains.

In some embodiments, only a portion of a Fab fragment may be used as an antigen binding domain. In some embodiments, only the light chain component (V_{L}+C_{L}) of a Fab may be used, or only the heavy chain component (V_{H}+C_{H}) of a Fab may be used. In some embodiments, a single-chain variable fragment (scFv), which is a fusion protein of the the V_{H} and V_{L} chains of the Fab variable region, may be used. In other embodiments, a linear antibody, which includes a pair of tandem Fd segments (V_{H}-C_{H}-V_{H}-C_{H}1), which, together with complementary light chain polypeptides form a pair of antigen binding regions, may be used.

In some embodiments, an antigen binding domain of the present disclosure includes for a target or antigen listed in Table 1, one, two, three, four, five, or all six of the CDR sequences listed in Table 1 for the listed target or antigen, as provided in further detail below Table 1.

TABLE 1

| | | CDR1-IMGT | CDR2-IMGT | CDR3-IMGT | CDR1-IMGT | CDR2-IMGT | CDR3-IMGT |
|---|---|---|---|---|---|---|---|
| Target | Antibody Name | (heavy) | (heavy) | (heavy) | (light) | (light) | (light) |
| B7-H3 | Enoblitzumab | GFTFSSFG | ISSDSSAI | GRGRENIYY | QNVDTN | SAS | QQYNNYPF |
| | | (SEQ ID NO: | (SEQ ID NO: | GSRLDY | (SEQ ID NO: | | T |
| | | 76) | 106) | (SEQ ID NO: | 171) | | (SEQ ID NO: |
| | | | | 137) | | | 201) |
| beta- | Gantenerumab | GFTFSSYA | INASGTRT | ARGKGNTH | QSVSSSY | GAS | LQIYNMPIT |
| amylold | | (SEQ ID NO: | (SEQ ID NO: | KPYGYVRYF | (SEQ ID NO: | | (SEQ ID NO: |
| | | 77) | 107) | DV | 172) | | 202) |
| | | | | (SEQ ID NO: | | | |
| | | | | 138) | | | |
| CCR4 | Mogamulizumab | GFIFSNYG | ISSASTYS | GRHSDGNF | RNIVHINGD | KVS | FQGSLLPW |
| | | (SEQ ID NO: | (SEQ ID NO: | AFGY | TY | | T |
| | | 78) | 108) | (SEQ ID NO: | (SEQ ID NO: | | (SEQ ID NO: |
| | | | | 139) | 173) | | 203) |
| CD19 | Inebilizumab | GFTFSSSW | IYPGDGDT | ARSGFITTV | ESVDTFGIS | EAS | QQSKEVPFT |
| | | (SEQ ID NO: | (SEQ ID NO: | RDFDY | F | | (SEQ ID NO: |
| | | 79) | 109) | (SEQ ID NO: | (SEQ ID NO: | | 204) |
| | | | | 140) | 174) | | |
| CD20 | Obinutuzumab | GYAFSYSW | IFPGDGDT | ARNVFDGY | KSLLHSNGI | QMS | AQNLELPYT |
| | | (SEQ ID NO: | (SEQ ID NO: | WLVY | TY | | (SEQ ID NO: |
| | | 80) | 110) | (SEQ ID NO: | (SEQ ID NO: | | 205) |
| | | | | 141) | 175) | | |
| CD20 | Ocaratuzumab | GRTFTSYN | AIYPLTGDT | ARSTYVGG | SSVPY | ATS | QQWLSNPP |
| | | MH | (SEQ ID NO: | DWQEDV | (SEQ ID NO: | | T |
| | | (SEQ ID NO: | 111) | (SEQ ID NO: | 176) | | (SEQ ID NO: |
| | | 81) | | 142) | | | 206) |
| CD20 | Rituximab | GYTFTSYN | IYPGNGDT | CARSTYYG | SSVSY | ATS | QQVVTSNPP |
| | | (SEQ ID NO: | (SEQ ID NO: | GDWYFNV | (SEQ ID NO: | | T |
| | | 82) | 112) | (SEQ ID NO: | 177) | | (SEQ ID NO: |
| | | | | 143) | | | 207) |
| CD20 | Ublituximab | GYTFTSYN | IYPGNGDT | ARYDYNYA | SSVSY | ATS | QQWTFNPP |
| | | (SEQ ID NO: | (SEQ ID NO: | MDY | (SEQ ID NO: | | T |
| | | 82) | 112) | (SEQ ID NO: | 177) | | (SEQ ID NO: |
| | | | | 144) | | | 208) |
| CD20 | Veltuzumab | GYTFTSYN | IYPGNGDT | ARSTYYGG | SSVSY | ATS | QQWTSNPP |
| | | (SEQ ID NO: | (SEQ ID NO: | DWYFDV | (SEQ ID NO: | | T |
| | | 82) | 112) | (SEQ ID NO: | 177) | | (SEQ ID NO: |
| | | | | 145) | | | 207) |
| CD22 | Epratuzumab | GYTFTSYW | INPRNDYT | ARRDITTFY | QSVLYSANH | WAS | HQYLSS |
| | | (SEQ ID NO: | (SEQ ID NO: | (SEQ ID NO: | KNY | | (SEQ NO: |
| | | 83) | 113) | 146) | (SEQ ID NO: | | 209) |
| | | | | | 178) | | |
| CD37 | Otlertuzumab | GYSFTGYN | IDPYYGGT | ARSVGPFD | ENVYSY | FAK | QHHSDNPW |
| | | (SEQ ID NO: | (SEQ ID NO: | S | (SEQ ID NO: | | T |
| | | 84) | 114) | (SEQ ID NO: | 179) | | (SEQ ID NO: |
| | | | | 147) | | | 210) |
| CD38 | Daratumumab | GETENSFA | ISGSGGGT | AKDKILWFG | QSVSSY | DAS | QQRSNWPP |
| | | (SEQ ID NO: | (SEQ ID NO: | EPVFDY | (SEQ ID NO: | | T |
| | | 85) | 115) | (SEQ ID NO: | 180) | | (SEQ ID NO: |
| | | | | 148) | | | 211) |
| CD38 | Isatuximab | GYTFTDYW | IYPGDGDT | ARGDYYGS | QDVSTV | SAS | QQHYSPPY |
| | | (SEQ ID NO: | (SEQ ID NO: | NSLDY | (SEQ ID NO: | | T |
| | | 86) | 109) | (SEQ ID NO: | 181) | | (SEQ ID NO: |
| | | | | 149) | | | 212) |
| CD3epsilon | Foralumab | GEKFSGYG | IWYDGSKK | ARQMGYWH | QSVSSY | DAS | QQRSNWPP |
| | | (SEQ ID NO: | (SEQ ID NO: | FDLW | (SEQ ID NO: | | LT |
| | | 87) | 116) | (SEQ ID NO: | 180) | | (SEQ ID NO: |
| | | | | 150) | | | 213) |
| CD52 | Alemtuzumab | GFTFTDFY | IRDKAKGYT | AREGHTAA | QNIDKY | NTN | LQHISRPRT |
| | | (SEQ ID NO: | T | PFDY | (SEQ ID NO: | | (SEQ ID NO: |
| | | 88) | (SEQ ID NO: | (SEQ ID NO: | 182) | | 214) |
| | | | 117) | 151) | | | |
| CD105 | Carotuximab | GETESDAW | IRSKASNHA | TRWRREFD | SSVSY | ATS | QQWSSNPL |
| | | (SEQ ID NO: | T | S | (SEQ ID NO: | | T |
| | | 89) | (SEQ ID NO: | (SEQ ID NO: | 177) | | (SEQ ID NO: |
| | | | 118) | 152) | | | 215) |
| CD147 | cHAb18 | GETESDAW | IRSANNHAP | TRDSTATH | QSVIND | TAS | QQDTSPP |
| | | (SEQ ID NO: | T | (SEQ ID NO: | (SEQ ID NO: | | (SEQ ID NO: |
| | | 89) | (SEQ ID NO: | 153) | 183) | | 216) |
| | | | 119) | | | | |
| c-Met | ABT-700 | GYIFTAYT | IKPNNGLA | ARSEITTEF | ESVDSYANS | RAS | QQSKEDPLT |
| | | (SEQ ID NO: | (SEQ ID NO: | DY | F | | (SEQ ID NO: |
| | | 90) | 120) | (SEQ ID NO: | (SEQ ID NO: | | 217) |
| | | | | 154) | 184) | | |
| CTLA-4 | Ipilimumab | GFTFSSYT | ISYDGNNK | ARTGWLGP | QSVGSSY | GAF | QQYGSSPW |
| | | (SEQ ID NO: | (SEQ ID NO: | FDY | (SEQ ID NO: | | T |
| | | 91) | 121) | (SEQ ID NO: | 185) | | (SEQ ID NO: |
| | | | | 155) | | | 218) |
| EGFR2 | Margetuximab | GENIKDTY | IYPTNGYT | SRVVGGDGF | QDVNTA | SAS | QQHYTTPPT |
| | | (SEQ ID NO: | (SEQ ID NO: | YAMDY | (SEQ ID NO: | | (SEQ ID NO: |
| | | 92) | 122) | (SEQ ID NO: | 186) | | 219) |
| | | | | 156) | | | |
| EGFR3 | Lumretuzumab | GYTFRSSY | IYAGTGSP | ARHRDYYS | QSVLNSGN | WAS | QSDYSYPYT |
| | | (SEQ ID NO: | (SEQ ID NO: | NSLTY | QKNY | | (SEQ ID NO: |
| | | 93) | 123) | (SEQ ID NO: | (SEQ ID NO: | | 220) |
| | | | | 157) | 187) | | |
| EphA3 | Ifabotuzumab | GYTFTGYW | IYPGSGNT | ARGGYYED | QGIISY | AAS | GQYANYPY |
| | | (SEQ ID NO: | (SEQ ID NO: | FDS | (SEQ ID NO: | | T |
| | | 94) | 124) | (SEQ ID NO: | 188) | | (SEQ ID NO: |
| | | | | 158) | | | 221) |
| GD3 | Ecromeximab | GFAFSHYA | ISSGGSGT | TRVKLGTYY | QDISNY | YSS | HQYSKLP |
| | | (SEQ ID NO: | (SEQ ID NO: | FDS | (SEQ ID NO: | | (SEQ ID NO: |
| | | 95) | 125) | (SEQ ID NO: | 189) | | 222) |
| | | | | 159) | | | |
| GPC3 | Codrituzumab | GYTFTDYE | LDPKTGDT | TRFYSYTY | QSLVHSNR | KVS | SQNTHVPPT |
| | | (SEQ ID NO: | (SEQ ID NO: | (SEQ ID NO: | NTY | | (SEQ ID NO: |
| | | 96) | 126) | 160) | (SEQ ID NO: | | 223) |
| | | | | | 190) | | |
| KIR2DL1/2/3 | Lirilumab | GGTFSFYA | FIPIFGAA | ARIPSGSYY | QSVSSY | DAS | QQRSNWMY |
| | | (SEQ ID NO: | (SEQ ID NO: | YDYDMDV | (SEQ ID NO: | | T |
| | | 97) | 127) | (SEQ ID NO: | 180) | | (SEQ ID NO: |
| | | | | 161) | | | 224) |
| MUC5AC | Ensituximab | GFSLSKFG | IWGDGST | VKPGGDY | SSISY | DTS | HQRDSYPW |
| | | (SEQ ID NO: | (SEQ ID NO: | (SEQ ID NO: | (SEQ ID NO: | | T |
| | | 98) | 128) | 162) | 191) | | (SEQ ID NO: |
| | | | | | | | 225) |
| phospha- | Bavituximab | GYSFTGYN | IDPYYGDT | VKGGYYGH | QDIGSS | ATS | LQYVSSPPT |
| tidyl- | | (SEQ ID NO: | (SEQ ID NO: | VVYFDV | (SEQ ID NO: | | (SEQ ID NO: |
| serine | | 84) | 129) | (SEQ ID NO: | 192) | | 226) |
| | | | | 163) | | | |
| RHD | Roledumab | GFTFKNYA | ISYDGRNI | ARPVRSRW | QDIRNY | AAS | QQYYNSPP |
| | | (SEQ ID NO: | (SEQ ID NO: | LQLGLEDAF | (SEQ ID NO: | | T |
| | | 99) | 130) | HI | 193) | | (SEQ ID NO: |
| | | | | (SEQ ID NO: | | | 227) |
| | | | | 164) | | | |
| SLAMF7 | Elotuzumab | GFDFSRYW | INPDSSTI | ARPDGNYW | QDVGIA | WAS | QQYSSYPY |
| | | (SEQ ID NO: | (SEQ ID NO: | YFDV | (SEQ ID NO: | | T |
| | | 100) | 131) | (SEQ ID NO: | 194) | | (SEQ ID NO: |
| | | | | 165) | | | 228) |
| HER2 | Trastuzumab | GFNIKDTY | IYPTNGYT | SRWGGDGF | QDVNTA | SAS | QQHYTTPPT |
| | | (SEQ ID NO: | (SEQ ID NO: | YAMDY | (SEQ ID NO: | | (SEQ ID NO: |
| | | 92) | 122) | (SEQ ID NO: | 186) | | 219) |
| | | | | 156) | | | |
| OX40 | Oxelumab | GFTFNSYA | ISGSGGFT | AKDRLVAPG | QGISSW | AAS | QQYNSYPY |
| | | (SEQ ID NO: | (SEQ ID NO: | TFDY | (SEQ ID NO: | | T |
| | | 101) | 132) | (SEQ ID NO: | 195) | | (SEQ ID NO: |
| | | | | 166) | | | 229) |
| PD-L1 | Avelumab | GFTFSSYI | IYPSGGIT | ARIKLGTVT | SSDVGGYN | DVS | SSYTSSSTR |
| | | (SEQ ID NO: | (SEQ ID NO: | TVDY | Y | | V |
| | | 102) | 133) | (SEQ ID NO: | (SEQ ID NO: | | (SEQ ID NO: |
| | | | | 167) | 196) | | 230) |
| CD135 | 4G8-SDIEM | SYWMH | EIDPSDSYK | AITTTPFDF | RASQSISNN | YSQSIS | QQSNTWPY |
| | | (SEQ ID NO: | DYNQKFKD | (SEQ ID NO: | LH | (SEQ ID NO: | T |
| | | 103) | (SEQ ID NO: | 168) | (SEQ ID NO: | 200) | (SEQ ID NO: |
| | | | 134) | | 197) | | 231) |
| HIV1 | VRC01LS | GYTELNCPI | GWMKPRG | ARYFFGSSP | SQYGSLAW | GGS | QQYEFFGQ |
| | | (SEQ ID NO: | GAVN | NWYFD | (SEQ ID NO: | | GT |
| | | 104) | (SEQ ID NO: | (SEQ ID NO: | 198) | | (SEQ ID NO: |
| | | | 135) | 169) | | | 232) |
| HER3 | KTN3379 | GFTFSYYYM | IGSSGGVTN | ARVGLGDA | SLSNIGLN | SRN | AAWDDSPP |
| | | Q | (SEQ ID NO: | FDIWQQ | (SEQ ID NO: | | G |
| | | (SEQ ID NO: | 136) | (SEQ ID NO: | 199) | | (SEQ ID NO: |
| | | 105) | | 170) | | | 233) |

The antigen binding domain of Fc-antigen binding domain construct 1 (110/104 in FIG. 1) can include the three heavy chain and the three light chain CDR sequences of any one of the antibodies listed in Table 1.

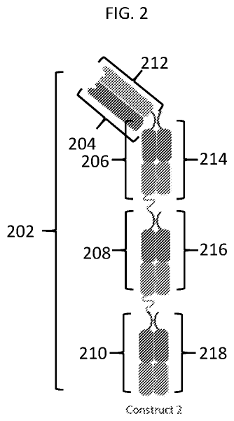

The antigen binding domain of Fc-antigen binding domain construct 2 (212/204 in FIG. 2) can include the three heavy chain and the three light chain CDR sequences of any one of the antibodies listed in Table 1.

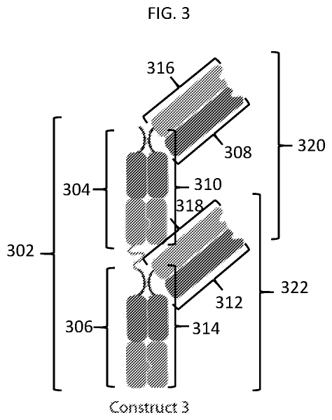

The antigen binding domains of Fc-antigen binding domain construct 3 (308/316 and 312/318 in FIG. 3) each can include the three heavy chain and the three light chain CDR sequences of any one of the antibodies listed in Table 1.

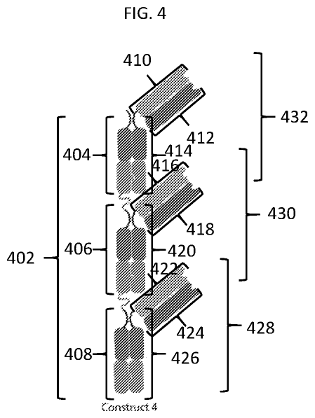

The antigen binding domains of Fc-antigen binding domain construct 4 (410/412, 416/418 and 422/424 in FIG. 4) each can include the three heavy chain and the three light chain CDR sequences of any one of the antibodies listed in Table 1.

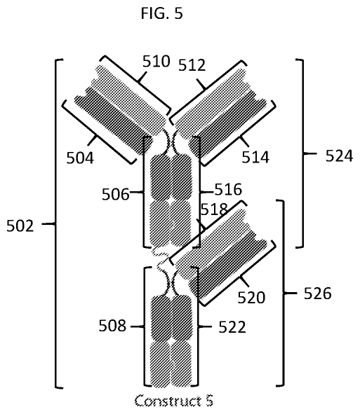

The antigen binding domains of Fc-antigen binding domain construct 5 (510/504, 512/514 and 518/520 in FIG. 5) each can include the three heavy chain and the three light chain CDR sequences of any one of the antibodies listed in Table 1.

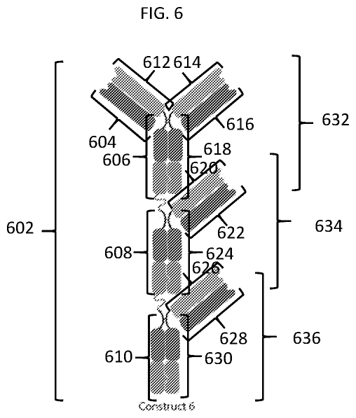

The antigen binding domains of Fc-antigen binding domain construct 6 (612/604, 614/616, 620/622, and 626/628 in FIG. 6) each can include the three heavy chain and the three light chain CDR sequences of any one of the antibodies listed in Table 1.

The antigen binding domains of Fc-antigen binding domain construct 7 (712/714 and 714/716 in FIG. 7) each can include the three heavy chain and the three light chain CDR sequences of any one of the antibodies listed in Table 1.

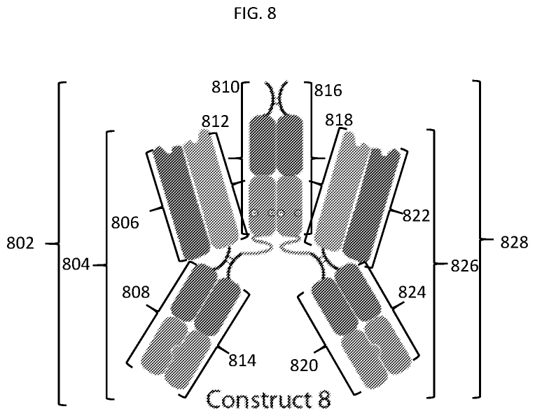

The antigen binding domains of Fc-antigen binding domain construct 8 (812/806 and 818/822 in FIG. 8) each can include the three heavy chain and the three light chain CDR sequences of any one of the antibodies listed in Table 1.

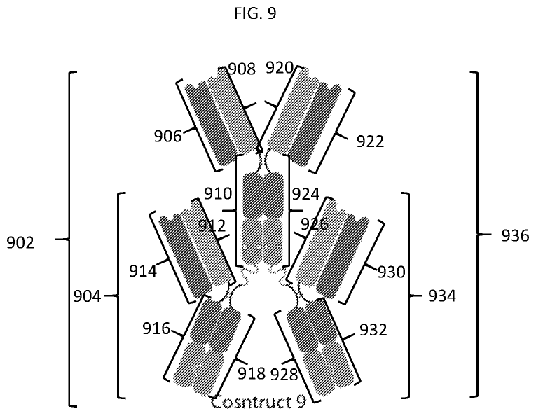

The antigen binding domains of Fc-antigen binding domain construct 9 (908/906, 920/922, 912/914, and 926/930 in FIG. 9) each can include the three heavy chain and the three light chain CDR sequences of any one of the antibodies listed in Table 1.

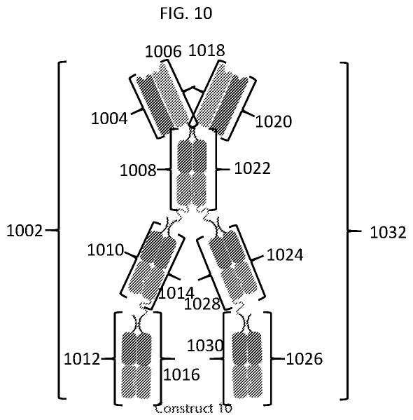

The antigen binding domains of Fc-antigen binding domain construct 10 (1006/1004 and 1018/1020 in FIG. 10) each can include the three heavy chain and the three light chain CDR sequences of any one of the antibodies listed in Table 1.

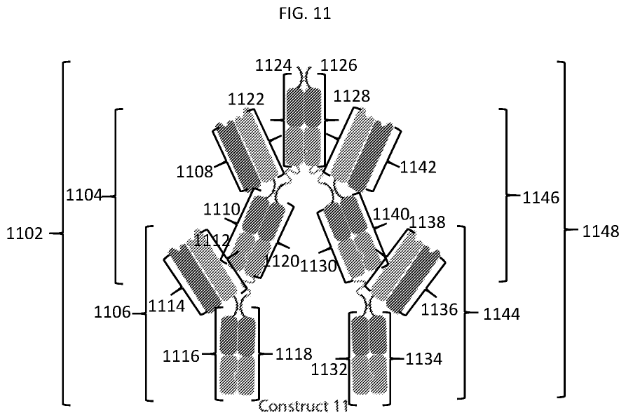

The antigen binding domains of Fc-antigen binding domain construct 11 (1112/1114, 1122/1108, 1128/1142, and 1138/1136 in FIG. 11) each can include the three heavy chain and the three light chain CDR sequences of any one of the antibodies listed in Table 1.

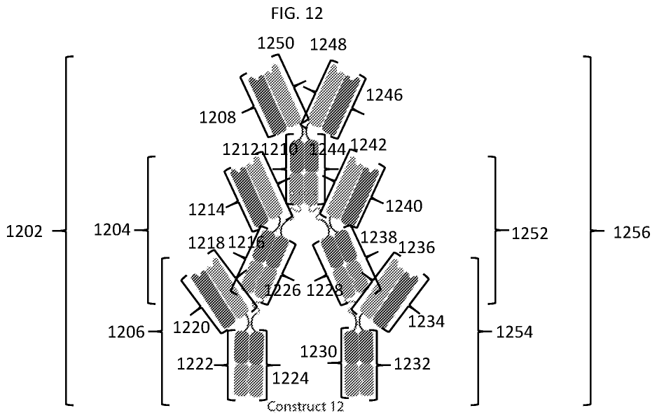

The antigen binding domains of Fc-antigen binding domain construct 12 (1218/1220, 1212/1214, 1250/1208, 1248/1246, 1242/1240, and 1236/1234 in FIG. 12) each can include the three heavy chain and the three light chain CDR sequences of any one of the antibodies listed in Table 1.

The antigen binding domains of Fc-antigen binding domain construct 13 (1310/1304 and 1314/1322 in FIG. 13) each can include the three heavy chain and the three light chain CDR sequences of any one of the antibodies listed in Table 1.

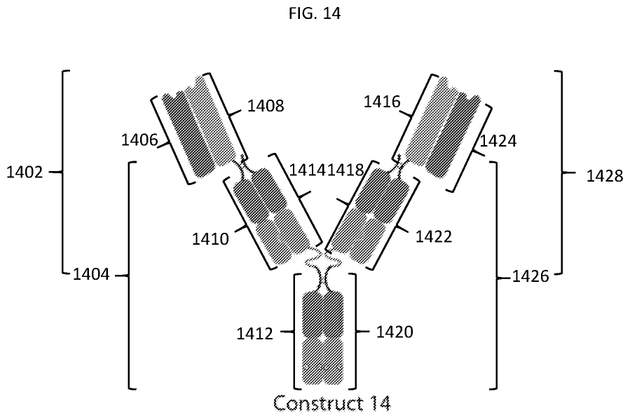

The antigen binding domains of Fc-antigen binding domain construct 14 (1408/1406 and 1416/1424 in FIG. 14) each can include the three heavy chain and the three light chain CDR sequences of any one of the antibodies listed in Table 1.

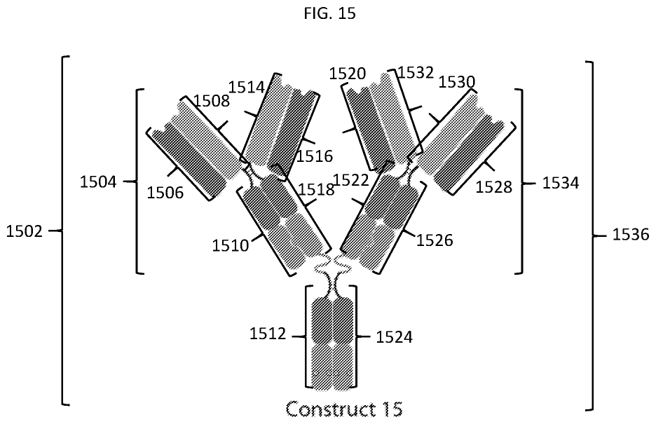

The antigen binding domains of Fc-antigen binding domain construct 15 (1508/1506, 1514/1516, 1532/1520, and 1530/1528 in FIG. 15) each can include the three heavy chain and the three light chain CDR sequences of any one of the antibodies listed in Table 1.

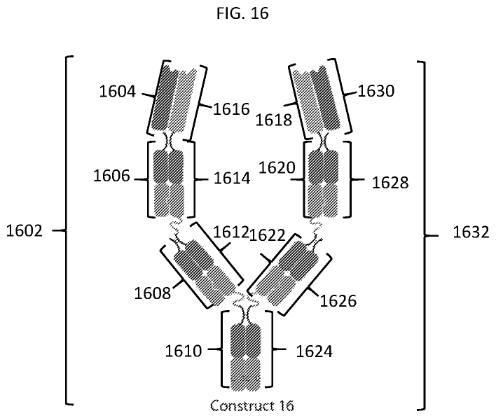

The antigen binding domains of Fc-antigen binding domain construct 16 (1616/1604 and 1618/1630 in FIG. 16) each can include the three heavy chain and the three light chain CDR sequences of any one of the antibodies listed in Table 1.

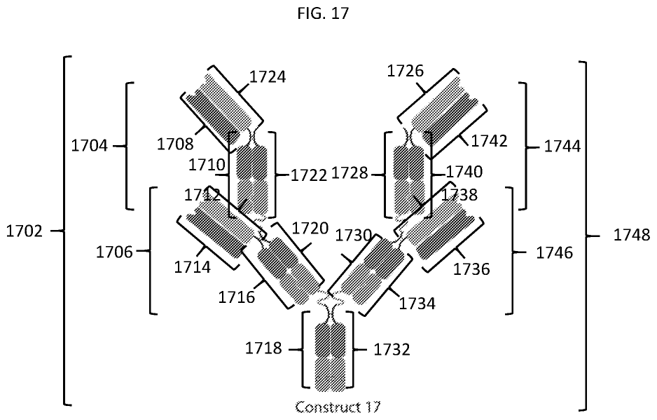

The antigen binding domains of Fc-antigen binding domain construct 17 (1712/1714, 1724/1708, 1726/1742, and 1738/1736 in FIG. 17) each can include the three heavy chain and the three light chain CDR sequences of any one of the antibodies listed in Table 1.

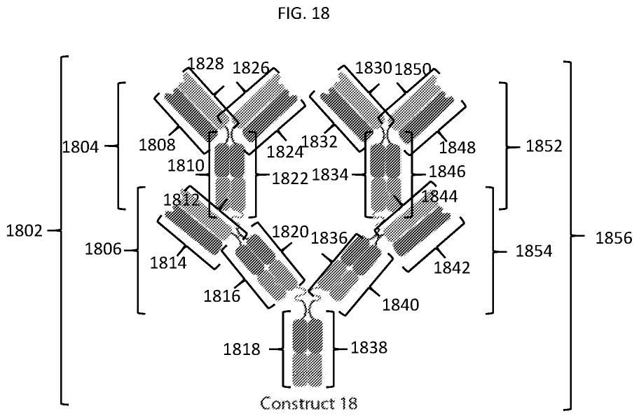

The antigen binding domains of Fc-antigen binding domain construct 18 (1812/1814, 1828/1808, 1826/1824, 1830/1832, 1850/1848, and 1844/1842 in FIG. 18) each can include the three heavy chain and the three light chain CDR sequences of any one of the antibodies listed in Table 1.

The antigen binding domains of Fc-antigen binding domain construct 19 (1914/1904 and 1920/1922 in FIG. 19) each can include the three heavy chain and the three light chain CDR sequences of any one of the antibodies listed in Table 1.

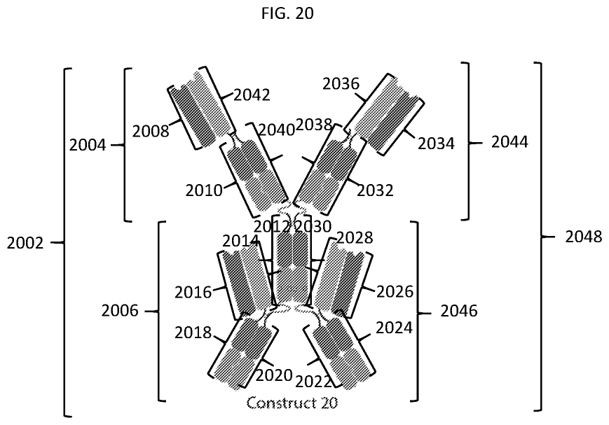

The antigen binding domains of Fc-antigen binding domain construct 20 (2014/2016, 2042/2008, 2036/2034, and 2028/2026 in FIG. 20) each can include the three heavy chain and the three light chain CDR sequences of any one of the antibodies listed in Table 1.

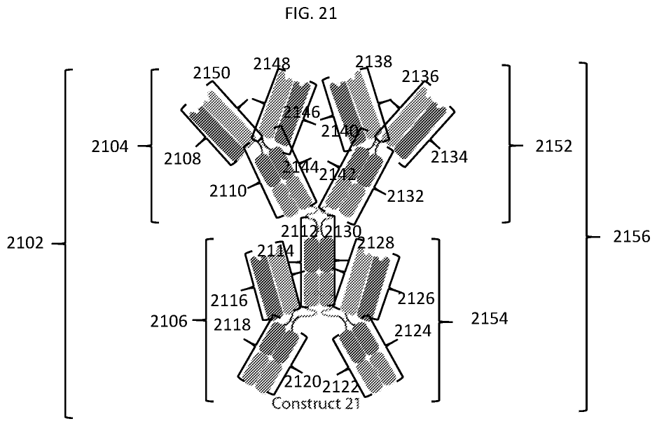

The antigen binding domains of Fc-antigen binding domain construct 21 (2114/2116, 2150/2108, 2148/2146, 2138/2140, 2136/2134, and 2128/2126 in FIG. 21) each can include the three heavy chain and the three light chain CDR sequences of any one of the antibodies listed in Table 1.

An antigen binding domain of Fc-antigen binding domain construct 22 (2204/2222 in FIG. 22) can include the three heavy chain and the three light chain CDR sequences of any one of the antibodies listed in Table 1.

An antigen binding domain of Fc-antigen binding domain construct 22 (each of 2218/2220 and 2212/2214 in FIG. 22) can include the three heavy chain and the three light chain CDR sequences of any one of the antibodies listed in Table 1.

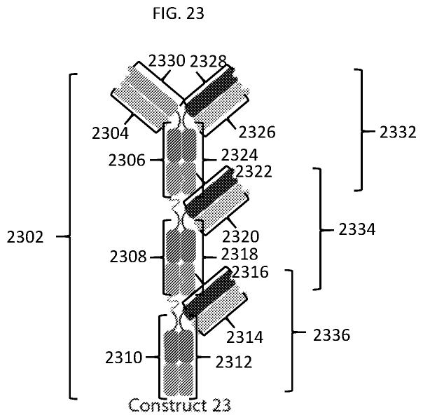

An antigen binding domain of Fc-antigen binding domain construct 23 (2330/2304 in FIG. 23) can include the three heavy chain and the three light chain CDR sequences of any one of the antibodies listed in Table 1.

An antigen binding domain of Fc-antigen binding domain construct 23 (each of 2328/2326, 2322/2320, and 2316/2314 in FIG. 23) can include the three heavy chain and the three light chain CDR sequences of any one of the antibodies listed in Table 1.

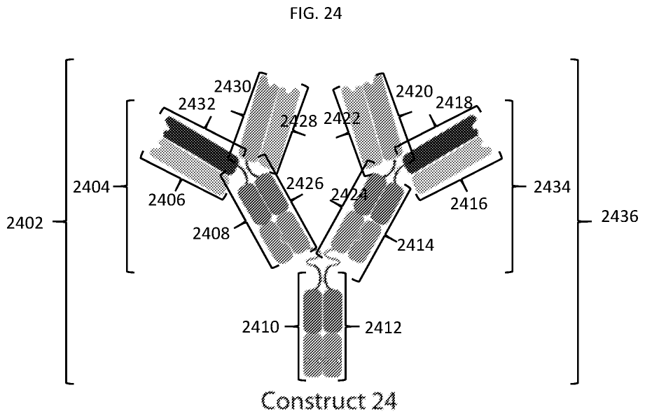

An antigen binding domain of Fc-antigen binding domain construct 24 (each of 2430/2428 and 2420/2422 in FIG. 24) can include the three heavy chain and the three light chain CDR sequences of any one of the antibodies listed in Table 1.

An antigen binding domain of Fc-antigen binding domain construct 24 (each of 2432/2406 and 2418/2416 in FIG. 24) can include the three heavy chain and the three light chain CDR sequences of any one of the antibodies listed in Table 1.

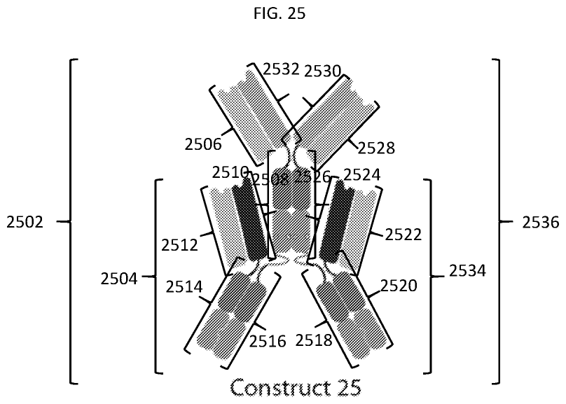

An antigen binding domain of Fc-antigen binding domain construct 25 (each of 2532/2506 and 2530/2528 in FIG. 25) can include the three heavy chain and the three light chain CDR sequences of any one of the antibodies listed in Table 1.

An antigen binding domain of Fc-antigen binding domain construct 25 (each of 2510/2512 and 2524/2522 in FIG. 25) can include the three heavy chain and the three light chain CDR sequences of any one of the antibodies listed in Table 1.

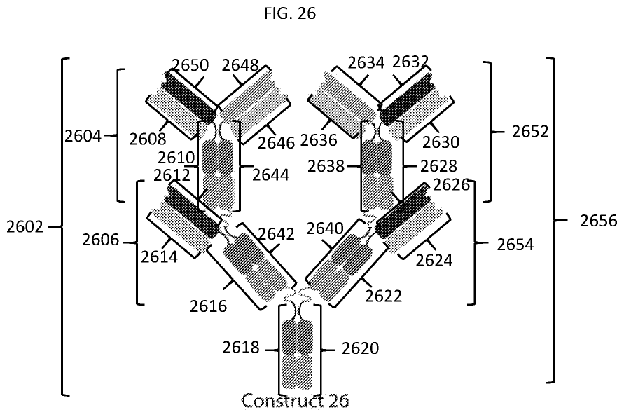

An antigen binding domain of Fc-antigen binding domain construct 26 (each of 2648/2646 and 2634/2636 in FIG. 26) can include the three heavy chain and the three light chain CDR sequences of any one of the antibodies listed in Table 1.

An antigen binding domain of Fc-antigen binding domain construct 26 (each of 2612/2614, 2650/2608, 2632/2630, and 2626/2624 in FIG. 26) can include the three heavy chain and the three light chain CDR sequences of any one of the antibodies listed in Table 1.

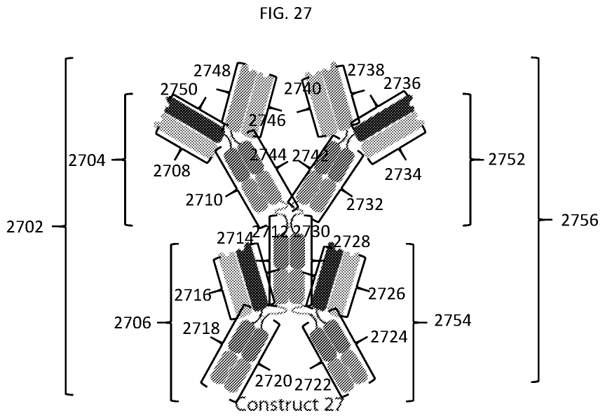

An antigen binding domain of Fc-antigen binding domain construct 27 (each of 2748/2746 and 2738/2740 in FIG. 27) can include the three heavy chain and the three light chain CDR sequences of any one of the antibodies listed in Table 1.

An antigen binding domain of Fc-antigen binding domain construct 27 (each of 2714/2716, 2750/2708, 2736/2734, and 2728/2726 in FIG. 27) can include the three heavy chain and the three light chain CDR sequences of any one of the antibodies listed in Table 1.

An antigen binding domain of Fc-antigen binding domain construct 28 (each of 2850/2808 and 2848/2846 in FIG. 27) can include the three heavy chain and the three light chain CDR sequences of any one of the antibodies listed in Table 1.

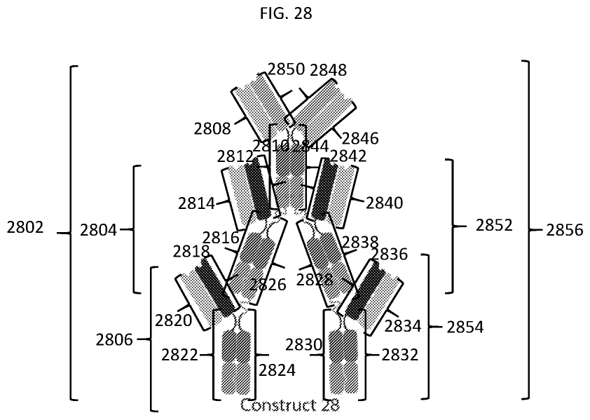

An antigen binding domain of Fc-antigen binding domain construct 28 (each of 2818/2820, 2812/2814, 2842/2840, and 2836/2834 in FIG. 28) can include the three heavy chain and the three light chain CDR sequences of any one of the antibodies listed in Table 1.

An antigen binding domain of Fc-antigen binding domain construct 29 (2918/2904 in FIG. 29) can include the three heavy chain and the three light chain CDR sequences of any one of the antibodies listed in Table 1.

An antigen binding domain of Fc-antigen binding domain construct 29 (2914/2912 in FIG. 29) can include the three heavy chain and the three light chain CDR sequences of any one of the antibodies listed in Table 1.

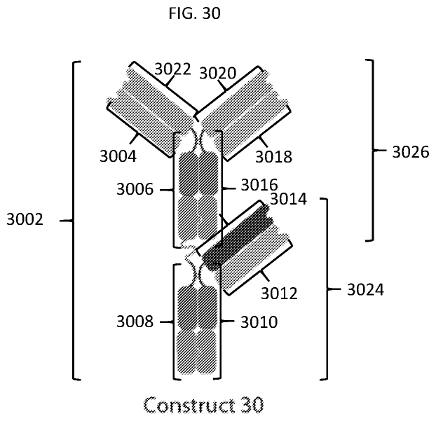

An antigen binding domain of Fc-antigen binding domain construct 30 (each of 3022/3004 and 3020/3018 in FIG. 30) can include the three heavy chain and the three light chain CDR sequences of any one of the antibodies listed in Table 1.

An antigen binding domain of Fc-antigen binding domain construct 30 (3014/3012 in FIG. 30) can include the three heavy chain and the three light chain CDR sequences of any one of the antibodies listed in Table 1.

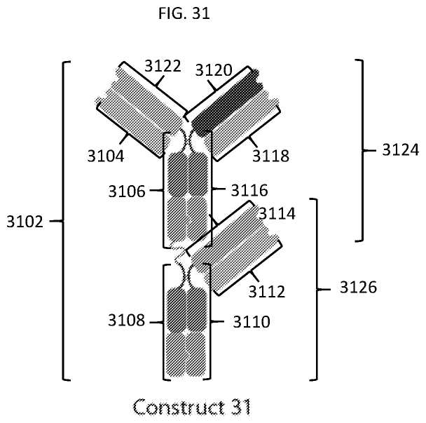

An antigen binding domain of Fc-antigen binding domain construct 31 (3122/3104 in FIG. 31) can include the three heavy chain and the three light chain CDR sequences of any one of the antibodies listed in Table 1.

An antigen binding domain of Fc-antigen binding domain construct 31 (3120/3118 in FIG. 31) can include the three heavy chain and the three light chain CDR sequences of any one of the antibodies listed in Table 1.

An antigen binding domain of Fc-antigen binding domain construct 31 (3114/3112 in FIG. 31) can include the three heavy chain and the three light chain CDR sequences of any one of the antibodies listed in Table 1.

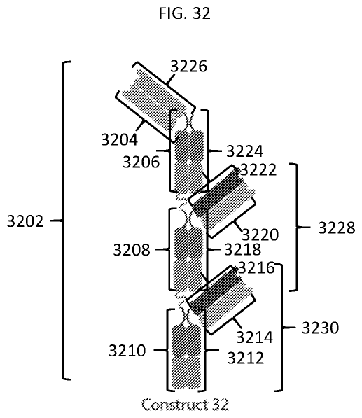

An antigen binding domain of Fc-antigen binding domain construct 32 (3226/3204 in FIG. 32) can include the three heavy chain and the three light chain CDR sequences of any one of the antibodies listed in Table 1.

An antigen binding domain of Fc-antigen binding domain construct 32 (each of 3222/3220 and 3216/3214 in FIG. 32) can include the three heavy chain and the three light chain CDR sequences of any one of the antibodies listed in Table 1.

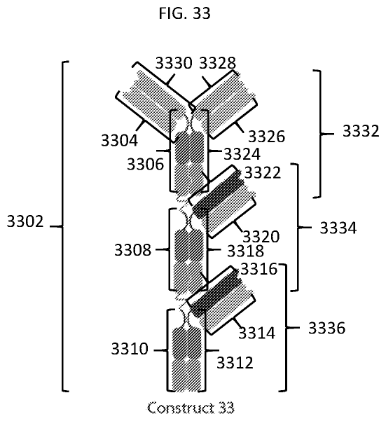

An antigen binding domain of Fc-antigen binding domain construct 33 (each of 3330/3304 and 3328/3326 in FIG. 33) can include the three heavy chain and the three light chain CDR sequences of any one of the antibodies listed in Table 1.

An antigen binding domain of Fc-antigen binding domain construct 33 (each of 3322/3320 and 3316/3314 in FIG. 33) can include the three heavy chain and the three light chain CDR sequences of any one of the antibodies listed in Table 1.

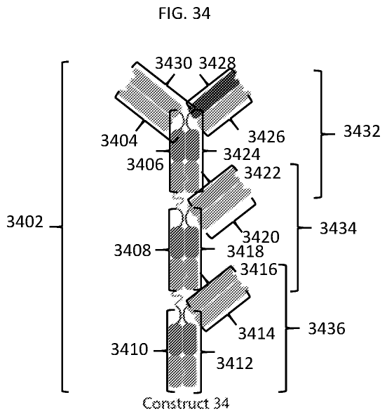

An antigen binding domain of Fc-antigen binding domain construct 34 (3430/3404 in FIG. 34) can include the three heavy chain and the three light chain CDR sequences of any one of the antibodies listed in Table 1.

An antigen binding domain of Fc-antigen binding domain construct 34 (3428/3426 in FIG. 34) can include the three heavy chain and the three light chain CDR sequences of any one of the antibodies listed in Table 1.

An antigen binding domain of Fc-antigen binding domain construct 34 (each of 3422/3420 and 3416/3414 in FIG. 34) can include the three heavy chain and the three light chain CDR sequences of any one of the antibodies listed in Table 1.

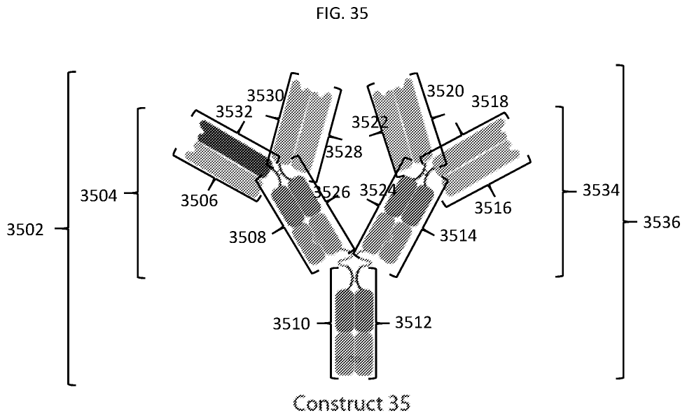

An antigen binding domain of Fc-antigen binding domain construct 35 (each of 3530/3528 and 3520/3522 in FIG. 35) can include the three heavy chain and the three light chain CDR sequences of any one of the antibodies listed in Table 1.

An antigen binding domain of Fc-antigen binding domain construct 35 (3532/3506 in FIG. 35) can include the three heavy chain and the three light chain CDR sequences of any one of the antibodies listed in Table 1.

An antigen binding domain of Fc-antigen binding domain construct 35 (3518/3516 in FIG. 35) can include the three heavy chain and the three light chain CDR sequences of any one of the antibodies listed in Table 1.

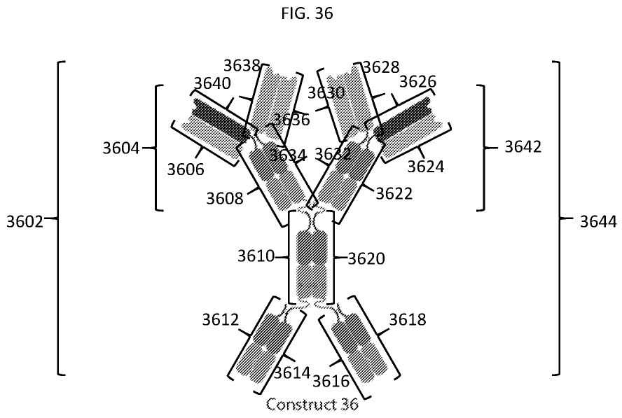

An antigen binding domain of Fc-antigen binding domain construct 36 (each of 3638/3636 and 3628/3620 in FIG. 36) can include the three heavy chain and the three light chain CDR sequences of any one of the antibodies listed in Table 1.

An antigen binding domain of Fc-antigen binding domain construct 36 (each of 3640/3606 and 3626/3624 in FIG. 36) can include the three heavy chain and the three light chain CDR sequences of any one of the antibodies listed in Table 1.

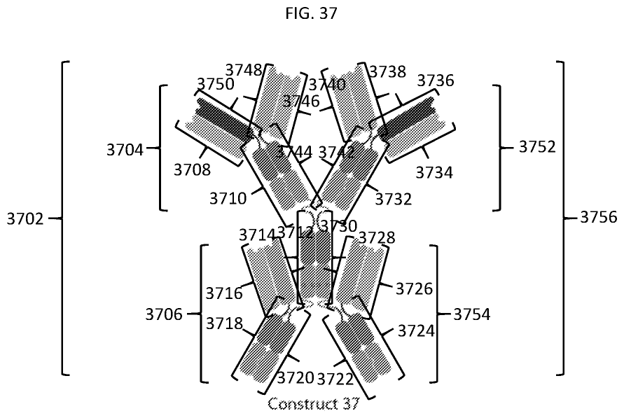

An antigen binding domain of Fc-antigen binding domain construct 37 (each of 3748/3746 and 3738/3740 in FIG. 37) can include the three heavy chain and the three light chain CDR sequences of any one of the antibodies listed in Table 1.

An antigen binding domain of Fc-antigen binding domain construct 37 (each of 3750/3708 and 3736/3734 in FIG. 37) can include the three heavy chain and the three light chain CDR sequences of any one of the antibodies listed in Table 1.

An antigen binding domain of Fc-antigen binding domain construct 37 (each of 3714/3716 and 3728/3726 in FIG. 37) can include the three heavy chain and the three light chain CDR sequences of any one of the antibodies listed in Table 1.

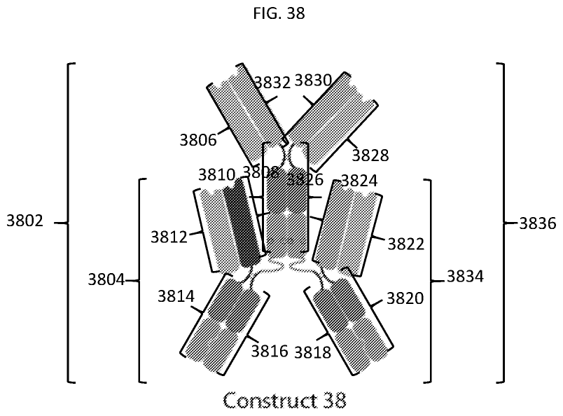

An antigen binding domain of Fc-antigen binding domain construct 38 (each of 3832/3806 and 3830/3822 in FIG. 38) can include the three heavy chain and the three light chain CDR sequences of any one of the antibodies listed in Table 1.

An antigen binding domain of Fc-antigen binding domain construct 38 (3810/3812 in FIG. 38) can include the three heavy chain and the three light chain CDR sequences of any one of the antibodies listed in Table 1.

An antigen binding domain of Fc-antigen binding domain construct 38 (3824/3822 in FIG. 38) can include the three heavy chain and the three light chain CDR sequences of any one of the antibodies listed in Table 1.

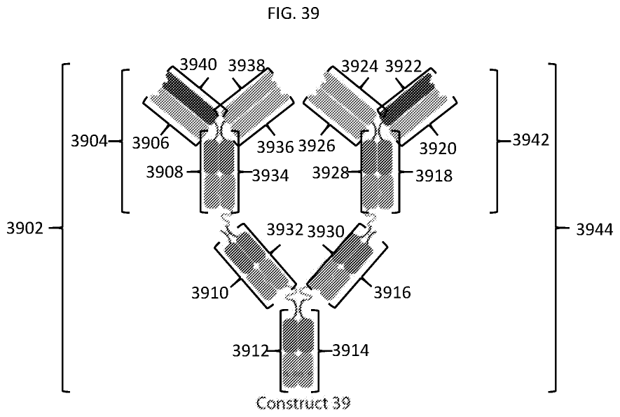

An antigen binding domain of Fc-antigen binding domain construct 39 (each of 3938/3936 and 3924/3926 in FIG. 39) can include the three heavy chain and the three light chain CDR sequences of any one of the antibodies listed in Table 1.

An antigen binding domain of Fc-antigen binding domain construct 39 (each of 3940/3906 and 3922/3920 in FIG. 39) can include the three heavy chain and the three light chain CDR sequences of any one of the antibodies listed in Table 1.

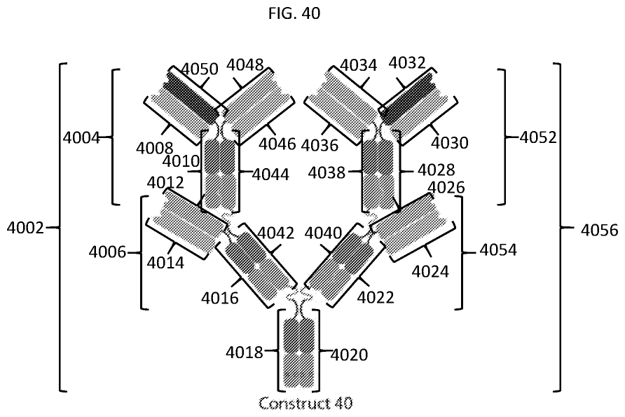

An antigen binding domain of Fc-antigen binding domain construct 40 (each of 4048/4046 and 4034/4036 in FIG. 40) can include the three heavy chain and the three light chain CDR sequences of any one of the antibodies listed in Table 1.

An antigen binding domain of Fc-antigen binding domain construct 40 (each of 4050/4008 and 4032/4030 in FIG. 40) can include the three heavy chain and the three light chain CDR sequences of any one of the antibodies listed in Table 1.

An antigen binding domain of Fc-antigen binding domain construct 40 (each of 4012/4014 and 4026/4024 in FIG. 40) can include the three heavy chain and the three light chain CDR sequences of any one of the antibodies listed in Table 1.

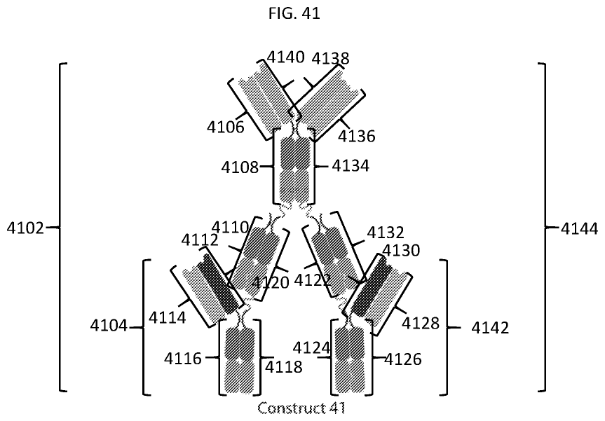

An antigen binding domain of Fc-antigen binding domain construct 41 (each of 4140/4106 and 4138/4136 in FIG. 41) can include the three heavy chain and the three light chain CDR sequences of any one of the antibodies listed in Table 1.

An antigen binding domain of Fc-antigen binding domain construct 41 (each of 4112/4114 and 4130/4128 in FIG. 41) can include the three heavy chain and the three light chain CDR sequences of any one of the antibodies listed in Table 1.

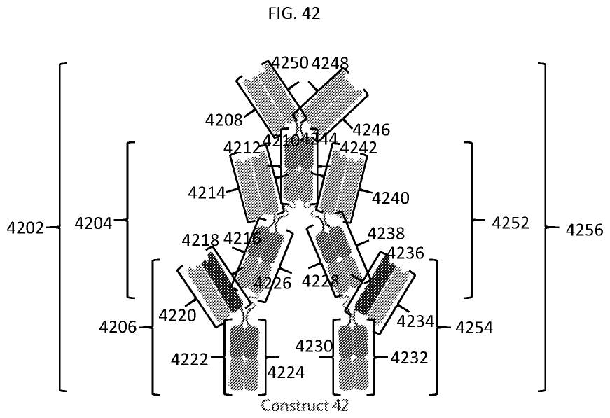

An antigen binding domain of Fc-antigen binding domain construct 42 (each of 4250/4208 and 4248/4246 in FIG. 42) can include the three heavy chain and the three light chain CDR sequences of any one of the antibodies listed in Table 1.

An antigen binding domain of Fc-antigen binding domain construct 42 (each of 4218/4220 and 4236/4234 in FIG. 42) can include the three heavy chain and the three light chain CDR sequences of any one of the antibodies listed in Table 1.

An antigen binding domain of Fc-antigen binding domain construct 42 (each of 4212/4214 and 4242/4240 in FIG. 42) can include the three heavy chain and the three light chain CDR sequences of any one of the antibodies listed in Table 1.

In some embodiments, the antigen binding domain (e.g., a Fab or a scFv) includes the V_{H} and V_{L} chains of an antibody listed in Table 2. In some embodiments, the Fab includes the CDRs contained in the V_{H} and V_{L} chains of an antibody listed in Table 2. In some embodiments, the Fab includes the CDRs contained in the V_{H} and V_{L} chains of an antibody listed in Table 2 and the remainder of the V_{H} and V_{L} sequences are at least 95% identical, at least 97% identical, at least 99% identical, or at least 99.5% identical to the V_{H} and V_{L} sequences of an antibody in Table 2.

TABLE 2

| Target | Antibody Name |
|---|---|
| AbGn-7 antigen | AbGn-7 |
| AMHR2 | GM-102 |
| B7-H3 | DS-5573a |
| CA19-9 | MVT-5873 |
| CAIX | Anti-CAIX |
| CD19 | XmAb5871 |
| CD33 | BI-838858 |
| CD37 | BI-836826 |
| CD38 | MOR-202 |
| CD47 | Anti-CD47 |
| CD70 | ARGX-110 |
| CD70 | ARGX-110 |
| CD98 | IGN-523 |
| CD147 | Metuzumab |
| CD157 | MEN-1112 |
| c-Met | ARGX-111 |
| EGFR2 | GT-Mab 7.3-GEX |
| EphA2 | DS-8895a |
| FGFR2 | FPA-144 |
| GM2 | BIW-8962 |
| HPA-1a | NAITgam |
| ICAM-1 | BI-505 |
| IL-3Ralpha | Talacotuzumab |
| JL-1 | Leukotuximab |
| kappa myeloma | MDX-1097 |
| antigen | |
| KIR32DL2 | IPH-4102 |
| LAG-3 | GSK-2381781 |
| P. aeruginosa | AR-104 |
| serotype O1 | |
| pGlu-abeta | PBD-C06 |
| TA-MUC1 | GT-MAB 2.5-GEX |

The antigen binding domain of Fc-antigen binding domain construct 1 (110/104 in FIG. 1) can include the V_{H} and V_{L} sequences of any one of the antibodies listed in Table 2.

The antigen binding domain of Fc-antigen binding domain construct 2 (212/204 in FIG. 2) can include the V_{H} and V_{L} sequences of any one of the antibodies listed in Table 2.

The antigen binding domains of Fc-antigen binding domain construct 3 (308/316 and 312/318 in FIG. 3) each can include the V_{H} and V_{L} sequences of any one of the antibodies listed in Table 2.

The antigen binding domains of Fc-antigen binding domain construct 4 (410/412, 416/418 and 422/424 in FIG. 4) each can include the V_{H} and V_{L} sequences of any one of the antibodies listed in Table 2.

The antigen binding domains of Fc-antigen binding domain construct 5 (510/504, 512/514 and 518/520 in FIG. 5) each can include the V_{H} and V_{L} sequences of any one of the antibodies listed in Table 2.

The antigen binding domains of Fc-antigen binding domain construct 6 (612/604, 614/616, 620/622, and 626/628 in FIG. 6) each can include the V_{H} and V_{L} sequences of any one of the antibodies listed in Table 2.

The antigen binding domains of Fc-antigen binding domain construct 7 (712/714 and 714/716 in FIG. 7) each can include the V_{H} and V_{L} sequences of any one of the antibodies listed in Table 2.

The antigen binding domains of Fc-antigen binding domain construct 8 (812/806 and 818/822 in FIG. 8) each can include the V_{H} and V_{L} sequences of any one of the antibodies listed in Table 2.

The antigen binding domains of Fc-antigen binding domain construct 9 (908/906, 920/922, 912/914, and 926/930 in FIG. 9) each can include the V_{H} and V_{L} sequences of any one of the antibodies listed in Table 2.

The antigen binding domains of Fc-antigen binding domain construct 10 (1006/1004 and 1018/1020 in FIG. 10) each can include the V_{H} and V_{L} sequences of any one of the antibodies listed in Table 2.

The antigen binding domains of Fc-antigen binding domain construct 11 (1112/1114, 1122/1108, 1128/1142, and 1138/1136 in FIG. 11) each can include the V_{H} and V_{L} sequences of any one of the antibodies listed in Table 2.

The antigen binding domains of Fc-antigen binding domain construct 12 (1218/1220, 1212/1214, 1250/1208, 1248/1246, 1242/1240, and 1236/1234 in FIG. 12) each can include the V_{H} and V_{L} sequences of any one of the antibodies listed in Table 2.

The antigen binding domains of Fc-antigen binding domain construct 13 (1310/1304 and 1314/1322 in FIG. 13) each can include the V_{H} and V_{L} sequences of any one of the antibodies listed in Table 2.

The antigen binding domains of Fc-antigen binding domain construct 14 (1408/1406 and 1416/1424 in FIG. 14) each can include the V_{H} and V_{L} sequences of any one of the antibodies listed in Table 2.

The antigen binding domains of Fc-antigen binding domain construct 15 (1508/1506, 1514/1516, 1532/1520, and 1530/1528 in FIG. 15) each can include the V_{H} and V_{L} sequences of any one of the antibodies listed in Table 2.

The antigen binding domains of Fc-antigen binding domain construct 16 (1616/1604 and 1618/1630 in FIG. 16) each can include the V_{H} and V_{L} sequences of any one of the antibodies listed in Table 2.

The antigen binding domains of Fc-antigen binding domain construct 17 (1712/1714, 1724/1708, 1726/1742, and 1738/1736 in FIG. 17) each can include the V_{H} and V_{L} sequences of any one of the antibodies listed in Table 2.

The antigen binding domains of Fc-antigen binding domain construct 18 (1812/1814, 1828/1808, 1826/1824, 1830/1832, 1850/1848, and 1844/1842 in FIG. 18) each can include the V_{H} and V_{L} sequences of any one of the antibodies listed in Table 2.

The antigen binding domains of Fc-antigen binding domain construct 19 (1914/1904 and 1920/1922 in FIG. 19) each can include the V_{H} and V_{L} sequences of any one of the antibodies listed in Table 2.

The antigen binding domains of Fc-antigen binding domain construct 20 (2014/2016, 2042/2008, 2036/2034, and 2028/2026 in FIG. 20) each can include the V_{H} and V_{L} sequences of any one of the antibodies listed in Table 2.

The antigen binding domains of Fc-antigen binding domain construct 21 (2114/2116, 2150/2108, 2148/2146, 2138/2140, 2136/2134, and 2128/2126 in FIG. 21) each can include the V_{H} and V_{L} sequences of any one of the antibodies listed in Table 2.

An antigen binding domain of Fc-antigen binding domain construct 22 (2204/2222 in FIG. 22) can include the VH and V_{L} sequences of any one of the antibodies listed in Table 2.

An antigen binding domain of Fc-antigen binding domain construct 22 (each of 2218/2220 and 2212/2214 in FIG. 22) can include the V_{H} and V_{L} sequences of any one of the antibodies listed in Table 2.

An antigen binding domain of Fc-antigen binding domain construct 23 (2330/2304 in FIG. 23) can include the VH and V_{L} sequences of any one of the antibodies listed in Table 2.

An antigen binding domain of Fc-antigen binding domain construct 23 (each of 2328/2326, 2322/2320, and 2316/2314 in FIG. 23) can include the V_{H} and V_{L} sequences of any one of the antibodies listed in Table 2.

An antigen binding domain of Fc-antigen binding domain construct 24 (each of 2430/2428 and 2420/2422 in FIG. 24) can include the V_{H} and V_{L} sequences of any one of the antibodies listed in Table 2.

An antigen binding domain of Fc-antigen binding domain construct 24 (each of 2432/2406 and 2418/2416 in FIG. 24) can include the V_{H} and V_{L} sequences of any one of the antibodies listed in Table 2.

An antigen binding domain of Fc-antigen binding domain construct 25 (each of 2532/2506 and 2530/2528 in FIG. 25) can include the V_{H} and V_{L} sequences of any one of the antibodies listed in Table 2.

An antigen binding domain of Fc-antigen binding domain construct 25 (each of 2510/2512 and 2524/2522 in FIG. 25) can include the V_{H} and V_{L} sequences of any one of the antibodies listed in Table 2.

An antigen binding domain of Fc-antigen binding domain construct 26 (each of 2648/2646 and 2634/2636 in FIG. 26) can include the V_{H} and V_{L} sequences of any one of the antibodies listed in Table 2.

An antigen binding domain of Fc-antigen binding domain construct 26 (each of 2612/2614, 2650/2608, 2632/2630, and 2626/2624 in FIG. 26) can include the V_{H} and V_{L} sequences of any one of the antibodies listed in Table 2.

An antigen binding domain of Fc-antigen binding domain construct 27 (each of 2748/2746 and 2738/2740 in FIG. 27) can include the V_{H} and V_{L} sequences of any one of the antibodies listed in Table 2.

An antigen binding domain of Fc-antigen binding domain construct 27 (each of 2714/2716, 2750/2708, 2736/2734, and 2728/2726 in FIG. 27) can include the V_{H} and V_{L} sequences of any one of the antibodies listed in Table 2.

An antigen binding domain of Fc-antigen binding domain construct 28 (each of 2850/2808 and 2848/2846 in FIG. 27) can include the V_{H} and V_{L} sequences of any one of the antibodies listed in Table 2.

An antigen binding domain of Fc-antigen binding domain construct 28 (each of 2818/2820, 2812/2814, 2842/2840, and 2836/2834 in FIG. 28) can include the V_{H} and V_{L} sequences of any one of the antibodies listed in Table 2.

An antigen binding domain of Fc-antigen binding domain construct 29 (2918/2904 in FIG. 29) can include the V_{H} and V_{L} sequences of any one of the antibodies listed in Table 2.

An antigen binding domain of Fc-antigen binding domain construct 29 (2914/2912 in FIG. 29) can include the VH and V_{L} sequences of any one of the antibodies listed in Table 2.

An antigen binding domain of Fc-antigen binding domain construct 30 (each of 3022/3004 and 3020/3018 in FIG. 30) can include the V_{H} and V_{L} sequences of any one of the antibodies listed in Table 2.

An antigen binding domain of Fc-antigen binding domain construct 30 (3014/3012 in FIG. 30) can include the V_{H} and V_{L} sequences of any one of the antibodies listed in Table 2.

An antigen binding domain of Fc-antigen binding domain construct 31 (3122/3104 in FIG. 31) can include the V_{H} and V_{L} sequences of any one of the antibodies listed in Table 2.

An antigen binding domain of Fc-antigen binding domain construct 31 (3120/3118 in FIG. 31) can include the V_{H} and V_{L} sequences of any one of the antibodies listed in Table 2.

An antigen binding domain of Fc-antigen binding domain construct 31 (3114/3112 in FIG. 31) can include the V_{H} and V_{L} sequences of any one of the antibodies listed in Table 2.

An antigen binding domain of Fc-antigen binding domain construct 32 (3226/3204 in FIG. 32) can include the V_{H} and V_{L} sequences of any one of the antibodies listed in Table 2.

An antigen binding domain of Fc-antigen binding domain construct 32 (each of 3222/3220 and 3216/3214 in FIG. 32) can include the V_{H} and V_{L} sequences of any one of the antibodies listed in Table 2.

An antigen binding domain of Fc-antigen binding domain construct 33 (each of 3330/3304 and 3328/3326 in FIG. 33) can include the V_{H} and V_{L} sequences of any one of the antibodies listed in Table 2.

An antigen binding domain of Fc-antigen binding domain construct 33 (each of 3322/3320 and 3316/3314 in FIG. 33) can include the V_{H} and V_{L} sequences of any one of the antibodies listed in Table 2.

An antigen binding domain of Fc-antigen binding domain construct 34 (3430/3404 in FIG. 34) can include the V_{H} and V_{L} sequences of any one of the antibodies listed in Table 2.

An antigen binding domain of Fc-antigen binding domain construct 34 (3428/3426 in FIG. 34) can include the V_{H} and V_{L} sequences of any one of the antibodies listed in Table 2.

An antigen binding domain of Fc-antigen binding domain construct 34 (each of 3422/3420 and 3416/3414 in FIG. 34) can include the V_{H} and V_{L} sequences of any one of the antibodies listed in Table 2.

An antigen binding domain of Fc-antigen binding domain construct 35 (each of 3530/3528 and 3520/3522 in FIG. 35) can include the V_{H} and V_{L} sequences of any one of the antibodies listed in Table 2.

An antigen binding domain of Fc-antigen binding domain construct 35 (3532/3506 in FIG. 35) can include the V_{H} and V_{L} sequences of any one of the antibodies listed in Table 2.

An antigen binding domain of Fc-antigen binding domain construct 35 (3518/3516 in FIG. 35) can include the V_{H} and V_{L} sequences of any one of the antibodies listed in Table 2.

An antigen binding domain of Fc-antigen binding domain construct 36 (each of 3638/3636 and 3628/3620 in FIG. 36) can include the V_{H} and V_{L} sequences of any one of the antibodies listed in Table 2.

An antigen binding domain of Fc-antigen binding domain construct 36 (each of 3640/3606 and 3626/3624 in FIG. 36) can include the V_{H} and V_{L} sequences of any one of the antibodies listed in Table 2.

An antigen binding domain of Fc-antigen binding domain construct 37 (each of 3748/3746 and 3738/3740 in FIG. 37) can include the V_{H} and V_{L} sequences of any one of the antibodies listed in Table 2.

An antigen binding domain of Fc-antigen binding domain construct 37 (each of 3750/3708 and 3736/3734 in FIG. 37) can include the V_{H} and V_{L} sequences of any one of the antibodies listed in Table 2.

An antigen binding domain of Fc-antigen binding domain construct 37 (each of 3714/3716 and 3728/3726 in FIG. 37) can include the V_{H} and V_{L} sequences of any one of the antibodies listed in Table 2.

An antigen binding domain of Fc-antigen binding domain construct 38 (each of 3832/3806 and 3830/3822 in FIG. 38) can include the V_{H} and V_{L} sequences of any one of the antibodies listed in Table 2.

An antigen binding domain of Fc-antigen binding domain construct 38 (3810/3812 in FIG. 38) can include the V_{H} and V_{L} sequences of any one of the antibodies listed in Table 2.

An antigen binding domain of Fc-antigen binding domain construct 38 (3824/3822 in FIG. 38) can include the V_{H} and V_{L} sequences of any one of the antibodies listed in Table 2.

An antigen binding domain of Fc-antigen binding domain construct 39 (each of 3938/3936 and 3924/3926 in FIG. 39) can include the V_{H} and V_{L} sequences of any one of the antibodies listed in Table 2.

An antigen binding domain of Fc-antigen binding domain construct 39 (each of 3940/3906 and 3922/3920 in FIG. 39) can include the V_{H} and V_{L} sequences of any one of the antibodies listed in Table 2.

An antigen binding domain of Fc-antigen binding domain construct 40 (each of 4048/4046 and 4034/4036 in FIG. 40) can include the V_{H} and V_{L} sequences of any one of the antibodies listed in Table 2.

An antigen binding domain of Fc-antigen binding domain construct 40 (each of 4050/4008 and 4032/4030 in FIG. 40) can include the V_{H} and V_{L} sequences of any one of the antibodies listed in Table 2.

An antigen binding domain of Fc-antigen binding domain construct 40 (each of 4012/4014 and 4026/4024 in FIG. 40) can include the V_{H} and V_{L} sequences of any one of the antibodies listed in Table 2.

An antigen binding domain of Fc-antigen binding domain construct 41 (each of 4140/4106 and 4138/4136 in FIG. 41) can include the V_{H} and V_{L} sequences of any one of the antibodies listed in Table 2.

An antigen binding domain of Fc-antigen binding domain construct 41 (each of 4112/4114 and 4130/4128 in FIG. 41) can include the V_{H} and V_{L} sequences of any one of the antibodies listed in Table 2.

An antigen binding domain of Fc-antigen binding domain construct 42 (each of 4250/4208 and 4248/4246 in FIG. 42) can include the V_{H} and V_{L} sequences of any one of the antibodies listed in Table 2.

An antigen binding domain of Fc-antigen binding domain construct 42 (each of 4218/4220 and 4236/4234 in FIG. 42) can include the V_{H} and V_{L} sequences of any one of the antibodies listed in Table 2.

An antigen binding domain of Fc-antigen binding domain construct 42 (each of 4212/4214 and 4242/4240 in FIG. 42) can include the V_{H} and V_{L} sequences of any one of the antibodies listed in Table 2.

The antigen binding domain of Fc-antigen binding domain construct 1 (110/104 in FIG. 1) can include the CDR sequences contained in the V_{H} and V_{L} sequences of any one of the antibodies listed in Table 2.

The antigen binding domain of Fc-antigen binding domain construct 2 (212/204 in FIG. 2) can include the CDR sequences contained in the V_{H} and V_{L} sequences of any one of the antibodies listed in Table 2.

The antigen binding domains of Fc-antigen binding domain construct 3 (308/316 and 312/318 in FIG. 3) each can include the CDR sequences contained in the V_{H} and V_{L} sequences of any one of the antibodies listed in Table 2.

The antigen binding domains of Fc-antigen binding domain construct 4 (410/412, 416/418 and 422/424 in FIG. 4) each can include the CDR sequences contained in the V_{H} and V_{L} sequences of any one of the antibodies listed in Table 2.

The antigen binding domains of Fc-antigen binding domain construct 5 (510/504, 512/514 and 518/520 in FIG. 5) each can include the CDR sequences contained in the V_{H} and V_{L} sequences of any one of the antibodies listed in Table 2.

The antigen binding domains of Fc-antigen binding domain construct 6 (612/604, 614/616, 620/622, and 626/628 in FIG. 6) each can include the CDR sequences contained in the V_{H} and V_{L} sequences of any one of the antibodies listed in Table 2.

The antigen binding domains of Fc-antigen binding domain construct 7 (712/714 and 714/716 in FIG. 7) each can include the CDR sequences contained in the V_{H} and V_{L} sequences of any one of the antibodies listed in Table 2.

The antigen binding domains of Fc-antigen binding domain construct 8 (812/806 and 818/822 in FIG. 8) each can include the CDR sequences contained in the V_{H} and V_{L} sequences of any one of the antibodies listed in Table 2.

The antigen binding domains of Fc-antigen binding domain construct 9 (908/906, 920/922, 912/914, and 926/930 in FIG. 9) each can include the CDR sequences contained in the V_{H} and V_{L} sequences of any one of the antibodies listed in Table 2.

The antigen binding domains of Fc-antigen binding domain construct 10 (1006/1004 and 1018/1020 in FIG. 10) each can include the CDR sequences contained in the V_{H} and V_{L} sequences of any one of the antibodies listed in Table 2.

The antigen binding domains of Fc-antigen binding domain construct 11 (1112/1114, 1122/1108, 1128/1142, and 1138/1136 in FIG. 11) each can include the CDR sequences contained in the V_{H} and V_{L} sequences of any one of the antibodies listed in Table 2.

The antigen binding domains of Fc-antigen binding domain construct 12 (1218/1220, 1212/1214, 1250/1208, 1248/1246, 1242/1240, and 1236/1234 in FIG. 12) each can include the CDR sequences contained in the V_{H} and V_{L} sequences of any one of the antibodies listed in Table 2.

The antigen binding domains of Fc-antigen binding domain construct 13 (1310/1304 and 1314/1322 in FIG. 13) each can include the CDR sequences contained in the V_{H} and V_{L} sequences of any one of the antibodies listed in Table 2.

The antigen binding domains of Fc-antigen binding domain construct 14 (1408/1406 and 1416/1424 in FIG. 14) each can include the CDR sequences contained in the V_{H} and V_{L} sequences of any one of the antibodies listed in Table 2.

The antigen binding domains of Fc-antigen binding domain construct 15 (1508/1506, 1514/1516, 1532/1520, and 1530/1528 in FIG. 15) each can include the CDR sequences contained in the V_{H} and V_{L} sequences of any one of the antibodies listed in Table 2.

The antigen binding domains of Fc-antigen binding domain construct 16 (1616/1604 and 1618/1630 in FIG. 16) each can include the CDR sequences contained in the V_{H} and V_{L} sequences of any one of the antibodies listed in Table 2.

The antigen binding domains of Fc-antigen binding domain construct 17 (1712/1714, 1724/1708, 1726/1742, and 1738/1736 in FIG. 17) each can include the CDR sequences contained in the V_{H} and V_{L} sequences of any one of the antibodies listed in Table 2.

The antigen binding domains of Fc-antigen binding domain construct 18 (1812/1814, 1828/1808, 1826/1824, 1830/1832, 1850/1848, and 1844/1842 in FIG. 18) each can include the CDR sequences contained in the V_{H} and V_{L} sequences of any one of the antibodies listed in Table 2.

The antigen binding domains of Fc-antigen binding domain construct 19 (1914/1904 and 1920/1922 in FIG. 19) each can include the CDR sequences contained in the V_{H} and V_{L} sequences of any one of the antibodies listed in Table 2.

The antigen binding domains of Fc-antigen binding domain construct 20 (2014/2016, 2042/2008, 2036/2034, and 2028/2026 in FIG. 20) each can include the CDR sequences contained in the V_{H} and V_{L} sequences of any one of the antibodies listed in Table 2.

The antigen binding domains of Fc-antigen binding domain construct 21 (2114/2116, 2150/2108, 2148/2146, 2138/2140, 2136/2134, and 2128/2126 in FIG. 21) each can include the CDR sequences contained in the V_{H} and V_{L} sequences of any one of the antibodies listed in Table 2.

An antigen binding domain of Fc-antigen binding domain construct 22 (2204/2222 in FIG. 22) can include the CDR sequences contained in the V_{H} and V_{L} sequences of any one of the antibodies listed in Table 2.

An antigen binding domain of Fc-antigen binding domain construct 22 (each of 2218/2220 and 2212/2214 in FIG. 22) can include the CDR sequences contained in the V_{H} and V_{L} sequences of any one of the antibodies listed in Table 2.

An antigen binding domain of Fc-antigen binding domain construct 23 (2330/2304 in FIG. 23) can include the CDR sequences contained in the V_{H} and V_{L} sequences of any one of the antibodies listed in Table 2.

An antigen binding domain of Fc-antigen binding domain construct 23 (each of 2328/2326, 2322/2320, and 2316/2314 in FIG. 23) can include the CDR sequences contained in the V_{H} and V_{L} sequences of any one of the antibodies listed in Table 2.

An antigen binding domain of Fc-antigen binding domain construct 24 (each of 2430/2428 and 2420/2422 in FIG. 24) can include the CDR sequences contained in the V_{H} and V_{L} sequences of any one of the antibodies listed in Table 2.

An antigen binding domain of Fc-antigen binding domain construct 24 (each of 2432/2406 and 2418/2416 in FIG. 24) can include the CDR sequences contained in the V_{H} and V_{L} sequences of any one of the antibodies listed in Table 2.

An antigen binding domain of Fc-antigen binding domain construct 25 (each of 2532/2506 and 2530/2528 in FIG. 25) can include the CDR sequences contained in the V_{H} and V_{L} sequences of any one of the antibodies listed in Table 2.

An antigen binding domain of Fc-antigen binding domain construct 25 (each of 2510/2512 and 2524/2522 in FIG. 25) can include the CDR sequences contained in the V_{H} and V_{L} sequences of any one of the antibodies listed in Table 2.

An antigen binding domain of Fc-antigen binding domain construct 26 (each of 2648/2646 and 2634/2636 in FIG. 26) can include the CDR sequences contained in the V_{H} and V_{L} sequences of any one of the antibodies listed in Table 2.

An antigen binding domain of Fc-antigen binding domain construct 26 (each of 2612/2614, 2650/2608, 2632/2630, and 2626/2624 in FIG. 26) can include the CDR sequences contained in the V_{H} and V_{L} sequences of any one of the antibodies listed in Table 2.

An antigen binding domain of Fc-antigen binding domain construct 27 (each of 2748/2746 and 2738/2740 in FIG. 27) can include the CDR sequences contained in the V_{H} and V_{L} sequences of any one of the antibodies listed in Table 2.

An antigen binding domain of Fc-antigen binding domain construct 27 (each of 2714/2716, 2750/2708, 2736/2734, and 2728/2726 in FIG. 27) can include the CDR sequences contained in the V_{H} and V_{L} sequences of any one of the antibodies listed in Table 2.

An antigen binding domain of Fc-antigen binding domain construct 28 (each of 2850/2808 and 2848/2846 in FIG. 27) can include the CDR sequences contained in the V_{H} and V_{L} sequences of any one of the antibodies listed in Table 2.

An antigen binding domain of Fc-antigen binding domain construct 28 (each of 2818/2820, 2812/2814, 2842/2840, and 2836/2834 in FIG. 28) can include the CDR sequences contained in the V_{H} and V_{L} sequences of any one of the antibodies listed in Table 2.

An antigen binding domain of Fc-antigen binding domain construct 29 (2918/2904 in FIG. 29) can include the CDR sequences contained in the V_{H} and V_{L} sequences of any one of the antibodies listed in Table 2.

An antigen binding domain of Fc-antigen binding domain construct 29 (2914/2912 in FIG. 29) can include the CDR sequences contained in the V_{H} and V_{L} sequences of any one of the antibodies listed in Table 2.

An antigen binding domain of Fc-antigen binding domain construct 30 (each of 3022/3004 and 3020/3018 in FIG. 30) can include the CDR sequences contained in the V_{H} and V_{L} sequences of any one of the antibodies listed in Table 2.

An antigen binding domain of Fc-antigen binding domain construct 30 (3014/3012 in FIG. 30) can include the CDR sequences contained in the V_{H} and V_{L} sequences of any one of the antibodies listed in Table 2.

An antigen binding domain of Fc-antigen binding domain construct 31 (3122/3104 in FIG. 31) can include the CDR sequences contained in the V_{H} and V_{L} sequences of any one of the antibodies listed in Table 2.

An antigen binding domain of Fc-antigen binding domain construct 31 (3120/3118 in FIG. 31) can include the CDR sequences contained in the V_{H} and V_{L} sequences of any one of the antibodies listed in Table 2.

An antigen binding domain of Fc-antigen binding domain construct 31 (3114/3112 in FIG. 31) can include the CDR sequences contained in the V_{H} and V_{L} sequences of any one of the antibodies listed in Table 2.

An antigen binding domain of Fc-antigen binding domain construct 32 (3226/3204 in FIG. 32) can include the CDR sequences contained in the V_{H} and V_{L} sequences of any one of the antibodies listed in Table 2.

An antigen binding domain of Fc-antigen binding domain construct 32 (each of 3222/3220 and 3216/3214 in FIG. 32) can include the CDR sequences contained in the V_{H} and V_{L} sequences of any one of the antibodies listed in Table 2.

An antigen binding domain of Fc-antigen binding domain construct 33 (each of 3330/3304 and 3328/3326 in FIG. 33) can include the CDR sequences contained in the V_{H} and V_{L} sequences of any one of the antibodies listed in Table 2.

An antigen binding domain of Fc-antigen binding domain construct 33 (each of 3322/3320 and 3316/3314 in FIG. 33) can include the CDR sequences contained in the V_{H} and V_{L} sequences of any one of the antibodies listed in Table 2.

An antigen binding domain of Fc-antigen binding domain construct 34 (3430/3404 in FIG. 34) can include the CDR sequences contained in the V_{H} and V_{L} sequences of any one of the antibodies listed in Table 2.

An antigen binding domain of Fc-antigen binding domain construct 34 (3428/3426 in FIG. 34) can include the CDR sequences contained in the V_{H} and V_{L} sequences of any one of the antibodies listed in Table 2.

An antigen binding domain of Fc-antigen binding domain construct 34 (each of 3422/3420 and 3416/3414 in FIG. 34) can include the CDR sequences contained in the V_{H} and V_{L} sequences of any one of the antibodies listed in Table 2.

An antigen binding domain of Fc-antigen binding domain construct 35 (each of 3530/3528 and 3520/3522 in FIG. 35) can include the CDR sequences contained in the V_{H} and V_{L} sequences of any one of the antibodies listed in Table 2.

An antigen binding domain of Fc-antigen binding domain construct 35 (3532/3506 in FIG. 35) can include the CDR sequences contained in the V_{H} and V_{L} sequences of any one of the antibodies listed in Table 2.

An antigen binding domain of Fc-antigen binding domain construct 35 (3518/3516 in FIG. 35) can include the CDR sequences contained in the V_{H} and V_{L} sequences of any one of the antibodies listed in Table 2.

An antigen binding domain of Fc-antigen binding domain construct 36 (each of 3638/3636 and 3628/3620 in FIG. 36) can include the CDR sequences contained in the V_{H} and V_{L} sequences of any one of the antibodies listed in Table 2.

An antigen binding domain of Fc-antigen binding domain construct 36 (each of 3640/3606 and 3626/3624 in FIG. 36) can include the CDR sequences contained in the V_{H} and V_{L} sequences of any one of the antibodies listed in Table 2.

An antigen binding domain of Fc-antigen binding domain construct 37 (each of 3748/3746 and 3738/3740 in FIG. 37) can include the CDR sequences contained in the V_{H} and V_{L} sequences of any one of the antibodies listed in Table 2.

An antigen binding domain of Fc-antigen binding domain construct 37 (each of 3750/3708 and 3736/3734 in FIG. 37) can include the CDR sequences contained in the V_{H} and V_{L} sequences of any one of the antibodies listed in Table 2.

An antigen binding domain of Fc-antigen binding domain construct 37 (each of 3714/3716 and 3728/3726 in FIG. 37) can include the CDR sequences contained in the V_{H} and V_{L} sequences of any one of the antibodies listed in Table 2.

An antigen binding domain of Fc-antigen binding domain construct 38 (each of 3832/3806 and 3830/3822 in FIG. 38) can include the CDR sequences contained in the V_{H} and V_{L} sequences of any one of the antibodies listed in Table 2.

An antigen binding domain of Fc-antigen binding domain construct 38 (3810/3812 in FIG. 38) can include the CDR sequences contained in the V_{H} and V_{L} sequences of any one of the antibodies listed in Table 2.

An antigen binding domain of Fc-antigen binding domain construct 38 (3824/3822 in FIG. 38) can include the CDR sequences contained in the V_{H} and V_{L} sequences of any one of the antibodies listed in Table 2.

An antigen binding domain of Fc-antigen binding domain construct 39 (each of 3938/3936 and 3924/3926 in FIG. 39) can include the CDR sequences contained in the V_{H} and V_{L} sequences of any one of the antibodies listed in Table 2.

An antigen binding domain of Fc-antigen binding domain construct 39 (each of 3940/3906 and 3922/3920 in FIG. 39) can include the CDR sequences contained in the V_{H} and V_{L} sequences of any one of the antibodies listed in Table 2.

An antigen binding domain of Fc-antigen binding domain construct 40 (each of 4048/4046 and 4034/4036 in FIG. 40) can include the CDR sequences contained in the V_{H} and V_{L} sequences of any one of the antibodies listed in Table 2.

An antigen binding domain of Fc-antigen binding domain construct 40 (each of 4050/4008 and 4032/4030 in FIG. 40) can include the CDR sequences contained in the V_{H} and V_{L} sequences of any one of the antibodies listed in Table 2.

An antigen binding domain of Fc-antigen binding domain construct 40 (each of 4012/4014 and 4026/4024 in FIG. 40) can include the CDR sequences contained in the V_{H} and V_{L} sequences of any one of the antibodies listed in Table 2.

An antigen binding domain of Fc-antigen binding domain construct 41 (each of 4140/4106 and 4138/4136 in FIG. 41) can include the CDR sequences contained in the V_{H} and V_{L} sequences of any one of the antibodies listed in Table 2.

An antigen binding domain of Fc-antigen binding domain construct 41 (each of 4112/4114 and 4130/4128 in FIG. 41) can include the CDR sequences contained in the V_{H} and V_{L} sequences of any one of the antibodies listed in Table 2.

An antigen binding domain of Fc-antigen binding domain construct 42 (each of 4250/4208 and 4248/4246 in FIG. 42) can include the CDR sequences contained in the V_{H} and V_{L} sequences of any one of the antibodies listed in Table 2.

An antigen binding domain of Fc-antigen binding domain construct 42 (each of 4218/4220 and 4236/4234 in FIG. 42) can include the CDR sequences contained in the V_{H} and V_{L} sequences of any one of the antibodies listed in Table 2.

An antigen binding domain of Fc-antigen binding domain construct 42 (each of 4212/4214 and 4242/4240 in FIG. 42) can include the CDR sequences contained in the V_{H} and V_{L} sequences of any one of the antibodies listed in Table 2.

The antigen binding domain of Fc-antigen binding domain construct 1 (110/104 in FIG. 1) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domain of Fc-antigen binding domain construct 2 (212/204 in FIG. 2) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 3 (308/316 and 312/318 in FIG. 3) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 4 (410/412, 416/418 and 422/424 in FIG. 4) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 5 (510/504, 512/514 and 518/520 in FIG. 5) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 6 (612/604, 614/616, 620/622, and 626/628 in FIG. 6) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 7 (712/714 and 714/716 in FIG. 7) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 8 (812/806 and 818/822 in FIG. 8) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 9 (908/906, 920/922, 912/914, and 926/930 in FIG. 9) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 10 (1006/1004 and 1018/1020 in FIG. 10) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 11 (1112/1114, 1122/1108, 1128/1142, and 1138/1136 in FIG. 11) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 12 (1218/1220, 1212/1214, 1250/1208, 1248/1246, 1242/1240, and 1236/1234 in FIG. 12) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 13 (1310/1304 and 1314/1322 in FIG. 13) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 14 (1408/1406 and 1416/1424 in FIG. 14) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 15 (1508/1506, 1514/1516, 1532/1520, and 1530/1528 in FIG. 15) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 16 (1616/1604 and 1618/1630 in FIG. 16) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 17 (1712/1714, 1724/1708, 1726/1742, and 1738/1736 in FIG. 17) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 18 (1812/1814, 1828/1808, 1826/1824, 1830/1832, 1850/1848, and 1844/1842 in FIG. 18) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 19 (1914/1904 and 1920/1922 in FIG. 19) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 20 (2014/2016, 2042/2008, 2036/2034, and 2028/2026 in FIG. 20) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

The antigen binding domains of Fc-antigen binding domain construct 21 (2114/2116, 2150/2108, 2148/2146, 2138/2140, 2136/2134, and 2128/2126 in FIG. 21) each can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 22 (2204/2222 in FIG. 22) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 22 (each of 2218/2220 and 2212/2214 in FIG. 22) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 23 (2330/2304 in FIG. 23) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 23 (each of 2328/2326, 2322/2320, and 2316/2314 in FIG. 23) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 24 (each of 2430/2428 and 2420/2422 in FIG. 24) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 24 (each of 2432/2406 and 2418/2416 in FIG. 24) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 25 (each of 2532/2506 and 2530/2528 in FIG. 25) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 25 (each of 2510/2512 and 2524/2522 in FIG. 25) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 26 (each of 2648/2646 and 2634/2636 in FIG. 26) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 26 (each of 2612/2614, 2650/2608, 2632/2630, and 2626/2624 in FIG. 26) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 27 (each of 2748/2746 and 2738/2740 in FIG. 27) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 27 (each of 2714/2716, 2750/2708, 2736/2734, and 2728/2726 in FIG. 27) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 28 (each of 2850/2808 and 2848/2846 in FIG. 27) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 28 (each of 2818/2820, 2812/2814, 2842/2840, and 2836/2834 in FIG. 28) can include the CDR sequences contained in the V and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 29 (2918/2904 in FIG. 29) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 29 (2914/2912 in FIG. 29) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 30 (each of 3022/3004 and 3020/3018 in FIG. 30) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 30 (3014/3012 in FIG. 30) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 31 (3122/3104 in FIG. 31) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 31 (3120/3118 in FIG. 31) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 31 (3114/3112 in FIG. 31) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 32 (3226/3204 in FIG. 32) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 32 (each of 3222/3220 and 3216/3214 in FIG. 32) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 33 (each of 3330/3304 and 3328/3326 in FIG. 33) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 33 (each of 3322/3320 and 3316/3314 in FIG. 33) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 34 (3430/3404 in FIG. 34) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 34 (3428/3426 in FIG. 34) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 34 (each of 3422/3420 and 3416/3414 in FIG. 34) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 35 (each of 3530/3528 and 3520/3522 in FIG. 35) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 35 (3532/3506 in FIG. 35) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 35 (3518/3516 in FIG. 35) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 36 (each of 3638/3636 and 3628/3620 in FIG. 36) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 36 (each of 3640/3606 and 3626/3624 in FIG. 36) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 37 (each of 3748/3746 and 3738/3740 in FIG. 37) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 37 (each of 3750/3708 and 3736/3734 in FIG. 37) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 37 (each of 3714/3716 and 3728/3726 in FIG. 37) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 38 (each of 3832/3806 and 3830/3822 in FIG. 38) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 38 (3810/3812 in FIG. 38) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 38 (3824/3822 in FIG. 38) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 39 (each of 3938/3936 and 3924/3926 in FIG. 39) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 39 (each of 3940/3906 and 3922/3920 in FIG. 39) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 40 (each of 4048/4046 and 4034/4036 in FIG. 40) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 40 (each of 4050/4008 and 4032/4030 in FIG. 40) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 40 (each of 4012/4014 and 4026/4024 in FIG. 40) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 41 (each of 4140/4106 and 4138/4136 in FIG. 41) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 41 (each of 4112/4114 and 4130/4128 in FIG. 41) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 42 (each of 4250/4208 and 4248/4246 in FIG. 42) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 42 (each of 4218/4220 and 4236/4234 in FIG. 42) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

An antigen binding domain of Fc-antigen binding domain construct 42 (each of 4212/4214 and 4242/4240 in FIG. 42) can include the CDR sequences contained in the V_{H} and V_{L} sequences, and the remainder of the V_{H} and V_{L} sequences are at least 95% identical, at least 97% identical, at least 99% identical, or at least 99.5% identical to the V_{H} and V_{L} sequences of any one of the antibodies listed in Table 2.

### IV. Dimerization Selectivity Modules

In the present disclosure, a dimerization selectivity module includes components or select amino acids within the Fc domain monomer that facilitate the preferred pairing of two Fc domain monomers to form an Fc domain. Specifically, a dimerization selectivity module is that part of the C_{H}3 antibody constant domain of an Fc domain monomer which includes amino acid substitutions positioned at the interface between interacting C_{H}3 antibody constant domains of two Fc domain monomers. In a dimerization selectivity module, the amino acid substitutions make favorable the dimerization of the two C_{H}3 antibody constant domains as a result of the compatibility of amino acids chosen for those substitutions. The ultimate formation of the favored Fc domain is selective over other Fc domains which form from Fc domain monomers lacking dimerization selectivity modules or with incompatible amino acid substitutions in the dimerization selectivity modules. This type of amino acid substitution can be made using conventional molecular cloning techniques well-known in the art, such as QuikChange® mutagenesis.

In some embodiments, a dimerization selectivity module includes an engineered cavity (described further herein) in the C_{H}3 antibody constant domain. In other embodiments, a dimerization selectivity module includes an engineered protuberance (described further herein) in the C_{H}3 antibody constant domain. To selectively form an Fc domain, two Fc domain monomers with compatible dimerization selectivity modules, e.g., one C_{H}3 antibody constant domain containing an engineered cavity and the other C_{H}3 antibody constant domain containing an engineered protuberance, combine to form a protuberance-into-cavity pair of Fc domain monomers. Engineered protuberances and engineered cavities are examples of heterodimerizing selectivity modules, which can be made in the C_{H}3 antibody constant domains of Fc domain monomers in order to promote favorable heterodimerization of two Fc domain monomers that have compatible heterodimerizing selectivity modules.

In other embodiments, an Fc domain monomer with a dimerization selectivity module containing positively-charged amino acid substitutions and an Fc domain monomer with a dimerization selectivity module containing negatively-charged amino acid substitutions may selectively combine to form an Fc domain through the favorable electrostatic steering (described further herein) of the charged amino acids. In some embodiments, an Fc domain monomer may include one of the following positively-charged and negatively-charged amino acid substitutions: K392D, K392E, D399K, K409D, K409E, K439D, and K439E. In one example, an Fc domain monomer containing a positively-charged amino acid substitution, e.g., D356K or E357K, and an Fc domain monomer containing a negatively-charged amino acid substitution, e.g., K370D or K370E, may selectively combine to form an Fc domain through favorable electrostatic steering of the charged amino acids. In another example, an Fc domain monomer containing E357K and an Fc domain monomer containing K370D may selectively combine to form an Fc domain through favorable electrostatic steering of the charged amino acids. In some embodiments, reverse charge amino acid substitutions may be used as heterodimerizing selectivity modules, wherein two Fc domain monomers containing different, but compatible, reverse charge amino acid substitutions combine to form a heterodimeric Fc domain. Specific dimerization selectivity modules are further listed, without limitation, in Tables 3 and 4 described further below.

In other embodiments, two Fc domain monomers include homodimerizing selectivity modules containing identical reverse charge mutations in at least two positions within the ring of charged residues at the interface between CH3 domains. Homodimerizing selectivity modules are reverse charge amino acid substitutions that promote the homodimerization of Fc domain monomers to form a homodimeric Fc domain. By reversing the charge of both members of two or more complementary pairs of residues in the two Fc domain monomers, mutated Fc domain monomers remain complementary to Fc domain monomers of the same mutated sequence, but have a lower complementarity to Fc domain monomers without those mutations. In one embodiment, an Fc domain includes Fc domain monomers including the double mutants K409D/D399K, K392D/D399K, E357K/K370E, D356K/K439D, K409E/D399K, K392E/D399K, E357K/K370D, or D356K/K439E. In another embodiment, an Fc domain includes Fc domain monomers including quadruple mutants combining any pair of the double mutants, e.g., K409D/D399K/E357K/K370E. Examples of homodimerizing selectivity modules are further shown in Tables 5 and 6.

In further embodiments, an Fc domain monomer containing (i) at least one reverse charge mutation and (ii) at least one engineered cavity or at least one engineered protuberance may selectively combine with another Fc domain monomer containing (i) at least one reverse charge mutation and (ii) at least one engineered protuberance or at least one engineered cavity to form an Fc domain. For example, an Fc domain monomer containing reversed charge mutation K370D and engineered cavities Y349C, T366S, L368A, and Y407V and another Fc domain monomer containing reversed charge mutation E357K and engineered protuberances S354C and T366W may selectively combine to form an Fc domain.

The formation of such Fc domains is promoted by the compatible amino acid substitutions in the C_{H}3 antibody constant domains. Two dimerization selectivity modules containing incompatible amino acid substitutions, e.g., both containing engineered cavities, both containing engineered protuberances, or both containing the same charged amino acids at the C_{H}3-C_{H}3 interface, will not promote the formation of a heterodimeric Fc domain.

Furthermore, other methods used to promote the formation of Fc domains with defined Fc domain monomers include, without limitation, the LUZ-Y approach (U.S. Patent Application Publication No. WO2011034605) which includes C-terminal fusion of a monomer α-helices of a leucine zipper to each of the Fc domain monomers to allow heterodimer formation, as well as strand-exchange engineered domain (SEED) body approach (Davis et al., Protein Eng Des Sel. 23:195-202, 2010) that generates Fc domain with heterodimeric Fc domain monomers each including alternating segments of IgA and IgG C_{H}3 sequences.

### V. Engineered Cavities and Engineered Protuberances

The use of engineered cavities and engineered protuberances (or the “knob-into-hole” strategy) is described by Carter and co-workers (Ridgway et al., Protein Eng. 9:617-612, 1996; Atwell et al., J Mol Biol. 270:26-35, 1997; Merchant et al., Nat Biotechnol. 16:677-681, 1998). The knob and hole interaction favors heterodimer formation, whereas the knob-knob and the hole-hole interaction hinder homodimer formation due to steric dash and deletion of favorable interactions. The “knob-into-hole” technique is also disclosed in U.S. Pat. No. 5,731,168.

In the present disclosure, engineered cavities and engineered protuberances are used in the preparation of the Fc-antigen binding domain constructs described herein. An engineered cavity is a void that is created when an original amino acid in a protein is replaced with a different amino acid having a smaller side-chain volume. An engineered protuberance is a bump that is created when an original amino acid in a protein is replaced with a different amino acid having a larger side-chain volume. Specifically, the amino acid being replaced is in the C_{H}3 antibody constant domain of an Fc domain monomer and is involved in the dimerization of two Fc domain monomers. In some embodiments, an engineered cavity in one C_{H}3 antibody constant domain is created to accommodate an engineered protuberance in another C_{H}3 antibody constant domain, such that both C_{H}3 antibody constant domains act as dimerization selectivity modules (e.g., heterodimerizing selectivity modules) (described above) that promote or favor the dimerization of the two Fc domain monomers. In other embodiments, an engineered cavity in one C_{H}3 antibody constant domain is created to better accommodate an original amino acid in another C_{H}3 antibody constant domain. In yet other embodiments, an engineered protuberance in one C_{H}3 antibody constant domain is created to form additional interactions with original amino acids in another C_{H}3 antibody constant domain.

An engineered cavity can be constructed by replacing amino acids containing larger side chains such as tyrosine or tryptophan with amino acids containing smaller side chains such as alanine, valine, or threonine. Specifically, some dimerization selectivity modules (e.g., heterodimerizing selectivity modules) (described further above) contain engineered cavities such as Y407V mutation in the C_{H}3 antibody constant domain. Similarly, an engineered protuberance can be constructed by replacing amino acids containing smaller side chains with amino acids containing larger side chains. Specifically, some dimerization selectivity modules (e.g., heterodimerizing selectivity modules) (described further above) contain engineered protuberances such as T366W mutation in the C_{H}3 antibody constant domain. In the present disclosure, engineered cavities and engineered protuberances are also combined with inter-C_{H}3 domain disulfide bond engineering to enhance heterodimer formation. In one example, an Fc domain monomer containing engineered cavities Y349C, T366S, L368A, and Y407V may selectively combine with another Fc domain monomer containing engineered protuberances S354C and T366W to form an Fc domain. In another example, an Fc domain monomer containing an engineered cavity with the addition of Y349C and an Fc domain monomer containing an engineered protuberance with the addition of S354C may selectively combine to form an Fc domain. Other engineered cavities and engineered protuberances, in combination with either disulfide bond engineering or structural calculations (mixed HA-TF) are included, without limitation, in Table 3.

TABLE 3

| | CH₃ antibody | CH₃ antibody | |
|---|---|---|---|
| | constant domain of Fc | constant domain of Fc | |
| Strategy | domain monomer 1 | domain monomer 2 | Reference |
| Engineered cavities | Y407T | T366Y | U.S. Pat. No. 8,216,805 |
| and protuberances | | | |
| (“knob-into-hole”) | | | |
| | Y407A | T366W | U.S. Pat. No. 8,216,805 |
| | F405A | T394W | U.S. Pat. No. 8,216,805 |
| | Y407T | T366Y | U.S. Pat. No. 8,216,805 |
| | T394S | F405W | U.S. Pat. No. 8,216,805 |
| | T394W:Y407T | T366Y:F405A | U.S. Pat. No. 8,216,805 |
| | T394S:Y407A | T366W:F405W | U.S. Pat. No. 8,216,805 |
| | T366W:T394S | F405W:Y407A | U.S. Pat. No. 8,216,805 |
| | F405T | T394Y | |
| Engineered cavities | T366S:L368A:Y407V:Y349C | T366W:S354C | Merchant et al., Nat. |
| and protuberances | | | Biotechnol. 16(7): 677- |
| (“knob-into-hole”), | | | 81, 1998 |
| S-S engineering | | | |
| Mixed HA-TF | S364H:F405A | Y349T:T394F | WO2006106905 |

Replacing an original amino acid residue in the C_{H}3 antibody constant domain with a different amino acid residue can be achieved by altering the nucleic acid encoding the original amino acid residue. The upper limit for the number of original amino acid residues that can be replaced is the total number of residues in the interface of the C_{H}3 antibody constant domains, given that sufficient interaction at the interface is still maintained.

Combining Engineered Cavities and Engineered Protuberances with Electrostatic Steering

Electrostatic steering can be combined with knob-in-hole technology to favor heterominerization, for example, between Fc domain monomers in two different polypeptides. Electrostatic steering, described in greater detail below, is the utilization of favorable electrostatic interactions between oppositely charged amino acids in peptides, protein domains, and proteins to control the formation of higher ordered protein molecules. Electrostatic steering can be used to promote either homodimerization or heterodimerization, the latter of which can be usefully combined with knob-in-hole technology. In the case of heterodimerization, different, but compatible, mutations are introduced in each of the Fc domain monomers which are to heterodimerize. Thus, an Fc domain monomer can be modified to include one of the following positively-charged and negatively-charged amino acid substitutions: D356K, D356R, E357K, E357R, K370D, K370E, K392D, K392E, D399K, K409D, K409E, K439D, and K439E. For example, one Fc domain monomer, for example, an Fc domain monomer having a cavity (Y349C, T366S, L368A and Y407V), can also include K370D mutation and the other Fc domain monomer, for example, an Fc domain monomer having a protuberance (S354C and T366W) can include E357K.

More generally, any of the cavity mutations (or mutation combinations): Y407T, Y407A, F405A, Y407T, T394S, T394W:Y407A, T366W:T394S, T366S:L368A:Y407V:Y349C, and S3364H:F405 can be combined with a mutation in Table 4 and any of the protuberance mutations (or mutation combinations): T366Y, T366W, T394W, F405W, T366Y:F405A, T366W:Y407A, T366W:S354C, and Y349T:T394F can be combined with a mutation in Table 4 that is paired with the Table 4 mutation used in combination with the cavity mutation (or mutation combination).

### VI. Electrostatic Steering

Electrostatic steering is the utilization of favorable electrostatic interactions between oppositely charged amino acids in peptides, protein domains, and proteins to control the formation of higher ordered protein molecules. A method of using electrostatic steering effects to alter the interaction of antibody domains to reduce for formation of homodimer in favor of heterodimer formation in the generation of bi-specific antibodies is disclosed in U.S. Patent Application Publication No. 2014-0024111.

In the present disclosure, electrostatic steering is used to control the dimerization of Fc domain monomers and the formation of Fc-antigen binding domain constructs. In particular, to control the dimerization of Fc domain monomers using electrostatic steering, one or more amino acid residues that make up the C_{H}3-C_{H}3 interface are replaced with positively- or negatively-charged amino acid residues such that the interaction becomes electrostatically favorable or unfavorable depending on the specific charged amino acids introduced. In some embodiments, a positively-charged amino acid in the interface, such as lysine, arginine, or histidine, is replaced with a negatively-charged amino acid such as aspartic acid or glutamic acid. In other embodiments, a negatively-charged amino acid in the interface is replaced with a positively-charged amino acid. The charged amino acids may be introduced to one of the interacting C_{H}3 antibody constant domains, or both. By introducing charged amino acids to the interacting C_{H}3 antibody constant domains, dimerization selectivity modules (described further above) are created that can selectively form dimers of Fc domain monomers as controlled by the electrostatic steering effects resulting from the interaction between charged amino acids.

In some embodiments, to create a dimerization selectivity module including reversed charges that can selectively form dimers of Fc domain monomers as controlled by the electrostatic steering effects, the two Fc domain monomers may be selectively formed through heterodimerization or homodimerization.

Heterodimerization of Fc Domain Monomers

Heterodimerization of Fc domain monomers can be promoted by introducing different, but compatible, mutations in the two Fc domain monomers, such as the charge residue pairs included, without limitation, in Table 4. In some embodiments, an Fc domain monomer may include one of the following positively-charged and negatively-charged amino acid substitutions: D356K, D356R, E357K, E357R, K370D, K370E, K392D, K392E, D399K, K409D, K409E, K439D, and K439E. In one example, an Fc domain monomer containing a positively-charged amino acid substitution, e.g., D356K or E357K, and an Fc domain monomer containing a negatively-charged amino acid substitution, e.g., K370D or K370E, may selectively combine to form an Fc domain through favorable electrostatic steering of the charged amino acids. In another example, an Fc domain monomer containing E357K and an Fc domain monomer containing K370D may selectively combine to form an Fc domain through favorable electrostatic steering of the charged amino acids.

For example, in an Fc-antigen binding domain construct having three Fc domains, two of the three Fc domains may be formed by the heterodimerization of two Fc domain monomers, as promoted by the electrostatic steering effects. A “heterodimeric Fc domain” refers to an Fc domain that is formed by the heterodimerization of two Fc domain monomers, wherein the two Fc domain monomers contain different reverse charge mutations (heterodimerizing selectivity modules) (see, e.g., mutations in Table 4) that promote the favorable formation of these two Fc domain monomers. In an Fc-antigen binding domain construct having three Fc domains—one carboxyl terminal “stem” Fc domain and two amino terminal “branch” Fc domains—each of the amino terminal “branch” Fc domains may be a heterodimeric Fc domain (also called a “branch heterodimeric Fc domain”) (e.g., a heterodimeric Fc domain formed by Fc domain monomers 106 and 114 or Fc domain monomers 112 and 116 in FIG. 1; a heterodimeric Fc domain formed by Fc domain monomers 206 and 214 or Fc domain monomers 212 and 216 in FIG. 2). A branch heterodimeric Fc domain may be formed by an Fc domain monomer containing E357K and another Fc domain monomer containing K370D.

TABLE 4

| Reverse charge mutation(s) in | Reverse charge mutation(s) in |
|---|---|
| C_{H}3 antibody constant domain of | C_{H}3 antibody constant domain of |
| Fc domain monomer 1 | Fc domain monomer 2 |
| K409D | D399K |
| K409D | D399R |
| K409E | D399K |
| K409E | D399R |
| K392D | D399K |
| K392D | D399R |
| K392E | D399K |
| K392E | D399R |
| K370D | E357K |
| K370D | E357R |
| K370E | E357K |
| K370E | E357R |
| K370D | D356K |
| K370D | D356R |
| K370E | D356K |
| K370E | D356R |
| K409D, K392D | D399K, E356K |
| K370E, K409D, K439E | E356K, E357K, D399K |

Homodimerization of Fc Domain Monomers

Homodimerization of Fc domain monomers can be promoted by introducing the same electrostatic steering mutations (homodimerizing selectivity modules) in both Fc domain monomers in a symmetric fashion. In some embodiments, two Fc domain monomers include homodimerizing selectivity modules containing identical reverse charge mutations in at least two positions within the ring of charged residues at the interface between C_{H}3 domains. By reversing the charge of both members of two or more complementary pairs of residues in the two Fc domain monomers, mutated Fc domain monomers remain complementary to Fc domain monomers of the same mutated sequence, but have a lower complementarity to Fc domain monomers without those mutations. Electrostatic steering mutations that may be introduced into an Fc domain monomer to promote its homodimerization are shown, without limitation, in Tables 5 and 6. In one embodiment, an Fc domain includes two Fc domain monomers each including the double reverse charge mutants (Table 5), e.g., K409D/D399K. In another embodiment, an Fc domain includes two Fc domain monomers each including quadruple reverse mutants (Table 6), e.g., K409D/D399K/K370D/E357K.

For example, in an Fc-antigen binding domain construct having three Fc domains, one of the three Fc domains may be formed by the homodimerization of two Fc domain monomers, as promoted by the electrostatic steering effects. A “homodimeric Fc domain” refers to an Fc domain that is formed by the homodimerization of two Fc domain monomers, wherein the two Fc domain monomers contain the same reverse charge mutations (see, e.g., mutations in Tables 5 and 6). In an Fc-antigen binding domain construct having three Fc domains—one carboxyl terminal “stem” Fc domain and two amino terminal “branch” Fc domains—the carboxy terminal “stem” Fc domain may be a homodimeric Fc domain (also called a “stem homodimeric Fc domain”). A stem homodimeric Fc domain may be formed by two Fc domain monomers each containing the double mutants K409D/D399K.

TABLE 5

Reverse charge mutation(s) in C_{H}3 antibody
constant domain of each of the two Fc domain
monomers in a homodimeric Fc domain
K409D/D399K
K409D/D399R
K409E/D399K
K409E/D399R
K392D/D399K
K392D/D399R
K392E/D399K
K392E/D399R
K370D/E357K
K370D/E357R
K370E/E357K
K370E/E357R
K370D/D356K
K370D/D356R
K370E/D356K
K370E/D356R

TABLE 6

| Reverse charge mutation(s) in | Reverse charge mutation(s) in |
|---|---|
| C_{H}3 antibody constant | C_{H}3 antibody constant |
| domain of each of the two Fc | domain of each of the two Fc |
| domain monomers in a | domain monomers in a |
| homodimeric Fc domain | homodimeric Fc domain |
| K409D/D399K/K370D/E357K | K392D/D399K/K370D/E357K |
| K409D/D399K/K370D/E357R | K392D/D399K/K370D/E357R |
| K409D/D399K/K370E/E357K | K392D/D399K/K370E/E357K |
| K409D/D399K/K370E/E357R | K392D/D399K/K370E/E357R |
| K409D/D399K/K370D/D356K | K392D/D399K/K370D/D356K |
| K409D/D399K/K370D/D356R | K392D/D399K/K370D/D356R |
| K409D/D399K/K370E/D356K | K392D/D399K/K370E/D356K |
| K409D/D399K/K370E/D356R | K392D/D399K/K370E/D356R |
| K409D/D399R/K370D/E357K | K392D/D399R/K370D/E357K |
| K409D/D399R/K370D/E357R | K392D/D399R/K370D/E357R |
| K409D/D399R/K370E/E357K | K392D/D399R/K370E/E357K |
| K409D/D399R/K370E/E357R | K392D/D399R/K370E/E357R |
| K409D/D399R/K370D/D356K | K392D/D399R/K370D/D356K |
| K409D/D399R/K370D/D356R | K392D/D399R/K370D/D356R |
| K409D/D399R/K370E/D356K | K392D/D399R/K370E/D356K |
| K409D/D399R/K370E/D356R | K392D/D399R/K370E/D356R |
| K409E/D399K/K370D/E357K | K392E/D399K/K370D/E357K |
| K409E/D399K/K370D/E357R | K392E/D399K/K370D/E357R |
| K409E/D399K/K370E/E357K | K392E/D399KZK370E/E357K |
| K409E/D399K/K370E/E357R | K392E/D399K/K370E/E357R |
| K409E/D399K/K370D/D356K | K392E/D399K/K370D/D356K |
| K409E/D399K/K370D/D356R | K392E/D399K/K370D/D356R |
| K409E/D399K/K370E/D356K | K392E/D399K/K370E/D356K |
| K409E/D399K/K370E/D356R | K392E/D399K/K370E/D356R |
| K409E/D399R/K370D/E357K | K392E/D399R/K370D/E357K |
| K409E/D399R/K370D/E357R | K392E/D399R/K370D/E357R |
| K409E/D399R/K370E/E357K | K392E/D399R/K370E/E357K |
| K409E/D399R/K370E/E357R | K392E/D399R/K370E/E357R |
| K409E/D399R/K370D/D356K | K392E/D399R/K370D/D356K |
| K409E/D399R/K370D/D356R | K392E/D399R/K370D/D356R |
| K409E/D399R/K370E/D356K | K392E/D399R/K370E/D356K |
| K409E/D399R/K370E/D356R | K392E/D399R/K370E/D356R |

### VII. Linkers

In the present disclosure, a linker is used to describe a linkage or connection between polypeptides or protein domains and/or associated non-protein moieties. In some embodiments, a linker is a linkage or connection between at least two Fc domain monomers, for which the linker connects the C-terminus of the C_{H}3 antibody constant domain of a first Fc domain monomer to the N-terminus of the hinge domain of a second Fc domain monomer, such that the two Fc domain monomers are joined to each other in tandem series. In other embodiments, a linker is a linkage between an Fc domain monomer and any other protein domains that are attached to it. For example, a linker can attach the C-terminus of the C_{H}3 antibody constant domain of an Fc domain monomer to the N-terminus of an albumin-binding peptide.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two Fc domain monomer, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

Spacer

In the present disclosure, a linker between two Fc domain monomers can be an amino acid spacer including 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5-200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids). In some embodiments, a linker between two Fc domain monomers is an amino acid spacer containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids). In some embodiments, a linker between two Fc domain monomers is an amino acid spacer containing 12-30 amino acids (e.g., 12, 13, 14, 15, 16, 17, 18, 19, 20, 21, 22, 23, 24, 25, 26, 27, 28, 29, or 30 amino acids). Suitable peptide spacers are known in the art, and include, for example, peptide linkers containing flexible amino acid residues such as glycine and serine. In certain embodiments, a spacer can contain motifs, e.g., multiple or repeating motifs, of GS, GGS, GGGGS (SEQ ID NO: 1), GGSG (SEQ ID NO: 2), or SGGG (SEQ ID NO: 3). In certain embodiments, a spacer can contain 2 to 12 amino acids including motifs of GS, e.g., GS, GSGS (SEQ ID NO: 4), GSGSGS (SEQ ID NO: 5), GSGSGSGS (SEQ ID NO: 6), GSGSGSGSGS (SEQ ID NO: 7), or GSGSGSGSGSGS (SEQ ID NO: 8). In certain other embodiments, a spacer can contain 3 to 12 amino acids including motifs of GGS, e.g., GGS, GGSGGS (SEQ ID NO: 9), GGSGGSGGS (SEQ ID NO: 10), and GGSGGSGGSGGS (SEQ ID NO: 11). In yet other embodiments, a spacer can contain 4 to 20 amino acids including motifs of GGSG (SEQ ID NO: 2), e.g., GGSGGGSG (SEQ ID NO: 12), GGSGGGSGGGSG (SEQ ID NO: 13), GGSGGGSGGGSGGGSG (SEQ ID NO: 14), or GGSGGGSGGGSGGGSGGGSG (SEQ ID NO: 15). In other embodiments, a spacer can contain motifs of GGGGS (SEQ ID NO: 1), e.g., GGGGSGGGGS (SEQ ID NO: 16) or GGGGSGGGGSGGGGS (SEQ ID NO: 17). In certain embodiments, a spacer is SGGGSGGGSGGGSGGGSGGG (SEQ ID NO: 18).

In some embodiments, a spacer between two Fc domain monomers contains only glycine residues, e.g., at least 4 glycine residues (e.g., 4-200, 4-180, 4-160, 4-140, 4-40, 4-100, 4-90, 4-80, 4-70, 4-60, 4-50, 4-40, 4-30, 4-20, 4-19, 4-18, 4-17, 4-16, 4-15, 4-14, 4-13, 4-12, 4-11, 4-10, 4-9, 4-8, 4-7, 4-6 or 4-5 glycine residues) (e.g., 4-200, 6-200, 8-200, 10-200, 12-200, 14-200, 16-200, 18-200, 20-200, 30-200, 40-200, 50-200, 60-200, 70-200, 80-200, 90-200, 100-200, 120-200, 140-200, 160-200, 180-200, or 190-200 glycine residues). In certain embodiments, a spacer has 4-30 glycine residues (e.g., 4, 5, 6, 7, 8, 9, 10, 11, 12, 13, 14, 15, 16, 17, 18, 19, 20, 21, 22, 23, 24, 25, 26, 27, 28, 29, or 30 glycine residues). In some embodiments, a spacer containing only glycine residues may not be glycosylated (e.g., O-linked glycosylation, also referred to as O-glycosylation) or may have a decreased level of glycosylation (e.g., a decreased level of O-glycosylation) (e.g., a decreased level of O-glycosylation with glycans such as xylose, mannose, sialic acids, fucose (Fuc), and/or galactose (Gal) (e.g., xylose)) as compared to, e.g., a spacer containing one or more serine residues (e.g., SGGGSGGGSGGGSGGGSGGG (SEQ ID NO: 18)).

In some embodiments, a spacer containing only glycine residues may not be O-glycosylated (e.g., O-xylosylation) or may have a decreased level of O-glycosylation (e.g., a decreased level of O-xylosylation) as compared to, e.g., a spacer containing one or more serine residues (e.g., SGGGSGGGSGGGSGGGSGGG (SEQ ID NO: 18)).

In some embodiments, a spacer containing only glycine residues may not undergo proteolysis or may have a decreased rate of proteolysis as compared to, e.g., a spacer containing one or more serine residues (e.g., SGGGSGGGSGGGSGGGSGGG (SEQ ID NO: 18)).

In certain embodiments, a spacer can contain motifs of GGGG (SEQ ID NO: 19), e.g., GGGGGGGG (SEQ ID NO: 20), GGGGGGGGGGGG (SEQ ID NO: 21), GGGGGGGGGGGGGGGG (SEQ ID NO: 22), or GGGGGGGGGGGGGGGGGGGG (SEQ ID NO: 23). In certain embodiments, a spacer can contain motifs of GGGGG (SEQ ID NO: 24), e.g., GGGGGGGGGG (SEQ ID NO: 25), or GGGGGGGGGGGGGGG (SEQ ID NO: 26). In certain embodiments, a spacer is GGGGGGGGGGGGGGGGGGGG (SEQ ID NO: 27).

In other embodiments, a spacer can also contain amino acids other than glycine and serine, e.g., GENLYFQSGG (SEQ ID NO: 28), SACYCELS (SEQ ID NO: 29), RSIAT (SEQ ID NO: 30), RPACKIPNDLKQKVMNH (SEQ ID NO: 31), GGSAGGSGSGSSGGSSGASGTGTAGGTGSGSGTGSG (SEQ ID NO: 32), AAANSSIDLISVPVDSR (SEQ ID NO: 33), or GGSGGGSEGGGSEGGGSEGGGSEGGGSEGGGSGGGS (SEQ ID NO: 34).

In certain embodiments in the present disclosure, a 12- or 20-amino acid peptide spacer is used to connect two Fc domain monomers in tandem series, the 12- and 20-amino acid peptide spacers consisting of sequences GGGSGGGSGGGS (SEQ ID NO: 35) and SGGGSGGGSGGGSGGGSGGG (SEQ ID NO: 18), respectively. In other embodiments, an 18-amino acid peptide spacer consisting of sequence GGSGGGSGGGSGGGSGGS (SEQ ID NO: 36) may be used.

In some embodiments, a spacer between two Fc domain monomers may have a sequence that is at least 75% identical (e.g., at least 77%, 79%, 81%, 83%, 85%, 87%, 89%, 91%, 93%, 95%, 97%, 99%, or 99.5% identical) to the sequence of any one of SEQ ID NOs: 1-36 described above. In certain embodiments, a spacer between two Fc domain monomers may have a sequence that is at least 80% identical (e.g., at least 82%, 85%, 87%, 90%, 92%, 95%, 97%, 99%, or 99.5% identical) to the sequence of any one of SEQ ID NOs: 17, 18, 26, and 27. In certain embodiments, a spacer between two Fc domain monomers may have a sequence that is at least 80% identical (e.g., at least 82%, 85%, 87%, 90%, 92%, 95%, 97%, 99%, or 99.5%) to the sequence of SEQ ID NO: 18 or 27.

### VII. Serum Protein-Binding Peptides

Binding to serum protein peptides can improve the pharmacokinetics of protein pharmaceuticals, and in particular the Fc-antigen binding domain constructs described here may be fused with serum protein-binding peptides

As one example, albumin-binding peptides that can be used in the methods and compositions described here are generally known in the art. In one embodiment, the albumin binding peptide includes the sequence DICLPRWGCLW (SEQ ID NO: 37). In some embodiments, the albumin binding peptide has a sequence that is at least 80% identical (e.g., 80%, 90%, or 100% identical) to the sequence of SEQ ID NO: 37.

In the present disclosure, albumin-binding peptides may be attached to the N- or C-terminus of certain polypeptides in the Fc-antigen binding domain construct. In one embodiment, an albumin-binding peptide may be attached to the C-terminus of one or more polypeptides in Fc constructs containing an antigen binding domain. In another embodiment, an albumin-binding peptide can be fused to the C-terminus of the polypeptide encoding two Fc domain monomers linked in tandem series in Fc constructs containing an antigen binding domain. In yet another embodiment, an albumin-binding peptide can be attached to the C-terminus of Fc domain monomer (e.g., Fc domain monomers 114 and 116 in FIG. 1; Fc domain monomers 214 and 216 in FIG. 2) which is joined to the second Fc domain monomer in the polypeptide encoding the two Fc domain monomers linked in tandem series. Albumin-binding peptides can be fused genetically to Fc-antigen binding domain constructs or attached to Fc-antigen binding domain constructs through chemical means, e.g., chemical conjugation. If desired, a spacer can be inserted between the Fc-antigen binding domain construct and the albumin-binding peptide. Without being bound to a theory, it is expected that inclusion of an albumin-binding peptide in an Fc-antigen binding domain construct of the disclosure may lead to prolonged retention of the therapeutic protein through its binding to serum albumin.

### VIII. Fc-Antigen Binding Domain Constructs

In general, the disclosure features Fc-antigen binding domain constructs having 2-10 Fc domains and one or more antigen binding domains attached. These may have greater binding affinity and/or avidity than a single wild-type Fc domain for an Fc receptor, e.g., FcγRIIa. The disclosure discloses methods of engineering amino acids at the interface of two interacting C_{H}3 antibody constant domains such that the two Fc domain monomers of an Fc domain selectively form a dimer with each other, thus preventing the formation of unwanted multimers or aggregates. An Fc-antigen binding domain construct includes an even number of Fc domain monomers, with each pair of Fc domain monomers forming an Fc domain. An Fc-antigen binding domain construct includes, at a minimum, two functional Fc domains formed from dimer of four Fc domain monomers and one antigen binding domain. The antigen binding domain may be joined to an Fc domain e.g., with a linker, a spacer, a peptide bond, a chemical bond or chemical moiety.

The Fc-antigen binding domain constructs can be assembled in many ways. The Fc-antigen binding domain constructs can be assembled from asymmetrical tandem Fc domains (FIG. 1-FIG. 6). The Fc-antigen binding domain constructs can be assembled from singly branched Fc domains, where the branch point is at the N-terminal Fc domain (FIG. 7-FIG. 12). The Fc-antigen binding domain constructs can be assembled from singly branched Fc domains, where the branch point is at the C-terminal Fc domain (FIG. 13-FIG. 18). The Fc-antigen binding domain constructs can be assembled from singly branched Fc domains, where the branch point is neither at the N- or C-terminal Fc domain (FIG. 19-FIG. 21). The Fc-antigen binding domain constructs can be assembled to form bispecific constructs using long and short chains with different antigen binding domain sequences (FIG. 22-FIG. 28). The Fc-antigen binding domain constructs can be assembled to form bispecific and trispecific constructs using chains with different sets of heterodimerization mutations (FIG. 19-FIG. 42) and different antigen binding domains. A bispecific Fc-antigen binding domain construct includes two different antigen biding domains. A trispecific Fc-antigen binding domain construct includes three different antigen binding domains.

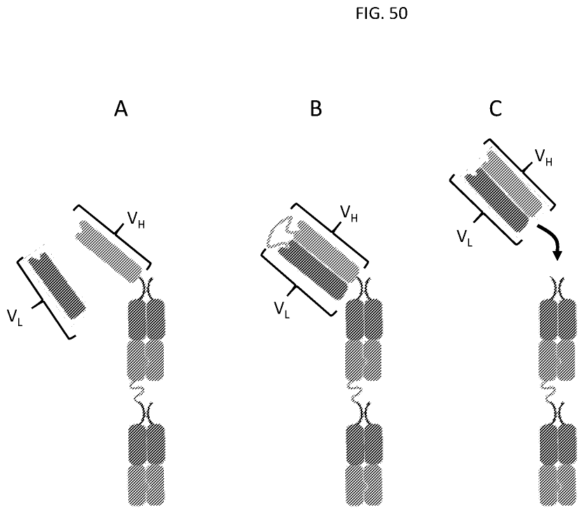

The antigen binding domain can be joined to the Fc-antigen binding domain construct in many ways. The antigen binding domain can be expressed as a fusion protein of an Fc chain. The heavy chain component of the antigen can be expressed as a fusion protein of an Fc chain and the light chain component can be expressed as a separate polypeptide (FIG. 50, panel A). In some embodiments, a scFv is used as an antigen binding domain. The scFv can be expressed as a fusion protein of the long Fc chain (FIG. 50, panel B). In some embodiments the heavy chain and light chain components are expressed separately and exogenously added to the Fc-antigen binding domain construct. In some embodiments, the antigen binding domain is expressed separately and later joined to the Fc-antigen binding domain construct with a chemical bond (FIG. 50, panel C).

In some embodiments, one or more Fc polypeptides in an Fc-antigen binding domain construct lack a C-terminal lysine residue. In some embodiments, all of the Fc polypeptides in an Fc-antigen binding domain construct lack a C-terminal lysine residue. In some embodiments, the absence of a C-terminal lysine in one or more Fc polypeptides in an Fc-antigen binding domain construct may improve the homogeneity of a population of an Fc-antigen binding domain construct (e.g., an Fc-antigen binding domain construct having three Fc domains), e.g., a population of an Fc-antigen binding domain construct having three Fc domains that is at least 85%, 90%, 95%, 98%, or 99% homogeneous.

In some embodiments, the N-terminal Asp in one or more of the first, second, third, fourth, fifth, or sixth polypeptides in an Fc-antigen binding domain construct described herein (e.g., polypeptides 102, 112, and 114 in FIGS. 1, 202, 214, 216 and 218 in FIGS. 2, 302, 320, and 322 in FIGS. 3, 402, 428, 430, and 432 in FIGS. 4, 502, 524, and 526 in FIGS. 5, 602, 632, 634, and 636 in FIGS. 6, 702, 708, 722, and 724 in FIGS. 7, 802, 804, 826, and 828 in FIGS. 8, 902, 904, 934, and 936 in FIGS. 9, 1002, 1010, 1012, 1024, 1026, and 1032 in FIGS. 10, 1102, 1104, 1106, 1144, 1146, and 1148 in FIGS. 11, 1202, 1204, 1206, 1252, 1254, and 1256 in FIG. 12, 1302, 1306 1320, and 1324 in FIGS. 13, 1402, 1404, 1426, and 1428 in FIGS. 14, 1502, 1504, 1534, and 1536 in FIGS. 15, 1602, 1606, 1608, 1626, 1628, and 1632 in FIGS. 16, 1702, 1704, 1706, 1744, 1746, and 1748 in FIGS. 17, 1802, 1804, 1806, 1852, 1854, and 1856 in FIGS. 18, 1902, 1906, 1910, 1924, 1928, and 1932 in FIGS. 19, 2002, 2004, 2006, 2044, 2046, and 2048 in FIGS. 20, 2102, 2104, 2106, 2152, 2154, and 2156 in FIGS. 21, 2202, 2222, and 2224 in FIGS. 22, 2302, 2332, 2334, and 2336 in FIGS. 23, 2402, 2404, 2434, and 2436 in FIGS. 24, 2502, 2504, 2534, and 2536 in FIGS. 25, 2602, 2604, 2606, 2652, 2654, and 2656 in FIGS. 26, 2702, 2704, 2706, 2752, 2754, and 2756 in FIGS. 27, 2802, 2804, 2806, 2852, 2854, and 2856 in FIGS. 28, 2902, 2916, and 2920 in FIGS. 29, 3002, 3024 and 3026 in FIGS. 30, 3102, 312, and 3126 in FIGS. 31, 3202, 3224, 3228, and 3230 in FIGS. 32, 3302, 3332, 3334, and 3336 in FIGS. 33, 3402, 3432, 3434, and 3436 in FIGS. 34, 3502, 3504, 3534, and 3536 in FIG. 35, 3602, 3604, 3612, 3618, 3642, and 3644 in FIGS. 36, 3702, 3704, 3706, 3752, 3754, and 3756 in FIGS. 37, 3802, 3804, 3834, and 3836 in FIGS. 38, 3902, 3904, 3910, 3916, 3942, and 3944 in FIGS. 39, 4002, 4004, 4006, 4052, 4054, and 4056 in FIGS. 40, 4102, 4104, 4110, 4132, 4142, and 4144 in FIGS. 41, 4202, 4204, 4206, 4252, 4254, and 4256 in FIG. 42) may be mutated to Gln.

For the exemplary Fc-antigen binding domain constructs described in the Examples herein. Fc-antigen binding domain constructs 1-28 may contain the E357K and K370D charge pairs in the Knobs and Holes subunits, respectively. Fc-antigen binding domain constructs 29-42 can use orthogonal electrostatic steering mutations that may contain E357K and K370D pairings, and also could include additional steering mutations. For Fc-antigen binding constructs 29-42 with orthogonal knobs and holes electrostatic steering mutations are required all but one of the orthogonal pairs, and may be included in all of the orthogonal pairs.

In some embodiments, if two orthogonal knobs and holes are required, the electrostatic steering modification for Knob1 may be E357K and the electrostatic steering modification for Hole1 may be K370D, and the electrostatic steering modification for Knob2 may be K370D and the electrostatic steering modification for Hole2 may be E357K. If a third orthogonal knob and hole is needed (e.g. for a tri-specific antibody) electrostatic steering modifications E357K and D399K may be added for Knob3 and electrostatic steering modifications K370D and K409D may be added for Hole3 or electrostatic steering modifications K370D and K409D may be added for Knob3 and electrostatic steering modifications E357K and D399K may be added for Hole3.

Any one of the exemplary Fc-antigen binding domain constructs described herein (e.g. Fc-antigen binding domain constructs 1-42) can have enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain, or can include a biological activity that is not exhibited by a construct having a single Fc domain and the antigen binding domain.

### IX. Host Cells and Protein Production

In the present disclosure, a host cell refers to a vehicle that includes the necessary cellular components, e.g., organelles, needed to express the polypeptides and constructs described herein from their corresponding nucleic acids. The nucleic acids may be included in nucleic acid vectors that can be introduced into the host cell by conventional techniques known in the art (transformation, transfection, electroporation, calcium phosphate precipitation, direct microinjection, etc.). Host cells can be of mammalian, bacterial, fungal or insect origin. Mammalian host cells include, but are not limited to, CHO (or CHO-derived cell strains, e.g., CHO-K1, CHO-DXB11 CHO-DG44), murine host cells (e.g., NS0, Sp2/0), VERY, HEK (e.g., HEK293), BHK, HeLa, COS, MDCK, 293, 3T3, W138, BT483, Hs578T, HTB2, BT20 and T47D, CRL7O3O and HsS78Bst cells. Host cells can also be chosen that modulate the expression of the protein constructs, or modify and process the protein product in the specific fashion desired. Different host cells have characteristic and specific mechanisms for the post-translational processing and modification of protein products. Appropriate cell lines or host systems can be chosen to ensure the correct modification and processing of the protein expressed.

For expression and secretion of protein products from their corresponding DNA plasmid constructs, host cells may be transfected or transformed with DNA controlled by appropriate expression control elements known in the art, including promoter, enhancer, sequences, transcription terminators, polyadenylation sites, and selectable markers. Methods for expression of therapeutic proteins are known in the art. See, for example, Paulina Balbas, Argelia Lorence (eds.) Recombinant Gene Expression: Reviews and Protocols (Methods in Molecular Biology), Humana Press; 2nd ed. 2004 edition (Jul. 20, 2004); Vladimir Voynov and Justin A. Caravella (eds.) Therapeutic Proteins: Methods and Protocols (Methods in Molecular Biology) Humana Press; 2nd ed. 2012 edition (Jun. 28, 2012).

### X. Purification

An Fc-antigen binding domain construct can be purified by any method known in the art of protein purification, for example, by chromatography (e.g., ion exchange, affinity (e.g., Protein A affinity), and size-exclusion column chromatography), centrifugation, differential solubility, or by any other standard technique for the purification of proteins. For example, an Fc-antigen binding domain construct can be isolated and purified by appropriately selecting and combining affinity columns such as Protein A column with chromatography columns, filtration, ultra filtration, salting-out and dialysis procedures (see, e.g., Process Scale Purification of Antibodies, Uwe Gottschalk (ed.) John Wiley & Sons, Inc., 2009; and Subramanian (ed.) Antibodies-Volume i-Production and Purification, Kluwer Academic/Plenum Publishers, New York (2004)).

In some instances, an Fc-antigen binding domain construct can be conjugated to one or more purification peptides to facilitate purification and isolation of the Fc-antigen binding domain construct from, e.g., a whole cell lysate mixture. In some embodiments, the purification peptide binds to another moiety that has a specific affinity for the purification peptide. In some embodiments, such moieties which specifically bind to the purification peptide are attached to a solid support, such as a matrix, a resin, or agarose beads. Examples of purification peptides that may be joined to an Fc-antigen binding domain construct include, but are not limited to, a hexa-histidine peptide, a FLAG peptide, a myc peptide, and a hemagglutinin (HA) peptide. A hexa-histidine peptide (HHHHHH (SEQ ID NO: 38)) binds to nickel-functionalized agarose affinity column with micromolar affinity. In some embodiments, a FLAG peptide includes the sequence DYKDDDDK (SEQ ID NO: 39). In some embodiments, a FLAG peptide includes integer multiples of the sequence DYKDDDDK in tandem series, e.g., 3xDYKDDDDK. In some embodiments, a myc peptide includes the sequence EQKLISEEDL (SEQ ID NO: 40). In some embodiments, a myc peptide includes integer multiples of the sequence EQKLISEEDL in tandem series, e.g., 3xEQKLISEEDL. In some embodiments, an HA peptide includes the sequence YPYDVPDYA (SEQ ID NO: 41). In some embodiments, an HA peptide includes integer multiples of the sequence YPYDVPDYA in tandem series, e.g., 3xYPYDVPDYA. Antibodies that specifically recognize and bind to the FLAG, myc, or HA purification peptide are well-known in the art and often commercially available. A solid support (e.g., a matrix, a resin, or agarose beads) functionalized with these antibodies may be used to purify an Fc-antigen binding domain construct that includes a FLAG, myc, or HA peptide.

For the Fc-antigen binding domain constructs. Protein A column chromatography may be employed as a purification process. Protein A ligands interact with Fc-antigen binding domain constructs through the Fc region, making Protein A chromatography a highly selective capture process that is able to remove most of the host cell proteins. In the present disclosure, Fc-antigen binding domain constructs may be purified using Protein A column chromatography as described in Example 2.

### XI. Pharmaceutical Compositions/Preparations

The disclosure features pharmaceutical compositions that include one or more Fc-antigen binding domain constructs described herein. In one embodiment, a pharmaceutical composition includes a substantially homogenous population of Fc-antigen binding domain constructs that are identical or substantially identical in structure. In various examples, the pharmaceutical composition includes a substantially homogenous population of any one of Fc-antigen binding domain constructs 1-42.

A therapeutic protein construct, e.g., an Fc-antigen binding domain construct described herein (e.g., an Fc-antigen binding domain construct having three Fc domains), of the present disclosure can be incorporated into a pharmaceutical composition. Pharmaceutical compositions including therapeutic proteins can be formulated by methods know to those skilled in the art. The pharmaceutical composition can be administered parenterally in the form of an injectable formulation including a sterile solution or suspension in water or another pharmaceutically acceptable liquid. For example, the pharmaceutical composition can be formulated by suitably combining the Fc-antigen binding domain construct with pharmaceutically acceptable vehicles or media, such as sterile water for injection (WFI), physiological saline, emulsifier, suspension agent, surfactant, stabilizer, diluent, binder, excipient, followed by mixing in a unit dose form required for generally accepted pharmaceutical practices. The amount of active ingredient included in the pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50, and the like commonly known in the art. Formulation methods for therapeutic protein products are known in the art, see e.g., Banga (ed.) Therapeutic Peptides and Proteins: Formulation, Processing and Delivery Systems (2d ed.) Taylor & Francis Group, CRC Press (2006).

### XII. Dosage

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, oral dosage forms such as ingestible solutions, drug release capsules, and the like. The appropriate dosage for the individual subject depends on the therapeutic objectives, the route of administration, and the condition of the patient. Generally, recombinant proteins are dosed at 1-200 mg/kg, e.g., 1-100 mg/kg, e.g., 20-100 mg/kg. Accordingly, it will be necessary for a healthcare provider to tailor and titer the dosage and modify the route of administration as required to obtain the optimal therapeutic effect.

### XIII. Complement-Dependent Cytotoxicity (CDC)

Fc-antigen binding domain constructs described in this disclosure are able to activate various Fc receptor mediated effector functions. One component of the immune system is the complement-dependent cytotoxicity (CDC) system, a part of the innate immune system that enhances the ability of antibodies and phagocytic cells to clear foreign pathogens. Three biochemical pathways activate the complement system: the classical complement pathway, the alternative complement pathway, and the lectin pathway, all of which entail a set of complex activation and signaling cascades.

In the classical complement pathway, IgG or IgM trigger complement activation. The C1q protein binds to these antibodies after they have bound an antigen, forming the C1 complex. This complex generates C1s esterase, which cleaves and activates the C4 and C2 proteins into C4a and C4b, and C2a and C2b. The C2a and C4b fragments then form a protein complex called C3 convertase, which cleaves C3 into C3a and C3b, leading to a signal amplification and formation of the membrane attack complex.

The Fc-antigen binding domain constructs of this disclosure are able to enhance CDC activity by the immune system.

CDC may be evaluated by using a colorimetric assay in which Raji cells (ATCC) are coated with a serially diluted antibody, Fc-antigen binding domain construct, or IVIg. Human serum complement (Quidel) can be added to all wells at 25% v/v and incubated for 2 h at 37° C. Cells can be incubated for 12 h at 37° C. after addition of WST-1 cell proliferation reagent (Roche Applied Science). Plates can then be placed on a shaker for 2 min and absorbance at 450 nm can be measured.

### XIV. Antibody-Dependent Cell-Mediated Cytotoxicity (ADCC)

The Fc-antigen binding domain constructs of this disclosure are also able to enhance antibody-dependent cell-mediated cytotoxicity (ADCC) activity by the immune system. ADCC is a part of the adaptive immune system where antibodies bind surface antigens of foreign pathogens and target them for death. ADCC involves activation of natural killer (NK) cells by antibodies. NK cells express Fc receptors, which bind to Fc portions of antibodies such as IgG and IgM. When the antibodies are bound to the surface of a pathogen-infected target cell, they then subsequently bind the NK cells and activate them. The NK cells release cytokines such as IFN-γ, and proteins such as perforin and granzymes. Perforin is a pore forming cytolysin that oligomerizes in the presence of calcium. Granzymes are serine proteases that induce programmed cell death in target cells. In addition to NK cells, macrophages, neutrophils and eosinophils can also mediate ADCC.

ADCC may be evaluated using a luminescence assay. Human primary NK effector cells (Hemacare) are thawed and rested overnight at 37° C. in lymphocyte growth medium-3 (Lonza) at 5×10⁵/mL. The next day, the human lymphoblastoid cell line Raji target cells (ATCC CCL-86) are harvested, resuspended in assay media (phenol red free RPMI, 10% FBSA, GlutaMAX™), and plated in the presence of various concentrations of each probe of interest for 30 minutes at 37° C. The rested NK cells are then harvested, resuspended in assay media, and added to the plates containing the anti-CD20 coated Raji cells. The plates are incubated at 37° C. for 6 hours with the final ratio of effector-to-target cells at 5:1 (5×10⁴ NK cells: 1×10⁴ Raji).

The CytoTox-Glo™ Cytotoxicity Assay kit (Promega) is used to determined ADCC activity. The CytoTox-Glo™ assay uses a luminogenic peptide substrate to measure dead cell protease activity which is released by cells that have lost membrane integrity e.g. lysed Raji cells. After the 6 hour incubation period, the prepared reagent (substrate) is added to each well of the plate and placed on an orbital plate shaker for 15 minutes at room temperature. Luminescence is measured using the PHERAstar F5 plate reader (BMG Labtech). The data is analyzed after the readings from the control conditions (NK cells+Raji only) are subtracted from the test conditions to eliminate background.

### XV. Antibody-Dependent Cellular Phagocytosis (ADCP)

The Fc-antigen binding domain constructs of this disclosure are also able to enhance antibody-dependent cellular phagocytosis (ADCP) activity by the immune system. ADCP, also known as antibody opsonization, is the process by which a pathogen is marked for ingestion and elimination by a phagocyte. Phagocytes are cells that protect the body by ingesting harmful foreign pathogens and dead or dying cells. The process is activated by pathogen-associated molecular patterns (PAMPS), which leads to NF-κB activation. Opsonins such as C3b and antibodies can then attach to target pathogens. When a target is coated in opsonin, the Fc domains attract phagocytes via their Fc receptors. The phagocytes then engulf the cells, and the phagosome of ingested material is fused with the lysosome. The subsequent phagolysosome then proteolytically digests the cellular material.

ADCP may be evaluated using a bioluminescence assay. Antibody-dependent cell-mediated phagocytosis (ADCP) is an important mechanism of action of therapeutic antibodies. ADCP can be mediated by monocytes, macrophages, neutrophils and dendritic cells via FcγRIIa (CD32a), FcγRI (CD64), and FcγRIIIa (CD16a). All three receptors can participate in antibody recognition, immune receptor clustering, and signaling events that result in ADCP; however, blocking studies suggest that FcγRIIa is the predominant Fcγ receptor involved in this process.

The FcγRIIa-H ADCP Reporter Bioassay is a bioluminescent cell-based assay that can be used to measure the potency and stability of antibodies and other biologics with Fc domains that specifically bind and activate FcγRIIa. The assay consists of a genetically engineered Jurkat T cell line that expresses the high-affinity human FcγRIIa-H variant that contains a Histidine (H) at amino acid 131 and a luciferase reporter driven by an NFAT-response element (NFAT-RE).

When co-cultured with a target cell and relevant antibody, the FcγRIIa-H effector cells bind the Fc domain of the antibody, resulting in FcγRIIa signaling and NFAT-RE-mediated luciferase activity. The bioluminescent signal is detected and quantified with a Luciferase assay and a standard luminometer.

### EXAMPLES

The following examples are put forth so as to provide those of ordinary skill in the art with a complete disclosure and description of how the methods and compounds claimed herein are performed, made, and evaluated, and are intended to be purely exemplary of the disclosure and are not intended to limit the scope of what the inventors regard as their disclosure.

### Example 1. Design and Purification of Fc-Antigen Binding Domain Construct 7 with an Anti-CD20 Antigen Binding Domain or an Anti-PD-L1 Antigen Binding Domain

### Protein Expression

Fc-antigen binding domain constructs are designed to increase folding efficiencies, to minimize uncontrolled association of subunits, which may create unwanted high molecular weight oligomers and multimers, and to generate compositions for pharmaceutical use that are substantially homogenous (e.g., at least 85%, 90%, 95%, 98%, or 99% homogeneous). With these goals in mind, a construct formed from a singly branched Fc domain where the branch point is at the N-terminal Fc domain was made as described below. Fc-antigen binding domain construct 7 (CD20)) and construct 7 (PD-L1) each include two distinct Fc domain monomer containing polypeptides (two copies of either an anti-CD20 long Fc chain (SEQ ID NO: 62) or an anti-PD-L1 long Fc chain (SEQ ID NO: 54), and two copies of a short Fc chain (SEQ ID NO: 63)), and two copies of either an anti-CD20 light chain polypeptide (SEQ ID NO: 61) or an anti-PD-L1 light chain polypeptide (SEQ ID NO: 49), respectively. The long Fc chain contains an Fc domain monomer with an E357K charge mutation and S354C and T366W protuberance-forming mutations (to promote heterodimerization) in a tandem series with a charge-mutated (K409D/D399K mutations) Fc domain monomer (to promote homodimerization), and either anti-CD20 VH and CH1 domains (Kabat positions 1-220) at the N-terminus (construct 7 (CD20) or anti-PD-L1 VH and CH1 domains (Kabat positions 1-220) at the N-terminus (construct 7 (PD-L1)). The short Fc chain contains an Fc domain monomer with a K370D charge mutation and Y349C, T366S, L368A, and Y407V cavity-forming mutations (to promote heterodimerization). The anti-CD20 light chain or anti-PD-L1 light chain can also be expressed fused to the N-terminus of the long Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences in Table 7 were encoded by three separate plasmids (one plasmid encoding the light chain (anti-CD20 or anti-PD-L1), one plasmid encoding the long Fc chain (anti-CD20 or anti-PD-L1) and one plasmid encoding the short Fc chain).

TABLE 7
Construct 7 (CD20) and Construct 7 (PD-L1) sequences

| | | Long Fc chain | |
|---|---|---|---|
| | | (with anti-CD20 or anti- | |
| Construct | Light chain | PD-L1 VH and CH1) | Short Fc chain |
| Construct 7 | SEQ ID NO: 61 | SEQ ID NO: 62 | SEQ ID NO: 63 |
| (CD20) | DIVMTQTPLSLPVTPGEPASI | QVQLVQSGAEVKKPGSSVKV | DKTHTCPPCPAPELLGGPSVF |
| | SCRSSKSLLHSNGITYLYWYL | SCKASGYAFSYSWINWVRQ | LFPPKPKDTLMISRTPEVTCV |
| | QKPGQSPQLLIYQMSNLVSG | APGQGLEWMGRIFPGDGDT | VVDVSHEDPEVKFNWYVDG |
| | VPDRFSGSGSGTDFTLKISRV | DYNGKFKGRVTITADKSTSTA | VEVHNAKTKPREEQYNSTYR |
| | EAEDVGVYYCAQNLELPYTF | YMELSSLRSEDTAVYYCARN | VVSULTVLHQDWLNGKEYK |
| | GGGTKVEIKRTVAAPSVFIFP | VFDGYWLVYWGQGTLVTVS | CKVSNKALPAPIEKTISKAKG |
| | PSDEQLKSGIASVVCLINNFY | SASTKGPSVFPLAPSSKSTSG | QPREPQVCILPPSRDELTKN |
| | PREAKVQWKVDNALQSGNS | GTAALGCLVKDYFPEPVTVS | QVSLSCAVDGFYPSDIAVEW |
| | QESVTEQDSKDSTYSLSSTLT | WNSGALTSGVHTFPAVLQSS | ESNGQPENNYKTTPPVLDSD |
| | LSKADYEKHKVYACEVTHQG | GLYSLSSVVTVPSSSLGTQTYI | GSFFLVSKLTVDKSRWQQG |
| | LSSPVTKSENRGEC | CNVNHKPSNTKVDKKVEPKS | NVFSCSVMHEALHNHYTQK |
| | | CDKTHTCPPCPAPELLGGPS | SLSLSPG |
| | | VELEPPKPKDTLMISRTPEVT | |
| | | CVVVDVSHEDPEVKENWYV | |
| | | DGVEVHNAKTKPREEQYNST | |
| | | YRVVSULTVLHQDWLNGKE | |
| | | YKCKVSNKALPAPIEKTISKAK | |
| | | GQPREPQVYTLPPSRDELTK | |
| | | NOVSLTCLVKGFYPSDIAVE | |
| | | WESNGQPENNYKTTPPVLKS | |
| | | DGSFFLYSDLTVDKSRWOQ | |
| | | GNVFSCSVMHEALHNHYTQ | |
| | | KSLSLSPGKGGGGGGGGGG | |
| | | GGGGGGGGGGDKTHTCPP | |
| | | CPAPELLGGPSVFLFPPKPKD | |
| | | TLMISRTPEVTCVVVDVSHE | |
| | | DPEVKFNWYVDGVEVHNAK | |
| | | TKPREEQYNSTYRVVSVLTVL | |
| | | HQDWLNGKEYKCKVSNKAL | |
| | | PAPIEKTISKAKGQPREPQVY | |
| | | TLPPCRDKLTKNQVSLWCLV | |
| | | KGFYPSDAVEWESNGQPEN | |
| | | NYKTTPPVLDSDGSFFLYSKL | |
| | | TVDKSRWQQGNVFSCSVM | |
| | | HEALHNHYTQKSLSLSPG | |
| Construct 7 | SEQ ID NO: 49 | SEQ ID NO: 54 | SEQ ID NO: 63 |
| (PD-L1) | QSALTQPASVSGSPGQSITIS | EVQLLESGGGLVQPGGSLRL | DKTHTCPPCPAPELLGGPSVF |
| | CTGTSSDVGGYNYVSWYQQ | SCAASGFTESSYIMMWVRQ | LFPPKPKDTLMISRTPEVTCV |
| | HPGKAPKLMIYDVSNRPSGV | APGKGLEWVSSIYPSGGITFY | VVDVSHEDPEVKFNWYVDG |
| | SNRFSGSKSGNTASLTISGLQ | ADTVKGRFTISRDNSKNTLYL | VEVHNAKTKPREEQYNSTYR |
| | AEDEADYYCSSYTSSSTRVFG | QMNSLRAEDTAVYYCARIKL | VVSVLTVLHQDWLNGKEYK |
| | TGTKVTVLGQPKANPTVTLF | GTVTTVDYWGQGTLVTVSS | CKVSNKALPAPIEKTISKAKG |
| | PPSSEELQANKATLVCLISDFY | ASTKGPSVFPLAPSSKSTSGG | QPREPQVCTLPPSRDELTKN |
| | PGAVTVAWKADGSPVKAGV | TAALGCLVKDYFPEPVTVSW | QVSLSCAVDGFYPSDIAVEW |
| | ETTKPSKQSNNKYAASSYLSL | NSGALTSGVHTFPAVLQSSG | ESNGQPENNYKTTPPVLDSD |
| | TPEQWKSHRSYSCQVTHEGS | LYSLSSVVTVPSSSLGTQTYIC | GSFFLVSKLTVDKSRWQQG |
| | TVEKTVAPTECS | NVNHKPSNTKVDKKVEPKSC | NVFSCSVMHEALHNHYTQK |
| | | DKTHTCPPCPAPELLGGPSVF | SLSLSPG |
| | | LFPPKPKDTLMISRTPEVTCV | |
| | | VVDVSHEDPEVKFNWYVDG | |
| | | VEVHNAKTKPREEQYNSTYR | |
| | | VVSVLTVLHODWLNGKEYK | |
| | | CKVSNKALPAPIEKTISKAKG | |
| | | QPREPQVYTLPPSRDELTKN | |
| | | QVSLTCLVKGFYPSDIAVEW | |
| | | ESNGOPENNYKTTPPVLKSD | |
| | | GSFFLYSDLTVDKSRWQQG | |
| | | NVFSCSVMHEALHNHYTQK | |
| | | SLSLSPGKGGGGGGGGGGG | |
| | | GGGGGGGGGDKTHTCPPCP | |
| | | APELLGGPSVFLFPPKPKDTL | |
| | | MISRTPEVTCVVVDVSHEDP | |
| | | EVKFNWYVDGVEVHNAKTK | |
| | | PREEQYNSTYRVVSVLTVLH | |
| | | QDWLNGKEYKCKVSNKALP | |
| | | APIEKTISKAKGQPREPQVYT | |
| | | LPPCRDKLTKNOVSLWCLVK | |
| | | GFYPSDIAVEWESNGQPEN | |
| | | NYKTTPPVLDSDGSFFLYSKL | |
| | | TVDKSRWQQGNVFSCSVM | |
| | | HEALHNHYTQKSLSLSPG | |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Sulfate-Polyacrylamide Gel Electrophoresis (SDS-PAGE)

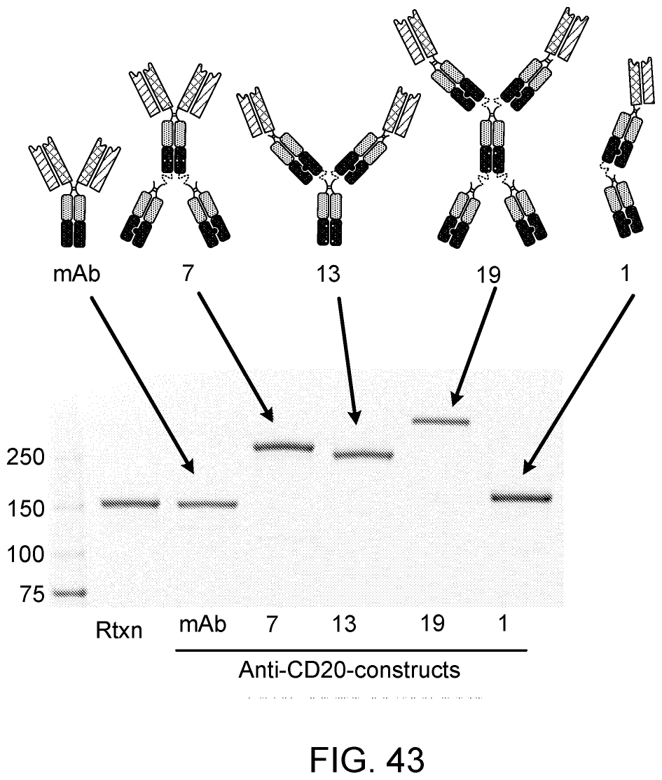

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad). Fc-antigen binding domain construct 7 (CD20) was shown to be pure (FIG. 43, lane 4).

### Example 2. Design and Purification of Fc-Antigen Binding Domain Construct 13 with an Anti-CD20 Antigen Binding Domain or an Anti-PD-L1 Antigen Binding Domain

### Protein Expression

A construct formed from a singly branched Fc domain where the branch point is at the C-terminal Fc domain was made as described below. Fc-antigen binding domain construct 13 (CD20) and construct 13 (PD-L1) each include two distinct Fc domain monomer containing polypeptides (two copies of either an anti-CD20 long Fc chain (any one of SEQ ID NOs: 64 and 67-69) or an anti-PD-L1 long Fc chain (any one of SEQ ID NOs: 58, 59, 60, and 65, and two copies of a short Fc chain (SEQ ID NO: 63)) and two copies of either an anti-CD20 light chain polypeptide (SEQ ID NO: 61) or an anti-PD-L1 light chain polypeptide (SEQ ID NO: 49), respectively. The long Fc chain contains a charge-mutated (K409D/D399K mutations) Fc domain monomer (to promote homodimerization) in a tandem series with an Fc domain monomer with an E357K charge mutation and S354C and T366W protuberance-forming mutations (to promote heterodimerization), and either anti-CD20 VH and CH1 domains (Kabat positions 1-220) at the N-terminus (construct 13 (CD20) or anti-PD-L1 VH and CH1 domains (Kabat positions 1-220) at the N-terminus (construct 13 (PD-L1)). The short Fc chain contains an Fc domain monomer with a K370D charge mutation and Y349C, T366S, L368A, and Y407V cavity-forming mutations (to promote heterodimerization). The anti-CD20 light chain or PD-L1 light chain can also be expressed fused to the N-terminus of the long Fc chain as part of an scFv. Four versions of construct 13 were made with the anti-CD20 heavy chain and with the anti-PD-L1 heavy chain, wherein each version carried a different sized glycine spacer (G4, G10, G15 or G20 linkers) between the Fc domain monomers in the long Fc chain polypeptide. DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for each of the following constructs were encoded by three separate plasmids (one plasmid encoding the light chain (anti-CD20 or anti-PD-L1), one plasmid encoding the long Fc chain (anti-CD20 or anti-PD-L1) and one plasmid encoding the short Fc chain):

TABLE 8
Construct 13 (CD20) and Construct 13 (PD-L1) sequences

| | | Long Fc chain | |
|---|---|---|---|
| | | (with anti-CD20 or | |
| Construct | Light chain | anti-PD-L1 VH and CH1) | Short Fc chain |
| Construct 13 | SEQ ID NO: 61 | SEQ ID NO: 64 | SEQ ID NO: 63 |
| (CD20), G₂₀ | DIVMTQTPLSLPVTPGEPASI | QVQLVQSGAEVKKPGSSVKV | DKTHTCPPCPAPELLGGPSVF |
| linker | SCRSSKSLLHSNGITYLYWYL | SCKASGYAFSYSWINWVRQ | LFPPKPKDTLMISRTPEVTCV |
| | QKPGQSPCILLIYQMSNLVSG | APGQGLEWMGRIFFPGDGDI | VVDVSHEDPEVKFNWYVDG |
| | VPDRFSGSGSGTDFTLKISRV | DYNGKFKGRVTITADKSTSTA | VEVHNAKTKPREEQYNSTYR |
| | EAEDVGVYYCAQNLELPYTF | YMELSSLRSEDTAVYYCARN | VVSVLTVLHCIDWLNGKEYK |
| | GGGTKVEIKRIVAAPSVFIFP | VFDGYWLVYWGQGILVTVS | CKVSNKALPAPIEKTISKAKG |
| | PSDEQLKSGTASVVCLLNNEY | SASTKGPSVFPLAPSSKSTSG | QPREPQVCTLPPSRDELTKN |
| | PREAKVQWKVDNALQSGNS | GTAALGCLVKDYFPEPVTVS | QVSLSCAVDGFYPSDIAVEW |
| | QESVTEQDSKDSTYSLSSTLT | WNSGALTSGVHTFPAVLQSS | ESNGQPENNYKTTPPVLDSD |
| | LSKADYEKHKVYACEVTHQG | GLYSLSSVVTVPSSSLGTQTYI | GSFELVSKLTVDKSRWQQG |
| | LSSPVTKSFNRGEC | CNVNHKPSNTKVDKKVEPKS | NVFSCSVMHEALHNHYTQK |
| | | CDKTHTCPPCPAPELLGGPS | SLSLSPG |
| | | VFLEPPKPKDTLMISRTPEVT | |
| | | CVVVDVSHEDPEVKFNWYV | |
| | | DGVEVHNAKTKPREEQYNST | |
| | | YRVVSVLTVLHQDWLNGKE | |
| | | YKCKVSNKALPAPIEKTISKAK | |
| | | GQPREPQVYTLPPCRDKLTK | |
| | | NQVSLWCLVKGFYPSDAVE | |
| | | WESNGQPENNYKTTPPVLD | |
| | | SDGSFFLYSKLTVDKSRWQQ | |
| | | GNVESCSM1HEALHNHYTQ | |
| | | KSLSLSPGKGGGGGGGGGG | |
| | | GGGGGGGGGGDKTHTCPP | |
| | | CPAPELLGGPSVFLEPPKPKD | |
| | | TLMISRTPEVTCVVVDVSHE | |
| | | DPEVKFNWYVDGVEVHNAK | |
| | | TKPREEQYNSTYRVVSVLTVL | |
| | | HQDWLNGKEYKCKVSNKAL | |
| | | PAPIEKTISKAKGQPREPQVY | |
| | | TLPPSRDELTKNQVSLTCLVK | |
| | | GFYPSDIAVEWESNGQPEN | |
| | | NYKTTPPVLKSDGSFFLYSDL | |
| | | TVDKSRWQQGNVFSCSVM | |
| | | HEALHNHYTQKSLSLSPG | |
| Construct 13 | SEQ ID NO: 61 | SEQ ID NO: 55 | SEQ ID NO: 63 |
| (CD20), G₁₅ | DIVMTQTPLSLPVTPGEPASI | QVQLVQSGAEVKKPGSSV | DKTHTCPPCPAPELLGGPSVF |
| linker | SCRSSICSLLHSNGITYLYWYL | KVSCKASGYAFSYSWINW | LFPPKPKDTLMISRTPEVTCV |
| | QKPGQSPQLLIYQMSNLVSG | VRQAPGQGLEWMGRIFP | VVDVSHEDPEVKFNWYVDG |
| | VPDRFSGSGSGTDFTLKISRV | GDGDTDYNGKFKGRVTIT | VEVHNAKTKPREEQYNSTYR |
| | EAEDVGVYYCAQNLELPYTF | ADKSTSTAYMELSSLRSED | VVSVLTVLHQDWLNGKEYK |
| | GGGTKVEIKRTVAAPSVFIFP | TAVYYCARNVFDGYWLVY | CKVSNKALPAPIEKTISKAKG |
| | PSDEQLKSGTASVVCLLNNFY | WGQGTLVTVSSASTKGPS | QPREPQVCTLPPSRDELTKN |
| | PRFAKVQWKVDNALQSGNS | VFPLAPSSKSTSGGTAALG | QVSLSCAVDGFYPSDIAVEW |
| | QESVTEQDSKDSTYSLSSILT | CLVKDYFPEPVTVSWNSG | ESNGQPENNYKTTPPVLDSD |
| | LSKADYEKHKVYACEVIHQG | ALTSGVHTFPAVLQSSGLY | GSFFLVSKLTVDKSRWQQG |
| | LSSPVTKSENRGEC | SLSSVVTVPSSSLGTQTYIC | NVFSCSVMHEALHNHYTQK |
| | | NVNHKPSNTKVDKKVEPK | SLSLSPG |
| | | SCDKTHTCPPCPAPELLGG | |
| | | PSVFLFPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPCRDKLTKNQVS | |
| | | LWCLVKGFYPSDIAVEWE | |
| | | SNGQPENNYKTTPPVLDS | |
| | | DGSFFLYSKLTVDKSRWQ | |
| | | QGNVFSCSVMHEALHNH | |
| | | YTQKSLSLSPGKGGGGGG | |
| | | GGGGGGGGGDKTHTCPP | |
| | | CPAPELLGGPSVFLFPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPSR | |
| | | DELTKNQVSLTCLVKGFYP | |
| | | SDIAVEWESNGQPENNYK | |
| | | TTPPVLKSDGSFFLYSDLTV | |
| | | DKSRWQQGNVFSCSVM | |
| | | HEALHNHYMKSLSLSPG | |
| Construct 13 | SEQ ID NO: 61 | SEQ ID NO: 56 | SEQ ID NO: 63 |
| (CD20), G₁₀ | DIVMTQTPLSLPVTPGEPASI | QVQLVQSGAEVKKPGSSV | DKTHTCPPCPAPELLGGPSVF |
| linker | SCRSSKSLLHSNGITYLYWYL | KVSCKASGYAFSYSWINW | LFPPKPKDTLMISRTPEVTCV |
| | QKPGQSPQLLIYQMSNLVSG | VRQAPGQGLEWMGRIFP | VVDVSHEDPEVKFNWYVDG |
| | VPDRFSGSGSGTDFTLKISRV | GDGDTDYNGKFKGRVTIT | VEVHNAKTKPREEQYNSTYR |
| | EAEDVGVYYCAQNLELPYTF | ADKSTSTAYMELSSLRSED | VVSVLTVLHQDWLNGKEYK |
| | GGGTKVEIKRTVAAPSVFIFP | TAVYYCARNVFDGYWLVY | CKVSNKALPAPIEKTISKAKG |
| | PSDEQLKSGTASVVCLLNNFY | WGQGTLVTVSSASTKGPS | QPREPQVCTLPPSRDELTKN |
| | PREAKVQWKVDNALQSGNS | VFPLAPSSKSTSGGTAALG | QVSLSCAVDGFYPSDIAVEW |
| | QESVTEQDSKDSTYSLSSTLT | CLVKDYFPEPVTVSWNSG | ESNGQPENNYKTTPPVLDSD |
| | LSKADYEKHKVYACEVTHQG | ALTSGVHTFPAVLQSSGLY | GSFFLVSKLTVDKSRWQQG |
| | LSSPVTKSFNRGEC | SLSSVVTVPSSSLGTQTYIC | NVFSCSVMHEALHNHYTQK |
| | | NVNHKPSNTKVDKKVEPK | SLSLSPG |
| | | SCDKTHTCPPCPAPELLGG | |
| | | PSVFLFPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPCRDKLTKNQVS | |
| | | LWCLVKGFYPSDIAVEWE | |
| | | SNGQPENNYKIIPPVLDS | |
| | | DGSFFLYSKLTVDKSRWQ | |
| | | QGNVFSCSVMHEALHNH | |
| | | YTQKSLSLSPGKGGGGGG | |
| | | GGGGDKTHTCPPCPAPEL | |
| | | LGGPSVFLFPPKPKDTLMI | |
| | | SRTPEVTCVVVDVSHEDP | |
| | | EVKFNWYVDGVEVHNAK | |
| | | TKPREEQYNSTYRVVSVLT | |
| | | VLHQDWLNGKEYKCKVS | |
| | | NKALPAPIEKTISKAKGQP | |
| | | REPQYYTLPPSRDELTKN | |
| | | QVSLTCLVKGFYPSDIAVE | |
| | | WESNGQPENNYKTTPPV | |
| | | LKSDGSFFLYSDLTVDKSR | |
| | | WQQGNVFSCSVMHEAL | |
| | | HNHYTQKSLSLSPG | |
| Construct 13 | SEQ ID NO: 61 | SEQ ID NO: 57 | SEQ ID NO: 63 |
| (CD20), G₄ | DIVMTQTPLSLPVTPGEPASI | QVQLVQSGAEVKKPGSSV | DKTHTCPPCPAPELLGGPSVF |
| linker | SCRSSKSLLHSNGITYLYWYL | KVSCKASGYAFSYSWINW | LFPPKPKDTLMISRTPEVTCV |
| | QKPGQSPQLLIYQMSNLVSG | VRQAPGQGLEWMGRIFP | VVDVSHEDPEVKFNWYVDG |
| | VPDRFSGSGSGTDFTLKISRV | GDGDTDYNGKFKGRVTIT | VEVHNAKTKPREEQYNSTYR |
| | EAEDVGVYYCAQNLELPYTF | ADKSTSTAYMELSSLRSED | VVSVLTVLHQDWLNGKEYK |
| | GGGTKVEIKRTVAAPSVFIFP | TAVYYCARNVFDGYWLVY | CKVSNKALPAPIEKTISKAKG |
| | PSDEQLKSGTASVVCLLNNFY | WGQGTLVTVSSASTKGPS | QPREPQVCTLPPSRDELTKN |
| | PREAKVQWKVDNALQSGNS | VFPLAPSSKSTSGGTAALG | QVSLSCAVDGFYPSDIAVEW |
| | QESVTEQDSKDSTYSLSSTLT | CLVKDYFPEPVTVSWNSG | ESNGQPENNYKTTPPVLDSD |
| | LSKADYEKHKVYACEVTHQG | ALTSGVHTFPAVLQSSGLY | GSFFLVSKLTVDKSRWQQG |
| | LSSPVTKSFNRGEC | SLSSVVTVPSSSLGTQTYIC | NVFSCSVMHEALHNHYTQK |
| | | NVNHKPSNTKVDKKVEPK | SLSLSPG |
| | | SCDKTHTCPPCPAPELLGG | |
| | | PSVFLFPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPCRDKLTKNQVS | |
| | | LWCLVKGFYPSDIAVEWE | |
| | | SNGQPENNYKTTPPVLDS | |
| | | DGSFFLYSKLTVDKSRWQ | |
| | | QGNVFSCSVMHEALHNH | |
| | | YTQKSLSLSPGKGGGGDK | |
| | | THTCPPCPAPELLGGPSVF | |
| | | LFPPKPKDTLMISRTPEVT | |
| | | CVVVDVSHEDPEVKFNW | |
| | | YVDGVEVHNAKTKPREEQ | |
| | | YNSTYRVVSVLTVLHQDW | |
| | | LNGKEYKCKVSNKALPAPI | |
| | | EKTISKAKGQPREPQVYTL | |
| | | PPSRDELTKNQVSLTCLVK | |
| | | GFYPSDIAVEWESNGQPE | |
| | | NNYKTTPPVLKSDGSFFLY | |
| | | SDLTVDKSRWQQGNVFS | |
| | | CSVMHEALHNHYTQKSLS | |
| | | LSPG | |
| Construct 13 | SEQ ID NO: 49 | SEQ ID NO: 58 | SEQ ID NO: 63 |
| (PD-L1), G₂₀ | QSALTQPASVSGSPGQSITIS | EVQLLESGGGLVQPGGSLRL | DKTHTCPPCPAPELLGGPSVF |
| linker | CTGTSSDVGGYNYVSWYQQ | SCAASGFTFSSYIMMWVRQ | LFPPKPKDTLMISRTPEVTCV |
| | HPGKAPKLMIYDVSNRPSGV | APGKGLEWVSSIYPSGGITFY | VVDVSHEDPEVKFNWYVDG |
| | SNRFSGSKSGNTASLTISGLQ | ADTVKGRFTISRDNSKNTLYL | VEVHNAKTKPREEQYNSTYR |
| | AEDEADYYCSSYTSSSTRVFG | QMNSLRAEDTAVYYCARIKL | VVSVLTVLHQDWLNGKEYK |
| | TGTKVTVLGQPKANPTVTLF | GTVTTVDYWGQGTLVTVSS | CKVSNKALPAPIEKTISKAKG |
| | PPSSEELQANKATLVCLISDFY | ASTKGPSVFPLAPSSKSTSGG | QPREPQVCTLPPSRDELTKN |
| | PGAVTVAWKADGSPVKAGV | TAALGCLVKDYFPEPVTVSW | QVSLSCAVDGFYPSDIAVEW |
| | ETTKPSKQSNNKYAASSYLSL | NSGALTSGVHTEPAVLQSSG | ESNGQPENNYKTTPPVLDSD |
| | TPEQWKSHRSYSCQVTHEGS | LYSLSSVVTVPSSSLGTQTYIC | GSFFLVSKLTVDKSRWQQG |
| | TVEKTVAPTECS | NVNHKPSNTKVDKKVEPKSC | NVFSCSVMHEALHNHYTQK |
| | | DKTHTCPPCPAPELLGGPSVF | SLSLSPG |
| | | LFPPKPKDTLMISRTPEVTCV | |
| | | VVDVSHEDPEVKFNWYVDG | |
| | | VEVHNAKTKPREEQYNSTYR | |
| | | VVSVLTVLHQDWLNGKEYK | |
| | | CKVSNKALPAPIEKTISKAKG | |
| | | QPREPQVYTLPPCRDKLTKN | |
| | | QVSLWCLVKGFYPSDIAVEW | |
| | | ESNGQPENNYKTTPPVLDSD | |
| | | GSFFLYSKLTVDKSRWQQGN | |
| | | VFSCSVMHEALHNHYTQKSL | |
| | | SLSPGKGGGGGGGGGGGG | |
| | | GGGGGGGGDKTHTCPPCPA | |
| | | PELLGGPSVFLFPPKPKDTLM | |
| | | ISRTPEVTCVVVDVSHEDPEV | |
| | | KFNWYVDGVEVHNAKTKPR | |
| | | EEQYNSTYRVVSVLTVLHQD | |
| | | WLNGKEYKCKVSNKALPAPI | |
| | | EKTISKAKGQPREPQVYTLPP | |
| | | SRDELTKNQVSLTCLVKGFYP | |
| | | SDIAVEWESNGQPENNYKTT | |
| | | PPVLKSDGSFRYSDLTVDKS | |
| | | RWQQGNVFSCSVMHEALH | |
| | | NHYTQKSLSLSPG | |
| Construct 13 | SEQ ID NO: 49 | SEQ ID NO: 59 | SEQ ID NO: 63 |
| (PD-L1), G₁₅ | QSALTQPASVSGSPGQSITIS | EVQLLESGGGLVQPGGSLRL | DKTHTCPPCPAPELLGGPSVF |
| linker | CTGTSSDVGGYNYVSWYQQ | SCAASGFTFSSYIMMWVRQ | LFPPKPKDILMISRTPEVTCV |
| | HPGKAPKLMIYDVSNRPSGV | APGKGLEWVSSIYPSGGITFY | VVDVSHEDPEVKFNWYVDG |
| | SNRFSGSKSGNTASLTISGLQ | ADTVKGRFTISRDNSKNTLYL | VEVHNAKTKPREEQYNSTYR |
| | AEDEADYYCSSYTSSSTRVFG | QMNSLRAEDTAVYYCARIKL | VVSVLIVLHQDWLNGKEYK |
| | TGTKVTVLGQPKANPTVTLF | GTVTTVDYWGQGTLVTVSS | CKVSNKALPAPIEKTISKAKG |
| | PPSSEELQANKATLVCLISDFY | ASTKGPSVFPLAPSSKSTSGG | QPREPQVCTLPPSRDELTKN |
| | PGAVTVAWKADGSPVKAGV | TAALGCLVKDYFPEPVTVSW | QVSLSCAVDGFYPSDIAVEW |
| | ETTKPSKQSNNKYAASSYLSL | NSGALTSGVHTFPAVLQSSG | ESNGQPENNYKTTPPVLDSD |
| | TPEQWKSHRSYSCQVTHEGS | LYSLSSVVTVPSSSLGTQTYIC | GSFFLVSKLTVDKSRWQQG |
| | TVEKTVAPTECS | NVNHKPSNTKVDKKVEPKSC | NVFSCSVMHEALHNHYTQK |
| | | DKTHTCPPCPAPELLGGPSVF | SLSLSPG |
| | | LFPPKPKDILMISRTPEVTCV | |
| | | VVDVSHEDPEVKFNWYVDG | |
| | | VEVHNAKTKPREEQYNSTYR | |
| | | VVSVLTVLHQDWLNGKEYK | |
| | | CKVSNKALPAPIEKTISKAKG | |
| | | QPREPQVYTLPPCRDKLTKN | |
| | | QVSLWCLVKGFYPSDIAVEW | |
| | | ESNGQPENNYKTTPPVLDSD | |
| | | GSFFLYSKLTVDKSRVVQQGN | |
| | | VFSCSVMHEALHNHYTQKSL | |
| | | SLSPGKGGGGGGGGGGGG | |
| | | GGGDKTHTCPPCPAPELLGG | |
| | | PSVFLFPPKPKDTLMISRTPE | |
| | | VTCVVVDVSHEDPEVKFNW | |
| | | YVDGVEVHNAKTKPREEQY | |
| | | NSTYRVVSVLTVLHQDWLN | |
| | | GKEYKCKVSNKALPAPIEKTIS | |
| | | KAKGQPREPQVYTLPPSRDE | |
| | | LTKNQVSLTCLVKGFYPSDIA | |
| | | VEWESNGQPENNYKTTPPV | |
| | | LKSDGSFFLYSDLTVDKSRW | |
| | | QQGNVESCSVMHEALHNHY | |
| | | TQKSLSLSPG | |
| Construct 13 | SEQ ID NO: 49 | SEQ ID NO: 60 | SEQ ID NO: 63 |
| (PD-L1), G₁₀ | QSALTQPASVSGSPGQSITIS | EVQLLESGGGLVQPGGSLRL | DKTHTCPPCPAPELLGGPSVF |
| linker | CTGTSSDVGGYNYVSWYQQ | SCAASGFTFSSYIMMWVRQ | LFPPKPKDILMISRTPEVTCV |
| | HPGKAPKLMIYDVSNRPSGV | APGKGLEWVSSIYPSGGITFY | VVDVSHEDPEVKFNWYVDG |
| | SNRFSGSKSGNTASLTISGLQ | ADTVKGRFTISRDNSKNTLYL | VEVHNAKTKPREEQYNSTYR |
| | AEDEADYYCSSYTSSSTRVFG | QMNSLRAEDTAVYYCARIKL | VVSVLIVLHQDWLNGKEYK |
| | TGTKVTVLGQPKANPTVTLF | GTVTTVDYWGQGTLVTVSS | CKVSNKALPAPIEKTISKAKG |
| | PPSSEELQANKATLVCLISDFY | ASTKGPSVFPLAPSSKSTSGG | QPREPQVCTLPPSRDELTKN |
| | PGAVTVAWKADGSPVKAGV | TAALGCLVKDYFPEPVTVSW | QVSLSCAVDGFYPSDIAVEW |
| | ETTKPSKQSNNKYAASSYLSL | NSGALTSGVHTFPAVLQSSG | ESNGQPENNYKTTPPVLDSD |
| | TPEQWKSHRSYSCQVTHEGS | LYSLSSVVTVPSSSLGTQTYIC | GSFFLVSKLTVDKSRWQQG |
| | TVEKTVAPTECS | NVNHKPSNTKVDKKVEPKSC | NVFSCSVMHEALHNHYTQK |
| | | DKTHTCPPCPAPELLGGPSVF | SLSLSPG |
| | | LFPPKPKDTLMISRTPEVTCV | |
| | | VVDVSHEDPEVKFNWYVDG | |
| | | VEVHNAKTKPREEQYNSTYR | |
| | | VVSVLTVLHQDWLNGKEYK | |
| | | CKVSNKALPAPIEKTISKAKG | |
| | | QPREPQVYTLPPCRDKLTKN | |
| | | QVSLWCLVKGFYPSDIAVEW | |
| | | ESNGQPENNYTTPPVLDSD | |
| | | GSFFLYSKLTVDKSRWQQGN | |
| | | VFSCSVMHEALHNHYTQKSL | |
| | | SLSPGKGGGGGGGGGGDKT | |
| | | HTCPPCPAPELLGGPSVFLFP | |
| | | PKPKDTLMISRTPEVTCVVV | |
| | | DVSHEDPEVKFNWYVDGVE | |
| | | VHNAKTKPREEQYNSTYRVV | |
| | | SVLTVLHQDWLNGKEYKCKV | |
| | | SNKALPAPIEKTISKAKGQPR | |
| | | EPQVYTLPPSRDELTKNQVSL | |
| | | TCLVKGFYPSDIAVEWESNG | |
| | | QPENNYKTTPPVLKSDGSFFL | |
| | | YSDLTVDKSRWQQGNVFSC | |
| | | SVMHEALHNHYTQKSLSLSP | |
| | | G | |
| Construct 13 | SEQ ID NO: 49 | SEQ ID NO: 65 | SEQ ID NO: 63 |
| (PD-L1), G₄ | QSALTQPASVSGSPGQSITIS | EVQLLESGGGLVQPGGSLRL | DKTHTCPPCPAPELLGGPSVF |
| linker | CTGTSSDVGGYNYVSWYQQ | SCAASGFTFSSYIMMWVRQ | LFPPKPKDTLMISRTPEVTCV |
| | HPGKAPKLMIYDVSNRPSGV | APGKGLEWVSSIYPSGGITFY | VVDVSHEDPEVKENWYVDG |
| | SNRFSGSKSGNTASLTISGLQ | ADTVKGRFTISRDNSKNTLYL | VEVHNAKTKPREEQYNSTYR |
| | AEDEADYYCSSYTSSSTRVFG | QMNSLRAEDTAVYYCARIKL | VVSVLTVLHQDWLNGKEYK |
| | TGTKVTVLGQPKANPTVTLF | GTVTTVDYWGQGTLVTVSS | CKVSNKALPAPIEKTISKAKG |
| | PPSSEELQANKATLVCLISDFY | ASTKGPSVFPLAPSSKSTSGG | QPREPQVCTLPPSRDELTKN |
| | PGAVTVAWKADGSPVKAGV | TAALGCLVKDYFPEPVTVSW | QVSLSCAVDGFYPSDAVEW |
| | ETTKPSKQSNNKYAASSYLSL | NSGALTSGVHTFPAVLQSSG | ESNGQPENNYKTTPPVLDSD |
| | TPEQWKSHRSYSCQVTHEGS | LYSLSSVVTVPSSSLGTQTYIC | GSFELVSKLIVDKSRWQQG |
| | TVEKTVAPTECS | NVNHKPSNTKVDKKVEPKSC | NVFSCSVMHEALHNHYTQK |
| | | DKTHTCPPCPAPELLGGPSVF | SLSLSPG |
| | | LFPPKPKDTLMISRTPEVTCV | |
| | | VVDVSHEDPEVKFNWYVDG | |
| | | VEVHNAKTKPREEQYNSTYR | |
| | | VVSVLTVLHQDWLNGKEYK | |
| | | CKVSNKALPAPIEKTISKAKG | |
| | | QPREPQVYTLPPCRDKLTKN | |
| | | QVSLWCLVKGFYPSDIAVEW | |
| | | ESNGQPENNYKTTPPVLDSD | |
| | | GSFFLYSKLTVDKSRWQQGN | |
| | | VFSCSVMHEALHNHYTQKSL | |
| | | SLSPGKGGGGDKTHTCPPCP | |
| | | APELLGGPSVFLFPPKPKDTL | |
| | | MISRTPEVTCVVVDVSHEDP | |
| | | EVKFNWYVDGVEVHNAKTK | |
| | | PREEQYNSTYRVVSVLTVLH | |
| | | QMAILNGKEYKCKVSNKALP | |
| | | APIEKTISKAKGQPREPQVYT | |
| | | LPPSRDELTKNQVSLTCLVKG | |
| | | FYPSDIAVEWESNGQPENNY | |
| | | KTTPPVLKSDGSFFLYSDLTV | |
| | | DKSRWQQGNVFSCSVMHE | |
| | | ALHNHYTQKSLSLSPG | |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad). Fc-antigen binding domain construct 13 (CD20) was shown to be pure (FIG. 43, lane 5).

### Example 3. Design and Purification of Fc-Antigen Binding Domain Construct 1

An unbranched construct formed from asymmetrical tandem Fc domains is made as described below. Fc-antigen binding domain construct 1 (FIG. 1) includes two distinct Fc domain monomer containing polypeptides (a long Fc chain and two copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4 (e.g., E357K) (to promote heterodimerization), and an antigen binding domain at the N-terminus. The antigen binding domain may be expressed as part of the same amino acid sequence as the long Fc chain (e.g., to form a scFv). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4 (e.g., K370D) (to promote heterodimerization). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and the long Fc chains are encoded by two separate plasmids. In this Example, and in each of the following Examples for Fc-antigen binding domain constructs 2-42, the cell may contain a third plasmid expressing an antibody variable light chain.

The expressed proteins are purified from the cell culture supernatant by Protein A-based affinity column chromatography, using a Poros MabCapture A (LifeTechnologies) column. Captured Fc-antigen binding domain constructs are washed with phosphate buffered saline (low-salt wash) and eluted with 100 mM glycine, pH 3. The eluate is quickly neutralized by the addition of 1 M TRIS pH 7.4 and sterile filtered through a 0.2 μm filter. The proteins are further fractionated by ion exchange chromatography using Poros XS resin (Applied Biosciences). The column is pre-equilibrated with 50 mM MES, pH 6 (buffer A), and the sample is eluted with a step gradient using 50 mM MES, 400 mM sodium chloride, pH 6 (buffer B) as the elution buffer. After ion exchange, the target fraction is buffer exchanged into PBS buffer using a 10 kDa cut-off polyether sulfone (PES) membrane cartridge on a tangential flow filtration system. The samples are concentrated to approximately 30 mg/mL and sterile filtered through a 0.2 μm filter.

Samples are denatured in Laemmli sample buffer (4% SDS, Bio-Rad) at 95° C. for 10 min. Samples are run on a Criterion TGX stain-free gel (4-15% polyacrylamide, Bio-Rad). Protein bands are visualized by UV illumination or Coommassie blue staining. Gels are imaged by ChemiDoc MP Imaging System (Bio-Rad). Quantification of bands is performed using Imagelab 4.0.1 software (Bio-Rad).

### Example 4. Design and Purification of Fc-Antigen Binding Domain Construct 2

An unbranched construct formed from asymmetrical tandem Fc domains is made as described below. Fc-antigen binding domain construct 2 (FIG. 2) includes two distinct Fc monomer containing polypeptides (a long Fc chain and three copies of a short Fc chain) and a light chain polypeptide. The long Fc chain contains three Fc domain monomers in a tandem series with an antigen binding domain at N-terminus, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4 (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D). DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 5. Design and Purification of Fc-Antigen Binding Domain Construct 3

A construct formed from asymmetrical tandem Fc domains is made as described below. Fc-antigen binding domain construct 3 (FIG. 3) includes two distinct Fc monomer containing polypeptides (a long Fc chain and two copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4 (e.g., E357K). The short Fc chain contains an Fc domain monomer with an engineered cavity that is made by introducing at least one cavity-forming mutation selected from Table 3 (e.g., the Y349C, T366S. L368A. and Y407V mutations), and, optionally, one or more reverse charge mutation selected from Table 4 (e.g., K370D), and an antigen binding domain at N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 6. Design and Purification of Fc-Antigen Binding Domain Construct 4

A construct formed from asymmetrical tandem Fc domains was made as described below. Fc-antigen binding domain construct 4 (FIG. 4) includes two distinct Fc monomer containing polypeptides (a long Fc chain and three copies of a short Fc chain) and a light chain polypeptide. The long Fc chain contains three Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutations selected from Table 4 (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4 (e.g., K370D), and an antigen binding domain at the N-terminus. DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 7. Design and Purification of Fc-Antigen Binding Domain Construct 5

A construct formed from asymmetrical tandem Fc domains is made as described below. Fc-antigen binding domain construct 5 (FIG. 5) includes two distinct Fc monomer containing polypeptides (a long Fc chain and two copies of a short Fc chain) and a light chain polypeptide. The long Fc chain contains two Fc domain monomers in a tandem series with an antigen binding domain at the N-terminus, wherein each Fc domain monomer has an engineered protuberance that is made by introducing at least one protuberance-forming mutation selected from Table 3 (e.g., the S354C and T366W mutations) and, optionally, one or more reverse charge mutations selected from Table 4 (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4 (e.g., K370D), and an antigen binding domain at N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 8. Design and Purification of Fc-Antigen Binding Domain Construct 6

A construct formed from asymmetrical tandem Fc domains is made as described below. Fc-antigen binding domain construct 6 (FIG. 6) includes two distinct Fc monomer containing polypeptides (a long Fc chain and three copies of a short Fc chain) and a light chain polypeptide. The long Fc chain contains three Fc domain monomers in a tandem series with an antigen binding domain at the N-terminus, wherein each Fc domain monomer has an engineered protuberance that is made by introducing at least one protuberance-forming mutation selected from Table 3 (e.g., the S354C and T366W mutations) and, optionally, one or more reverse charge mutations selected from Table 4 (e.g., E357K). The short Fc chain contains an Fc domain monomer with an engineered cavity that is made by introducing at least one cavity-forming mutation selected from Table 3 (e.g., the Y349C, T366S, L368A, and Y407V mutations), and, optionally, a reverse charge mutation selected from Table 4 (e.g., K370D), and an antigen binding domain at N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 9. Design and Purification of Fc-Antigen Binding Domain Construct 7

A construct formed from a singly branched Fc domain where the branch point is at the N-terminal Fc domain was made as described below. Fc-antigen binding domain construct 7 (FIG. 7) includes two distinct Fc monomer containing polypeptides (two copies of a long Fc chain and two copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D). DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 10. Design and Purification of Fc-Antigen Binding Domain Construct 8

A construct formed from a singly branched Fc domain where the branch point is at the N-terminal Fc domain was made as described below. Fc-antigen binding domain construct 8 (FIG. 8) includes two distinct Fc monomer containing polypeptides (two copies of a long Fc chain and two copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations). The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain at the N-terminus. DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 11. Design and Purification of Fc-Antigen Binding Domain Construct 9

A construct formed from a singly branched Fc domain where the branch point is at the N-terminal Fc domain was made as described below. Fc-antigen binding domain construct 9 (FIG. 9) includes two distinct Fc monomer containing polypeptides (two copies of a long Fc chain and two copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain at the N-terminus. DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 12. Design and Purification of Fc-Antigen Binding Domain Construct 10

A construct formed from a singly branched Fc domain where the branch point is at the N-terminal Fc domain was made as described below. Fc-antigen binding domain construct 10 (FIG. 10) includes two distinct Fc monomer containing polypeptides (two copies of a long Fc chain and four copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D). DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 13. Design and Purification of Fc-Antigen Binding Domain Construct 11

A construct formed from a singly branched Fc domain where the branch point is at the N-terminal Fc domain is made as described below. Fc-antigen binding domain construct 11 (FIG. 11) includes two distinct Fc monomer containing polypeptides (two copies of a long Fc chain and four copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations) at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen-binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 14. Design and Purification of Fc-Antigen Binding Domain Construct 12

A construct formed from a singly branched Fc domain where the branch point is at the N-terminal Fc domain is made as described below. Fc-antigen binding domain construct 12 (FIG. 12) includes two distinct Fc monomer containing polypeptides (two copies of a long Fc chain and four copies of a short Fc chain) and a light chain polypeptide. The long Fc chain contains two Fc domain monomers in a tandem series, wherein each Fc domain monomer has an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen-binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 15. Design and Purification of Fc-Antigen Binding Domain Construct 13

A construct formed from a singly branched Fc domain where the branch point is at the C-terminal Fc domain was made as described below. Fc-antigen binding domain construct 13 (FIG. 13) includes two distinct Fc monomer containing polypeptides (two copies of a long Fc chain and two copies of a short Fc chain) and a light chain polypeptide. The long Fc chain contains an Fc domain monomer with reverse charge mutations selected from Table 4 or Table 5 (e.g., the K409D/D399K mutations), in a tandem series with an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain at the N-terminus. The short Fc chain contains an Fc domain monomer with an engineered cavity that is made by introducing at least one cavity-forming mutation selected from Table 3 (e.g., the Y349C, T366S. L368A, and Y407V mutations), and, optionally, one or more reverse charge mutation selected from Table 4 (e.g., K370D). DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 16. Design and Purification of Fc-Antigen Binding Domain Construct 14

A construct formed from a singly branched Fc domain where the branch point is at the C-terminal Fc domain is made as described below. Fc-antigen binding domain construct 14 (FIG. 14) includes two distinct Fc monomer containing polypeptides (two copies of a long Fc chain and two copies of a short Fc chain) and a light chain polypeptide. The long Fc chain contains an Fc domain monomer with reverse charge mutations selected from Table 4 or Table 5 (e.g., the K409D/D399K mutations), in a tandem series with an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K) at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 17. Design and Purification of Fc-Antigen Binding Domain Construct 15

A construct formed from a singly branched Fc domain where the branch point is at the C-terminal Fc domain is made as described below. Fc-antigen binding domain construct 15 (FIG. 15) includes two distinct Fc monomer containing polypeptides (two copies of a long Fc chain and two copies of a short Fc chain) and a light chain polypeptide. The long Fc chain contains an Fc domain monomer with reverse charge mutations selected from Table 4 or Table 5 (e.g., the K409D/D399K mutations), in a tandem series with an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain at the N-terminus. The short Fc chain contains an Fc domain monomer with an engineered cavity that is made by introducing at least one cavity-forming mutation selected from Table 3 (e.g., the Y349C, T366S. L368A, and Y407V mutations), and, optionally, one or more reverse charge mutation selected from Table 4 (e.g., K370D), and an antigen binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 18. Design and Purification of Fc-Antigen Binding Domain Construct 16

A construct formed from a singly branched Fc domain where the branch point is at the C-terminal Fc domain was made as described below. Fc-antigen binding domain construct 16 (FIG. 16) includes two distinct Fc monomer containing polypeptides (two copies of a long Fc chain and four copies of a short Fc chain) and a light chain polypeptide. The long Fc chain contains an Fc domain monomer with reverse charge mutations selected from Table 4 or Table 5 (e.g., the K409D/D399K mutations), in a tandem series with two Fc domain monomers, each with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D). DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 19. Design and Purification of Fc-Antigen Binding Domain Construct 17

A construct formed from a singly branched Fc domain where the branch point is at the C-terminal Fc domain is made as described below. Fc-antigen binding domain construct 17 (FIG. 17) includes two distinct Fc monomer containing polypeptides (two copies of a long Fc chain and four copies of a short Fc chain) and a light chain polypeptide. The long Fc chain contains an Fc domain monomer with reverse charge mutations selected from Table 4 or Table 5 (e.g., the K409D/D399K mutations), in a tandem series with two Fc domain monomers, each with an engineered protuberance that is made by introducing at least one protuberance-forming mutation selected from Table 3 (e.g., the S354C and T366W mutations) and, optionally, one or more reverse charge mutation selected from Table 4 (e.g., E357K), at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and antigen binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 20. Design and Purification of Fc-Antigen Binding Domain Construct 18

A construct formed from a singly branched Fc domain where the branch point is at the C-terminal Fc domain is made as described below. Fc-antigen binding domain construct 18 (FIG. 18) includes two distinct Fc monomer containing polypeptides (two copies of a long Fc chain and four copies of a short Fc chain) and a light chain polypeptide. The long Fc chain contains an Fc domain monomer with reverse charge mutations selected from Table 4 or Table 5 (e.g., the K409D/D399K mutations), in a tandem series with two Fc domain monomers, each with an engineered protuberance that Is made by introducing at least one protuberance-forming mutation selected from Table 3 (e.g., the S354C and T366W mutations) and, optionally, one or more reverse charge mutation selected from Table 4 (e.g., E357K), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 21. Design and Purification of Fc-Antigen Binding Domain Construct 19

A construct formed from a singly branched Fc domain where the branch point is neither at the N- or C-terminal Fc domain was made as described below. Fc-antigen binding domain construct 19 (FIG. 19) includes two distinct Fc monomer containing polypeptides (two copies of a long Fc chain and four copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and another Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D). DNA sequences were optimized for expression in mammalian cells and cloned into the pcDNA3.4 mammalian expression vector. The DNA plasmid constructs were transfected via liposomes into human embryonic kidney (HEK) 293 cells. The amino acid sequences for the short and long Fc chains were encoded by two separate plasmids. The expressed proteins were purified as in Example 3.

### Example 22. Design and Purification of Fc-Antigen Binding Domain Construct 20

A construct formed from a singly branched Fc domain where the branch point is at the C-terminal Fc domain is made as described below. Fc-antigen binding domain construct 20 (FIG. 20) includes two distinct Fc monomer containing polypeptides (two copies of a long Fc chain and four copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and another Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 23. Design and Purification of Fc-Antigen Binding Domain Construct 21

A construct formed from a singly branched Fc domain where the branch point is at the C-terminal Fc domain is made as described below. Fc-antigen binding domain construct 21 (FIG. 21) includes two distinct Fc monomer containing polypeptides (two copies of a long Fc chain and four copies of a short Fc chain) and a light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), another Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 24. Design and Purification of Fc-Antigen Binding Domain Construct 22

A bispecific construct formed using long and short Fc chains with different antigen binding domains is made as described below. Fc-antigen binding domain construct 22 (FIG. 22) includes two distinct Fc monomer containing polypeptides (a long Fc chain and two copies of a short Fc chain) and either two distinct light chain polypeptides or a common light chain polypeptide. The long Fc chain contains two Fc domain monomers, each with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series and an antigen binding domain of a first specificity at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 25. Design and Purification of Fc-Antigen Binding Domain Construct 23

A bispecific construct formed using long and short Fc chains with different antigen binding domains is made as described below. Fc-antigen binding domain construct 23 (FIG. 23) includes two distinct Fc monomer containing polypeptides (a long Fc chain and three copies of a short Fc chain) and either two distinct light chain polypeptides or a common light chain polypeptide. The long Fc chain contains three Fc domain monomers, each with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series and an antigen binding domain of a first specificity at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 26. Design and Purification of Fc-Antigen Binding Domain Construct 24

A bispecific construct formed using long and short Fc chains with different antigen binding domains is made as described below. Fc-antigen binding domain construct 24 (FIG. 24) includes two distinct Fc monomer containing polypeptides (two copies of a long Fc chain and two copies of a short Fc chain) and either two distinct light chain polypeptides or a common light chain polypeptide. The long Fc chain contains an Fc domain monomer with reverse charge mutations selected from Table 4 or Table 5 (e.g., the K409D/D399K mutations) in a tandem series with an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain of a first specificity at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 27. Design and Purification of Fc-Antigen Binding Domain Construct 25

A bispecific construct formed using long and short Fc chains with different antigen binding domains is made as described below. Fc-antigen binding domain construct 25 (FIG. 25) includes two distinct Fc monomer containing polypeptides (two copies of a long Fc chain and two copies of a short Fc chain) and either two distinct light chain polypeptides or a common light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and an antigen binding domain of a first specificity at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 28. Design and Purification of Fc-Antigen Binding Domain Construct 26

A bispecific construct formed using long and short Fc chains with different antigen binding domains is made as described below. Fc-antigen binding domain construct 26 (FIG. 26) includes two distinct Fc monomer containing polypeptides (two copies of a long Fc chain and four copies of a short Fc chain) and either two distinct light chain polypeptides or a common light chain polypeptide. The long Fc chain contains an Fc domain monomer with reverse charge mutations selected from Table 4 or Table 5 (e.g., the K409D/D399K mutations), in tandem series with two Fc domain monomers, each with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain of a first specificity at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 29. Design and Purification of Fc-Antigen Binding Domain Construct 27

A bispecific construct formed using long and short Fc chains with different antigen binding domains is made as described below. Fc-antigen binding domain construct 27 (FIG. 27) includes two distinct Fc monomer containing polypeptides (two copies of a long Fc chain and four copies of a short Fc chain) and either two distinct light chain polypeptides or a common light chain polypeptide. The long Fc chain contains an Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), another protuberance-containing Fc domain monomer with an engineered protuberance that is made by introducing at least one protuberance-forming mutation selected from Table 3 (e.g., the S354C and T366W mutations) and, optionally, one or more reverse charge mutation selected from Table 4 (e.g., E357K), and an antigen binding domain of a first specificity at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and an antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 30. Design and Purification of Fc-Antigen Binding Domain Construct 28

A bispecific construct formed using long and short Fc chains with different antigen binding domains is made as described below. Fc-antigen binding domain construct 28 (FIG. 28) includes two distinct Fc monomer containing polypeptides (two copies of a long Fc chain and four copies of a short Fc chain) and either two distinct light chain polypeptides or a common light chain polypeptide. The long Fc chain contains two Fc domain monomers, each with an engineered protuberance that is made by introducing at least one protuberance-forming mutation selected from Table 3 (e.g., the S354C and T366W mutations) and, optionally, one or more reverse charge mutation selected from Table 4 (e.g., E357K), in a tandem series with an Fc domain monomer with reverse charge mutations selected from Table 4 or Table 5 (e.g., the K409D/D399K mutations), and an antigen binding domain of a first specificity at the N-terminus. The short Fc chain contains an Fc domain monomer with an engineered cavity that is made by introducing at least one cavity-forming mutation selected from Table 3 (e.g., the Y349C, T366S, L368A, and Y407V mutations), and, optionally, one or more reverse charge mutation selected from Table 4 (e.g., K370D), and antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by two separate plasmids. The expressed proteins are purified as in Example 3.

### Example 31. Design and Purification of Fc-Antigen Binding Domain Construct 29

A bispecific construct formed using long and short Fc chains with different antigen binding domains and two different sets of heterodimerization mutations is made as described below. Fc-antigen binding domain construct 29 (FIG. 29) includes three distinct Fc monomer containing polypeptides (a long Fc chain, and two distinct short Fc chains) and either two distinct light chain polypeptides or a common light chain polypeptide. The long Fc chain contains two Fc domain monomers, each with a different set of protuberance-forming mutations selected from Table 3 (heterodimerization mutations), and, optionally, one or more reverse charge mutation selected from Table 4, in a tandem series with an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set off mutations in the first short Fc chain, and, optionally, one or more reverse charge mutation selected from Table 4. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 32. Design and Purification of Fc-Antigen Binding Domain Construct 30

A bispecific construct formed using long and short Fc chains with different antigen binding domains and two different sets of heterodimerization mutations is made as described below. Fc-antigen binding domain construct 30 (FIG. 30) includes three distinct Fc monomer containing polypeptides (a long Fc chain, and two distinct short Fc chains) and either two distinct light chain polypeptides or a common light chain polypeptide. The long Fc chain contains two Fc domain monomers, each with a different set of protuberance-forming mutations selected from Table 3 (heterodimerization mutations), and, optionally, one or more reverse charge mutation selected from Table 4, in a tandem series with an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set off mutations in the first short Fc chain, and, optionally, one or more reverse charge mutation selected from Table 4, and an antigen binding domain of a first specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 33. Design and Purification of Fc-Antigen Binding Domain Construct 31

A trispecific construct formed using long and short Fc chains with different antigen binding domains and two different sets of heterodimerization mutations is made as described below. Fc-antigen binding domain construct 31 (FIG. 31) includes three distinct Fc monomer containing polypeptides (a long Fc chain, and two distinct short Fc chains) and either three or two distinct light chain polypeptides or a common light chain polypeptide. The long Fc chain contains two Fc domain monomers, each with a different set of protuberance-forming mutations selected from Table 3 (heterodimerization mutations), and, optionally, one or more reverse charge mutation selected from Table 4, in a tandem series with an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set off mutations in the first short Fc chain, and, optionally, one or more reverse charge mutation selected from Table 4, and an antigen binding domain of a third specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences are for the short and long Fc chains encoded by three separate plasmids. The expressed proteins are purified as in Example 3.

### Example 34. Design and Purification of Fc-Antigen Binding Domain Construct 32

A bispecific construct formed using long and short Fc chains with different antigen binding domains and two different sets of heterodimerization mutations is made as described below. Fc-antigen binding domain construct 32 (FIG. 32) includes three distinct Fc monomer containing polypeptides (a long Fc chain, two copies of one short Fc chain, and one copy of a second short Fc chain) and either two distinct light chain polypeptides or a common light chain polypeptide. The long Fc chain contains three Fc domain monomers, each with a set of protuberance-forming mutations selected from Table 3 (heterodimerization mutations), and, optionally, one or more reverse charge mutation selected from Table 4, (the third Fc domain monomer with a different set of heterodimerization mutations than the first two) in a tandem series with an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set off mutations in the first short Fc chain, and, optionally, one or more reverse charge mutation selected from Table 4. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 35. Design and Purification of Fc-Antigen Binding Domain Construct 33

A bispecific construct formed using long and short Fc chains with different antigen binding domains and two different sets of heterodimerization mutations is made as described below. Fc-antigen binding domain construct 33 (FIG. 33) includes three distinct Fc monomer containing polypeptides (a long Fc chain, and two copies of a first short Fc chain, and one copy of a second short Fc chain) and either two distinct light chain polypeptides or a common light chain polypeptide. The long Fc chain contains three Fc domain monomers, each with a set of protuberance-forming mutations selected from Table 3 (heterodimerization mutations), and, optionally, one or more reverse charge mutation selected from Table 4, (the third Fc domain monomer with a different set of heterodimerization mutations than the first two) in a tandem series with an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set off mutations in the first short Fc chain, and, optionally, one or more reverse charge mutation selected from Table 4, and an antigen binding domain of a first specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 36. Design and Purification of Fc-Antigen Binding Domain Construct 34

A trispecific construct formed using long and short Fc chains with different antigen binding domains and two different sets of heterodimerization mutations is made as described below. Fc-antigen binding domain construct 34 (FIG. 34) includes three distinct Fc monomer containing polypeptides (a long Fc chain, two copies of a first short Fc chain, and one copy of a second short Fc chain) and either three or two distinct light chain polypeptides or a common light chain polypeptide. The long Fc chain contains three Fc domain monomers, each with a set of protuberance-forming mutations selected from Table 3 (heterodimerization mutations), and, optionally, one or more reverse charge mutation selected from Table 4, (the third Fc domain monomer with a different set of heterodimerization mutations than the first two) in a tandem series with an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set off mutations in the first short Fc chain, and, optionally, one or more reverse charge mutation selected from Table 4, and an antigen binding domain of a third specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 37. Design and Purification of Fc-Antigen Binding Domain Construct 35

A trispecific construct formed using long and short Fc chains with different antigen binding domains and two different sets of heterodimerization mutations is made as described below. Fc-antigen binding domain construct 35 (FIG. 35) includes four distinct Fc monomer containing polypeptides (two distinct long Fc chains, and two distinct short Fc chains) and either three or two distinct light chain polypeptides or a common light chain polypeptide. The first long Fc chain contains an Fc domain monomer with reverse charge mutations selected from Table 4 or Table 5 (e.g., the K409D/D399K mutations), in a tandem series with an Fc domain monomer with a first set of protuberance-forming mutations selected from Table 3 (heterodimerization mutations), and, optionally, one or more reverse charge mutation selected from Table 4, and an antigen binding domain of a first specificity at the N-terminus. The second long Fc chain contains an Fc domain monomer with reverse charge mutations selected from Table 4 or Table 5 (e.g., the K409D/D399K mutations), in a tandem series with an Fc domain monomer with a second set of protuberance-forming mutations selected from Table 3 (heterodimerization mutations) different from the first set of mutations in the first long Fc chain, and, optionally, one or more reverse charge mutation selected from Table 4, and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and antigen binding domain of a second specificity at the N-terminus. The second short Fc chain contains an Fc domain monomer with a second set of cavity-forming mutations selected from Table 3 (heterodimerization mutations) different from the first set of mutations in the first short Fc chain, and, optionally, one or more reverse charge mutation selected from Table 4, and an antigen binding domain of a third specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by four separate plasmids. The expressed proteins are purified as in Example 3.

### Example 38. Design and Purification of Fc-Antigen Binding Domain Construct 36

A bispecific construct formed using long and short Fc chains with different antigen binding domains and two different sets of heterodimerization mutations is made as described below. Fc-antigen binding domain construct 36 (FIG. 36) includes three distinct Fc monomer containing polypeptides (two copies of a long Fc chain, and two copies each of two distinct short Fc chains) and either two distinct light chain polypeptides or a common light chain polypeptide. The long Fc chain contains an Fc domain monomer with a first set of protuberance-forming mutations selected from Table 3 (heterodimerization mutations), and, optionally, one or more reverse charge mutation selected from Table 4, in a tandem series with an Fc domain monomer with reverse charge mutations selected from Table 4 or Table 5 (e.g., the K409D/D399K mutations), a second Fc domain monomer with a second set of protuberance-forming mutations selected from Table 3 (heterodimerization mutations), and, optionally, one or more reverse charge mutation selected from Table 4, and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4. The second short Fc chain contains an Fc domain monomer with a second set of cavity-forming mutations selected from Table 3 (heterodimerization mutations) different from the first set of mutations in the first short Fc chain, and, optionally, one or more reverse charge mutation selected from Table 4, and an antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 39. Design and Purification of Fc-Antigen Binding Domain Construct 37

A trispecific construct formed using long and short Fc chains with different antigen binding domains and two different sets of heterodimerization mutations is made as described below. Fc-antigen binding domain construct 37 (FIG. 37) includes three distinct Fc monomer containing polypeptides (two copies of a long Fc chain, and two copies each of two distinct short Fc chains) and either three or two distinct light chain polypeptides or a common light chain polypeptide. The long Fc chain contains an Fc domain monomer with a first set of protuberance-forming mutations selected from Table 3 (heterodimerization mutations), and, optionally, one or more reverse charge mutation selected from Table 4, in a tandem series with an Fc domain monomer with reverse charge mutations selected from Table 4 or Table 5 (e.g., the K409D/D399K mutations), a second Fc domain monomer with a second set of protuberance-forming mutations selected from Table 3 (heterodimerization mutations), and, optionally, one or more reverse charge mutation selected from Table 4, and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set of mutations in the first short Fc chain, and, optionally, one or more reverse charge mutation selected from Table 4, and an antigen binding domain of a third specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 40. Design and Purification of Fc-Antigen Binding Domain Construct 38

A trispecific construct formed using long and short Fc chains with different antigen binding domains and two different sets of heterodimerization mutations is made as described below. Fc-antigen binding domain construct 38 (FIG. 38) includes four distinct Fc monomer containing polypeptides (two distinct long Fc chains, and two distinct short Fc chains) and either three or two distinct light chain polypeptides or a common light chain polypeptide. The first long Fc chain contains an Fc domain monomer with a first set of protuberance-forming mutations selected from Table 3 (heterodimerization mutations), and, optionally, one or more reverse charge mutation selected from Table 4, in a tandem series with a Fc domain monomer with reverse charge mutations selected from Table 4 or Table 5 (e.g., the K409D/D399K mutations), and an antigen binding domain of a first specificity at the N-terminus. The second long Fc chain contains an Fc domain monomer with a second set of protuberance-forming mutations selected from Table 3 (heterodimerization mutations) different from the first set of mutations in the first long Fc chain, and, optionally, one or more reverse charge mutation selected from Table 4, in a tandem series with an Fc domain monomer with reverse charge mutations selected from Table 4 or Table 5 (e.g., the K409D/D399K mutations), and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains a Fc domain monomer with a second set of cavity-forming mutations selected from Table 3 (heterodimerization mutations) different from the first set of mutations in the first short Fc chain, and, optionally, one or more reverse charge mutation selected from Table 4, and an antigen binding domain of a third specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by four separate plasmids. The expressed proteins are purified as in Example 3.

### Example 41. Design and Purification of Fc-Antigen Binding Domain Construct 39

A bispecific construct formed using long and short Fc chains with different antigen binding domains and two different sets of heterodimerization mutations is made as described below. Fc-antigen binding domain construct 39 (FIG. 39) includes three distinct Fc monomer containing polypeptides (two copies of a long Fc chain, and two copies each of two distinct short Fc chains) and either two distinct light chain polypeptides or a common light chain polypeptide. The long Fc chain contains an Fc domain monomer with reverse charge mutations selected from Table 4 or Table 5 (e.g., the K409D/D399K mutations), in a tandem series with an Fc domain monomer with a first set of protuberance-forming mutations selected from Table 3 (heterodimerization mutations), and, optionally, one or more reverse charge mutation selected from Table 4, a second Fc domain monomer with a second set of protuberance-forming mutations selected from Table 3 (heterodimerization mutations), and, optionally, one or more reverse charge mutation selected from Table 4, and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4. The second short Fc chain contains an Fc domain monomer with a second set of cavity-forming mutations selected from Table 3 (heterodimerization mutations) different from the first set of mutations in the first short Fc chain, and, optionally, one or more reverse charge mutation selected from Table 4, and an antigen binding domain of a second specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 42. Design and Purification of Fc-Antigen Binding Domain Construct 40

A trispecific construct formed using long and short Fc chains with different antigen binding domains and two different sets of heterodimerization mutations is made as described below. Fc-antigen binding domain construct 40 (FIG. 40) includes three distinct Fc monomer containing polypeptides (two copies of a long Fc chain, and two copies each of two distinct short Fc chains) and either three or two distinct light chain polypeptides or a common light chain polypeptide. The long Fc chain contains an Fc domain monomer with reverse charge mutations selected from Table 4 or Table 5 (e.g., the K409D/D399K mutations), in a tandem series with an Fc domain monomer with a first set of protuberance-forming mutations selected from Table 3 (heterodimerization mutations), and, optionally, one or more reverse charge mutation selected from Table 4, a second Fc domain monomer with a second set of protuberance-forming mutations selected from Table 3 (heterodimerization mutations), and, optionally, one or more reverse charge mutation selected from Table 4, and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of second specificity at the N-terminus. The second short Fc chain contains an Fc domain monomer with a second set of cavity-forming mutations selected from Table 3 (heterodimerization mutations) different from the first set of mutations in the first short Fc chain, and, optionally, one or more reverse charge mutation selected from Table 4, and an antigen binding domain of a third specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 43. Design and Purification of Fc-Antigen Binding Domain Construct 41

A bispecific construct formed using long and short Fc chains with different antigen binding domains and two different sets of heterodimerization mutations is made as described below. Fc-antigen binding domain construct 41 (FIG. 41) includes three distinct Fc monomer containing polypeptides (two copies of a long Fc chain, and two copies each of two distinct short Fc chains) and either two distinct light chain polypeptides or a common light chain polypeptide. The long Fc chain contains two Fc domain monomers, each with a different set of protuberance-forming mutations selected from Table 3 (heterodimerization mutations), and, optionally, one or more reverse charge mutation selected from Table 4, in a tandem series with an Fc domain monomer with reverse charge mutations selected from Table 4 or Table 5 (e.g., the K409D/D399K mutations), and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains a cavity-containing Fc domain monomer with a second set of cavity-forming mutations selected from Table 3 (heterodimerization mutations) different from the first set of mutations in the first short Fc chain, and, optionally, one or more reverse charge mutation selected from Table 4. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 44. Design and Purification of Fc-Antigen Binding Domain Construct 42

A trispecific construct formed using long and short Fc chains with different antigen binding domains and two different sets of heterodimerization mutations is made as described below. Fc-antigen binding domain construct 42 (FIG. 42) includes three distinct Fc monomer containing polypeptides (two copies of a long Fc chain, and two copies each of two distinct short Fc chains) and either three or two distinct light chain polypeptides or a common light chain polypeptide. The long Fc chain contains two Fc domain monomers, each with a different set of protuberance-forming mutations selected from Table 3 (heterodimerization mutations), and, optionally, one or more reverse charge mutation selected from Table 4, in a tandem series with an Fc domain monomer with reverse charge mutations selected from Table 4 or Table 5 (e.g., the K409D/D399K mutations), and an antigen binding domain of a first specificity at the N-terminus. The first short Fc chain contains an Fc domain monomer with a first set of cavity-forming mutations selected from Table 3 (heterodimerization mutations), and, optionally, one or more reverse charge mutation selected from Table 4, and an antigen binding domain of a second specificity at the N-terminus. The second short Fc chain contains an Fc domain monomer with a second set of cavity-forming mutations selected from Table 3 (heterodimerization mutations) different from the first set of mutations in the first short Fc chain, and, optionally, one or more reverse charge mutation selected from Table 4, and an antigen binding domain of a third specificity at the N-terminus. DNA sequences are optimized for expression in mammalian cells and cloned into the pcDNA3.4 mammalian expression vector. The DNA plasmid constructs are transfected via liposomes into human embryonic kidney (HEK) 293 cells. The amino acid sequences for the short and long Fc chains are encoded by three separate plasmids. The expressed proteins are purified as in Example 3.

### Example 45. Expression of Fc-Antigen Binding Domain Constructs

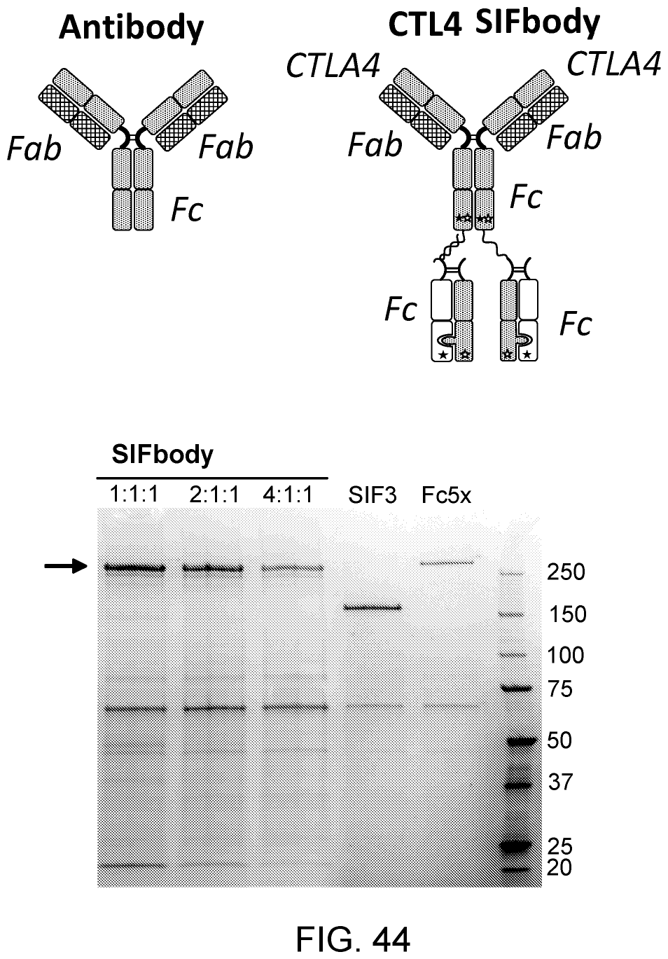

A Fc-antigen binding domain construct was designed with three Fc domains and a CTLA4 Fab domain at the N-terminus. DNA sequences were optimized for expression in mammalian cells and cloned into the pcDNA3.4 mammalian expression vector. The DNA plasmid constructs were transfected via liposomes into human embryonic kidney (HEK) 293 cells. The expressed proteins were purified as in example 1. The proteins were run on a non-reducing SDS-PAGE gel (FIG. 44). The S31 and S3Y SIF-body proteins are hexamers including three different proteins, each of which was expressed from a different plasmid. The numbers above the lanes represent ratios of plasmid DNAs that were transfected into cells for expression. A ratio of 2:1:1 indicates that 0.5 mg SIF-body Long Chain plasmid, 0.25 mg of SIF Short Chain plasmid and 0.25 mg of Fab Light chain were present in the mixture that was used to transfect I liter of cells. In a 1:1:1 ratio 0.33 mg of each plasmid was used. In the 4:1:1 ratio 0.67 mg long chain, 0.167 mg short chain and 0.167 mg light chain were used.

Samples were denatured in Laemmli sample buffer (4% SDS. Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). The constructs were shown express similar to parent antibodies and were purified with techniques similar to SIF3 with minimal impurities.

### Example 46. SPR Assay to Measure Fc Binding to Targets and Fcγ Receptors

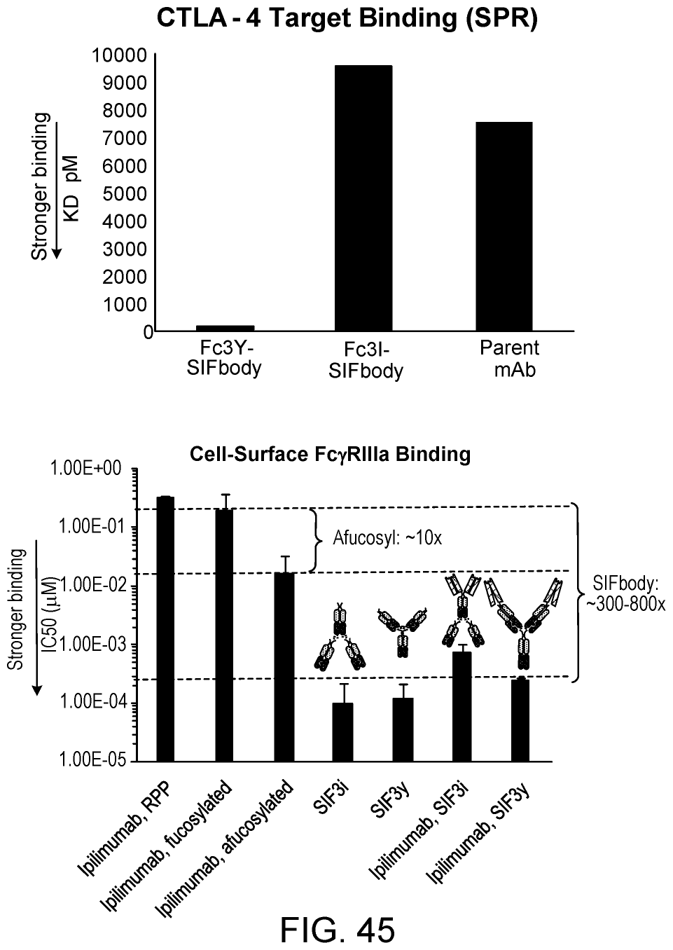

Two constructs containing anti-CTLA-4 antigen binding domains were created based on the design for constructs 7 and 13. The parent mAb and the antigen binding domains of both Fc-antigen binding domain constructs contain the CDRs from Ipilimumab. An SPR assay was performed to measure target binding to CTLA-4 (FIG. 45). In the left panel, a dissociation constant was measured for the parent mAB, the Fc3Y-Fc-antigen binding domain constructs (modified version of construct 13 with Ipilimumab antigen-binding domain), and the Fc3I-Fc-antigen binding domain constructs (modified version of construct 7 with Ipilimumab antigen-binding domain). The Fc3Y-Fc showed enhanced binding of the CTLA-4 target relative to the parent mAb while the Fc3I-Fc showed similar binding of the CTLA-4 target relative to the parent mAb. In the right panel, the results are shown from another SPR assay used to measure FcγRIIa binding of different Fc-antigen binding domain constructs. Three versions of Ipilimumab were tested and afucosylation was found to enhance binding ˜10-fold. Four Fc-antigen binding domain constructs were tested, SIF31, SIF3Y, and both constructs with the corresponding antigen binding domains containing the Ipilimumab CDRs. All four constructs showed ˜300-800-fold enhanced FcγRIIa binding relative to the parent mAb.

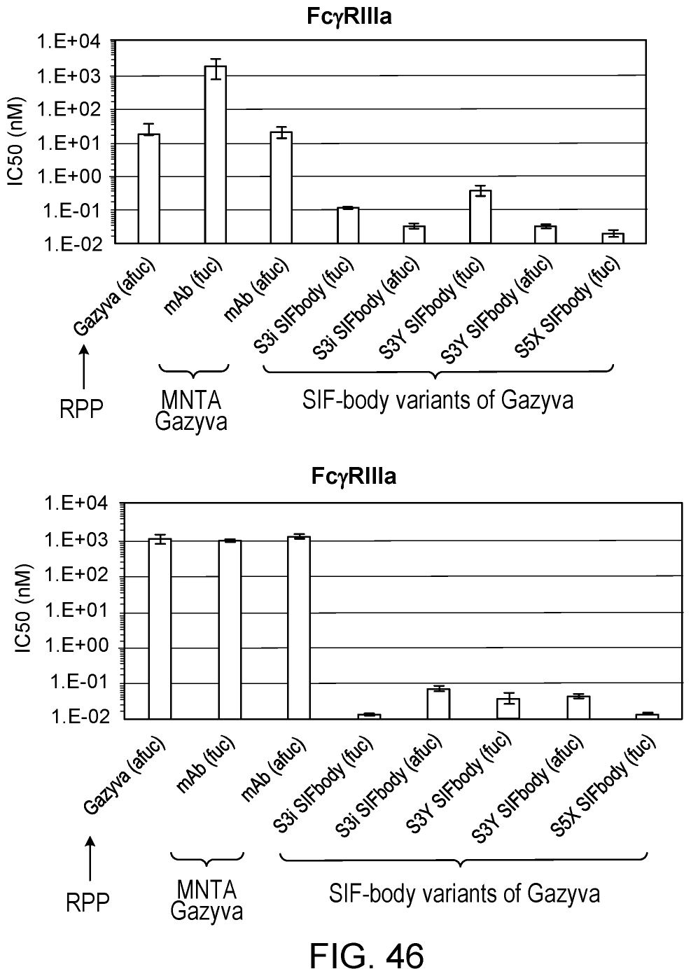

Two constructs containing anti-CD20 antigen binding domains were created based on the design for Fc-antigen binding domain constructs 7 and 13. The parent mAb and the antigen binding domains of both Fc-antigen binding domain constructs contain the CDRs from Gazyva (obinutuzumab). An SPR assay was performed to measure binding to both FcγIIIa and FcγIIa (FIG. 46). In the left panel, afucosylation enhanced binding to FcγIIIa ˜10-fold while all Fc-antigen binding domain constructs showed >100-fold enhanced binding to FcγIIIa relative to the mAb. In the right panel, afucosylation had little effect on binding to FcγIIa, but all Fc-antigen binding domain constructs showed >100-fold enhanced binding to FcγIIa relative to the mAb.

### Example 47. CDC, ADCP, and ADCC Activation by Fc-Antigen Binding Domain Constructs

Three assays were used to test the activation of CDC, ADCP, and ADCC pathways by parent mAbs and various Fc-antigen binding domain constructs. Four constructs were created containing the CDRs from Gazyva (obinutuzumab), an anti-CD20 monoclonal antibody. Both fucosylated and afucosylated mAbs were made as well as S3Y (structure of Construct 13, FIG. 13, as described in Example 2) and SAI (structure of Construct 7, FIG. 7, as described in Example 1) Fc-antigen binding domain constructs. A CDC assay was performed as follows:

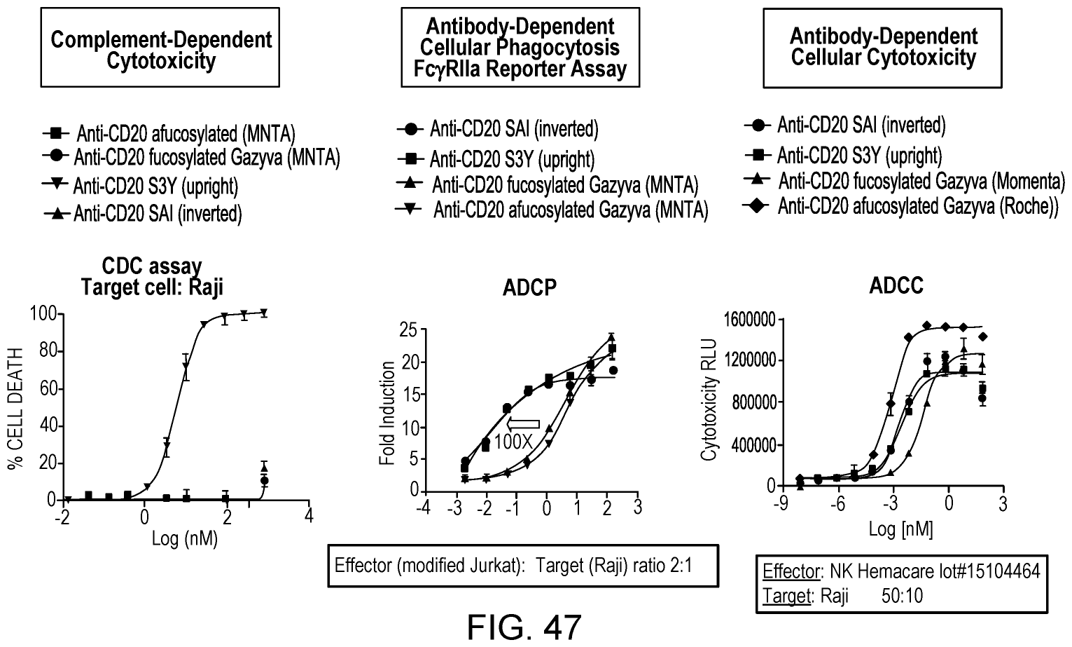

1. The target cells used in the anti-CD20 CDC assay are the Raji lymphoblastoid human B cell line (ATCC CCL-86). Raji cells were removed from suspension culture by centrifugation and resuspended in X-VIVO 15 media at 6×10⁵ cells/ml.
2. The Raji cells were transferred to a 96 well flat-bottom assay plate in a volume of 100 μl per well (6×10⁴ cells/well).
3. Each of the anti-CD20 monoclonal antibodies (mAbs) and SIF Bodies were diluted to 3.33 μM in X-VIVO 15 media. Serial 1:3 dilutions were then performed with each of the anti-CD20 mAbs and SIF Bodies in 1.5 ml polypropylene tubes resulting in an 11 point dilution series.
4. Each dilution of the anti-CD20 mAbs and SIF Bodies was transferred at 50 μl/well to the appropriate wells in the assay plate.
5. Immediately following the transfer of the anti-CD20 mAbs and SIF Bodies, 50 μl of normal human serum complement were transferred to each well of the assay plate.
6. The assay plate was incubated at 37° C. and 5% CO₂ for 2 h.
7. Following the 2 h incubation, 20 μl of WST-1 proliferation reagent was added to each well of the assay plate.
8. The plate was returned to the 37° C., 5% CO₂ incubator for 14 h.
9. Following the 14 h incubation, the plate was shaken for 1 min on a plate shaker and the absorbance of the wells was immediately determined at 450 nm with 600 nm correction using a spectrophotometer.
In a CDC assay in which the target cells were Raji (FIG. 47, left panel), the S3Y (construct 13 (CD20)) construct was able to mediate cytotoxicity, while the other constructs were not.

An ADCP assay was performed as follows:

The FcγRIIa-H ADCP Reporter Bioassay, Complete Kit (Promega Cat. # G9901), is a bioluminescent cell-based assay that can be used to measure the potency and stability of antibodies and other biologics with Fc domains that specifically bind and activate FcγRIIa. The assay consisted of a genetically engineered Jurkat T cell line that expresses the high-affinity human FcgRIIa-H variant that contains a Histidine (H) at amino acid 131 and a luciferase reporter driven by an NFAT-response element (NFAT-RE). When co-cultured with a target cell and relevant antibody, the FcγRIIa-H effector cells bound the Fc domain of the antibody, resulting in FcγRIIa signaling and NFAT-RE-mediated luciferase activity. The bioluminescent signal was detected and quantified using Bio-Glo™ Luciferase Assay System and a standard luminometer, increasing concentrations of anti-CD20 Abs and SIFbodies (construct 7 (CD20) or construct 13 (CD20)) were incubated with Raji (CD20+) target cells and Fc, increasing concentrations of anti-CD20 Abs and SIFbodies were incubated with Rap (CD20+) target cells and FcγRIIa-H effector cells (2:1 E:T ratio; approx. 35,000 effector 15,000 target cells) at the indicated concentrations in FIG. 47 middle panel. Incubation proceeded for 6 h at 37° C. Bio-Glo™. The Reagent was added, and luminescence was measured in a PHERAstar FS instrument. Data were fitted to a 4PL curve using GraphPad Prism software RIIa-H effector cells (2:1 E:T ratio; approximately 35,000 effector:15,000 target cells) at the indicated concentrations in FIG. 47 middle panel. Incubation proceeded for 6 h at 37° C. Bio-Glo™ Reagent was added, and luminescence was measured in a PHERAstar FS instrument. Data were fitted to a 4PL curve using GraphPad Prism software (FIG. 47, middle panel). Both the SAI (construct 7 (CD20)) and S3Y (construct 13 (CD20)) constructs showed enhanced potency >100-fold relative to the mAbs.

An ADCC assay was performed as follows:

Human primary NK effector cells (Hemacare) were thawed and rested overnight at 37° C. in lymphocyte growth medium-3 (Lonza) at 5×10⁵/mL. The next day, the human lymphoblastoid cell line Raji target cells (ATCC CCL-86) were harvested, resuspended in assay media (phenol red free RPMI, 10% FBSA, GlutaMAX™), and plated in the presence of various concentrations of each probe of interest for 30 minutes at 37° C. The rested NK cells were then harvested, resuspended in assay media, and added to the plates containing the anti-CD20 coated Raji cells. The plates were incubated at 37° C. for 6 hours with the final ratio of effector-to-target cells at 5:1 (5×10 NK cells: 1×10⁴ Raji).

The CytoTox-Glo™ Cytotoxicity Assay kit (Promega) was used to determined ADCC activity. The CytoTox-Glo™ assay uses a luminogenic peptide substrate to measure dead cell protease activity which is released by cells that have lost membrane integrity e.g. lysed Raji cells. After the 6 hour incubation period, the prepared reagent (substrate) was added to each well of the plate and placed on an orbital plate shaker for 15 minutes at room temperature. Luminescence was measured using the PHERAstar F5 plate reader (BMG Labtech). The data was analyzed after the readings from the control conditions (NK cells+Raji only) were subtracted from the test conditions to eliminate background. (FIG. 47, right panel). Both the SAI (construct 7 (CD20)) and S3Y (construct 13 (CD20)) constructs showed enhanced cytotoxicity relative to the fucosylated mAb and similar cytotoxicity relative to the afucosylated mAb.

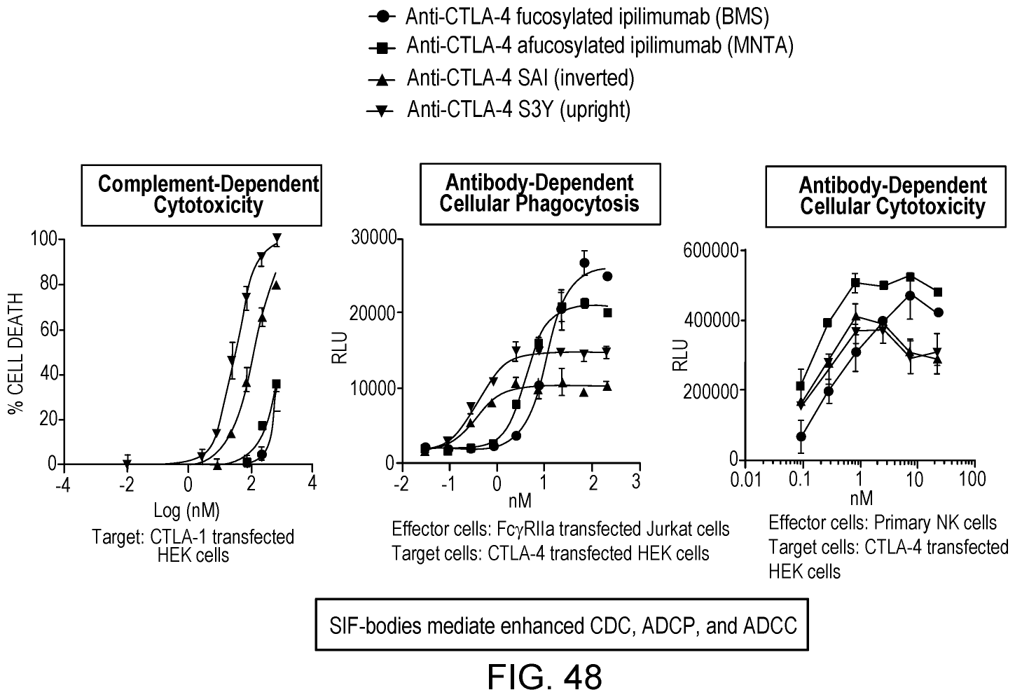

A similar set of assays was performed using constructs based on the Ipilimumab antibody. Four constructs were created containing the CDRs from Ipilimumab, an anti-CTLA-4 monoclonal antibody. Both fucosylated and afucosylated mAbs were made as well as S3Y (construct 13 (CTLA-4)) and SAI (construct 7 (CTLA-4)) Fc-antigen binding domain constructs. In a CDC assay in which the target cells were CTLA-4 transfected HEK cells (FIG. 48, left panel), the SAI (construct 7 (CTLA-4)) and S3Y (construct 13 (CTLA-4)) constructs showed enhanced cytotoxicity relative to the two mAb constructs. ADCP activation was tested with an assay targeting CTLA-4 transfected HEK cells (FIG. 48, middle panel). Both the SAI (construct 7 (CTLA-4)) and S3Y (construct 13 (CTLA-4)) constructs showed enhanced phagocytosis relative to the two mAbs. ADCC was assayed using CTLA-4 transfected HEK target cells (FIG. 48, right panel). Both the SAI (construct 7 (CTLA-4)) and S3Y (construct 13 (CTLA-4)) constructs showed enhanced cytotoxicity relative to the fucosylated mAb and similar cytotoxicity relative to the afucosylated mAb.

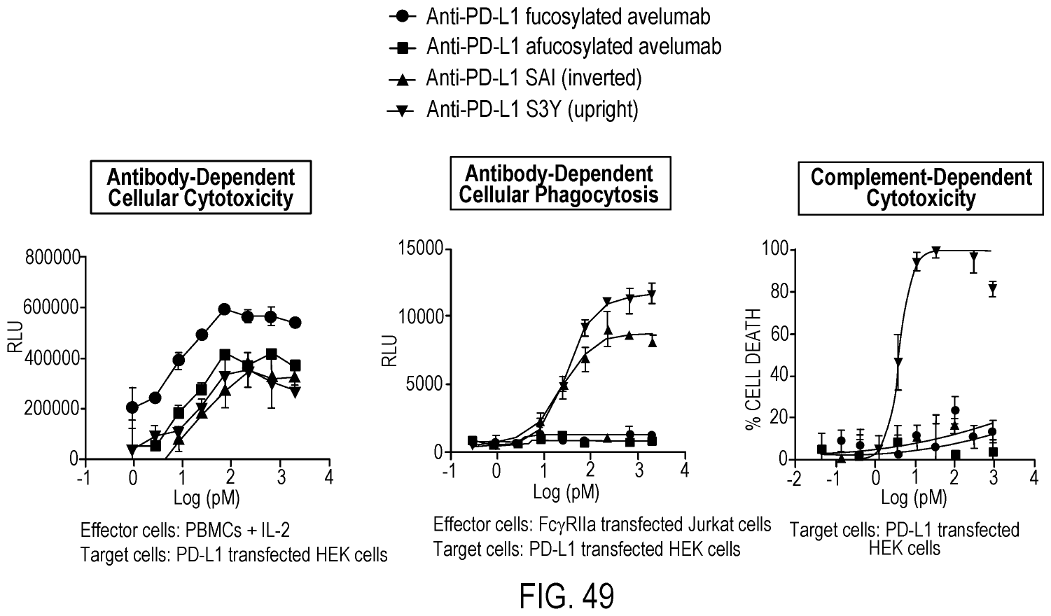

A similar set of assays was performed using constructs based on the antibody. Four constructs were created containing the CDRs from an anti-PD-L1 monoclonal antibody. Both fucosylated and afucosylated mAbs were made as well as S3Y (construct 13 (PD-L1)) and SAI (construct 7 (PD-L1)) Fc-antigen binding domain constructs. ADCC was assayed using PD-L1 transfected HEK target cells (FIG. 49, left panel). Both the SAI (construct 7 (PD-L1)) and S3Y (construct 13 (PD-L1)) constructs showed similar cytotoxicity as both the fucosylated and afucosylated mAbs. ADCP activation was tested with an assay targeting PD-L1 transfected HEK cells (FIG. 49, middle panel). Both the SAI (construct 7 (PD-L1)) and S3Y (construct 13 (PD-L1)) constructs activated phagocytosis whereas neither mAbs did. In a CDC assay targeting PD-L1 transfected HEK cells (FIG. 49, right panel), the S3Y (construct 13 (PD-L1)) construct was able to mediate cytotoxicity while the other constructs did not.

### Example 48. Experimental Assays Used to Characterize Fc-Antigen Binding Domain Constructs

### Peptide and Glycopeptide Liquid Chromatography-MS/MS

The proteins were diluted to 1 μg/μL in 6M guanidine (Sigma). Dithiothreitol (DTT) was added to a concentration of 10 mM, to reduce the disulfide bonds under denaturing conditions at 65° C. for 30 min. After cooling on ice, the samples were incubated with 30 mM iodoacetamide (IAM) for 1 h in the dark to alkylate (carbamidomethylate) the free thiols. The protein was then dialyzed across a 10-kDa membrane into 25 mM ammonium bicarbonate buffer (pH 7.8) to remove IAM, DTT and guanidine. The protein was digested with trypsin in a Barocycler (NEP 2320; Pressure Biosciences, Inc.). The pressure was cycled between 20,000 psi and ambient pressure at 37° C. for a total of 30 cycles in 1 h. LC-MS/MS analysis of the peptides was performed on an Ultimate 3000 (Dionex) Chromatography System and an Q-Exactive (Thermo Fisher Scientific) Mass Spectrometer. Peptides were separated on a BEH PepMap (Waters) Column using 0.1% FA in water and 0.1% FA in acetonitrile as the mobile phases. The singly xylosylated linker peptide was targeted based on the doubly charged ion (z=2) m/z 842.5 with a quadrupole isolation width of ±1.5 Da.

### Intact Mass Spectrometry

The protein was diluted to a concentration of 2 μg/μL in the running buffer consisting of 78.98% water, 20% acetonitrile, 1% formic acid (FA), and 0.02% trifluoroacetic acid. Size exclusion chromatography separation was performed on two Zenix-C SEC-300 (Sepax Technologies, Newark, Del.) 2.1×350 mm in tandem for a total length column length of 700 mm. The proteins were eluted from the SEC column using the running buffer described above at a flow rate of 80 μL/min. Mass spectra were acquired on an QSTAR Elite (Applied Biosystems) Q-ToF mass spectrometer operated in positive mode. The neutral masses under the individual size fractions were deconvoluted using Bayesian peak deconvolution by summing the spectra across the entire width of the chromatographic peak.

### Capillary Electrophoresis-Sodium Dodecyl Sulfate (CE-SDS) Assay

Samples were diluted to 1 mg/mL and mixed with the HT Protein Express denaturing buffer (PerkinElmer). The mixture was incubated at 40° C. for 20 min. Samples were diluted with 70 μL of water and transferred to a 96-well plate. Samples were analyzed by a Caliper GXII instrument (PerkinElmer) equipped with the HT Protein Express LabChip (PerkinElmer). Fluorescence intensity was used to calculate the relative abundance of each size variant.

### Non-Reducing SDS-PAGE

Samples were denatured in Laemmli sample buffer (4% SDS. Bo-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Complement Dependent Cytotoxicity (CDC)

CDC was evaluated by a colorimetric assay in which Raji cells (ATCC) were coated with serially diluted Rituximab, Fc construct 4, or IVIg. Human serum complement (Quidel) was added to all wells at 25% v/v and incubated for 2 h at 37° C. Cells were incubated for 12 h at 37° C. after addition of WST-1 cell proliferation reagent (Roche Applied Science). Plates were placed on a shaker for 2 min and absorbance at 450 nm was measured.

### Example 49. Optimization of Heterodimerization by Knob-into-Hole Technology

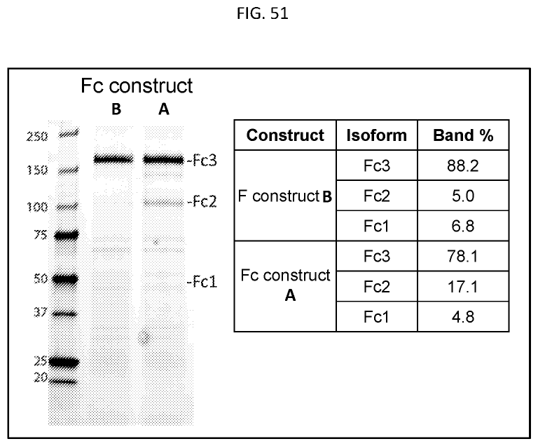

Plasmids expressing the long and short polypeptides of an Fc construct with three Fc domains and knob-into-hole mutations in the two “branch” subunits (Fc construct A), or the long and short polypeptides of an Fc construct with three Fc domains and knob-into-hole and electrostatic steering mutations in the two “branch” subunits (Fc construct B), were transfected into HEK293 cells. Following seven days in culture, cells were cleared by centrifugation and raw media supernatants were separated by non-reducing SDS-PAGE (FIG. 10). Densitometric analysis of the visualized protein bands revealed that Fc construct A having three Fc domains and Fc construct B having three Fc domains (Fc3) are expressed at similar levels. However, the constructs for Fc construct A expressed significantly higher levels of contaminating dimer (Fc2) species (FIG. 51). Both sets of constructs expressed similar levels of the monomer species (Fc1). Additional bands present in the image represent media components that are present in mock transfected controls.

These results indicate that having both electrostatic steering mutations that promote heterodimerization and knob-into-hole mutations that promote heterodimerization in the “branch” subunits enhances formation of a heterodimeric Fc domain in an Fc construct, optimizes the assembly of an Fc construct having three Fc domains, and improves the homogeneity of the composition containing the Fc construct.

### Example 50. Electrostatic Steering for Control of Homodimerization

To minimize off-register association of subunits, which generates unwanted high molecular weight oligomers and multimers, mutations that favor heterodimerization (e.g., knobs and holes) were introduced into the “branch” subunits. These amino acid substitutions preserve the attraction of knobs subunits for the holes counterparts and at the same time hinder association between knobs subunits. Because the knobs mutations also inhibit assembly with wild-type Fc sequences, it calls into question the necessity of including additional mutations to further reduce affinity of the “stem” Fc subunits for the knobs and holes “branch” subunits. To address this question, an Fc construct long polypeptide was generated which contained a wild-type Fc domain monomer sequence in the carboxyl terminal “stem” subunit and an Fc domain monomer carrying knob mutations in the amino terminal “branch” subunit. The corresponding short polypeptide was the Fc domain monomer carrying hole mutations. This Fc construct is based on the sequences of the polypeptides in Fc construct A, but has a wild-type Fc domain monomer sequence in the carboxyl terminal “stem” subunit in each of the long polypeptides.

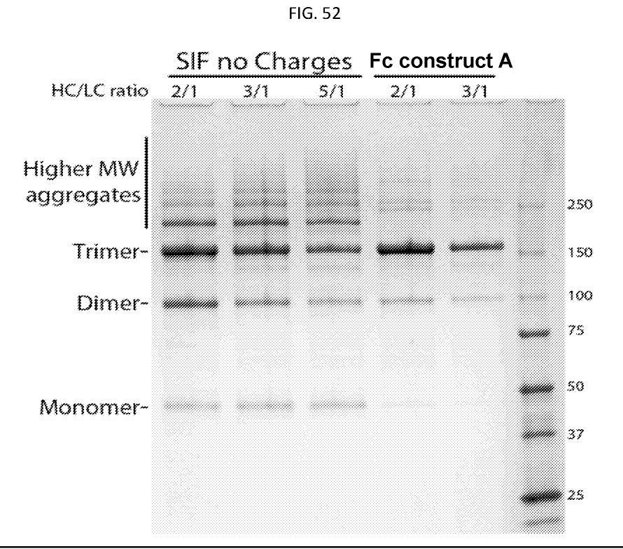

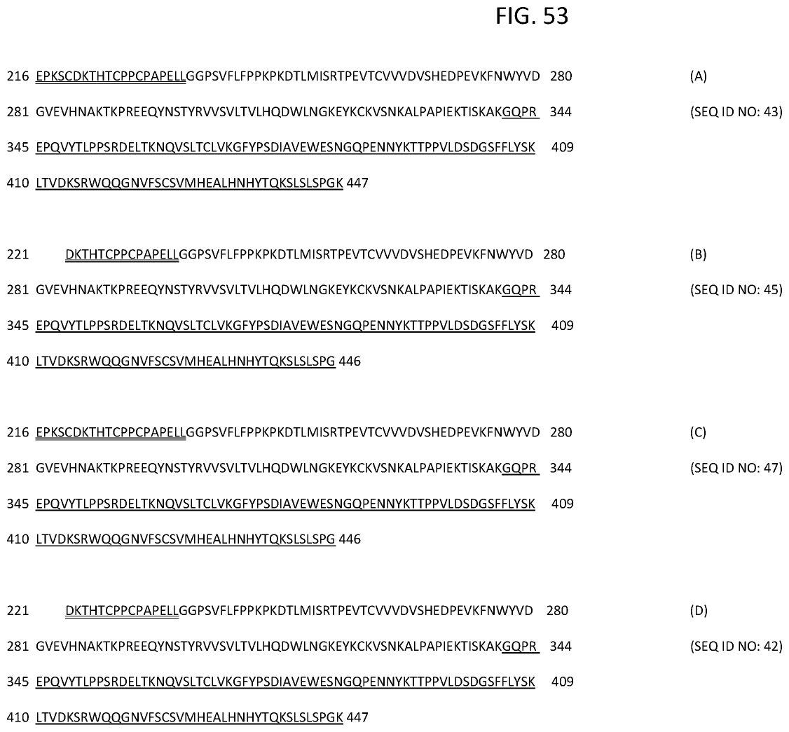

HEK293 cells were co-transfected with plasmids expressing Fc construct A (which has homodimerizing electrostatic steering mutations in the Fc domain monomer in the carboxyl terminal “stem” subunit in each of the long polypeptides), or an Fc construct based on Fc construct A in which the Fc domain monomer in the carboxyl terminal “stem” subunit in each of the long polypeptides was replaced with a wild-type Fc domain monomer sequence (SEQ ID NO: 42). Following seven days in culture, cells were cleared by centrifugation and raw media supernatants were separated by non-reducing SDS-PAGE. Imaging of stained proteins revealed that the Fc construct without electrostatic steering mutations in the “stem” subunits (labeled “No electrostatic steering” (lanes 1-3) in FIG. 52) contained much higher levels of monomer (Fc1) and dimer (Fc2) than the Fc construct A counterpart (labeled “With electrostatic steering” (lanes 4 and 5) in FIG. 52). Furthermore, a much larger number of bands higher in molecular weight than the trimer can be detected (lanes 1-3 in FIG. 52).

These results confirm that having electrostatic steering mutations that promote homodimerization in the “stem” subunits further enhances formation of a homodimeric Fc domain in the Fc construct, optimizes the assembly of an Fc construct having three Fc domains, and improves the homogeneity of the composition containing the Fc construct.

### Example 51. Design and Purification of Fc-Antigen Binding Domain Construct 4 with an Anti-CD20 Antigen Binding Domain or an Anti-PD-L1 Antigen Binding Domain

### Protein Expression

A construct formed from asymmetrical tandem Fc domains was made as described below. Fc-antigen binding domain construct 4 (CD20) and construct 4 (PD-L1) each includes two distinct Fc domain monomer containing polypeptides (a long Fc chain (SEQ ID NO: 66), and three copies of either an anti-CD20 short Fc chain (SEQ ID NO: 67) or an anti-PD-L1 Fc chain (SEQ ID NO: 68)) and three copies of either an anti-CD20 light chain polypeptide (SEQ ID NO: 61) or an anti-PD-L1 light chain polypeptide (SEQ ID NO: 49), respectively. The long Fc chain contains three Fc domain monomers in a tandem series, wherein each Fc domain monomer has an E357K charge mutation and S354C and T366W protuberance-forming mutations (to promote heterodimerization). The short Fc chain contains an Fc domain monomer with a K370D charge mutation and Y349C. T366S, L368A, and Y407V cavity-forming mutations (to promote heterodimerization), and either anti-CD20 VH and CH1 domains (Kabat positions 1-220) at the N-terminus (construct 4 (CD20) or anti-PD-L1 VH and CH1 domains (Kabat positions 1-220) at the N-terminus (construct 4 (PD-L1)). The anti-CD20 light chain or PD-L1 light chain can also be expressed fused to the N-terminus of the short Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 9 were encoded by three separate plasmids (one plasmid encoding the light chain (anti-CD20 or anti-PD-L1), one plasmid encoding the long Fc chain and one plasmid encoding the short Fc chain (anti-CD20 or anti-PD-L1)):

TABLE 9
Construct 4 (CD20) and Construct 4 (PD-L1) sequences

| | | | Short Fc chain |
|---|---|---|---|
| | | | (with anti-CD20 or |
| Construct | Light chain | Long Fc chain | anti-PD-L1 VH and CH1) |
| Construct 4 | SEQ ID NO: 61 | SEQ ID NO: 66 | SEQ ID NO: 67 |
| (CD20) | DIVMTQTPLSLPVTPGEPASI | DKTHTCPPCPAPELLGGPS | QVQLVQSGAEVKKPGSSV |
| | SCRSSKSLLHSNGITYLYWYL | VFLFPPKPKDTLMISRTPE | KVSCKASGYAFSYSWINW |
| | QKPGQSPQLLIYQMSNLVSG | VTCVVVDVSHEDPEVKFN | VRQAPGQGLEWMGRIFP |
| | VPDRFSGSGSGTDFTLKISRV | WYVDGVEVHNAKTKPRE | GDGDTDYNGKFKGRVTIT |
| | EAEDVGVYYCAQNLELPYTF | EQYNSTYRVVSVLTVLHQ | ADKSTSTAYMELSSLRSED |
| | GGGTKVEIKRTVAAPSVFIFP | DWLNGKEYKCKVSNKALP | TAVYYCARNVFDGYWLVY |
| | PSDEQLKSGTASVVCLLNNFY | APIEKTISKAKGQPREPQV | WGQGTLVTVSSASTKGPS |
| | PREAKVQWKVDNALQSGNS | YTLPPCRDKLTKNQVSLW | VFPLAPSSKSTSGGTAALG |
| | QESVTEQDSKDSTYSLSSTLT | CLVKGFYPSDIAVEWESN | CLVKDYPEPVTVSWNSG |
| | LSKADYEKHKVYACEVTHQG | GQPENNYKTTPPVLDSDG | ALTSGVHTFPAVLQSSGLY |
| | LSSPVTKSFNRGEC | SFFLYSKLTVDKSRWQQG | SLSSVVTVPSSSLGTQTYIC |
| | | NVFSCSVMHEALHNHYT | NVNHKPSNTKVDKKVEPK |
| | | QKSLSLSPGKGGGGGGG | SCDKTHTCPPCPAPELLGG |
| | | GGGGGGGGGGGGGDKT | PSVFLFPPKPKDTLMISRT |
| | | HTCPPCPAPELLGGPSVFL | PEVTCVVVDVSHEDPEVK |
| | | FPPKPKDTLMISRTPEVTC | FNWYVDGVEVHNAKTKP |
| | | VVVDVSHEDPEVKFNWY | REEQYNSTYRVVSVLTVLH |
| | | VDGVEVHNAKTKPREEQY | QDWLNGKEYKCKVSNKA |
| | | NSTYRVVSVLTVLHQDWL | LPAPIEKTISKAKGQPREP |
| | | NGKEYKCKVSNKALPAPIE | QVCTLPPSRDELTKNQVSL |
| | | KTISKAKGQPREPQVYTLP | SCAVDGFYPSDIAVEWES |
| | | PCRDKLTKNQVSLWCLVK | NGQPENNYKTTPPVLDSD |
| | | GFYPSDIAVEWESNGQPE | GSFFLVSKLTVDKSRWQQ |
| | | NNYKTTPPVLDSDGSFFLY | GNVFSCSVMHEALHNHY |
| | | SKLTVDKSRWQQGNVFS | TQKSLSLSPG |
| | | CSVMHEALHNHYTQKSLS | |
| | | LSPGKGGGGGGGGGGG | |
| | | GGGGGGGGGDKTHTCPP | |
| | | CPAPELLGGPSVFLFPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYCKVSNKALPAPIEKTIS | |
| | | KAKGQPREPQVYTLPPCR | |
| | | DKLTKNQVSLWCLVKGFY | |
| | | PSDIAVEWESNGQPENNY | |
| | | KTTPPVLDSDGSFFLYSKLT | |
| | | VDKSRWQQGNVFSCSV | |
| | | MHEALHNHYTQKSLSLSP | |
| | | G | |
| Construct 4 | SEQ ID NO: 49 | SEQ ID NO: 66 | SEQ ID NO: 68 |
| (PD-Li) | QSALTQPASVSGSPGQSIT | DKTHTCPPCPAPELLGGPS | EVQLLESGGGLVQPGGSL |
| | ISCTGTSSDVGGYNYVSW | VFLFPPKPKDTLMISRTPE | RLSCAASGHTSSYIMMW |
| | YQQHPGKAPKLMIYDVSN | VTCVVVDVSHEDPEVKFN | VRQAPGKGLEWVSSIYPS |
| | RPSGVSNRFSGSKSGNTA | WYVDGVEVHNAKTKPRE | GGITHADTVKGRFTISRD |
| | SLTISGLQAEDEADYYCSS | EQYNSTYRVVSVLTVLHQ | NSKNTLYLQMNSLRAEDT |
| | TISSSTRVFGTGTKVTVLG | DWLNGKEYKCKVSNKALP | AVYYCARIKLGTVTTVDY |
| | QPKANPIATILFPPSSEELQ | APIEKTISKAKGQPREPQV | WGQGTLVTVSSASTKGPS |
| | ANKATLVCLISDFYPGAVT | YTLPPCRDKLTKNQVSLW | VFPLAPSSKSTSGGTAALG |
| | VAWKADGSPVKAGVEII | CLVKGFYPSDIAVEWESN | CLVKDYFPEPVTVSWNSG |
| | KPSKQSNNKYAASSYLSLT | GQPENNYKTTPPVLDSDG | ALTSGVHTFPAVLQSSGLY |
| | PEQWKSHRSYSCQVTHE | SFFLYSKLTVDKSRWQQG | SLSSVVTVPSSSLGTQTYIC |
| | GSTVEKTVAPTECS | NVFSCSVMHEALHNHYT | NVNHKPSNTKVDKKVEPK |
| | | QKSLSLSPGKGGGGGGG | SCDKTHTCPPCPAPELLGG |
| | | GGGGGGGGGGGGGDKT | PSVFLFPPKPKDTLMISRT |
| | | HTCPPCPAPELLGGPSVFL | PEVTCVVVDVSHEDPEVK |
| | | FPPKPKDTLMISRTPEVTC | FNWYVDGVEVHNAKTKP |
| | | VVVDVSHEDPEVKFNWY | REEQYNSTYRVVSVLTVLH |
| | | VDGVEVHNAKTKPREEQY | QDWLNGKEYKCKVSNKA |
| | | NSTYRVVSVLTVLHQDWL | LPAPIEKTISKAKGQPREP |
| | | NGKEYKCKVSNKALPAPIE | QVCTLPPSRDELTKNQVSL |
| | | KTISKAKGQPREPQVYTLP | SCAVDGFYPSDIAVEWES |
| | | PCRDKLTKNQVSLANCLVK | NGQPENNYKTTPPVLDSD |
| | | GFYPSDIAVEWESNGQPE | GSFFLVSKLTVDKSRWOD |
| | | NNYKTTPPVLDSDGSFFLY | GNVFSCSVMHEALHNHY |
| | | SKLTVDKSRWQQGNVFS | TQKSLSLSPG |
| | | CSVMHEALHNHYTQKSLS | |
| | | LSPGKGGGGGGGGGGG | |
| | | GGGGGGGGGDKTHTCPP | |
| | | CPAPELLGGPSVFLFPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPCR | |
| | | DKLTKNQVSLANCLVKGFY | |
| | | PSDIAVEWESNGQPENNY | |
| | | KTTPPVLDSDGSFELYSKLT | |
| | | VDKSRWQQGNVFSCSV | |
| | | MHEALHNHYTQKSLSLSP | |
| | | G | |

The expressed proteins were purified from the cell culture supernatant by Protein A-based affinity column chromatography, using a Poros MabCapture A (Life Technologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Suffate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 52. Design and Purification of Fc-Antigen Binding Domain Construct 8 with an Anti-CD20 Antigen Binding Domain or an Anti-PD-L1 Antigen Binding Domain

### Protein Expression

A construct formed from a singly branched Fc domain where the branch point is at the N-terminal Fc domain was made as described below. Fc-antigen binding domain construct 8 (CD20) and construct 8 (PD-L1) each include two distinct Fc domain monomer containing polypeptides (two copies of a long Fc chain (SEQ ID NO: 69), and two copies of either an anti-CD20 short Fc chain (SEQ ID NO: 67) or an anti-PD-L1 short Fc chain (SEQ ID NO: 68)) and copies of either an anti-CD20 light chain polypeptide (SEQ ID NO: 61) or an anti-PD-L1 light chain polypeptide (SEQ ID NO: 49), respectively. The long Fc chain contains an Fc domain monomer with an E357K charge mutation and S354C and T366W protuberance-forming mutations (to promote heterodimerization) in a tandem series with an Fc domain monomer with reverse charge mutations K409D and D399K (to promote homodimerization). The short Fc chain contains an Fc domain monomer with a K370D charge mutation and Y349C, T366S, L368A, and Y407V cavity-forming mutations (to promote heterodimerization), and either anti-CD20 VH and CH1 domains (Kabat positions 1-220) at the N-terminus (construct 8 (CD20) or anti-PD-L1 VH and CH1 domains (Kabat positions 1-220) at the N-terminus (construct 8 (PD-L1)). The anti-CD20 light chain or PD-L1 light chain can also be expressed fused to the N-terminus of the short Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 10 were encoded by three separate plasmids (one plasmid encoding the light chain (anti-CD20 or anti-PD-L1), one plasmid encoding the long Fc chain and one plasmid encoding the short Fc chain (anti-CD20 or anti-PD-L1)):

TABLE 10
Construct 8 (CD20) and Construct 8 (PD-L1) sequences

| | | | Short Fc chain |
|---|---|---|---|
| | | | (with anti-CD20 or |
| Construct | Light chain | Long Fc chain | anti-PD-L1 VH and CH1) |
| Construct 8 | SEQ ID NO: 61 | SEQ ID NO: 69 | SEQ ID NO: 67 |
| (CD20) | DIVMTQTPLSLPVTPGEPA | DKTHTCPPCPAPELLGGPS | QVQLVQSGAEVKKPGSSVKV |
| | SISCRSSKSLLHSNGITYLY | VFLFPPKPKDTLMISRTPE | SCKASGYARSYSWINWVRQ |
| | WYLQKPGQSPOLLIYQMS | VTCVVVDVSHEDPEVKFN | APGQGLEWMGRIFPGDGDT |
| | NLVSGVPDRFSGSGSGTD | WYVDGVEVHNAKTKPRE | DYNGKFKGRVTITADKSTSTA |
| | FTLKISRVEAEDVGVYYCA | EQYNSTYRVVSVLTVLHQ | YMELSSLRSEDTAVYYCARN |
| | QNLELPYTEGGGTKVEIKR | DWLNGKEYKCKVSNKALP | VEDGYWLVYWGQGTLVTVS |
| | TVAAPSVFIFPPSDEQLKS | APIEKTISKAKGQPREPQV | SASTKGPSVFPLAPSSKSTSG |
| | GTASVVCLLNNEYPREAK | YTLPPSRDELTKNQVSLTC | GTAALGCLVKDYFPEPVTVS |
| | VQWKVDNALQSGNSQES | LVKGFYPSDIAVEWESNG | WNSGALTSGVHTFPAVLQSS |
| | VTEQDSKDSTYSLSSTLTLS | QPENNYKTTPPVLKSDGS | GLYSLSSVVTVPSSSLGTQTYI |
| | KADYEKHKVYACEVTHQG | FFLYSDLTVDKSRWQQGN | CNVNHKPSNTKVDKKVEPKS |
| | LSSPVTKSFNRGEC | VFSCSVMHEALHNHYTQ | CDKIHTCPPCPAPELLGGPS |
| | | KSLSLSPGKGGGGGGGG | VFLFPPKPKDTLVHSRTPEVT |
| | | GGGGGGGGGGGGDKTH | CVVVDVSHEDPEVKFNWYV |
| | | TCPPCPAPELLGGPSVFLF | DGVEVHNAKTKPREEQYNST |
| | | PPKPKDTLMISRTPEVTCV | YRVVSVLTVLHODWLNGKE |
| | | VVDVSHEDPEVKFNWYV | YKCKVSNKALPAPIEKTISKAK |
| | | DGVEVHNAKTKPREEQY | GQPREPQVCTLPPSRDELTK |
| | | NSTYRVVSVLTVLHQDWL | NQVSLSCAVDGFYPSDIAVE |
| | | NGKEYKCKVSNKALPAPIE | WESNGOPENNYKTTPPVLD |
| | | KTISKAKGQPREPQVYTLP | SDGSFFLVSKLTVDKSRWQQ |
| | | PCRDKLTKNQVSLWCLVK | GNVESCSVMHEALHNHYM |
| | | GFYPSDIAVEWESNGQPE | KSLSLSPG |
| | | NNYKTTPPVLDSDGSFFLY | |
| | | SKLTVDKSRWQQGNVES | |
| | | CSVMHEALHNHYTQKSLS | |
| | | LSPG | |
| Construct 8 | SEQ ID NO: 49 | SEQ ID NO: 69 | SEQ ID NO: 68 |
| (PD-L1) | QSALTQPASVSGSPGQSIT | DKTHTCPPCPAPELLGGPSVF | EVQLLESGGGLVQPGGSL |
| | ISCTGTSSDVGGYNYVSW | LFPPKPKDTLMISRTPEVTCV | RLSCAASGFTFSSYIMMW |
| | YQQHPGKAPKLMIYDVSN | VVDVSHEDPEVKFNWYVDG | VRQAPGKGLEWVSSIYPS |
| | RPSGVSNRFSGSKSGNTA | VEVHNAKTKPREEQYNSTYR | GGITFYADTVKGRFTISRD |
| | SLTISGLQAEDEADYYCSS | VVSVLTVLHQDWLNGKEYK | NSKNTLYLQMNSLRAEDT |
| | YtSSSTRVFGTETKVTVLG | CKVSNKALPAPIEKTISKAKG | AVYYCARIKLGTVtTVDY |
| | QPKANPTVTLFPPSSEELQ | QPREPQVYTLPPSRDELTKN | WGQGTLVTVSSASTKGPS |
| | ANKATLVCLISDFYPGAVT | QVSLTCLVKGFYPSDIAVEW | VFPLAPSSKSTSGGTAALG |
| | VAWKADGSPVKAGVETT | ESNGQPENNYKTTPPVLKSD | CLVKDYFPEPVTVSWNSG |
| | KPSKQSNNKYAASSYLSLT | GSFFLYSDLTVDKSRVVQQG | ALTSGVHTFPAVLQSSGLY |
| | PEQWKSHRSYSCQVTHE | NVFSCSVMHEALHNHYTQK | SLSSVVTVPSSSLGTQTYIC |
| | GSTVEKTVAPTECS | SLSLSPGKGGGGGGGGGGG | NVNHKPSNTKVDKKVEPK |
| | | GGGGGGGGGDKTHTCPPCP | SCDKTHTCPPCPAPELLGG |
| | | APELLGGPSVFLFPPKPKDTL | PSVFLFPPKPKDTLMISRT |
| | | MISRTPEVTCVVVDVSHEDP | PEVTCVVVDVSHEDPEVK |
| | | EVKFNWYVDGVEVHNAKTK | FNWYVDGVEVHNAKTKP |
| | | PREEQYNSTYRVVSVLTVLH | REEQYNSTYRVVSVLTVLH |
| | | QDWLNGKEYKCKVSNKALP | QDWLNGKEYKCKVSNKA |
| | | APIEKTISKAKGQPREPQVYT | LPAPIEKTISKAKGOPREP |
| | | LPPCRDKLTKNQVSLWCLVK | QVCTLPPSRDELTKNQVSL |
| | | GFYPSDIAVEWESNGQPEN | SCAVDGFYPSDIAVEWES |
| | | NYKTTPPVLDSDGSFFLYSKL | NGQPENNYKTTPPVLDSD |
| | | TVDKSRWQQGNVFSCSVM | GSFFLVSKLTVDKSRWQQ |
| | | HEALHNHYTQKSLSLSPG | GNVFSCSVMHEALHNHY |
| | | | TQKSLSLSPG |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 53. Design and Purification of Fc-Antigen Binding Domain Construct 9 with an Anti-CD20 Antigen Binding Domain or an Anti-PD-L1 Antigen Binding Domain

### Protein Expression

A construct formed from a singly branched Fc domain where the branch point is at the N-terminal Fc domain was made as described below. Fc-antigen binding domain construct 9 (CD20) and construct 9 (PD-L1) each include two distinct Fc domain monomer containing polypeptides (two copies of either an anti-CD20 long Fc chain (SEQ ID NO: 62) or an anti-PD-L1 long Fc chain (SEQ ID NO: 54), and two copies of either an anti-CD20 short Fc chain (SEQ ID NO: 67) or an anti-PD-L1 short Fc chain (SEQ ID NO: 68)) and copies of either an anti-CD20 light chain polypeptide (SEQ ID NO: 61) or an anti-PD-L1 light chain polypeptide (SEQ ID NO: 49), respectively. The long Fc chain contains an Fc domain monomer with an E357K charge mutation and S354C and T366W protuberance-forming mutations (to promote heterodimerization) in a tandem series with an Fc domain monomer with reverse charge mutations K409D and D399K (to promote homodimerization), and either anti-CD20 VH and CH1 domains (Kabat positions 1-220) at the N-terminus (construct 9 (CD20) or anti-PD-L1 VH and CH1 domains (Kabat positions 1-220) at the N-terminus (construct 9 (PD-L1)). The short Fc chain contains an Fc domain monomer with a K370D charge mutation and Y349C, T366S, L368A, and Y407V cavity-forming mutations (to promote heterodimerization), and either an anti-CD20 heavy chain at the N-terminus (construct 9 (CD20) or an anti-PD-L1 heavy chain at the N-terminus (construct 9 (PD-L1)). The anti-CD20 light chain or PD-L1 light chain can also be expressed fused to the N-terminus of the long Fc chain and/or short Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 11 were encoded by three separate plasmids (one plasmid encoding the light chain (anti-CD20 or anti-PD-L1), one plasmid encoding the long Fc chain (anti-CD20 or anti-PD-L1) and one plasmid encoding the short Fc chain (anti-CD20 or anti-PD-L1)):

TABLE 11
Construct 9 (CD20) and Construct 9 (PD-L1) sequences

| | | Long Fc chain | Short Fc chain |
|---|---|---|---|
| | | (with anti-CD20 or | (with anti-CD20 or |
| Construct | Light chain | anti-PD-L1 VH and CH1) | anti-PD-L1 VH and CH1) |
| Construct 9 | SEQ ID NO: 61 | SEQ ID NO: 62 | SEQ ID NO: 67 |
| (CD20) | DIVMTQTPLSLPVTPGEPA | QVQLVQSGAEVKKPGSSV | QVQLVQSGAEVKKPGSSV |
| | SISCRSSKSLLHSNGITYLY | KVSCKASGYAFSYSWINW | KVSCKASGYAFSYSWINW |
| | WYLQKPGQSPQLLIYQMS | VRQAPGQGLEWMGRIFP | VRQAPGQGLEWMGRIFP |
| | NLVSGVPDRFSGSGSGTD | GDGDTDYNGKFKGRVTIT | GDGDTDYNGKFKGRVTIT |
| | FTLKISRVEAEDVGVYYCA | ADKSTSTAYMELSSLRSED | ADKSTSTAYMELSSLRSED |
| | QNLELPYTFGGGTKVEIKR | TAVYYCARNVFDGYWLVY | TAVYYCARNVFDGYWLVY |
| | TVAAPSVFIFPPSDEQLKS | WGOGTLVTVSSASTKGPS | WGQGTLVTVSSASTKGPS |
| | GTASVVCLLNNFYPREAK | VFPLAPSSKSTSGGTAALG | VFPLAPSSKSTSGGTAALG |
| | VQWKVDNALQSGNSQES | CLVKDYFPEPVTVSWNSG | CLVKDYFPEPVTVSWNSG |
| | VTEQDSKDSTYSLSSTLTLS | ALTSGVHTFPAVLQSSGLY | ALTSGVHTFPAVLQSSGLY |
| | KADYEKHKVYACEVTHQG | SLSSVVTVPSSSLGTQTYIC | SLSSVVTVPSSSLGTOTYIC |
| | LSSPVTKSENRGEC | NVNHKPSNTKVDKKVEPK | NVNHKPSNTKVDKKVEPK |
| | | SCDKTHTCPPCPAPELLGG | SCDKTHTCPPCPAPELLGG |
| | | PSVFLFPPKPKDTLMISRT | PSVFLFPPKPKDTLMISRT |
| | | PEVTCVVVDVSHEDPEVK | PEVTCVVVDVSHEDPEVK |
| | | FNWYVDGVEVHNAKTKP | FNWYVDGVEVHNAKTKP |
| | | REEQYNSTYRVVSVLTVLH | REEQYNSTYRVVSVLTVLH |
| | | QDWLNGKEYKCKVSNKA | QDWLNGKEYKCKVSNKA |
| | | LPAPIEKTISKAKGQPREP | LPAPIEKTISKAKGQPREP |
| | | QVYTLPPSRDELTKNQVSL | QVCTLPPSRDELTKNQVSL |
| | | TCLVKGFYPSDIAVEWES | SCAVDGFYPSDIAVEWES |
| | | NGQPENNYKTTPPVLKSD | NGQPENNYKTTPPVLDSD |
| | | GSFFLYSDLTVDKSRWQQ | GSFELVSKLTVDKSRWQQ |
| | | GNVFSCSVMHEALHNHY | GNVFSCSVMHEALHNHY |
| | | TQKSLSLSPGKGGGGGGG | TQKSLSLSPG |
| | | GGGGGGGGGGGGGDKT | |
| | | HTCPPCPAPELLGGPSVFL | |
| | | FPPKPKDTLMISRTPEVTC | |
| | | VVVDVSHEDPEVKFNWY | |
| | | VDGVEVHNAKTKPREEQY | |
| | | NSTYRVVSVLTVLHQDWL | |
| | | NGKEYKCKVSNKALPAPIE | |
| | | KTISKAKGQPREPQVYTLP | |
| | | PCRDKLTKNQVSLWCLVK | |
| | | GFYPSDIAVEWESNGQPE | |
| | | NNYKTTPPVLDSDGSFFLY | |
| | | SKLTVDKSRWQQGNVFS | |
| | | CSVMHEALHNHYTQKSLS | |
| | | LSPG | |
| Construct 9 | SEQ ID NO: 49 | SEQ. ID NO: 54 | SEC), ID NO: 68 |
| (PD-L1) | QSALTQPASVSGSPGQSIT | EVQLLESGGGLVQPGGSL | EVQLLESGGGLVQPGGSL |
| | ISCTGTSSDVGGYNYVSW | RLSCAASGFTESSYIMMW | RLSCAASGFTESSYIMMW |
| | YQQHPGKAPKLMIYDVSN | VRQAPGKGLEWVSSIYPS | VRQAPGKGLEWVSSIYPS |
| | RPSGVSNRFSGSKSGNTA | GGITFYADTVKGRFTISRD | GGITFYADTVKGRFTISRD |
| | SLTISGLQAEDEADYYCSS | NSKNTLYLQMNSLRAEDT | NSKNTLYLQMNSLRAEDT |
| | YTSSSTRVEGTGTKVTVLG | AVYYCARIKLGTVTTVDY | AVYYCARIKLGTVTTVDY |
| | QPKANPTVTLFPPSSEELQ | WGQGTIATTVSSASTKGPS | WGQGTLATIVSSASTKGPS |
| | ANKATLVCLISDFYPGAVT | VFPLAPSSKSTSGGTAALG | VFPLAPSSKSTSGGTAALG |
| | VAWKADGSPVKAGVETT | CLVKDYFPEPVTVSWNSG | CLVKDYFPEPVTVSWNSG |
| | KPSKQSNNKYAASSYLSLT | ALTSGVHTFPAVLQSSGLY | ALTSGVHTFPAVIQSSGLY |
| | PEQWKSHRSYSCQVTHE | SLSSVVTVPSSSLGTQTYIC | SLSSVVTVPSSSLGTQTYIC |
| | GSTVEKTVAPTECS | NVNHKPSNTKVDKKVEPK | NVNHKPSNTKVDKKVEPK |
| | | SCDKTHTCPPCPAPELLGG | SCDKTHTCPPCPAPELLGG |
| | | PSVFLFPPKPKDTLMISRT | PSVFLFPPKPKDTLMISRT |
| | | PEVTCVVVDVSHEDPEVK | PEVTCVVVDVSHEDPEVK |
| | | FNWYVDGVEVHNAKTKP | FNWYVDGVEVHNAKTKP |
| | | REEQYNSTYRVVSVLTVLH | REEQYNSTYRVVSVLTVLH |
| | | QDWLNGKEYKCKVSNKA | QDWLNGKEYKCKVSNKA |
| | | LPAPIEKTISKAKGQPREP | LPAPIEKTISKAKGQPREP |
| | | QVYTLPPSRDELTKNQVSL | QVCTLPPSRDELTKNQVSL |
| | | TCLVKGFYPSDIAVEWES | SCAVDGFYPSDIAVEWES |
| | | NGQPENNYKTTPPVLKSD | NGQPENNYKTTPPVLDSD |
| | | GSFFLYSDLTVDKSRWQQ | GSFFLVSKLTVDKSRWQQ |
| | | GNVFSCSVMHEALHNHY | GNVFSCSVMHEALHNHY |
| | | TQKSLSLSPGKGGGGGGG | TQKSLSLSPG |
| | | GGGGGGGGGGGGGDKT | |
| | | HTCPPCPAPELLGGPSVFL | |
| | | FPPKPKDTLMISRTPEVTC | |
| | | VVVDVSHEDPEVKFNWY | |
| | | VDGVEVHNAKTKPREEQY | |
| | | NSTYRVVSVLTVLHQDWL | |
| | | NGKEYKCKVSNKALPAPIE | |
| | | KTISKAKGQPREPQVYTLP | |
| | | PCRDKLTKNQVSLWCLVK | |
| | | GFYPSDIAVEWESNGQPE | |
| | | NNYKTTPPVLDSDGSFFLY | |
| | | SKLTVDKSRWQQGNVFS | |
| | | CSVMHEALHNHYTQKSLS | |
| | | LSPG | |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 54. Design and Purification of Fc-Antigen Binding Domain Construct 10 with an Anti-CD20 Antigen Binding Domain or an Anti-PD-L1 Antigen Binding Domain

### Protein Expression

A construct formed from a singly branched Fc domain where the branch point is at the N-terminal Fc domain was made as described below. Fc-antigen binding domain construct 10 (CD20) and construct 10 (PD-L1) each include two distinct Fc domain monomer containing polypeptides (two copies of either an anti-CD20 long Fc chain (SEQ ID NO: 70) or an anti-PD-L1 long fc chain (SEQ ID NO: 71), and four copies of a short Fc chain (SEQ ID NO: 63)) and copies of either an anti-CD20 light chain polypeptide (SEQ ID NO: 61) or an anti-PD-L1 light chain polypeptide (SEQ ID NO: 49), respectively. The long Fc chain contains two Fc domain monomers in a tandem series, wherein each Fc domain monomer has an E357K charge mutation and S354C and T366W protuberance-forming mutations (to promote heterodimerization), in tandem series with an Fc domain monomer with reverse charge mutations K409D and D399K (to promote homodimerization), and either anti-CD20 VH and CH1 domains (Kabat positions 1-220) at the N-terminus (construct 10 (CD20) or anti-PD-L1 VH and CH1 domains (Kabat positions 1-220) at the N-terminus (construct 10 (PD-L1)). The short Fc chain contains an Fc domain monomer with a K370D charge mutation and Y349C, T366S, L368A, and Y407V cavity-forming mutations (to promote heterodimerization). The anti-CD20 light chain or PD-L1 light chain can also be expressed fused to the N-terminus of the long Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 12 were encoded by three separate plasmids (one plasmid encoding the light chain (anti-CD20 or anti-PD-L1), one plasmid encoding the long Fc chain (anti-CD20 or anti-PD-L1) and one plasmid encoding the short Fc chain:

TABLE 12
Construct 10 (CD20) and Construct 10 (PD-L1) sequences

| | | Long Fc chain | |
|---|---|---|---|
| | | (with anti-CD20 or | |
| Construct | Light chain | anti-PD-L1 VH and CH1) | Short Fc chain |
| Construct 10 | SEQ ID NO: 61 | SEQ ID NO: 70 | SEQ ID NO: 63 |
| (CD20) | DIVMTQTPLSLPVTPGEPA | QVQLVQSGAEVKKPGSSV | DKTHTCPPCPAPELLGGPS |
| | SISCRSSKSLLHSNGITYLY | KVSCKASGYAFSYSWINW | VFLFPPKPKDTLMISRTPE |
| | WYLQKPGQSPCILLIYQMS | VRQAPGQGLEWMGRIFP | VTCVVVDVSHEDPEVKFN |
| | NLVSGVPDRFSGSGSGTD | GDGDTDYNGKFKGRVTIT | WYVDGVEVHNAKTKPRE |
| | FTLKISRVEAEDVGVYYCA | ADKSTSTAYMELSSLRSED | EQYNSTYRVVSVLTVLHQ |
| | ONLELPYTFGGGTKVEIKR | TAVYYCARNVFDGYWLVY | DWLNGKEYKCKVSNKALP |
| | TVAAPSVFIFPPSDEQLKS | WGQGTLVTVSSASTKGPS | APIEKTISKAKGQPREPQV |
| | GTASVVCLLNNFYPREAK | VFPLAPSSKSTSGGTAALG | CTLPPSRDELTKNQVSLSC |
| | VQWKVDNALQSGNSQES | CLVKDYFPEPVTVSWNSG | AVDGFYPSDIAVEWESNG |
| | VTEQDSKDSTYSLSSTLTLS | ALTSGVHTFPAVLQSSGLY | QPENNYKTTPPVLDSDGS |
| | KADYEKHKVYACEVTHQG | SLSSVVTVPSSSLGTQTYIC | FFLVSKLTVDKSRWQQGN |
| | LSSPVTKSFNRGEC | NVNHKPSNTKVDKKVEPK | VFSCSVMHEALHNHYTQ |
| | | SCDKTHTCPPCPAPELLGG | KSLSLSPG |
| | | PSVFLFPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPSRDELTKNQVSL | |
| | | TCLVKGFYPSDIAVEWES | |
| | | NGQPENNYKTTPPVLKSD | |
| | | GSFFLYSDLTVDKSRWQQ | |
| | | GNVFSCSVMHEALHNHY | |
| | | TQKSLSLSPGKGGGGGGG | |
| | | GGGGGGGGGGGGGDKT | |
| | | HTCPPCPAPELLGGPSVFL | |
| | | FPPKPKDTLMISRTPEVTC | |
| | | VVVDVSHEDPEVKFNWY | |
| | | VDGVEVHNAKTKPREEQY | |
| | | NSTYRVVSVLTVLHQDWL | |
| | | NGKEYKCKVSNKALPAPIE | |
| | | KTISKAKGQPREPQVYTLP | |
| | | PCRDKLTKNQVSLWCLVK | |
| | | GFYPSDIAVEWESNGQPE | |
| | | NNYKTTPPVLDSDGSFFLY | |
| | | SKLTVDKSRWQQGNVFS | |
| | | CSVMHEALHNHYTQKSLS | |
| | | LSPGKGGGGGGGGGGG | |
| | | GGGGGGGGGDKTHTCPP | |
| | | CPAPELLGGPSVFLFPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPCR | |
| | | DKLTKNQVSLWCLVKGFY | |
| | | PSDIAVEWESNGQPENNY | |
| | | KTTPPVLDSDGSFFLYSKLT | |
| | | VDKSRWQQGNVFSCSV | |
| | | MHEALHNHYTQKSLSLSP | |
| | | G | |
| Construct 10 | SEQ ID NO: 49 | SEQ ID NO: 71 | SEQ ID NO: 63 |
| (PD-L1) | QSALTQPASVSGSPGQSIT | EVQLLESGGGLVQPGGSL | DKIHTCPPCPAPELLGGPS |
| | ISCTGTSSDVGGYNYVSW | RLSCAASGFIFSSYIMMW | VFLFPPIKPKDTLMISRTPE |
| | YQQHPGKAPKLMIYDVSN | VRQAPGKGLEWVSSIYPS | VTCVVVDVSHEDPEVKFN |
| | RPSGVSNRFSGSKSGNTA | GGITHADIVKGRFTISRD | WYVDGVEVHNAKTKPRE |
| | SLTISGLQAEDEADYYCSS | NSKNTLYLQMNSLRAEDT | EQYNSTYRVVSVLTVLHQ |
| | YISSSTRVFGTGIKVIVLG | AVYYCARIKLGIVTIVDY | DWLNGKEYKCKVSNKALP |
| | QPKANPTVTLFPPSSEELQ | WGQGTLVTVSSASTKGPS | APIEKTISKAKGQPREPQV |
| | ANKATLVCLISDFYPGAVT | VFPLAPSSKSTSGGTAALG | CTLPPSRDELIKNQVSLSC |
| | VAWKADGSPVKAGVEII | CLVKDYFPEPVTVSWNSG | AVDGFYPSDIAVEWESNG |
| | KPSKQSNNKYAASSYLSLT | ALTSGVHTFPAVLQSSGLY | QPENNYKTTPPVLDSDGS |
| | PEQWKSHRSYSCQVTHE | SLSSVVTVPSSSLGTQTYIC | FTLVSKLTVDKSRWQQGN |
| | GSTVEKTVAPTECS | NVNHKPSNTKVDKKVEPK | VESCSVMHEALHNHYTQ |
| | | SCDKTHTCPPCPAPELLGG | KSLSLSPG |
| | | PSVFLFPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPSRDELTKNQVSL | |
| | | TCLVKGFYPSDIAVEWES | |
| | | NGQPENNYKTTPPVLKSD | |
| | | GSFFLYSDLTVDKSRWQQ | |
| | | GNVESCSVMHEALHNHY | |
| | | TQKSLSLSPGKGGGGGGG | |
| | | GGGGGGGGGGGGGDKT | |
| | | HTCPPCPAPELLGGPSVFL | |
| | | FPPKPKDTLMISRTPEVTC | |
| | | VVVDVSHEDPEVKFNWY | |
| | | VDGVEVHNAKTKPREEQY | |
| | | NSTYRVVSVLTVLHQDWL | |
| | | NGKEYKCKVSNKALPAPIE | |
| | | KTISKAKGQPREPQVYTLP | |
| | | PCRDKLTKNQVSLMICLVK | |
| | | GFYPSDIAVEWESNGQPE | |
| | | NNYKTTPPVLDSDGSFFLY | |
| | | SKLTVDKSRWQQGNVFS | |
| | | CSVMHEALHNHYTQKSLS | |
| | | LSPGKGGGGGGGGGGG | |
| | | GGGGGGGGGDKTHTCPP | |
| | | CPAPELLGGPSVFLFPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPCR | |
| | | DKLTKNQVSLWCLVKGFY | |
| | | PSDIAVEWESNGQPENNY | |
| | | KTTPPVLDSDGSFFLYSKLT | |
| | | VDKSRWQQGNVFSCSV | |
| | | MHEALHNHYTQKSLSLSP | |
| | | G | |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Suffate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 55. Design and Purification of Fc-Antigen Binding Domain Construct 16 with an Anti-CD20 Antigen Binding Domain or an Anti-PD-L1 Antigen Binding Domain

### Protein Expression

A construct formed from a singly branched Fc domain where the branch point is at the C-terminal Fc domain was made as described below. Fc-antigen binding domain construct 16 (CD20) and construct 4 (PD-L1) each includes two distinct Fc domain monomer containing polypeptides (two copies of either an anti-CD20 long Fc chain (SEQ ID NO: 72) or an anti-PD-L1 long Fc chain (SEQ ID NO: 73), and four copies of a short Fc chain (SEQ ID NO: 63)) and three copies of either an anti-CD20 light chain polypeptide (SEQ ID NO: 61) or an anti-PD-L1 light chain polypeptide (SEQ ID NO: 49), respectively. The long Fc chain contains an Fc domain monomer with reverse charge mutations K409D and D399K (to promote homodimenzation) in a tandem series with two Fc domain monomers, in tandem, that each have an E357K charge mutation and S354C and T366W protuberance-forming mutations (to promote heterodimerization), and either anti-CD20 VH and CH1 domains (Kabat positions 1-220) at the N-terminus (construct 10 (CD20) or anti-PD-L1 VH and CH1 domains (Kabat positions 1-220) at the N-terminus (construct 10 (PD-L1)). The short Fc chain contains an Fc domain monomer with a K370D charge mutation and Y349C, T366S, L368A, and Y407V cavity-forming mutations (to promote heterodimerization). The anti-CD20 light chain or PD-L1 light chain can also be expressed fused to the N-terminus of the long Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 13 were encoded by three separate plasmids (one plasmid encoding the light chain (anti-CD20 or anti-PD-L1), one plasmid encoding the long Fc chain (anti-CD20 or anti-PD-L1) and one plasmid encoding the short Fc chain:

TABLE 13
Construct 16 (CD20) and Construct 16 (PD-L1) sequences

| | | Long Fc chain | |
|---|---|---|---|
| | | (with anti-CD20 or anti- | |
| Construct | Light chain | PD-L1 VH and CH1) | Short Fc chain |
| Construct 16 | SEQ ID NO: 61 | SEQ ID NO: 72 | SEQ ID NO: 63 |
| (CD20) | DIVMTQTPLSLPVTPGEPA | QVQLVQSGAEVKKPGSSV | DKTHTCPPCPAPELLGGPS |
| | SISCRSSKSLLHSNGITYLY | KVSCKASGYAFSYSWINW | VFLEPPKPKDTLMISRTPE |
| | WYLQKPGQSPQLLIYQMS | VRQAPGQGLEWMGRIFP | VTCVVVDVSHEDPEVKFN |
| | NLVSGVPDRFSGSGSGTD | GDGDTDYNGKFKGRVTIT | WYVDGVEVHNAKTKPRE |
| | FTLKISRVEAEDVGVYYCA | ADKSTSTAYMELSSLRSED | EQYNSTYRVVSVLTVLHQ |
| | QNLELPYTFGGGTKVEIKR | TAVYYCARNVFDGYWLVY | DWLNGKEYKCKVSNKALP |
| | TVAAPSVFIFPPSDEQLKS | WGQGTIATTVSSASTKGPS | APIEKTISKAKGQPREPQV |
| | GTASVVCLLNNFYPREAK | VFPLAPSSKSTSGGTAALG | CTLPPSRDELTKNQVSLSC |
| | VQWKVDNALQSGNSQES | CLVKDYFPEPVTVSWNSG | AVDGFYPSDIAVEWESNG |
| | VTEQDSKDSTYSLSSTLTLS | ALTSGVHTFPAVLQSSGLY | QPENNYKTTPPVLDSDGS |
| | KADYEKHKVYACEVTHQG | SLSSVVTVPSSSLGTQTYIC | FELVSKLIVDKSRWQQGN |
| | LSSPVTKSFNRGEC | NVNHKPSNTKVDKKVEPK | VFSCSVMHEALHNHYTQ |
| | | SCDKTHTCPPCPAPELLGG | KSLSLSPG |
| | | PSVFLEPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVKSNKA | |
| | | LPAPIEKTISKAKGQPREP | |
| | | QVYTLPPCRDKLTKNOVS | |
| | | LWCLVKGFYPSDIAVEWE | |
| | | SNGQPENNYKTTPPVLDS | |
| | | DGSFELYSKLTs/DIKSRWQ | |
| | | QGNVFSCSVMHEALHNH | |
| | | YTQKSLSLSPGKGGGGGG | |
| | | GGGGGGGGGGGGGGDK | |
| | | THTCPPCPAPELLGGPSVF | |
| | | LFPPKPKDTLMISRTPEVT | |
| | | CVVVDVSHEDPEVKFNW | |
| | | YVDGVEVHNAKTKPREEQ | |
| | | YNSTYRVVSVLTVLHQDW | |
| | | LNGKEYKCKVSNKALPAPI | |
| | | EKTISKAKGQPREPQVYTL | |
| | | PPCRDKLTKNQVSLWCLV | |
| | | KGFYPSDIAVEWESNGOP | |
| | | ENNYKTTPPVLDSDGSFFL | |
| | | YSKLTVDKSRWQQGNVF | |
| | | SCSVMHEALHNHYTQKSL | |
| | | SLSPGKGGGGGGGGGGG | |
| | | GGGGGGGGGDKTHTCPP | |
| | | CPAPELLGGPSVFLEPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPSR | |
| | | DELTKNQVSLTCLVKGFYP | |
| | | SDIAVEWESNGQPENNYK | |
| | | TTPPVLKSDGSFFLYSDLTV | |
| | | DKSRWQQGNVESCSVM | |
| | | HEALHNHYTQKSLSLSPG | |
| Construct 16 | SEQ ID NO: 49 | SEQ ID NO: 73 | SEQ ID NO: 63 |
| (PD-L1) | QSALTQPASVSGSPGQSIT | EVQLLESGGGLVQPGGSL | DKTHTCPPCPAPELLGGPS |
| | ISCTGTSSDVGGYNYVSW | RLSCAASGFTESSYIMMW | VFLEPPKPKDTLMISRTPE |
| | YQQHPGKAPKLMIYDVSN | VRQAPGKGLEWVSSIYPS | VTCVVVDVSHEDPEVKFN |
| | RPSGVSNRFSGSKSGNTA | GGITFYADTVKGRFTISRD | WYVDGVEVHNAKTKPRE |
| | SLTISGLOAEDEADYYCSS | NSKNTLYLQMNSLRAEDT | EQYNSTYRVVSVLTVLHQ |
| | YTSSSTRVFGTGTKVTVLG | AVYYCARIKLGTVTTVDY | DWLNGKEYKCKVSNKALP |
| | QPKANPTVTLFPPSSEELQ | WGQGTLVTVSSASTKGPS | APIEKTISKAKGQPREPQV |
| | ANKATLVCLISDFYPGAVT | VFPLAPSSKSTSGGTAALG | CTLPPSRDELTKNQVSLSC |
| | VAWKADGSPVKAGVETT | CLVKDYFPEPVTVSWNSG | AVDGFYPSDIAVEWESNG |
| | KPSKQSNNKYAASSYLSLT | ALTSGVHTFPAVLQSSGLY | OPENNYKTTPPVLDSDGS |
| | PEQWKSHRSYSCQVTHE | SLSSVVTVPSSSLGTQTYIC | FELVSKLTVDKSRWQQGN |
| | GSTVEKTVAPTECS | NVNHKPSNTKVDKKVEPK | VESCSVMHEALHNHYTQ |
| | | SCDKTHTCPPCPAPHIGG | KSLSLSPG |
| | | PSVFLEPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGOPREP | |
| | | QVYTLPPCRDKLTKNQVS | |
| | | LWCLVKGFYPSDIAVEWE | |
| | | SNGQPENNYKIIPPVLDS | |
| | | DGSFFLYSKLTVDKSRWQ | |
| | | QGNVFSCSVMHEALHNH | |
| | | YTQKSLSLSPGKGGGGGG | |
| | | GGGGGGGGGGGGGGDK | |
| | | THTCPPCPAPELLGGPSVF | |
| | | LFPPKPKDTLMISRTPEVT | |
| | | CVVVDVSHEDPEVKFNW | |
| | | YVDGVEVHNAKTKPREEQ | |
| | | YNSTYRVVSVLTVLHQDW | |
| | | LNGKEYKCKVSNKALPAPI | |
| | | EKTISKAKGQPREPQVYTL | |
| | | PPCRDKLTKNQVSLWCLV | |
| | | KGFYPSDIAVEWESNGQP | |
| | | ENNYKTTPPVLDSDGSFFL | |
| | | YSKLTVDKSRWQQGNVF | |
| | | SCSVMHEALHNHYTQKSL | |
| | | SLSPGKGGGGGGGGGGG | |
| | | GGGGGGGGGDKTHTCPP | |
| | | CPAPELLGGPSVFLEPPKP | |
| | | KDTLMISRTPEVTCVVVD | |
| | | VSHEDPEVKFNWYVDGV | |
| | | EVHNAKTKPREEQYNSTY | |
| | | RVVSVLTVLHQDWLNGK | |
| | | EYKCKVSNKALPAPIEKTIS | |
| | | KAKGQPREPQVYTLPPSR | |
| | | DELTKNQVSLTCLVKGFYP | |
| | | SDIAVEWESNGQPENNYK | |
| | | TTPPVLKSDGSFFLYSDLTV | |
| | | DKSRWQQGNVFSCSVM | |
| | | HEALHNHYTQKSLSLSPG | |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 56. Design and Purification of Fc-Antigen Binding Domain Construct 19 with an Anti-CD20 Antigen Binding Domain or an Anti-PD-L1 Antigen Binding Domain

### Protein Expression

A construct formed from a singly branched Fc domain where the branch point is at neither the N-terminal or C-terminal Fc domain was made as described below. Fc-antigen binding domain construct 19 (CD20) and construct 19 (PD-L1) each include two distinct Fc domain monomer containing polypeptides (two copies of either an anti-CD20 long Fc chain (SEQ ID NO: 74) or an anti-PD-L1 long Fc chain (SEQ ID NO: 75), and four copies of a short Fc chain (SEQ ID NO: 63)) and copies of either an anti-CD20 light chain polypeptide (SEQ ID NO: 61) or an anti-PD-L1 light chain polypeptide (SEQ ID NO: 49), respectively. The long Fc chain contains an Fc domain monomer with an E357K charge mutation and S354C and T366W protuberance-forming mutations (to promote heterodimerization), in a tandem series with an Fc domain monomer with reverse charge mutations K409D and D399K (to promote homodimerization), in a tandem series with an Fc domain monomer with an E357K charge mutation and S354C and T366W protuberance-forming mutations (to promote heterodimerization), and either anti-CD20 VH and CH1 domains (Kabat positions 1-220) at the N-terminus (construct 19 (CD20) or anti-PD-L1 VH and CH1 domains (Kabat positions 1-220) at the N-terminus (construct 19 (PD-L1)). The short Fc chain contains an Fc domain monomer with a K370D charge mutation and Y349C, T366S, L368A, and Y407V cavity-forming mutations (to promote heterodimerization). The anti-CD20 light chain or anti-PD-L1 light chain can also be expressed fused to the N-terminus of the long Fc chain as part of an scFv. DNA sequences were optimized for expression in mammalian cells and cloned into the pcDNA3.4 mammalian expression vector. The DNA plasmid constructs were transfected via liposomes into human embryonic kidney (HEK) 293 cells. The following amino acid sequences for each construct in Table 14 were encoded by three separate plasmids (one plasmid encoding the light chain (anti-CD20 or anti-PD-L1), one plasmid encoding the long Fc chain (anti-CD20 or anti-PD-L1) and one plasmid encoding the short Fc chain:

TABLE 15
Construct 19 (CD20) and Construct 19 (PD-L1) sequences

| | | Long Fc chain | |
|---|---|---|---|
| | | (with anti-CD20 or | |
| Construct | Light chain | anti-PD-L1 VH and CH1) | Short Fc chain |
| Construct 19 | SEQ ID NO: 61 | SEQ ID NO: 74 | SEQ ID NO: 63 |
| (CD20) | DIVMTQTPLSLPVIPGEPA | QVQLVQSGAEVKKPGSSV | DKTHTCPPCPAPELLGGPS |
| | SISCRSSKSLLHSNGITYLY | KVSCKASGYAFSYSWINW | VFLEPPKPKDTLMISRTPE |
| | WYLQKPGQSPQLLIYQMS | VRQAPGQGLEWMGRIFP | VTCVVVDVSHEDPEVKFN |
| | NLVSGVPDRFSGSGSGTD | GDGDTDYNGKEKGRVTIT | WYVDGVEVHNAKTKPRE |
| | FTLKISRVEAEDVGVYYCA | ADKSTSTAYMELSSLRSED | EQYNSTYRVVSVLTVLHQ |
| | QNLELPYTFGGGIKVEIKR | TAVYYCARNVFDGYWLVY | DWLNGKEYKCKVSNKALP |
| | TVAAPSVFIFPPSDEQLKS | WGQGTLVIVSSASTKGPS | APIEKTISKAKGQPREPQV |
| | GTASVVCLLNNFYPREAK | VFPLAPSSKSTSGGTAALG | CTLPPSRDELTKNQVSLSC |
| | VQWKVDNALQSGNSQES | CLVKDYFPEPVTVSWNSG | AVDGFYPSDIAVEWESNG |
| | VTEQDSKDSTYSLSSTLTLS | ALTSGVHITPAVLQSSGLY | QPENNYKTTPPVLDSDGS |
| | KADYEKHKVYACEVTHQG | SLSSVVTVPSSSLGTQTYIC | FTLVSKLTVDKSRWQQGN |
| | LSSPVTKSFNRGEC | NVNHKPSNTKVDKKVEPK | VFSCSVMHEALHNHYTQ |
| | | SCDKTHTCPPCPAPELLGG | KSLSLSPG |
| | | PSVFLFPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPCRDKLTKNQVS | |
| | | LWCLVKGFYPSDIAVEWE | |
| | | SNGQPENNYKTTPPVLDS | |
| | | DGSFFLYSKLTVDKSRWQ | |
| | | QGNVESCSVMHEALHNH | |
| | | YTQKSLSLSPGKGGGGGG | |
| | | GGGGGGGGGGGGGGDK | |
| | | THTCPPCPAPELLGGPSVF | |
| | | LEPPKPKDTLNIISRTPEVT | |
| | | CVVVDVSHEDPEVKFNW | |
| | | YVDGVEVHNAKTKPREEQ | |
| | | YNSTYRVVSVLTVLHQDW | |
| | | LNGKEYKCKVSNKALPAPI | |
| | | EKTISKAKGQPREPQVYTL | |
| | | PPSRDELTKNQVSLTCLVK | |
| | | GFYPSDIAVEWESNGQPE | |
| | | NNYKTTPPVLKSDGSFFLY | |
| | | SDLTVDKSRWQQGNVES | |
| | | CSVMHEALHNHYTQKSLS | |
| | | LSPGGKGGGGGGGGGG | |
| | | GGGGGGGGGGDKTHTC | |
| | | PPCPAPELLGGPSVFLEPP | |
| | | KPKDTLMISRTPEVTCVVV | |
| | | DVSHEDPEVKFNWYVDG | |
| | | VEVHNAKTKPREEQYNST | |
| | | YRVVSVLTVLHQDWLNG | |
| | | KEYKCKVSNKALPAPIEKTI | |
| | | SKAKGQPREPQVYTLPPC | |
| | | RDKLTKNQVSLWCLVKGF | |
| | | YPSDIAVEWESNGQPENN | |
| | | YKTTPPVLDSDGSFFLYSKL | |
| | | TVDKSRWQQGNVFSCSV | |
| | | MHEALHNHYTQKSLSLSP | |
| | | G | |
| Construct 19 | SEQ ID NO: 49 | SEQ ID NO: 75 | SEQ ID NO: 63 |
| (PD-L1) | QSALTQPASVSGSPGQSIT | EVQLLESGGGLVQPGGSL | DKTHTCPPCPAPELLGGPS |
| | ISCTGTSSDVGGYNYVSW | RLSCAASGFTESSYIMMW | VFLEPPKPKDTLMISRTPE |
| | YQQHPGKAPKLMIYDVSN | VRQAPGKGLEWVSSIYPS | VTCVVVDVSHEDPEVKFN |
| | RPSGVSNRFSGSKSGNTA | GGITFYADTVKGRFTISRD | WYVDGVEVHNAKTKPRE |
| | SLTISGLQAEDEADYYCSS | NSKNTLYLQMNSLRAEDT | EQYNSTYRVVSVLTVLHQ |
| | TISSSTRVFGTGTKVTVLG | AVYYCARIKLGTVTTVDY | DWLNGKEYKCKVSNKALP |
| | QPKANPTVTLFPPSSEELQ | WGQGTLVIVSSASTKGPS | APIEKTISKAKGQPREPQV |
| | ANKATLVCLISDFYPGAVT | VFPLAPSSKSTSGGTAALG | CTLPPSRDELTKNQVSLSC |
| | VAWKADGSPVKAGVEII | CLVKDYFPEPVTVSWNSG | AVDGFYPSDIAVEWESNG |
| | KPSKQSNNKYAASSYLSLT | ALTSGVHTFPAVLOSSGLY | QPENNYKTTPPVLDSDGS |
| | PEQWKSHRSYSCQVTHE | SLSSVVTVPSSSLGTQTYIC | FTLVSKLTVDKSRWQQGN |
| | GSTVEKTVAPTECS | NVNHKPSNTKVDKKVEPK | VESCSVMHEALHNHYTQ |
| | | SCDKTHTCPPCPAPELLGG | KSLSLSPG |
| | | PSVFLFPPKPKDTLMISRT | |
| | | PEVTCVVVDVSHEDPEVK | |
| | | FNWYVDGVEVHNAKTKP | |
| | | REEQYNSTYRVVSVLTVLH | |
| | | QDWLNGKEYKCKVSNKA | |
| | | LPAPIEKTISKAKGQPREP | |
| | | QVYTLPPCRDKLTKNQVS | |
| | | LWCLVKGFYPSDIAVEWE | |
| | | SNGQPENNYKTTPPVLDS | |
| | | DGSFELYSKLTVDKSRWQ | |
| | | QGNVFSCSVMHEALHNH | |
| | | YTQKSISISPGKGGGGGG | |
| | | GGGGGGGGGGGGGGDK | |
| | | THTCPPCPAPELLGGPSVF | |
| | | LFPPKPKDTLMISRTPEVT | |
| | | CVVVDVSHEDPEVKFNW | |
| | | YVDGVEVHNAKTKPREEQ | |
| | | YNSTYRVVSVLTVLHQDW | |
| | | LNGKEYKCKVSNKALPAPI | |
| | | EKTISKAKGQPREPQVYTL | |
| | | PPSRDELTKNQVSLTCLVK | |
| | | GFYPSDIAVEWESNGQPE | |
| | | NNYKTTPPVLKSDGSFFLY | |
| | | SDLTVDKSRWQQGNVFS | |
| | | CSVMHEALHNHYTQKSLS | |
| | | LSPGGKGGGGGGGGGG | |
| | | GGGGGGGGGGDKTHTC | |
| | | PPCPAPELLGGPSVFLFPP | |
| | | KPKDTLMISRTPEVTCVVV | |
| | | DVSHEDPEVKFNWYVDG | |
| | | VEVHNAKTKPREEQYNST | |
| | | YRVVSVLTVLHQDWLNG | |
| | | KEYKCKVSNKALPAPIEKTI | |
| | | SKAKGQPREPQVYTLPPC | |
| | | RDKLTKNQVSLWCLVKGF | |
| | | YPSDIAVEWESNGQPENN | |
| | | YKTTPPVLDSDGSFFLYSKL | |
| | | TVDKSRWQQGNVFSCSV | |
| | | MHEALHNHYTQKSLSLSP | |
| | | G | |

The expressed proteins were purified from the cell culture supernatant by Protein A-based affinity column chromatography, using a Poros MabCapture A (LifeTechnologies) column. Captured Fc-antigen binding domain constructs were washed with phosphate buffered saline (low-salt wash) and eluted with 100 mM glycine, pH 3. The eluate was quickly neutralized by the addition of 1 M TRIS pH 7.4 and sterile filtered through a 0.2 μm filter. The proteins were further fractionated by ion exchange chromatography using Poros XS resin (Applied Biosciences). The column was pre-equilibrated with 50 mM MES, pH 6 (buffer A), and the sample was eluted with a step gradient using 50 mM MES, 400 mM sodium chloride, pH 6 (buffer B) as the elution buffer. After ion exchange, the target fraction was buffer exchanged into PBS buffer using a 10 kDa cut-off polyether sulfone (PES) membrane cartridge on a tangential flow filtration system. The samples were concentrated to approximately 30 mg/mL and sterile filtered through a 0.2 μm filter.

### Non-Reducing Sodium Dodecyl Sulfate-Polyacrylamide Gel Electrophoresis (SDS-PAGE)

Samples were denatured in Laemmli sample buffer (4% SDS, Bio-Rad) at 95° C. for 10 min. Samples were run on a Criterion TGX stain-free gel (4-15% polyacrylamide, Bio-Rad). Protein bands were visualized by UV illumination or Coommassie blue staining. Gels were imaged by ChemiDoc MP Imaging System (Bio-Rad). Quantification of bands was performed using Imagelab 4.0.1 software (Bio-Rad).

### Example 57. Complement-Dependent Cytotoxicity (CDC) Activation by Anti-CD20 Fc Constructs

A CDC assay was developed to test the degree to which anti-CD20 Fc constructs enhance CDC activity relative to an anti-CD20 monoclonal antibody, obinutuzumab. Anti-CD20 Fc constructs 4, 7, 8, 9, 10, 13, and 19 having the CDRs of Gazyva were produced as described in Examples 1, 2, and 51-56. Four versions of Construct 13 (CD20) were created that varied only in the size of the glycine spacer between the long chain Fc monomers of the long chain (G₄, G₁₀, G₁₅ and G₂₀ linkers). Each anti-CD20 Fc construct, and the obinutuzumab monoclonal antibody, was tested in a CDC assay performed as follows:

Daudi cells grown in RPMI-1640 supplemented with 10% heat-inactivated FBS were pelleted, washed 1× with ice-cold PBS and resuspended in RPMI-1640 containing 0.1% BSA at a concentration of 1.0×10⁶ viable cells per mL. Fifty microliters of this cell suspension was added to all wells (except plate edges) of 96-well plates. Plates were kept on ice until all additions had been made. Test articles were serially diluted four-fold from a starting concentration of 450 nM in RPMI-1640+ BSA. A total of ten concentrations was tested for each test article. Fifty microliters each was added to plated Daudi cells. Normal or C1q-depleted human complement serum (Quidel, San Diego, Calif.) was diluted 1:5 in RPMI-1640+ BSA. Fifty microliters each was added to plated Daudi cells. Six normal serum control wells received cells, media only (no treatment) and 1/5 normal serum (Normal Background). Three of these wells also received 16.5 μL Triton X-100 (Promega, Madison, Wis.) (Normal Lysis Control). C1q-depleted Background and Lysis Controls were similarly prepared. PBS was added to all plate edge wells. Plates were incubated for 2 h at 37° C. After 2 h, 50 μL pre-warmed Alamar blue (Thermo, Waltham, Mass.) was added to all wells (expect plate edges). Plates were returned to the incubator overnight (18 h at 37° C.). After 18 h fluorescence was measured in a FlexStation 3. Plates were top-read using 544/590 Ex/Em filters and Auto Cut-Off. Means were calculated for Normal Background, Normal Lysis Control, C1q-depleted Background and C1q-depleted Lysis Control wells. Percent cell lysis was calculated as: % Cell Lysis=(RFU Test−RFU Background)/(RFU Lysis Control−RFU Background)*100. The EC50 (nM) was determined for each construct.

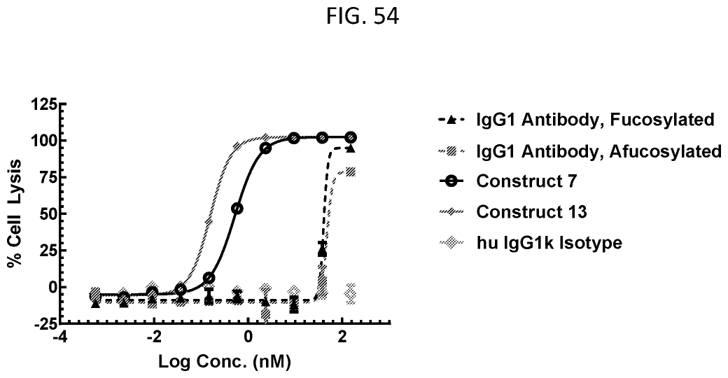

As depicted in Table 15, anti-CD20 Fc constructs induced CDC in Daudi cells and demonstrated greater potency in enhancing cytotoxicity relative to the obinutuzumabmonoclonal antibody, as evidenced by lower EC50 values. FIG. 54 illustrates results for anti-CD20 construct 7 and anti-CD20 construct 13, and shows that each Fc construct induces greater cell lysis at lower concentrations relative to both fucosylated anti-CD20 IgG1 antibody and Gazyva controls.

TABLE 15
Potency of anti-CD20 Fc constructs to induce CDC in Daudi cells

| | | EC50 (nM) | | |
|---|---|---|---|---|
| Construct¹ | n | Range | Mean | SD |
| IgG1 Antibody, | 5 | 38-65 | 47 | 11 |
| Fucosylated | | | | |
| S3I-AA-OBI | 5 | 0.39-0.70 | 0.55 | 0.12 |
| Construct 7 | | | | |
| (anti-CD20) | | | | |
| S5I-AA-OBI | 1 | 0.88 | 0.88 | N/A |
| Construct 10 | | | | |
| (anti-CD20) | | | | |
| S3W-AA-OBI | 3 | 0.15-0.18 | 0.17 | 0.017 |
| Construct 8² | | | | |
| (anti-CD20) | | | | |
| S3A-AA-OBI | 3 | 0.19-0.20 | 0.19 | 0.0036 |
| Construct 9 | | | | |
| (anti-CD20) | | | | |
| S3Y-AA-OBI4 | 3 | 0.11-0.19 | 0.16 | 0.045 |
| Construct 13 | | | | |
| (anti-CD20), | | | | |
| G₄ linker | | | | |
| S3Y-AA-OBI10 | 3 | 0.11-0.19 | 0.16 | 0.047 |
| Construct 13 | | | | |
| (anti-CD20), | | | | |
| G¹⁰ linker | | | | |
| S3Y-AA-OBI15 | 4 | 0.13-0.36 | 0.22 | 0.098 |
| Construct 13 | | | | |
| (anti-CD20), | | | | |
| G¹⁵ linker | | | | |
| S3Y-AA-OBI | 4 | 0.13-0.22 | 0.18 | 0.046 |
| Construct 13 | | | | |
| (anti-CD20), | | | | |
| G²⁰ linker | | | | |
| S5X-AA-OBI | 1 | 0.20 | 0.20 | N/A |
| Construct 19 | | | | |
| (anti-CD20) | | | | |
| S3L-AAA-OBI3 | 4 | 0.12-0.18 | 0.15 | 0.031 |
| Construct 4 | | | | |
| (anti-CD20) | | | | |

¹All constructs included G20 linkers unless otherwise noted.
²Construct contains a spontaneous E388D mutation.

### Example 58. Complement-Dependent Cytotoxicity (CDC) Activation by Anti-PD-L1 Fc Constructs

A CDC assay was developed to test the degree to which anti-PD-L1 Fc constructs enhance CDC activity relative to an anti-PD-L1 monoclonal antibody, avelumab (Bavencio). Anti-PD-L1 Fc constructs 7, 8, 10, 13, and 19 having the CDRs of avelumab were produced as described in Examples 1, 2, and 51-56. Four versions of Construct 13 (PD-L1) were created that varied only in the size of the glycine spacer between the long chain Fc monomers of the long chain (G₄, G₁₀, G₁₅ and G₂₀ linkers). Each anti-PD-L1 Fc construct, and the avelumab monoclonal antibody, was tested in a CDC assay performed as follows:

The Human Embryonic Kidney (HEK) cell line transfected to stably express the human PD-L1 gene (CrownBio) were cultured in DMEM, 10% FBS, and 2 μg/mL puromycin as the selection marker. The cells were harvested and diluted in X-Vivo-15 media without genetecin or phenol red (Lonza). One hundred μl of HEK-PD-L1 cells at 6×10⁵ cells/mL were plated in a 96 well tissue culture treated flat bottom plate (BD Falcon). The Fc constructs and antibodies were serially diluted 1:3 in X-Vivo-15 media. Fifty μL of the diluted constructs were added to the wells on top of the target cells. Fifty μl of undiluted Human Serum Complement (Quidel Corporation) were added to each of the wells. The assay plate was then incubated for 2 h at 37° C. After the 2 h incubation 20 μL of WST-1 Cell Proliferation Reagent (Roche Diagnostics Corp) were added to each well and incubated overnight at 37° C. The next morning the assay plate was placed on a plate shaker for 2-5 min. Absorbance was measured at 450 nm with correction at 600 nm on a spectrophotometer (Molecular Devices SPECTRAmax M2). The EC50 (nM) was determined for each construct.

As depicted in Table 16, some of the anti-PD-L1 Fc constructs induced CDC in HEK cells that express human PD-L1.

TABLE 16
Potency of anti-PD-L1 Fc constructs to induce
CDC in PD-L1 expressing HEK cells

| | | EC50 (nM) | | |
|---|---|---|---|---|
| Construct¹ | n | Range | Mean | SD |
| IgG1 Antibody, | 7 | No CDC | No CDC | N/A |
| Fucosylated | | activity³ | activity³ | |
| IgG1 Antibody, | 1 | No CDC | No CDC | N/A |
| Afucosylated | | activity³ | activity³ | |
| S3I-AA-AVE | 6 | No CDC | No CDC | N/A |
| Construct 7² | | activity³ | activity³ | |
| (anti-PD-L1) | | | | |
| S5I-AA-AVE | 2 | No CDC | No CDC | N/A |
| Construct 10 | | activity³ | activity³ | |
| (anti-PD-L1) | | | | |
| S3W-AA-AVE | 3 | 1.2-2.4 | 1.7 | 0.63 |
| Construct 8² | | | | |
| (anti-PD-L1) | | | | |
| S3Y-AA-AVE4 | 2 | 0.43-0.84 | 0.64 | 0.29 |
| Construct 13 | | | | |
| (anti-PD-L1), | | | | |
| G₄ linker | | | | |
| S3Y-AA-AVE10 | 2 | 0.58-1.0 | 0.81 | 0.33 |
| Construct 13 | | | | |
| (anti-PD-L1), | | | | |
| G₁₀ linker | | | | |
| S3Y-AA-AVE15 | 2 | 0.56-1.1 | 0.85 | 0.41 |
| Construct 13 | | | | |
| (anti-PD-L1), | | | | |
| G₁₅ linker | | | | |
| S3Y-AA-AVE | 15 | 0.38-3.6 | 1.4 | 1.2 |
| Construct 13 | | | | |
| (anti-PD-L1), | | | | |
| G₂₀ linker | | | | |
| S5X-AA-AVE | 3 | 0.88-3.4 | 1.9 | 1.4 |
| Construct 19 | | | | |
| (anti-PD-L1) | | | | |

¹All constructs included G20 linkers unless otherwise noted.
²Construct contains a spontaneous E388D mutation.
³Construct did not produce measurable CDC under the assay conditions.

### Example 59. Antibody-Dependent Cellular Phagocytosis (ADCP) Activation by Anti-CD20 Fc Constructs

ADCP Reporter Assay

An ADCP reporter assay was developed to test the degree to which anti-CD20 Fc constructs activate FcγRIIa signaling, thereby enhancing ADCP activity, relative to an anti-CD20 monoclonal obinutuzumab antibody (Gazyva). Anti-CD20 Fc constructs 4, 7, 8, 9, 10, 13, and 19 having the CDRs of Gazyva were produced as described in Examples 1, 2, and 51-56. Four versions of Construct 13 (CD20) in which the glycine spacer between the long chain Fc monomers varied in size (G4, G10, G15 and G20 linkers) were tested. Each anti-CD20 Fc construct, and fucosylated and afucosylated obinutuzumab monodonal antibodies, were tested in an ADCC reporter assay performed as follows:

Raji target cells (1.5×10⁴ cells/well) and Jurkat/FcγRIIa-H effector cells (Promega) (3.5×10⁴ cells/well) were resuspended in RPMI 1640 Medium supplemented with 4% low IgG serum (Promega) and seeded in a 96-well plate with serially diluted anti-CD20 Fc constructs. After incubation for 6 h at 37° C. in 5% CO₂, the luminescence was measured using the Bio-Glo Luciferase Assay Reagent (Promega) according to the manufacturer's protocol using a PHERAstar FS luminometer (BMG LABTECH).

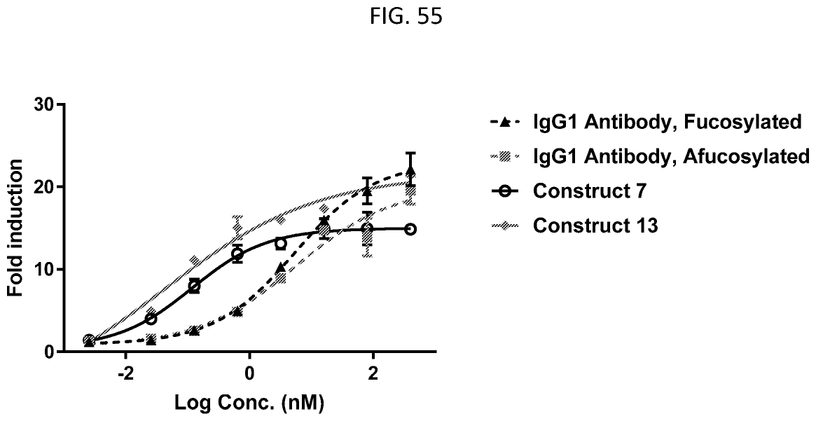

As depicted in Table 17, anti-CD20 Fc constructs induced FcγRIIa signaling in an ADCP reporter assay and demonstrated greater potency in enhancing ADCP activity relative to the obinutuzumab monodonal antibody, as evidenced by lower EC50 values. FIG. 55 illustrates results for anti-CD20 construct 7 and anti-CD20 construct 13, and shows that each Fc construct induces greater FcγRIIa signaling at lower concentrations relative to both fucosylated and afucosylated anti-CD20 IgG1 antibody controls.

TABLE 17
Potency of anti-CD20 Fc constructs to induce
FcγRIIa signaling in an ADCP reporter assay

| | | EC50 (nM) | | |
|---|---|---|---|---|
| Construct¹ | n | Range | Mean | SD |
| IgG1 Antibody, | 6 | 4.5-10.8 | 7.1 | 2.2 |
| Fucosylated | | | | |
| IgG1 Antibody, | 3 | 5.5-6.1 | 5.8 | 0.3 |
| Afucosylated | | | | |
| S3I-AA-OBI | 6 | 0.0016-0.12 | 0.067 | 0.0409 |
| Construct 7 | | | | |
| (anti-CD20) | | | | |
| S5I-AA-GBI | 1 | 0.11 | 0.11 | N/A |
| Construct 10 | | | | |
| (anti-CD20) | | | | |
| S3W-AA-OBI | 1 | 0.087 | 0.087 | N/A |
| Construct 8² | | | | |
| (anti-CD20) | | | | |
| S3A-AA-OBI | 1 | 0.026 | 0.026 | N/A |
| Construct 9 | | | | |
| (anti-CD20) | | | | |
| S3Y-AA-OBI4 | 1 | 0.061 | 0.061 | N/A |
| Construct 13 | | | | |
| (anti-CD20), G₄ | | | | |
| linker | | | | |
| S3Y-AA-OBI10 | 1 | 0.056 | 0.056 | N/A |
| Construct 13 | | | | |
| (anti-CD20), | | | | |
| G₁₀ linker | | | | |
| S3Y-AA-OBI15 | 1 | 0.074 | 0.074 | N/A |
| Construct 13 | | | | |
| (anti-CD20), | | | | |
| G₁₅ linker | | | | |
| S3Y-AA-OBI | 5 | 0.041-0.14 | 0.064 | 0.041 |
| Construct 13 | | | | |
| (anti-CD20), | | | | |
| G₂₀ linker | | | | |
| S5X-AA-OBI | 1 | 0.045 | 0.045 | N/A |
| Construct 19 | | | | |
| (anti-CD20) | | | | |
| S3L-AAA-OBI3 | 1 | 0.055 | 0.055 | N/A |
| Construct 4 | | | | |
| (anti-CD20) | | | | |

¹All constructs included G20 linkers unless otherwise noted.
²Construct contains a spontaneous E388D mutation.

ADCP Secondary Assay

Anti-CD20 Fc constructs 7, 8, 9, 13 (G20 linker), and 19 were tested in an additional ADCP assay to confirm the ADCP reporter assay results. Each anti-CD20 Fc construct, and fucosylated and afucosylated obinutuzumab monoclonal antibodies, were tested in an ADCC assay performed as follows: Monocytes were purified from frozen PBMCs and cultured in bags in the presence of M-CSF. IL-10 was added to the culture bags 2 days before the differentiated M2c macrophages were used in an ADCP assay. The total culture time for the monocytes/macrophages was 8 days. Anti-CD20 Fc constructs were 10-fold serially diluted and incubated with KILR Raji cells for 30 min. Macrophages were resuspended in assay medium in phenol red free RPMI (Life Technologies) containing 10% Super Low IgG Defined FBS (heat inactivated) from (Hydone), and added to the plate(s) containing the coated KILR Raji cells at a 8:1 (M2c) Effector:Target ratio and incubated for 24 hours. After the incubation time the PathHunter® ProLabel®/ProLink™ Detection Kit reagents were added to the plate(s) and read on the PHERAstar FS luminometer (BMG LABTECH) after a 60 min incubation at room temperature.

The results depicted in Table 18 demonstrate that anti-CD20 Fc constructs induced FcγRIIa signaling in the secondary ADCP assay and had greater potency in enhancing ADCP activity relative to fucosylated or afucosylated obinutuzumab monoclonal antibody, as evidenced by lower EC50 values. The results from the secondary ADCP assay were consistent with the results of the ADCP reporter assay.

TABLE 18
Potency of anti-CD20 Fc constructs
to induce ADCP in KILR Raji cells

| | | EC50 (nM) | | |
|---|---|---|---|---|
| Construct¹ | n | Range | Mean | SD |
| IgG1 Antibody, | 8 | 0.00053-1.8 | 0.82 | 0.82 |
| Fucosylated | | | | |
| IgG1 Antibody, | 4 | 0.0015-0.96 | 0.37 | 0.51 |
| Afucosylated | | | | |
| S3I-AA-OBI | 8 | 0.00058-0.27 | 0.042 | 0.095 |
| Construct 7 | | | | |
| (anti-CD20) | | | | |
| S3W-AA-OBI | 2 | 0.00015-0.053 | 0.026 | 0.037 |
| Construct 8² | | | | |
| (anti-CD20) | | | | |
| S3A-AA-OBI | 2 | 0.0020-0.089 | 0.045 | 0.061 |
| Construct 9 | | | | |
| (anti-CD20) | | | | |
| S3Y-AA-OBI | 4 | 0.000063-0.076 | 0.019 | 0.038 |
| Construct 13 | | | | |
| (anti-CD20) | | | | |
| S5X-AA-OBI | 2 | 0.00038-0.026 | 0.013 | 0.018 |
| Construct 19 | | | | |
| (anti-CD20) | | | | |

¹All constructs included G20 linkers unless otherwise noted.
²Construct contains a spontaneous E388D mutation.

### Example 60. Antibody-Dependent Cellular Phagocytosis (ADCP) Activation by Anti-PD-L1 Fc Constructs

ADCP Reporter Assay

An ADCP reporter assay was developed to test the degree to which anti-PD-L1 Fc constructs activate FcγRIIa signaling, thereby enhancing ADCP activity, relative to an anti-PD-L1 monoclonal antibody, avelumab (Bavencio). Anti-PD-L1 Fc constructs 4, 7, 8, 9, 10, 13, 16, and 19 having the CDRs of avelumab were produced as described in Examples 1, 2, and 51-56. Four versions of Construct 13 (PD-L1) in which the glycine spacer between the long chain Fc monomers varied in size (G4, G10, G15 and G20 linkers) were tested. Each anti-PD-L1 Fc construct, and fucosylated and afucosylated avelumab monodonal antibodies, were tested in an ADCC reporter assay performed as follows:

Target HEK-PD-L1 cells (1.5×10⁴ cells/well) and effector Jurkat/FcγRIIa-H cells (Promega) (3.5×10⁴ cells/well) were resuspended in RPMI 1640 Medium supplemented with 4% low IgG serum (Promega) and seeded in a 96-well plate with serially diluted anti-PD-L1 Fc constructs. After incubation for 6 hours at 37° C. in 5% CO2, the luminescence was measured using the Bio-Glo Luciferase Assay Reagent (Promega) according to the manufacturer's protocol using a PHERAstar FS luminometer (BMG LABTECH).

As depicted in Table 19, anti-PD-L1 Fc constructs induced FcγRIIa signaling in an ADCP reporter assay.

TABLE 19
Potency of anti-PD-L1 Fc constructs to induce
FcγRIIa signaling in an ADCP reporter assay

| Construct | | EC50 (nM) | | |
|---|---|---|---|---|
| Number¹ | n | Range | Mean | SD |
| IgG1 Antibody, | 6 | No effect³ | No effect³ | N/A |
| Fucosylated | | | | |
| IgG1 Antibody, | 1 | No effect³ | No effect³ | N/A |
| Afucosylated | | | | |
| S3I-AA-AVE | 6 | 0.012-0.036 | 0.026 | 0.012 |
| Construct 7² | | | | |
| (anti-PD-L1) | | | | |
| S5I-AA-AVE | 1 | 0.031 | 0.031 | N/A |
| Construct 10 | | | | |
| (anti-PD-L1) | | | | |
| S3W-AA-AVE | 1 | 0.028 | 0.028 | N/A |
| Construct 8² | | | | |
| (anti-PD-L1) | | | | |
| S3A-AA-AVE | 1 | 0.026 | 0.026 | N/A |
| Construct 9² | | | | |
| (anti-PD-L1) | | | | |
| S3Y-AA-AVE4 | 1 | 0.05 | 0.05 | N/A |
| Construct 13 | | | | |
| (anti-PD-L1), | | | | |
| G₄ linker | | | | |
| S3Y-AA-AVE10 | 1 | 0.085 | 0.085 | N/A |
| Construct 13 | | | | |
| (anti-PD-L1), | | | | |
| G₁₀ linker | | | | |
| S3Y-AA-AVE15 | 1 | 0.05 | 0.05 | N/A |
| Construct 13 | | | | |
| (anti-PD-L1), | | | | |
| G₁₅ linker | | | | |
| S3Y-AA-AVE | 6 | 0.027-0.052 | 0.038 | 0.01 |
| Construct 13 | | | | |
| (anti-PD-L1), | | | | |
| G₂₀ linker | | | | |
| S5X-AA-AVE | 1 | 0.033 | 0.033 | N/A |
| Construct 19 | | | | |
| (anti-PD-L1) | | | | |
| S5Y-AA-AVE | 1 | 0.04 | 0.04 | N/A |
| Construct 16 | | | | |
| (anti-PD-L1) | | | | |
| S3L-3AAA-AVE | 1 | 0.028 | 0.028 | N/A |
| Construct 4 | | | | |
| (anti-PD-L1) | | | | |

¹All constructs included G20 linkers unless otherwise noted.
²Construct contains a spontaneous E388D mutation.
³Construct did not induce measurable FcγRIIa signaling under the assay conditions.

ADCP Secondary Assay

Anti-PD-L1 Fc constructs 8, 9, and 13 (G20 linker) were tested in an additional ADCP assay to confirm the ADCP reporter assay results. Each anti-PD-L1 Fc construct, and fucosylated avelumab monoclonal antibody, were tested in an ADCC assay performed as follows:

M2c macrophages were seeded in a 96 well U-bottom ultra-low binding plate (Costar, 7007) at 2×10⁵ cells per well and allowed to equilibrate for at least 1 hour at 37° C., 5% CO2 humidified incubator. HEK293 PD-L1 cells were stained with calcein-AM (Invitrogen, C-3100) according to the manufacturer's protocol and pre-incubated with anti-PD-L1 constructs diluted 5-fold from 6.7 nM for 15 minutes at room temperature. They were then combined with macrophages at an effector:target ratio of 3:1 and incubated for 2 hours at 37° C., 5% CO2 incubator. The cells were transferred to a V-bottom 96 well plate for staining followed by washing with FACS buffer (PBS+2% FBS). Pooled cells were blocked using Fc block (Biolegend, 422302) and stained with anti-CD11 b-APC Ab (Biolegend, 301310) at 4° C. for 1 hour. Cells were washed with FACS buffer and read on BD FACS Verse. Analysis was done using FlowJo. Doublets were removed from calculation by FSC-H vs FSC-A plot. Cells that were positive for calcein-AM and CD11 b were considered as phagocytic events or double positive macrophages (DP). Percent phagocytosis was calculated by calculating (DP cells/Total target cells)*100.

The results depicted in Table 20 demonstrate that anti-PD-L1 Fc constructs induced ADCP in a secondary assay and had greater potency in enhancing ADCP activity relative to fucosylated avelumab monoclonal antibody, as evidenced by lower EC50 values. The results from the secondary ADCP assay were consistent with the results of the ADCP reporter assay.

TABLE 20
Potency of anti-PD-L1 Fc constructs to induce ADCP in a
FACS-based assay with HEK-PD-L1 cells and M2c macrophages

| Construct | | EC50 (nM) | | |
|---|---|---|---|---|
| Number¹ | n | Range | Mean | SD |
| IgG1 Antibody, | 1 | 0.211 | 0.211 | N/A |
| Fucosylated | | | | |
| S3W-AA-AVE | 1 | 0.054 | 0.054 | N/A |
| Construct 8² | | | | |
| (anti-PD-L1) | | | | |
| S3A-AA-AVE | 2 | 0.00097-0.0061 | 0.0035 | 0.0036 |
| Construct 9² | | | | |
| (anti-PD-L1) | | | | |
| S3Y-AA-AVE | 2 | 0.01947-0.05635 | 0.03791 | 0.026078 |
| Construct 13 | | | | |
| (anti-PD-L1), | | | | |
| G₂₀ linker | | | | |

¹All constructs included G20 linkers unless otherwise noted.
²Construct contains a spontaneous E388D mutation.

### Example 61. Antibody-Dependent Cell-Mediated Cytotoxicity (ADCC) Activation by Anti-CD20 Fc Constructs

ADCC Reporter Assay

An ADCC reporter assay was developed to test the degree to which anti-CD20 Fc constructs induce FcγRIIIa signaling and enhance ADCC activity relative to an anti-CD20 monoclonal antibody obinutuzumab (Gazyva). Anti-CD20 Fc constructs 4, 7, 8, 9, 10, 13, and 19 having the CDRs of Gazyva were produced as described in Examples 1, 2, and 51-56. Four versions of construct 13 (CD20) in which the glycine spacer between the long chain Fc monomers varied in size (G₄, G₁₀, G₁₅ and G₂₀ linkers) were tested. Each anti-CD20 Fc construct, and fucosylated and afucosylated obinutuzumab monoclonal antibodies, were tested in an ADCC reporter assay performed as follows:

Raji target cells (1.25×10⁴ cells/well) and Jurkat/FcγRIIa effector cells (Promega) (7.45×104 cells/well) were resuspended in RPMI 1640 Medium supplemented with 4% low IgG serum (Promega) and seeded in a 96-well plate with serially diluted anti-CD20 Fc constructs. After incubation for 6 hours at 37° C. in 5% CO2, the luminescence was measured using the Bio-Glo Luciferase Assay Reagent (Promega) according to the manufacturer's protocol using a PHERAstar FS luminometer (BMG LABTECH).

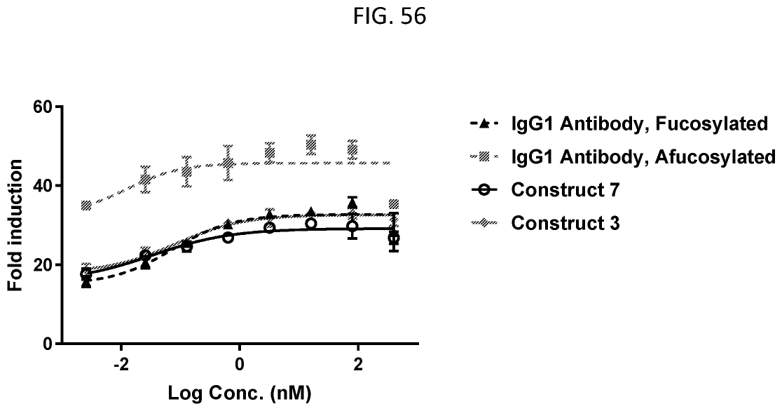

As depicted in Table 21, anti-CD20 Fc constructs induced FcγRIIa signaling in an ADCC reporter assay and demonstrated greater potency in enhancing ADCC activity relative to fucosylated obinutuzumab monoclonal antibody, as evidenced by lower EC50 values. FIG. 56 illustrates results for anti-CD20 construct 7 and anti-CD20 construct 13, and shows that each Fc construct induces greater FcγRIIa signaling at lower concentrations relative to the fucosylated anti-CD20 antibody, but were not as effective as the afuosylated anti-CD20 antibody.

TABLE 21
Potency of anti-CD20 Fc constructs to induce
FcγRIIIa signaling in an ADCC reporter assay

| | | EC50 (nM) | | |
|---|---|---|---|---|
| Construct¹ | n | Range | Mean | SD |
| IgG1 Antibody, | 6 | 4.5-10.8 | 7.1 | 2.2 |
| Fucosylated | | | | |
| IgG1 Antibody, | 3 | 5.5-6.1 | 5.8 | 0.3 |
| Afucosylated | | | | |
| S3I-AA-OBI | 6 | 0.0016-0.12 | 0.067 | 0.041 |
| Construct 7 | | | | |
| (anti-CD20) | | | | |
| S5I-AA-OBI | 1 | 0.11 | 0.11 | N/A |
| Construct 10 | | | | |
| (anti-CD20) | | | | |
| S3W-AA-OBI | 1 | 0.087 | 0.087 | N/A |
| Construct 8² | | | | |
| (anti-CD20) | | | | |
| S3A-AA-OBI | 1 | 0.026 | 0.026 | N/A |
| Construct 9 | | | | |
| (anti-CD20) | | | | |
| S3Y-AA-OBI4 | 1 | 0.061 | 0.061 | N/A |
| Construct 13 | | | | |
| (anti-CD20), | | | | |
| G₄ linker | | | | |
| S3Y-AA-OBI10 | 1 | 0.056 | 0.056 | N/A |
| Construct 13 | | | | |
| (anti-CD20), | | | | |
| G₁₀ linker | | | | |
| S3Y-AA-OBI15 | 1 | 0.074 | 0.074 | N/A |
| Construct 13 | | | | |
| (anti-CD20), | | | | |
| G₁₅ linker | | | | |
| S3Y-AA-OBI | 5 | 0.041-0.14 | 0.064 | 0.041 |
| Construct 13 | | | | |
| (anti-CD20), | | | | |
| G²⁰ linker | | | | |
| S5X-AA-OBI | 1 | 0.045 | 0.045 | N/A |
| Construct 19 | | | | |
| (anti-CD20) | | | | |
| S3L-AAA-OBI3 | 1 | 0.055 | 0.055 | N/A |
| Construct 4 | | | | |
| (anti-CD20) | | | | |

¹All constructs included G20 linkers unless otherwise noted.
²Construct contains a spontaneous E388D mutation.

ADCC Secondary Assay

Anti-CD20 Fc constructs 7, 8, 9, 13 (G20 linker), and 19 were tested in an additional ADCC assay to confirm the ADCC reporter assay results. Each anti-CD20 Fc construct, and fucosylated Gazyva monoclonal antibody, were tested in an ADCC assay performed as follows:

NK cells were thawed, resuspended at 250,000 cells/ml in Lonza LGM media and cultured overnight. Probes are serially diluted (10-fold) and incubated with KILR Raji cells at a 5:1 effector:target ratio (50,000:10,000 or 25,000:5,000: donor dependent) for 30 min. NK cells were counted, resuspended in assay medium in phenol red free RPMI (Life Technologies) containing 10% Super Low IgG Defined FBS (heat inactivated) from (Hyclone), and added to the plate(s) containing the coated KILR Raji cells. After 5 h 30 min the KILR detection reagents were mixed and added to the plate(s). Plates were read on the PHERAstar FS luminometer (BMG LABTECH) after a 30 min incubation at RT.

The results depicted in Table 22 demonstrate that anti-CD20 Fc constructs induced FcγRIIIa signaling in the secondary ADCC assay and had greater potency in enhancing ADCC activity relative to fucosylated Gazyva monoclonal antibody, as evidenced by lower EC50 values. The results from the secondary ADCC assay are consistent with the results of the ADCC reporter assay.

TABLE 22
Potency of anti-CD20 Fc constructs
to induce ADCC in KILR Rajii cells.

EC50 (nM)

| Construct¹ | n | Range | Mean | SD |
|---|---|---|---|---|
| IgG1 Antibody, | 10 | 0.00043-0.17 | 0.033 | 0.052 |
| Fucosylated | | | | |
| S3I-AA-OBI | 10 | 0.0000021-0.014 | 0.0029 | 0.0046 |
| Construct 7 | | | | |
| (anti-CD20) | | | | |
| S3W-AA-OBI | 2 | 0.000036-0.016 | 0.0078 | 0.011 |
| Construct 8² | | | | |
| (anti-CD20) | | | | |
| S3A-AA-OBI | 2 | 0.00018-0.011 | 0.0057 | 0.0079 |
| Construct 9 | | | | |
| (anti-CD20) | | | | |
| S3Y-AA-OBI | 4 | 0.00036-0.22 | 0.010 | 0.011 |
| Construct 14 | | | | |
| (anti-CD20), | | | | |
| G²⁰ linker | | | | |
| S5X-AA-OBI | 2 | 0.0000041-0.0045 | 0.0022 | 0.0032 |
| Construct 19 | | | | |
| (anti-CD20) | | | | |

¹All constructs included G20 linkers unless otherwise noted.
²Construct contains a spontaneous E388D mutation.

### Example 62. Antibody-Dependent Cell-Mediated Cytotoxicity (ADCC) Activation by Anti-PD-L1 Fc Constructs

ADCC Reporter Assay

An ADCC reporter assay was developed to test the degree to which anti-PD-L1 Fc constructs induce FcγRIIIa signaling and enhance ADCC activity relative to an anti-PD-L1 monoclonal antibody, avelumab (Bavencio). Anti-PD-L1 Fc constructs 4, 7, 8, 10, 13, 16, and 19 having the CDRs of avelumab were produced as described in Examples 1, 2, and 51-56. Four versions of construct 13 (PD-L1) in which the glycine spacer between the long chain Fc monomers varied in size (G₄, G₁₀, G₁₅ and G₂₀ linkers) were tested. Each anti-PD-L1 Fc construct, and fucosylated avelumab monoclonal antibodies, were tested in an ADCC reporter assay performed as follows:

Target HEK-PD-L1 cells (1.25×10⁴ cells/well) and effector Jurkat/FcγRIIIa cells (Promega) (7.45×10⁴ cells/well) were resuspended in RPMI 1640 Medium supplemented with 4% low IgG serum (Promega) and seeded in a 96-well plate with serially diluted anti-PD-L1 constructs. After incubation for 6 hours at 37° C. in 5% CO2, the luminescence was measured using the Bio-Glo Luciferase Assay Reagent (Promega) according to the manufacturer's protocol using a PHERAstar FS luminometer (BMG LABTECH).

As depicted in Table 23, anti-PD-L1 Fc constructs induced FcγRIIIa signaling in an ADCC reporter assay.

TABLE 23
Potency of anti-PD-L1 Fc constructs to induce
FcγRIIIa signaling in an ADCC reporter assay

| Construct | | EC50 (nM) | | |
|---|---|---|---|---|
| Number¹ | n | Range | Mean | SD |
| IgG1 Antibody, | 5 | 0.037-0.056 | 0.049 | 0.008 |
| Fucosylated | | | | |
| S3I-AA-AVE | 6 | 0.023-0.05 | 0.039 | 0.012 |
| Construct 7² | | | | |
| (anti-PD-L1) | | | | |
| S5I-AA-AVE | 1 | 0.025 | 0.025 | N/A |
| Construct 10 | | | | |
| (anti-PD-L1) | | | | |
| S3W-AA-AVE | 1 | 0.034 | 0.034 | N/A |
| Construct 8² | | | | |
| (anti-PD-L1) | | | | |
| S3Y-AA-AVE4 | 1 | 0.041 | 0.041 | N/A |
| Construct 14 | | | | |
| (anti-PD-L1), | | | | |
| G₄ linker | | | | |
| S3Y-AA-AVE10 | 1 | 0.062 | 0.062 | N/A |
| Construct 14 | | | | |
| (anti-PD-L1), | | | | |
| G¹⁰ linker | | | | |
| S3Y-AA-AVE15 | 1 | 0.044 | 0.044 | N/A |
| Construct 14 | | | | |
| (anti-PD-L1), | | | | |
| G₁₅ linker | | | | |
| S3Y-AA-AVE | 6 | 0.025-0.044 | 0.032 | 0.008 |
| Construct 14 | | | | |
| (anti-PD-L1), | | | | |
| G₂₀ linker | | | | |
| S5X-AA-AVE | 1 | 0.027 | 0.027 | N/A |
| Construct 19 | | | | |
| (anti-PD-L1) | | | | |
| S5Y-AA-AVE | 1 | 0.032 | 0.032 | N/A |
| Construct 16 | | | | |
| (anti-PD-L1) | | | | |

¹All constructs included G20 linkers unless otherwise noted.
²Construct contains a spontaneous E388D mutation.

ADCC Secondary Assay

Anti-PD-L1 Fc constructs 8, 9, 13 (G20 linker), and 19 were tested in an additional ADCC assay to confirm the ADCC reporter assay results. Each anti-PD-L1 Fc construct, and fucosylated and afucosylated avelumab monoclonal antibody, were tested in an ADCC assay performed as follows:

The ADCC A549-KILR assay was performed according to the manufacturer's directions (DiscoverX). The A549-KILR cell line was grown in tissue culture flasks using the AssayComplete™ Cell Culture Kit-105. The cells were harvested using AssayComplete™ Cell Detachment Reagent, adjusted to 2×10⁵ cells/mL with AssayComplete™ Cell Plating 39 Reagent and dispensed at 50 μL/well (1×10⁴ cells) into 96-well white bottom tissue culture treated plates. Anti-PD-L1 constructs were diluted to 11 nM in AssayComplete™ Cell Plating 39 Reagent immediately before serial dilutions (1:4) were performed. The diluted constructs were added to the wells at 10 μL/well and the assay plate was incubated at 37° C. with 5% CO₂ for 30 minutes. Frozen NK cells (Hemacare) were thawed and resuspended at 1×10⁸ cells/mL using AssayComplete™ Cell Plating 39 Reagent. Following the 30-minute incubation, the NK cells were added at 50 UL/well (5×10⁴ cells/well) to the assay plate. A positive control using afucosylated anti-PD-L1 IgG1 antibody and a negative control consisting of NK cells co-cultured with A549-KILR cells in the absence of antibody were also included. The assay plate was then incubated at 37° C. with 5% CO₂ for 3 hours. Immediately following the incubation, 100 μL/well of the KILR Detection Working Solution (comprised of KILR Detection Reagents 1, 2, and 3 mixed at a volume ratio of 4:1:1) was added to each well. The assay plate was subsequently incubated at RT for 30 minutes before the level of luminescence was determined using a PHERAstar FS luminometer (BMG LABTECH).

The results depicted in Table 24 demonstrate that anti-PD-L1 Fc constructs induced FcγRIIIa signaling in the secondary ADCC assay. The results from the secondary ADCC assay were consistent with the results of the ADCC reporter assay.

TABLE 24
Potency of anti-PD-L1 Fc constructs
to induce ADCC in KILR-A549 cells

| Construct | | EC50 (nM) | | |
|---|---|---|---|---|
| Number¹ | n | Range | Mean | SD |
| IgG1 Antibody, | 1 | 0.017 | 0.017 | N/A |
| Fucosylated | | | | |
| IgG1 Antibody, | 8 | 0.00016-0.011 | 0.0054 | 0.0041 |
| Afucosylated | | | | |
| S3W-AA-AVE | 1 | 0.0018 | 0.0018 | N/A |
| Construct 8² | | | | |
| (anti-PD-L1) | | | | |
| S3A-AA-AVE | 1 | 0.00074 | 0.00074 | N/A |
| Construct 9² | | | | |
| (anti-PD-L1) | | | | |
| S3Y-AA-AVE | 3 | 0.0042-0.011 | 0.0068 | 0.0035 |
| Construct 13 | | | | |
| (anti-PD-L1) | | | | |
| S5X-AA-AVE | 2 | 0.000070-0.0012 | 0.00065 | 0.00082 |
| Construct 19 | | | | |
| (anti-PD-L1) | | | | |

¹All constructs included G20 linkers unless otherwise noted.
²Construct contains a spontaneous E388D mutation.

### Example 63. Depletion of CD19+ B Cells in Human Blood by Anti-CD20 Fc Constructs

A whole blood peripheral blood mononuclear cell (PMBC) depletion assay was used to determine if an anti-CD20 Fc construct containing the CDRs from obinutuzumab (Gazyva) in fucosylated and afucosylated versions of anti-CD20 construct 13 could be used to deplete CD19+ B cells. CD19 is a B cell specific surface antigen, and leukemias and lymphomas derived from B cells express CD19. Human whole blood was collected in EDTA tubes and 100 μL incubated with serial dilutions, ranging from 2.96 μM to 0.3 μM, of VivoTag 645-labeled anti-CD20 Fc constructs for 10 minutes at 37° C. An immune cell-specific antibody cocktail, containing fluorescein isothiocyanate (FITC) anti-IgD, phycoerythrin (PE) anti-CD11c, allophycocyanin-H7 (APC/H7) anti-CD14, PerCp Cy5.5 anti-CD19, phycoerythrin-Cyanine7 (PE/Cy7) anti-CD56 and Pacific Blue (Pac Blue) anti-CD3 antibodies (BioLegend) was added to blood cells and stained for an additional 30 minutes at 37° C. After staining, red blood cells (RBCs) were lysed twice with ammonium chloride solution (STEMCELL technologies) for 10 minutes at room temperature (RT) and the cells were washed in PBS. Cells were resuspended in 200 μL FACS buffer (PBS containing 2% FBS) and samples were acquired in a FACSVerse™ instrument (BD Biosciences). A leukocyte gate was set using forward scatter (FSC) and side scatter (SSC) and B cells were characterized as SSClow, FSCint, CD11c-, CD14-, CD56-, CD3-, and CD19+, IgD+.

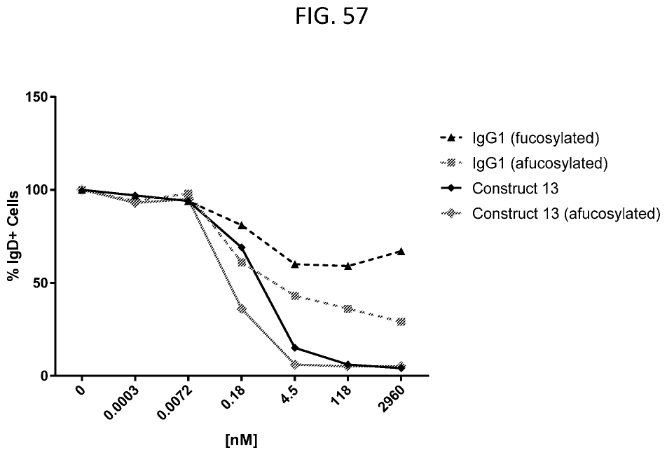

FIG. 57 shows that both fucosylated and afucosylated construct 13 (anti-CD20) depleted CD19+ B cells (% IgD+ cells) at lower concentrations (nM) than the fucosylated and afucosylated anti-CD20 IgG1 monodonal antibody (obinutuzumab). The afucosylated anti-CD20 construct 13 depleted CD19+ B cells more effectively than the fucosylated anti-CD20 Construct 13. Similarly, the afucosylated anti-CD20 monodonal antibody more effectively depleted CD19+ B cells than the fucosylated antibody.

### Example 64. Tumor Growth Reduction in a Mouse Lymphoma Model by an Anti-CD20 Fc Construct

A mouse disseminated tumor model for human lymphoma was used to test the effects of a single dose or multiple doses of an anti-CD20 Fc construct (anti-CD20 Construct 13) on disease progression and therapeutic response by bioluminescence imaging. CB17-severe combined immunodeficiency (SCID) mice (female, 6-7 weeks old with an average weight of 20 grams, strain 236 from Charles River Laboratories) were housed in animal care facility for 48 hours prior to use according to IACUC protocol. Water and food were provided ad libitum. All experiments were approved by an institutional animal ethics committee. Mice were checked daily for signs of discomfort and for general appearance. For tumor xenograft model, 5×106 luciferase-expressing human Burkitt's lymphoma Daudi cells (Daudi-Luc) were injected intravenously through the tail vein. Prior to injection, Daudi-luc cells showed cell surface expression of CD20 and CD38. Mice were staged 1 hour after tumor cell implantation for randomization into various treatment groups (7 mice/group) based on the bioluminescence signal (concentrated mainly in the lungs at such an early time point). Five hours after tumor cell injection, each group of mice was treated intraperitoneally with a single dose of an agent (daratumumab (an anti-CD38 monodonal antibody) at 18 mg/kg; obinutuzumab (Gazyva, an anti-CD20 IgG1 monoclonal antibody) at 18 mg/kg; anti-CD20 construct 13 at 30 mg/kg, molar equivalent of IgG1 dose) similar to clinical dose ranges. Saline was administered to a group of mice as a negative control. In a separate arm, anti-CD20 construct 13 was administered at 30 mg/kg every 2 days for a total of 6 doses (i.e., mice were treated with anti-CD20 construct 13 at 1, 3, 5, 7, 9, and 11 days following tumor cell injection). Temporal changes in the tumor growth was determined by bioluminescense imaging on IVIS spectrum (PerkinElmer). For imaging, mice were anesthetized followed by D-luciferin injection (150 μL of 15 mg/mL stock solution/mouse). Mice were then placed in a light tight box for imaging with a charge-coupled device detector. Photons emitted from the luciferase-expressing cells in mice were counted over an exposure period. Raw data of radiance as total flux (photons/second) was calculated and quantitative image analysis was performed using Living Image Software (PerkinElmer). Under illumination, black-and-white images were acquired for anatomical reference for drawing region of interest. Light emission collected from the dorsal and ventral surfaces was integrated over the total body areas of individual mice and plotted in time as a measure of tumor mass development. The integrated bioluminescense light intensity representing tumor load was measured and data are presented as mean±SEM (7 mice/group).

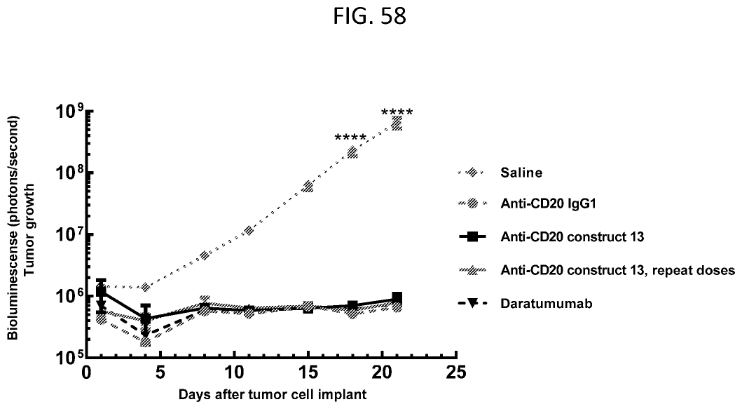

FIG. 58 demonstrates that anti-CD20 construct 13 greatly restricts tumor development in a disseminated lymphoma model. A single dose of anti-CD20 construct 13 was as effective as daratumumab and obinutuzumab (Gazyva) in reducing tumor growth over the course of 21 days following tumor cell transplantation. Points labeled with **** in the saline group had p values of <0.0001 relative to corresponding treatment groups. Multiple doses of anti-CD20 construct 13 were about as effective as a single dose of the construct in this model assay. Mice tolerated multiple doses of the construct 13 well and displayed no visible side effects (data not shown).

### Other Embodiments

All publications, patents, and patent applications mentioned in this specification are incorporated herein by reference to the same extent as if each independent publication or patent application was specifically and individually indicated to be incorporated by reference.

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims.

Other embodiments are within the claims.

What is claimed is:

1. An Fc-antigen binding domain construct comprising enhanced effector function, wherein the Fc-antigen binding domain construct comprises an antigen binding domain and a first Fc domain joined to a second Fc domain by a linker, wherein the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain.

2. A polypeptide comprising an antigen binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a C_{H}3 domain,
wherein at least one Fc domain monomer comprises mutations forming an engineered protuberance.

3. The polypeptide of claim 2 wherein the antigen binding domain comprises an antibody heavy chain variable domain.

4. The polypeptide of claim 2 wherein the antigen binding domain comprises an antibody light chain variable domain.

5. The polypeptide of claim 2 wherein the first IgG1 Fc domain monomer comprises two or four reverse charge mutations and the second IgG1 Fc domain monomer comprises mutations forming an engineered protuberance.

6. The polypeptide of claim 2 wherein the first IgG1 Fc domain monomer comprises mutations forming an engineered protuberance and the second IgG1 Fc domain monomer comprises two or four reverse charge mutations.

7. The polypeptide of claim 2 wherein both the first IgG1 Fc domain monomer and the second IgG constant domain monomer comprise mutations forming an engineered protuberance.

8. The polypeptide of claim 2 comprising a third linker and a third IgG1 Fc domain monomer wherein the first IgG1 Fc domain monomer, the second IgG1 Fc domain monomer and the third IgG1 Fc domain monomer each comprise mutations forming an engineered protuberance.

9. The polypeptide of claim 2 comprising a third linker and a third IgG1 Fc domain monomer wherein both the first IgG1 Fc domain monomer and the second IgG1 Fc domain monomer each comprise mutations forming an engineered protuberance and the third IgG1 Fc domain monomer comprises two or four reverse charge mutations.

10. The polypeptide of claim 2 comprising a third linker and third IgG1 Fc domain monomer wherein both the first IgG1 Fc domain monomer and the third IgG1 Fc domain monomer each comprise mutations forming an engineered protuberance and the second IgG domain monomer comprises two or four reverse charge mutations.

11. The polypeptide of claim 2 comprising a third linker and a third IgG1 Fc domain monomer wherein both the second IgG1 Fc domain monomer and the third IgG1 Fc domain monomer each comprise mutations forming an engineered protuberance and the first IgG domain monomer comprises two or four reverse charge mutations.

12. The polypeptide of claim 2, wherein the IgG1 Fc domain monomers comprising mutations forming an engineered protuberance further comprise one, two or three reverse charge mutations.

13.-29. (canceled)

30. The polypeptide of claim 2, wherein the CH2 domains of each Fc domain monomer independently comprise the amino acid sequence: GGPSVFLFPPKPKDTLMISRTPEVTCVVVDVSHEDPEVKFNWYVDGVEVHNAKTKPREE QYNSTYRVVSVLTVLHQDWLNGKEYKCKVSNKALPAPIEKTISKAK with no more than two single amino acid deletions or substitutions.

31.-33. (canceled)

34. The polypeptide of claim 2, wherein the CH3 domains of each Fc domain monomer independently comprise the amino acid sequence: GQPREPQVYTLPPSRDELTKNQVSLTCLVKGFYPSDIAVEWESNGQPENNYKTTPPVLD SDGSFFLYSK LTVDKSRWQQGNVFSCSVMHEALHNHYTQKSLSLSPG with no more than 10 single amino acid substitutions.

35.-44. (canceled)

45. The polypeptide of claim 2, wherein the antigen binding domain comprises a VH domain and a CH1 domain.

46. The polypeptide of claim 45, wherein the antigen binding domain further comprises a VL domain.

47. The polypeptide of claim 45, wherein the VH domain comprises a set of CDR-H1, CDR-H2 and CDR-H3 sequences set forth in Table 1.

48. The polypeptide of claim 45, wherein the VH domain comprises CDR-H1, CDR-H2, and CDR-H3 of a VH domain comprising a sequence of an antibody set forth in Table 2.

49.-61. (canceled)

62. A polypeptide comprising: an antigen binding domain; a linker; a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain,
wherein at least one Fc domain monomer comprises one, two or three reverse charge amino acid mutations.

63.-64. (canceled)

65. The polypeptide of claim 62 wherein the first IgG1 Fc domain monomer comprises a set of two reverse charge mutations selected from those in Table 5 or a set of four reverse charge mutation selected from those in Table 6 and the second IgG1 Fc domain monomer comprises one, two or three reverse charge amino acid mutations selected from Table 4.

66.-133. (canceled)

134. A polypeptide comprising: a first IgG1 Fc domain monomer comprising a hinge domain, a CH2 domain and a CH3 domain; a second linker; a second IgG1 Fc domain monomer comprising a hinge domain, a CH2 domain and a CH3 domain; an optional third linker; and an optional third IgG1 Fc domain monomer comprising a hinge domain, a CH2 domain and a CH3 domain,
wherein at least one Fc domain monomer comprises one, two or three reverse charge amino acid mutations.

135.-206. (canceled)

207. An Fc-antigen binding domain construct comprising:
a) a first polypeptide comprising
i) a first Fc domain monomer,
ii) a second Fc domain monomer, and
iii) a linker joining the first Fc domain monomer and the second Fc domain monomer;
b) a second polypeptide comprising a third Fc domain monomer;
c) a third polypeptide comprising a fourth Fc domain monomer; and
d) an antigen binding domain joined to the first polypeptide, second polypeptide, or third polypeptide;
wherein the first Fc domain monomer and the third Fc domain monomer combine to form a first Fc domain and the second Fc domain monomer and the fourth Fc domain monomer combine to form a second Fc domain, and wherein the Fc-antigen binding domain construct comprises a biological activity that is not exhibited by a construct having a single Fc domain and the antigen binding domain.

208.-209. (canceled)

210. An Fc-antigen binding domain construct comprising:
a) a first polypeptide comprising
i) a first Fc domain monomer,
ii) a second Fc domain monomer, and
iii) a spacer joining the first Fc domain monomer and the second Fc domain monomer;
b) a second polypeptide comprising a third Fc domain monomer;
c) a third polypeptide comprising a fourth Fc domain monomer; and
d) an antigen binding domain joined to the first polypeptide, second polypeptide, or third polypeptide;
wherein the first Fc domain monomer and the third Fc domain monomer combine to form a first Fc domain and the second Fc domain monomer and the fourth Fc domain monomer combine to form a second Fc domain.

211-287. (canceled)

288. An Fc-antigen binding domain construct comprising:
a) a first polypeptide comprising
i) a first Fc domain monomer,
ii) a second Fc domain monomer, and
iii) a linker joining the first Fc domain monomer and the second Fc domain monomer;
b) a second polypeptide comprising a third Fc domain monomer;
c) a third polypeptide comprising a fourth Fc domain monomer; and
d) a first antigen binding domain joined to the first polypeptide; and
e) a second antigen binding domain joined to the second polypeptide and/or third polypeptide;
wherein the first Fc domain monomer and the third Fc domain monomer combine to form a first Fc domain and the second Fc domain monomer and the fourth Fc domain monomer combine to form a second Fc domain, wherein the first and the second antigen binding domains bind different antigens, and wherein the Fc-antigen binding domain construct has enhanced effector function in an antibody-dependent cytotoxicity (ADCC) assay, an antibody-dependent cellular phagocytosis (ADCP) and/or complement-dependent cytotoxicity (CDC) assay relative to a construct having a single Fc domain and the antigen binding domain.

289. An Fc-antigen binding domain construct comprising:
a) a first polypeptide comprising
i) a first Fc domain monomer,
ii) a second Fc domain monomer, and
iii) a linker joining the first Fc domain monomer and the second Fc domain monomer;
b) a second polypeptide comprising a third Fc domain monomer;
c) a third polypeptide comprising a fourth Fc domain monomer;
d) a first antigen binding domain joined to the first polypeptide; and
e) a second antigen binding domain joined to the second polypeptide and/or third polypeptide;
wherein the first Fc domain monomer and the third Fc domain monomer combine to form a first Fc domain and the second Fc domain monomer and the fourth Fc domain monomer combine to form a second Fc domain, and wherein the first and the second antigen binding domains bind different antigens, and wherein the Fc-antigen binding domain construct comprises a biological activity that is not exhibited by a construct having a single Fc domain and the antigen binding domain.

290. An Fc-antigen binding domain construct comprising:
a) a first polypeptide comprising
i) a first Fc domain monomer,
ii) a second Fc domain monomer, and
iii) a spacer joining the first Fc domain monomer and the second Fc domain monomer;
b) a second polypeptide comprising a third Fc domain monomer;
c) a third polypeptide comprising a fourth Fc domain monomer; and
d) a first antigen binding domain joined to the first polypeptide; and
e) a second antigen binding domain joined to the second polypeptide and/or third polypeptide;
wherein the first Fc domain monomer and the third Fc domain monomer combine to form a first Fc domain and the second Fc domain monomer and the fourth Fc domain monomer combine to form a second Fc domain, and wherein the first and the second antigen binding domains bind different antigens.

291-317. (canceled)


SEQUENCE LISTING (80 sequences)

SEQ ID NO: 1 - protein, 5 aa, Artificial Sequence
  Linker sequence
GGGGS

SEQ ID NO: 2 - protein, 4 aa, Artificial Sequence
  Linker sequence
GGSG

SEQ ID NO: 3 - protein, 4 aa, Artificial Sequence
  Linker sequence
SGGG

SEQ ID NO: 4 - protein, 4 aa, Artificial Sequence
  Linker sequence
GSGS

SEQ ID NO: 5 - protein, 6 aa, Artificial Sequence
  Linker sequence
GSGSGS

SEQ ID NO: 6 - protein, 8 aa, Artificial Sequence
  Linker sequence
GSGSGSGS

SEQ ID NO: 7 - protein, 10 aa, Artificial Sequence
  Linker sequence
GSGSGSGSGS

SEQ ID NO: 8 - protein, 12 aa, Artificial Sequence
  Linker sequence
GSGSGSGSGSGS

SEQ ID NO: 9 - protein, 6 aa, Artificial Sequence
  Linker sequence
GGSGGS

SEQ ID NO: 10 - protein, 9 aa, Artificial Sequence
  Linker sequence
GGSGGSGGS

SEQ ID NO: 11 - protein, 12 aa, Artificial Sequence
  Linker sequence
GGSGGSGGSGGS

SEQ ID NO: 12 - protein, 8 aa, Artificial Sequence
  Linker sequence
GGSGGGSG

SEQ ID NO: 13 - protein, 12 aa, Artificial Sequence
  Linker sequence
GGSGGGSGGGSG

SEQ ID NO: 14 - protein, 16 aa, Artificial Sequence
  Linker sequence
GGSGGGSGGGSGGGSG

SEQ ID NO: 15 - protein, 20 aa, Artificial Sequence
  Linker sequence
GGSGGGSGGGSGGGSGGGSG

SEQ ID NO: 16 - protein, 10 aa, Artificial Sequence
  Linker sequence
GGGGSGGGGS

SEQ ID NO: 17 - protein, 15 aa, Artificial Sequence
  Linker sequence
GGGGSGGGGSGGGGS

SEQ ID NO: 18 - protein, 20 aa, Artificial Sequence
  Linker sequence
SGGGSGGGSGGGSGGGSGGG

SEQ ID NO: 19 - protein, 4 aa, Artificial Sequence
  Linker sequence
GGGG

SEQ ID NO: 20 - protein, 8 aa, Artificial Sequence
  Linker sequence
GGGGGGGG

SEQ ID NO: 21 - protein, 12 aa, Artificial Sequence
  Linker sequence
GGGGGGGGGGGG

SEQ ID NO: 22 - protein, 16 aa, Artificial Sequence
  Linker sequence
GGGGGGGGGGGGGGGG

SEQ ID NO: 23 - protein, 20 aa, Artificial Sequence
  Linker sequence
GGGGGGGGGGGGGGGGGGGG

SEQ ID NO: 24 - protein, 5 aa, Artificial Sequence
  Linker sequence
GGGGG

SEQ ID NO: 25 - protein, 10 aa, Artificial Sequence
  Linker sequence
GGGGGGGGGG

SEQ ID NO: 26 - protein, 15 aa, Artificial Sequence
  Linker sequence
GGGGGGGGGGGGGGG

SEQ ID NO: 27 - protein, 20 aa, Artificial Sequence
  Linker sequence
GGGGGGGGGGGGGGGGGGGG

SEQ ID NO: 28 - protein, 10 aa, Artificial Sequence
  Linker sequence
GENLYFQSGG

SEQ ID NO: 29 - protein, 8 aa, Artificial Sequence
  Linker sequence
SACYCELS

SEQ ID NO: 30 - protein, 5 aa, Artificial Sequence
  Linker sequence
RSIAT

SEQ ID NO: 31 - protein, 17 aa, Artificial Sequence
  Linker sequence
RPACKIPNDLKQKVMNH

SEQ ID NO: 32 - protein, 36 aa, Artificial Sequence
  Linker sequence
GGSAGGSGSGSSGGSSGASGTGTAGGTGSGSGTGSG

SEQ ID NO: 33 - protein, 17 aa, Artificial Sequence
  Linker sequence
AAANSSIDLISVPVDSR

SEQ ID NO: 34 - protein, 36 aa, Artificial Sequence
  Linker sequence
GGSGGGSEGGGSEGGGSEGGGSEGGGSEGGGSGGGS

SEQ ID NO: 35 - protein, 12 aa, Artificial Sequence
  Linker sequence
GGGSGGGSGGGS

SEQ ID NO: 36 - protein, 18 aa, Artificial Sequence
  Linker sequence
GGSGGGSGGGSGGGSGGS

SEQ ID NO: 37 - protein, 11 aa, Artificial Sequence
  Albumin binding peptide
DICLPRWGCLW

SEQ ID NO: 38 - protein, 6 aa, Artificial Sequence
  Hexa-histidine peptide
HHHHHH

SEQ ID NO: 39 - protein, 8 aa, Artificial Sequence
  FLAG peptide
DYKDDDDK

SEQ ID NO: 40 - protein, 10 aa, Artificial Sequence
  Myc peptide
EQKLISEEDL

SEQ ID NO: 41 - protein, 9 aa, Artificial Sequence
  HA peptide
YPYDVPDYA

SEQ ID NO: 42 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 474 aa, Artificial Sequence
  Long chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 44 - protein, 227 aa, Artificial Sequence
  Fc domain monomer, short chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 473 aa, Artificial Sequence
  Long chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 46 - protein, 226 aa, Artificial Sequence
  Fc domain monomer, short chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 47 - protein, 468 aa, Artificial Sequence
  Long chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 48 - protein, 226 aa, Artificial Sequence
  Fc domain monomer, short chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 49 - protein, 473 aa, Artificial Sequence
  Long polypeptide, construct 4/AA
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 50 - protein, 227 aa, Artificial Sequence
  Fc domain monomer, short chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 227 aa, Artificial Sequence
  Fc domain monomer, short chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 52 - protein, 225 aa, Artificial Sequence
  Fc domain monomer, short chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSP

SEQ ID NO: 53 - protein, 227 aa, Artificial Sequence
  Fc domain monomer, short chain polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 474 aa, Artificial Sequence
  Fc construct long polypeptide with N-terminal Asp to Gln
QKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 55 - protein, 227 aa, Artificial Sequence
  Fc construct short polypeptide with N-terminal Asp to Gln
QKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - protein, 473 aa, Artificial Sequence
  Fc construct long polypeptide with N-terminal Asp to Gln
QKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 57 - protein, 226 aa, Artificial Sequence
  Fc construct short polypeptide with N-terminal Asp to Gln
QKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 58 - protein, 468 aa, Artificial Sequence
  Fc construct long polypeptide with N-terminal Asp to Gln
QKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 59 - protein, 226 aa, Artificial Sequence
  Fc construct short polypeptide with N-terminal Asp to Gln
QKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 60 - protein, 473 aa, Artificial Sequence
  Fc construct long polypeptide with N-terminal Asp to Gln
QKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 61 - protein, 226 aa, Artificial Sequence; identical to SEQ ID NO: 48
  Short polypeptide

SEQ ID NO: 62 - protein, 473 aa, Artificial Sequence
  Long polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 63 - protein, 226 aa, Artificial Sequence
  Short polypeptide construct 6/A1
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 64 - protein, 473 aa, Artificial Sequence
  Long peptide construct 6/A1
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 65 - protein, 473 aa, Artificial Sequence
  Long polypeptide
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 66 - protein, 473 aa, Artificial Sequence
  Long polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 67 - protein, 473 aa, Artificial Sequence
  Long polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 68 - protein, 473 aa, Artificial Sequence
  Long protein
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 69 - protein, 473 aa, Artificial Sequence
  Long polypeptide
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 70 - protein, 226 aa, Artificial Sequence
  Short polypeptide construct 16/CC
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 71 - protein, 473 aa, Artificial Sequence
  Long protein
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 72 - protein, 473 aa, Artificial Sequence
  Long protein
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 73 - protein, 226 aa, Artificial Sequence
  Short polypeptide construct 18/Q1
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 74 - protein, 473 aa, Artificial Sequence
  Long polypeptide
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 75 - protein, 473 aa, Artificial Sequence
  Long polypeptide
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 76 - protein, 473 aa, Artificial Sequence
  Long polypeptide
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 77 - protein, 473 aa, Artificial Sequence
  Long polypeptide
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 78 - protein, 473 aa, Artificial Sequence
  Long polypeptide Construct 18/Q1
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 79 - protein, 473 aa, Artificial Sequence
  Long polypeptide construct 19/Q2
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 80 - protein, 32 aa, Artificial Sequence
  Linker sequence
GGSGGGSGGGSGGGGGSGGGGSGGGGSGGGGS